(19) United States
(12) Patent Application Publication
(10) Pub. No.: US 2025/0170100 A1
(43) Pub. Date: May 29, 2025

(54) MONOACYLGLYCEROL LIPASE INHIBITORS AND USE THEREOF FOR THE TREATMENT OF ANXIETY AND RELATED CONDITIONS

(71) Applicant: Psy Therapeutics, Inc., Boston, MA (US)

(72) Inventors: Jacob Matthew Hooker, Winchester, MA (US); Sachin Patel, Nashville, TN (US); Alan John Cross, Thornton, PA (US)

(73) Assignee: Psy Therapeutics, Inc., Boston, MA (US)

(21) Appl. No.: 18/724,766

(22) PCT Filed: Dec. 29, 2022
(86) PCT No.: PCT/US2022/082585

§ 371 Date: Jun. 27, 2024

Related U.S. Application Data
(60) Provisional application No. 63/294,752, filed on Dec. 29, 2021.

(51) Int. Cl. A61K 31/4155; A61K 31/397; A61K 31/4178; A61K 31/4196; A61K 31/4245

(52) U.S. Cl. CPC A61K 31/4155; A61K 31/397; A61K 31/4178; A61K 31/4196; A61K 31/4245

(57) ABSTRACT

Provided herein are methods of using reversible monoacylglycerol lipase (MAGL) inhibitors for the treatment of anxiety and related conditions, including Generalized anxiety disorder, panic disorder, social anxiety disorder, obsessive-compulsive disorder, post-traumatic stress disorder, separation anxiety disorder, anxious depression, and phobias, the methods comprising administering to the subject a therapeutically effective amount of a compound.

### CROSS-REFERENCE TO RELATED APPLICATIONS

This patent application claims the benefit under 35 U.S.C. § 119 (e) of U.S. Provisional Patent Application No. 63/294,752 filed Dec. 29, 2021, which is incorporated herein by reference in its entirety.

### TECHNICAL FIELD

The present disclosure relates to compounds and methods for inhibiting monoacylglycerol lipase (MAGL), useful for the treatment and management of anxiety and related conditions.

### BACKGROUND

MAGL is the principal enzyme responsible for the in vivo degradation of 2-arachidonoyl glycerol (2-AG), an endogenous ligand of the cannabinoid receptors (e.g., CB1 and CB2). MAGL inhibition increases accumulation of the CB1/2 receptor agonist 2-arachidonoyl glycerol (2-AG), and reduces arachidonic acid (AA) and prostaglandin levels in the brain and peripheral tissues. Irreversible MAGL inhibitor compounds, such as JZL-184, increase brain and peripheral 2-AG and reduce brain AA, however tolerance can develop with chronic irreversible MAGL inhibition. Covalent interactions with MAGL could lead to irreversible enzymatic inhibition, with potential for immune-mediated toxicity.

There remains a need for reversible MAGL inhibitors useful for the treatment of MAGL-mediated diseases or disorders, including the development of therapeutic compounds with improved control of dose and exposure. Such compounds can be developed through clinical trials as compounds for the treatment of anxiety, post-traumatic stress disorder (PTSD) and/or treatment of various MAGL-mediated conditions.

Thus, there is a need in the art for compounds that can potently, selectively and reversibly inhibit MAGL and for means for treating these conditions or disorders that are associated with or linked to endocannabinoid signaling activities. The present disclosure addresses these and other unfulfilled needs in the art.

### SUMMARY

In some embodiments, a reversible MAGL inhibitor is used to increase 2-AG within the central nervous system. The invention is based in part on the discovery that a reversible MAGL inhibitor can transiently increase 2-AG in the brain of certain animal models. In some embodiments, methods of transiently increasing 2-AG in the brain comprise the administration of a reversible MAGL inhibitor compound where the pharmacodynamic half-life (e.g., as measured by the transient increase in 2-AG in the brain) is within less than twice the pharmacokinetic half-life of the compound (e.g., as measured by the half-life of the compound in the plasma). In some embodiments, a method of treatment comprises oral administration of a reversible MAGL inhibitor to a subject in a therapeutically effective amount resulting in the transient increase of 2-AG in the brain of the subject that is characterized by a ratio of less than 2 between the half-life of transient 2-AG increase in the brain and the plasma half-life of the compound in the blood plasma. In contrast, the Applicants have not observed the coupling of the pharmacodynamic response of a covalent small molecule MAGL inhibitor (such as JZL 184) to its pharmacokinetic properties.

The endogenous cannabinoid 2-arachidonoylglycerol (2-AG) is a lipid-signaling molecule in the central nervous system (CNS) that acts as an agonist of the CB1 receptor and the primary endogenous ligand for the CB2 receptor. CB1 is the primary target of 49-tetrahydrocannabinol, and anxiety reduction and stress relief have been reported as central motives for chronic cannabis use. In addition, 2-AG levels have been shown to increase in the amygdala in response to repeated stress exposure, suggesting a physiologic role for this system in guarding against the development of stress-induced pathology. Furthermore, studies examining genetic and pharmacological inhibition of 2-AG synthesis demonstrate increased anxiety-like behavior in rodent models, whereas pharmacological augmentation of 2AG levels have been shown to decrease anxiety-like behavior. 2-AG is primarily hydrolyzed by the enzyme monoacylglycerol lipase (MAGL) and inhibition of MAGL has been shown to enhance 2-AG signaling in the brain. Numerous studies have recently demonstrated that MAGL inhibition decreases anxiety-like and depressive-like behaviors under both basal and high environmentally aversive conditions.

Compounds for the transient inhibition of 2-AG in the central nervous system are useful in the treatment of MAGL-mediated diseases, including the treatment of anxiety disorders. In some embodiments, Selective MAGL Inhibitor compounds disclosed herein can be used for the transient inhibition of 2-AG in the central nervous system are useful in the treatment of MAGL-mediated diseases, including the treatment of anxiety disorders. In some embodiments, Reversible MAGL Inhibitor compounds disclosed herein can be used for the transient inhibition of 2-AG in the central nervous system and are useful in the treatment of MAGL-mediated diseases, including the treatment of anxiety disorders. In some embodiments, Selective and Reversible MAGL Inhibitor compounds disclosed herein can be used for the transient inhibition of 2-AG in the central nervous system and are useful in the treatment of MAGL-mediated diseases, including the treatment of anxiety disorders.

In some embodiments, a method of treating a MAGL-mediated disease in a subject in need thereof, the method comprises administering to the subject a therapeutically effective amount of a compound disclosed herein, such as a compound selected from the group consisting of:
(2,4-difluoro-5-hydroxyphenyl) {6-[3-methyl-1-(o-tolyl)-5-pyrazolyl]-2-aza-2-spiro[3.3]heptyl}methanone (Compound 126);
4-hydroxy-2-({6-[3-methyl-1-(o-tolyl)-5-pyrazolyl]-2-aza-2-spiro[3.3]heptyl}carbonyl)benzonitrile (Compound 128);
[4-fluoro-2-(2,2,2-trifluoroethoxy)phenyl]{6-[3-methyl-1-(o-tolyl)-5-pyrazolyl]-2-aza-2-spiro[3.3]heptyl}methanone (Compound 178);
(2-fluoro-5-hydroxyphenyl) {6-[1-(5-fluoro-2-tolyl)-3-methyl-5-pyrazolyl]-2-aza-2-spiro[3.3]heptyl}methanone (Compound 365);
(2-fluoro-5-hydroxyphenyl) (6-{3-methyl-1-[o-(trifluoromethyl)phenyl]-5-pyrazolyl}-2-aza-2-spiro[3.3]heptyl) methanone (Compound 366);
(2-fluoro-5-hydroxyphenyl) (6-{5-(trifluoromethyl)-3-[o-(trifluoromethyl)phenyl]-1-pyrazolyl}-2-aza-2-spiro[3.3]heptyl) methanone (Compound 414);
(2-cyclopropoxy-4-fluorophenyl) (6-{5-(trifluoromethyl)-3-[o-(trifluoromethyl)phenyl]-1-pyrazolyl}-2-aza-2-spiro[3.3]heptyl) methanone (Compound 451);
(2-fluoro-5-hydroxyphenyl) {6-[3-(o-fluorophenyl)-4-(trifluoromethyl)-1-pyrazolyl]-2-aza-2-spiro[3.3]heptyl}methanone (Compound 473);
{6-[5-(2-chloro-5-fluorophenyl)-4-methyl-1-pyrazolyl]-2-aza-2-spiro[3.3]heptyl}(2-fluoro-5-hydroxyphenyl) methanone (Compound 519);
{6-[3-(2-chloro-5-fluorophenyl)-4-methyl-1-pyrazolyl]-2-aza-2-spiro[3.3]heptyl}(2-fluoro-5-hydroxyphenyl) methanone (Compound 520); and
{6-[5-(2,5-difluorophenyl)-4-methyl-1-pyrazolyl]-2-aza-2-spiro[3.3]heptyl}(2-fluoro-5-hydroxyphenyl) methanone (Compound 476), or a pharmaceutically acceptable salt thereof.

In still another aspect, the disclosure provides a method for treating a monoglycerol lipase mediated disease or disorder. Generally, the method comprises administering to a subject in need thereof a therapeutically effective amount of a compound herein.

In some embodiments, methods of treating the anxiety disorder are provided. In some embodiments, the anxiety disorder is selected from the group consisting of: (a) Generalized anxiety disorder, (b) panic disorder, (c) social anxiety disorder, (d) obsessive-compulsive disorder (OCD), (e) post-traumatic stress disorder (PTSD), (f) separation anxiety disorder, (g) anxious depression, and (h) phobias. In some embodiments, a method of treating an anxiety disorder selected from the group consisting of: (a) Generalized anxiety disorder, (GAD) (b) a panic disorder, (c) a social anxiety disorder, (d) an obsessive-compulsive disorder (OCD), (e) post-traumatic stress disorder (PTSD), (f) separation anxiety disorder, (OCD), anxious depression, and (h) phobias, in a subject in need thereof, the method comprising administering to the subject a therapeutically effective amount of a compound selected from the group consisting of
(2,4-difluoro-5-hydroxyphenyl) {6-[3-methyl-1-(o-tolyl)-5-pyrazolyl]-2-aza-2-spiro[3.3]heptyl}methanone (Compound 126);
4-hydroxy-2-({6-[3-methyl-1-(o-tolyl)-5-pyrazolyl]-2-aza-2-spiro[3.3]heptyl}carbonyl)benzonitrile (Compound 128);
[4-fluoro-2-(2,2,2-trifluoroethoxy)phenyl]{6-[3-methyl-1-(o-tolyl)-5-pyrazolyl]-2-aza-2-spiro[3.3]heptyl}methanone (Compound 178);
(2-fluoro-5-hydroxyphenyl) {6-[1-(5-fluoro-2-tolyl)-3-methyl-5-pyrazolyl]-2-aza-2-spiro[3.3]heptyl}methanone (Compound 365);
(2-fluoro-5-hydroxyphenyl) (6-{3-methyl-1-[o-(trifluoromethyl)phenyl]-5-pyrazolyl}-2-aza-2-spiro[3.3]heptyl) methanone (Compound 366);
(2-fluoro-5-hydroxyphenyl) (6-{5-(trifluoromethyl)-3-[o-(trifluoromethyl)phenyl]-1-pyrazolyl}-2-aza-2-spiro[3.3]heptyl) methanone (Compound 414);
(2-cyclopropoxy-4-fluorophenyl) (6-{5-(trifluoromethyl)-3-[o-(trifluoromethyl)phenyl]-1-pyrazolyl}-2-aza-2-spiro[3.3]heptyl) methanone (Compound 451);
(2-fluoro-5-hydroxyphenyl) {6-[3-(o-fluorophenyl)-4-(trifluoromethyl)-1-pyrazolyl]-2-aza-2-spiro[3.3]heptyl}methanone (Compound 473);
{6-[S-(2-chloro-5-fluorophenyl)-4-methyl-1-pyrazolyl]-2-aza-2-spiro[3.3]heptyl}(2-fluoro-5-hydroxyphenyl) methanone (Compound 519);
{6-[3-(2-chloro-S-fluorophenyl)-4-methyl-1-pyrazolyl]-2-aza-2-spiro[3.3]heptyl}(2-fluoro-5-hydroxyphenyl) methanone (Compound 520); and
{6-[5-(2,5-difluorophenyl)-4-methyl-1-pyrazolyl]-2-aza-2-spiro[3.3]heptyl}(2-fluoro-5-hydroxyphenyl) methanone (Compound 476), or a pharmaceutically acceptable salt thereof.

In some embodiments, the anxiety disorder is Generalized anxiety disorder (GAD). In some embodiments, the anxiety disorder a panic disorder. In some embodiments, the anxiety disorder a social anxiety disorder. In some embodiments, the anxiety disorder is an obsessive-compulsive disorder (OCD). In some embodiments, the anxiety disorder is post-traumatic stress disorder (PTSD). In some embodiments, the anxiety disorder is separation anxiety disorder. In some embodiments, the anxiety disorder is a phobia.

In some embodiments, a method of reversibly inhibiting MAGL comprising contacting the MAGL with a compound selected from the group consisting of:
(2,4-difluoro-5-hydroxyphenyl) {6-[3-methyl-1-(o-tolyl)-5-pyrazolyl]-2-aza-2-spiro[3.3]heptyl}methanone (Compound 126);
4-hydroxy-2-({6-[3-methyl-1-(o-tolyl)-5-pyrazolyl]-2-aza-2-spiro[3.3]heptyl}carbonyl)benzonitrile (Compound 128);
[4-fluoro-2-(2,2,2-trifluoroethoxy)phenyl]{6-[3-methyl-1-(o-tolyl)-5-pyrazolyl]-2-aza-2-spiro[3.3]heptyl}methanone (Compound 178);
(2-fluoro-5-hydroxyphenyl) {6-[1-(5-fluoro-2-tolyl)-3-methyl-5-pyrazolyl]-2-aza-2-spiro[3.3]heptyl}methanone (Compound 365);
(2-fluoro-5-hydroxyphenyl) (6-{3-methyl-1-[o-(trifluoromethyl)phenyl]-5-pyrazolyl}-2-aza-2-spiro[3.3]heptyl) methanone (Compound 366),
(2-fluoro-5-hydroxyphenyl) (6-{5-(trifluoromethyl)-3-[o-(trifluoromethyl)phenyl]-1-pyrazolyl}-2-aza-2-spiro[3.3]heptyl) methanone (Compound 414);
(2-cyclopropoxy-4-fluorophenyl) (6-{5-(trifluoromethyl)-3-[o-(trifluoromethyl)phenyl]-1-pyrazolyl}-2-aza-2-spiro[3.3]heptyl) methanone (Compound 451);
(2-fluoro-5-hydroxyphenyl) {6-[3-(o-fluorophenyl)-4-(trifluoromethyl)-1-pyrazolyl]-2-aza-2-spiro[3.3]heptyl}methanone (Compound 473);
{6-[5-(2-chloro-S-fluorophenyl)-4-methyl-1-pyrazolyl]-2-aza-2-spiro[3.3]heptyl}(2-fluoro-5-hydroxyphenyl) methanone (Compound 519);
{6-[3-(2-chloro-5-fluorophenyl)-4-methyl-1-pyrazolyl]-2-aza-2-spiro[3.3]heptyl}(2-fluoro-5-hydroxyphenyl) methanone (Compound 520); and
{6-[5-(2,5-difluorophenyl)-4-methyl-1-pyrazolyl]-2-aza-2-spiro[3.3]heptyl}(2-fluoro-5-hydroxyphenyl) methanone (Compound 476), or a pharmaceutically acceptable salt thereof.

In some embodiments, the administration of the compound transiently increases the level of 2-AG in the brain of the subject (e.g., within about 30 minutes after the oral administration of the compound to the subject). In some embodiments, the administration of the compound transiently increases the level of 2-AG in the brain of the subject. In some embodiments, the half-life of the transient increase of the 2-AG in the brain of the subject is less than twice the half-life of the compound in the blood plasma of the subject.

### BRIEF DESCRIPTION OF THE FIGURES

FIG. 1A is a bar graph showing both the plasma and brain concentrations of a Reversible Selective MAGL Inhibitor in a murine model as disclosed in Example 18.

FIG. 1B is a bar graph showing 2-AG measurement in the brain of a murine model after administration of a Reversible Selective MAGL Inhibitor as described in Example 18.

FIG. 2 is a scatter plot graph of the 2-AG concentration at varying brain concentrations of a Reversible Selective MAGL Inhibitor in a murine model as described in Example 18.

FIG. 3A is a graph showing the brain concentration after the administration of Compound 74 and a comparator compound JZL-184 in a mouse model; FIG. 3B is a graph showing the corresponding brain concentration of 2-AG after the administration of Compound 74 and a comparator compound JZL-184 in the animal model.

### DETAILED DESCRIPTION

Methods of treating anxiety, and related conditions with compounds that reversibly inhibit MAGL are provided. Applicants have discovered uses of certain chemical compounds that are Reversible MAGL Inhibitor Compounds and Selective MAGL Inhibitor Compounds. In some embodiments, the compound is a Reversible MAGL Inhibitor Compound. In some embodiments, the compound is a Selective MAGL Inhibitor Compound. In some embodiments, the compound is both a Reversible MAGL Inhibitor Compound and a Selective MAGL Inhibitor Compound.

In some embodiments, the inventions disclosed herein are based in part on the Applicant discovery of the use of reversible Selective MAGL Inhibitor Compounds to transiently increase the level of 2-AG in the brain. In some embodiments, the administration of a reversible Selective MAGL Inhibitor Compound increases the level of 2-AG in the brain of a subject with a half-life that is within twice the half-life of the blood plasma half-life of the reversible Selective MAGL Inhibitor Compound. In some embodiments, the use of a reversible Selective MAGL Inhibitor Compound of Formula (I-A), Formula (I-B), Formula (I-B-1), Formula (I-B-2), Formula (II) or Formula (III) is disclosed, for increasing the level of 2-AG in the brain of a subject with a half-life that is less than twice the corresponding blood plasma half-life for the reversible Selective MAGL Inhibitor Compound in the subject.

Considerable indirect evidence points to the endocannabinoid (eCB) system as a target for anxiety, and related disorders. Clinical studies on the effects of phytocannabinoids support a relationship to anxiety, stress and obsessive disorders. Preclinical studies based on augmenting the levels of the eCB, 2-AG, by inhibiting the hydrolyzing enzyme monoacylglycerol lipase (MAGL) suggest this could be an effective route to develop new treatments to alleviate excessive anxiety and promote stress-coping.

In some embodiments, compounds disclosed herein are useful for the treatment of neurological disorders in the CNS, including anxiety disorders. Anxiety disorders can be characterized by an excessive or inappropriate aroused state characterized by feelings of apprehension, uncertainty, or fear. They are classified according to the severity and duration of their symptoms and specific affective characteristics. Categories include: (1) Generalized anxiety disorder, (2) panic disorder, (3) phobias, (4) obsessive-compulsive disorder, (5) post-traumatic stress disorder, and (6) separation anxiety disorder. The standard treatment for most anxiety disorders is a combination of cognitive-behavioural therapy with antidepressant medication.

In some embodiments, a method of treatment comprises administering a therapeutically effective amount of a compound that is a Reversible MAGL Inhibitor and a Selective MAGL Inhibitor to a patient in need thereof. In some embodiments, methods of treatment are provided.

### Compounds

In some embodiments, the disclosure provides certain compounds of Formula (I-A) that are both a Selective MAGL Inhibitor Compound and a Reversible MAGL Inhibitor Compound as defined herein, or a pharmaceutically acceptable salt thereof:

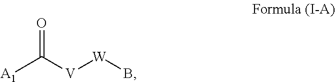

wherein
   A₁ is an aryl or heteroaryl optionally substituted with one or more Ra;
   each Ra is independently halogen, cyano, lower alkyl optionally substituted with one or more halogen, cycloalkyl, aminoalkyl, carboxy, carboxamide, or ˜OR₆;
   R₆ is hydrogen, lower alkyl or lower cycloalkyl optionally substituted with one or more halogen;
   V is selected from

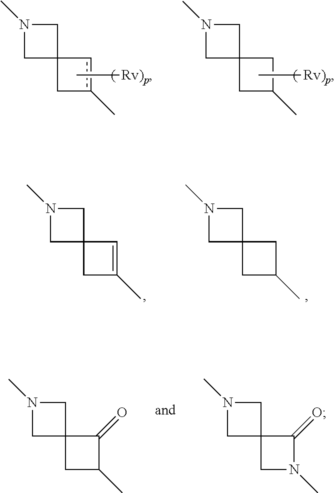

each p is independently 0, 1, 2, 3 or 4;
each Rv is independently hydrogen, halogen, or alkyl optionally substituted with one or more halogen;
W is -A₂-, —C(O)—, C(O)-A₂-, —C(O)N(R₁₀)— and —C(O)N(R₁₀)-A₂-;
A₂ is a 5-member heteroaryl ring optionally substituted with one or more R₃₀;
each R₃₀ is lower alkyl;
R₁₀ is hydrogen or lower alkyl;
B is 5- or 6-member aryl or heteroaryl optionally substituted with one or more R_{b} of or —OR_{b}; and
each R_{b} is independently halogen, cyano, lower alkyl optionally substituted with one or more halogen, cycloalkyl, aminoalkyl, carboxy, or carboxamide.

In some embodiments, methods of treatment comprise the administration or use of a compound that is both a Selective MAGL Inhibitor Compound and a Reversible MAGL Inhibitor Compound of Formula (I-A), wherein A₁ is a 6-member aryl or heteroaryl ring comprising at least one nitrogen; A₂ is a 5-member heteroaryl ring comprising at least one nitrogen heteroatom, and B is a 5- or 6-member aryl or B is a 5- or 6-member heteroaryl ring comprising at least one nitrogen atom, wherein each heteroaryl ring in A₁, A₂ and B comprises one or more heteroatoms selected from the group consisting of nitrogen, oxygen and sulfur.

In some embodiments, A₁ in Formula (I-A) is a 6-member aryl or heteroaryl optionally substituted with one or more Rₐ. In some embodiments, A₁ in Formula (I-A) is phenyl optionally substituted with one or more Rₐ. In some embodiments, A₁ in Formula (I-A) is pyridine optionally substituted with one or more Rₐ. In some embodiments, A₁ in Formula (I-A) is phenyl optionally substituted with one or more Rₐ.

Each Rₐ substitution of A₁ of Formula (I-A) can be the same or different. Each Rₐ in Formula (I-A) is independently halogen, cyano, lower alkyl optionally substituted with one or more halogen, cycloalkyl, aminoalkyl, carboxy, carboxamide, or —OR₆; and each R₆ in Formula (I-A) is independently hydrogen, lower alkyl or lower cycloalkyl optionally substituted with one or more halogen. In some embodiments, the halogen in Ra in Formula (I-A) is F or Cl. In some embodiments, the halogen in Ra in Formula (I-A) is F. In some embodiments, the halogen in Rₐ in Formula (I-A) is F or CL. In some embodiments, the lower alkyl in Ra in Formula (I-A) is (C₁-C₄) alkyl. In some embodiments, the lower alkyl in Ra in Formula (I-A) is methyl optionally substituted with one or more F. In some embodiments, Rₐ in Formula (I-A) is CHF₂, CH₂F, CF₃-cylopropyl, aminoalkyl (including azridinyl), carboxy, carboxamide, formamide, and amide. In some embodiments, cycloalkyl in Rₐ in Formula (I-A) is cyclopropyl. In some embodiments, Rₐ in Formula (I-A) is —NRxCORy or —CONRx or NRxCO, wherein Rx and Ry are each independently hydrogen or lower alkyl. In some embodiments, Rₐ in Formula (I-A) is —NRxCORy or —CONRx or NRxCO, wherein Rx and Ry are each independently (C₁-C₄) alkyl or hydrogen. In some embodiments, Rₐ in Formula (I-A) is —NRxCORy or —CONRx or NRxCO, wherein Rx and Ry are each independently methyl. In some embodiments, Rₐ in Formula (I-A) is —NRxCORy or —CONRx or NRxCO, wherein Rx and Ry are each independently hydrogen.

In some embodiments, V in Formula (I-A) is

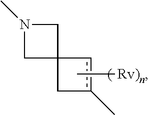

where n and each Rv is as defined above. In some embodiments, V in Formula (I-A) is

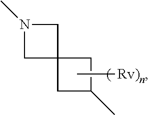

where n and each Rv is as defined above. In some embodiments, V in Formula (I-A) is

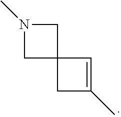

In some embodiments, V in Formula (I-A) is

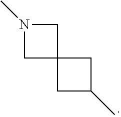

In some embodiments, V in Formula (I-A) is

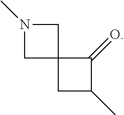

In some embodiments, V in Formula (I-A) is

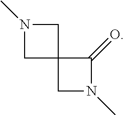

In some embodiments, V in Formula (I-A) is

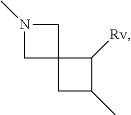

wherein Rv is as defined herein with respect to Formula (I-A).

In some embodiments, each Rv in Formula (I-A) is independently hydrogen, halogen, or alkyl optionally substituted with one or more halogen. In some embodiments, one or more Rv in Formula (I-A) is hydrogen. In some embodiments, one or more Rv in Formula (I-A) is F or Cl. In some embodiments, one or more Rv in Formula (I-A) is F. In some embodiments, one or more Ry in Formula (I-A) is alkyl optionally substituted with one or more F. In some embodiments, one or more Rv in Formula (I-A) is lower alkyl optionally substituted with one or more F. In some embodiments, one or more Rv in Formula (I-A) is (C₁-C₄) alkyl optionally substituted with one or more F. In some embodiments, one or more Rv in Formula (I-A) is CF₃. In some embodiments, each Rv in Formula (I-A) is —O—Rᵥ₂ where Rᵥ₂ is hydrogen or alkyl optionally substituted with one or more halogen. In some embodiments, each Rv in Formula (I-A) is —O—Rᵥ₂ where Rᵥ₂ is hydrogen or lower alkyl optionally substituted with one or more halogen. In some embodiments, each Rv in Formula (I-A) is —O—Rᵥ₂ where Rᵥ₂ is hydrogen or lower alkyl optionally substituted with one or more F. In some embodiments, each Rv in Formula (I-A) is —O—Rᵥ₂ where Rᵥ₂ is hydrogen. In some embodiments, each Rv in Formula (I-A) is —O—Rᵥ₂ where Rᵥ₂ is lower alkyl optionally substituted with one or more halogen. In some embodiments, each Rv in Formula (I-A) is F. In some embodiments, each Rv in Formula (I-A) is methyl optionally substituted with one or more F. In some embodiments, each Rv in Formula (I-A) is methyl.

In some embodiments, each Rv in Formula (I-A) is —O—Rᵥ₂ where Rᵥ₂ is (C₁-C₄) alkyl optionally substituted with one or more halogen. In some embodiments, each Rv in Formula (I-A) is —O—Rᵥ₂ where Rᵥ₂ is (C₁-C₄) alkyl optionally substituted with one or more F. In some embodiments, each Rv in Formula (I-A) is —O—Rᵥ₂ where Rᵥ₂ is methyl optionally substituted with one or more F.

In some embodiments, each n with respect to Rv in Formula (I-A) is 0, 1, 2, 3 or 4. In some embodiments, each n with respect to Rv in Formula (I-A) is 0. In some embodiments, each n with respect to Rv in Formula (I-A) is 1. In some embodiments, each n with respect to Rv in Formula (I-A) is 2. In some embodiments, each n with respect to Rv in Formula (I-A) is 3. In some embodiments, each n with respect to Rv in Formula (I-A) is 4.

In some embodiments, V in Formula (I-A) is

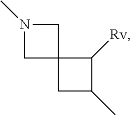

wherein Rv is halogen, lower alkyl optionally substituted with one or more halogen, or —O—Rᵥ₂ where Rᵥ₂ is (C₁-C₄) alkyl optionally substituted with one or more halogen. In some embodiments, V in Formula (I-A) is

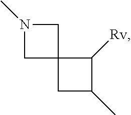

wherein Rv is F, (C₁-C₄) alkyl optionally substituted with one or more F, or —O—Rᵥ₂ where Rᵥ₂ is methyl optionally substituted with one or more halogen. In some embodiments, V in Formula (I-A) is

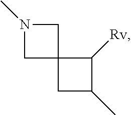

wherein Rv is F, methyl optionally substituted with one or more F, or —O—Rᵥ₂ where Rᵥ₂ is methyl optionally substituted with one or more halogen. In some embodiments, V in Formula (I-A) is

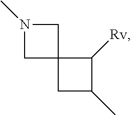

wherein Rv is F, methyl, ethyl, —CF₃, or —O—Rᵥ₂ where Rᵥ₂ is methyl optionally substituted with one or more halogen.

In some embodiments, W in Formula (I-A) is A₂, wherein A₂ is as defined above. In some embodiments, W in Formula (I-A) is —C(O)— or —C(O)N(R₁₀)—, wherein R₁₀ is as defined above with respect to Formula (I-A). In some embodiments, W in Formula (I-A) is —C(O)—. In some embodiments, W in Formula (I-A) is —C(O)N(R₁₀)—, wherein R₁₀ is as defined above with respect to Formula (I-A).

In some embodiments, W in Formula (I-A) is A₂, and W in Formula (I-B) is A, wherein A or A₂ is a 5-member heteroaryl comprising one or more heteroatoms selected from nitrogen, oxygen and sulfur, and optionally substituted with one or more R₃₀ as defined above. In some W in Formula (I-A) or Formula (I-B) is a 5-member heteroaryl comprising one or more nitrogen heteroatoms and optionally further comprising one or more additional heteroatoms selected from nitrogen, oxygen and sulfur, and optionally substituted with one or more R₃₀ as defined above. In some W in Formula (I-A) or Formula (I-B) is a 5-member heteroaryl comprising one or more nitrogen heteroatoms and optionally further comprising one or two additional heteroatoms selected from nitrogen, oxygen and sulfur, and optionally substituted with one or more R₃₀ as defined above. In some W in Formula (I-A) or Formula (I-B) is selected from the group consisting of:

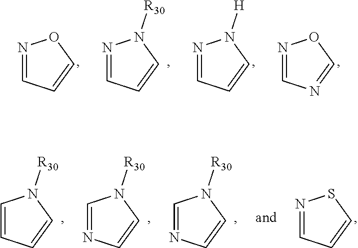

wherein R₃₀ is as defined herein.

In some embodiments, A₂ in Formula (I-A) is a 5-member heteroaryl ring optionally substituted with one or more R₃₀, wherein R₃₀ is (C₁-C₄) alkyl. In some embodiments, A₂ in Formula (I-A) is a 5-member heteroaryl ring optionally substituted with one or more R₃₀, wherein R₃₀ is methyl.

In some embodiments, each R₁₀ in Formula (I-A) can be the same or different. In some embodiments, each R₁₀ in Formula I-A is hydrogen or (C₁-C₄) alkyl. In some embodiments, one or more R₁₀ in Formula I-A is hydrogen. In some embodiments, one or more R₁₀ in Formula I-A is (C₁-C₄) alkyl. In some embodiments, one or more R₁₀ in Formula I-A is methyl.

In some embodiments, B in Formula (I-A) is 5- or 6-member aryl or heteroaryl optionally substituted with one or more R_{b}; and each R_{b} is independently halogen, cyano, lower alkyl optionally substituted with one or more halogen, cycloalkyl, aminoalkyl, carboxy, carboxamide, or —OR₆; and Re is hydrogen, lower alkyl or lower cycloalkyl optionally substituted with one or more halogen.

In some embodiments, B in Formula (I-A) is phenyl or 5- or 6-member heteroaryl comprising one or more heteroatoms selected from N, O and S, wherein the B group is optionally substituted with one or more Rₐ; and each R_{b} is independently halogen, cyano, (C₁-C₄) alkyl optionally substituted with one or more halogen, (C₃-C₆) cycloalkyl, 3-6 member heterocycloalkyl, aminoalkyl, carboxy, carboxamide, or —OR₆; and R₆ is hydrogen, (C₁-C₄)alkyl or (C₃-C₆) cycloalkyl optionally substituted with one or more halogen. In some embodiments, any one or more halogen within the B group in Formula (I-A) is F.

In some embodiments, B in Formula (I-A) is phenyl optionally substituted with one or more R_{b}; and each R_{b} is independently F, Cl, cyano, (C₁-C₄) alkyl optionally substituted with one or more F or Cl, (C₃-C₆) cycloalkyl, 3-6 member heterocycloalkyl, aminoalkyl, carboxy, carboxamide, or —OR₆; and R₆ is hydrogen, (C₁-C₄)alkyl or (C₃-C₆) cycloalkyl optionally substituted with one or more F or Cl.

In some embodiments, B in Formula (I-A) is 5-member heteroaryl comprising one or more heteroatoms selected from N, O and S, optionally substituted with one or more R_{b}; and each R_{b} is independently F, Cl, cyano, (C₁-C₄) alkyl optionally substituted with one or more F or Cl, (C₃-C₆) cycloalkyl, 3-6 member heterocycloalkyl, aminoalkyl, carboxy, carboxamide, or —OR₆; and R₆ is hydrogen, (C₁-C₄)alkyl or (C₃-C₆) cycloalkyl optionally substituted with one or more F or Cl. In some embodiments, B in Formula (I-A) is 5-member heteroaryl comprising nitrogen and 0, 1 or 2 additional heteroatoms selected from N, O and S, optionally substituted with one or more R_{b}; and each R_{b} is independently F, Cl, cyano, (C₁-C₄) alkyl optionally substituted with one or more F or Cl, (C₃-C₆) cycloalkyl, 3-6 member heterocycloalkyl, aminoalkyl, carboxy, carboxamide, or —OR₆; and R₆ is hydrogen, (C₁-C₄)alkyl or (C₃-C₆) cycloalkyl optionally substituted with one or more F or Cl.

In some embodiments, B in Formula (I-A) is 6-member heteroaryl comprising one or more heteroatoms selected from N, O and S, optionally substituted with one or more R_{b}; and each R_{b} is independently F, Cl, cyano, (C₁-C₄)alkyl optionally substituted with one or more F or CI, (C₃-C₆) cycloalkyl, 3-6 member heterocycloalkyl, aminoalkyl, carboxy, carboxamide, or —OR₆; and R₆ is hydrogen, (C₁-C₄)alkyl or (C₃-C₆) cycloalkyl optionally substituted with one or more F or Cl. In some embodiments, B in Formula (I-A) is 6-member heteroaryl comprising nitrogen and 0, 1 or 2 additional heteroatoms selected from N, O and S, optionally substituted with one or more R_{b}; and each R_{b} is independently F, Cl, cyano, (C₁-C₄)alkyl optionally substituted with one or more F or Cl, (C₃-C₆) cycloalkyl, 3-6 member heterocycloalkyl, aminoalkyl, carboxy, carboxamide, or —OR₆; and R₆ is hydrogen, (C₁-C₄)alkyl or (C₃-C₆) cycloalkyl optionally substituted with one or more F or Cl.

In some embodiments, B in Formula (I-A) is substituted with 0, 1, 2, 3, 4 or 4 R_{b} that are each the same or different from each other. In some embodiments, one or more R_{b} in Formula (I-A) is F. In some embodiments, one or more R_{b} in Formula (I-A) is Cl. In some embodiments, one or more R_{b} in Formula (I-A) is —CN. In some embodiments, one or more R_{b} in Formula (I-A) is (C₁-C₄) alkyl optionally substituted with one or more F or Cl. In some embodiments, one or more R_{b} in Formula (I-A) is (C₁-C₄) alkyl optionally substituted with one or more F. In some embodiments, one or more R_{b} in Formula (I-A) is methyl optionally substituted with one or more F or Cl. In some embodiments, one or more R_{b} in Formula (I-A) is methyl optionally substituted with one or more F. In some embodiments, one or more R_{b} in Formula (I-A) is (C₃-C₆) cycloalkyl. In some embodiments, one or more R_{b} in Formula (I-A) is cyclopropyl. In some embodiments, one or more R_{b} in Formula (I-A) is cyclobutyl. In some embodiments, one or more R_{b} in Formula (I-A) is cyclohexyl. In some embodiments, one or more R_{b} in Formula (I-A) is a 3-member heterocycloalkyl group comprising an O, N or S heteroatom. In some embodiments, one or more R_{b} in Formula (I-A) is a 4-member heterocycloalkyl group comprising one or more O, N or S heteroatoms. In some embodiments, one or more R_{b} in Formula (I-A) is a 5-member heterocycloalkyl group comprising one or more O, N or S heteroatoms. In some embodiments, one or more R_{b} in Formula (I-A) is a 6-member heterocycloalkyl group comprising one or more O, N or S heteroatoms. In some embodiments, one or more R_{b} in Formula (I-A) is an aminoalkyl. In some embodiments, one or more R_{b} in Formula (I-A) is carboxy group. In some embodiments, one or more R_{b} in Formula (I-A) is OR₆, and R₆ is hydrogen, (C₁-C₄)alkyl or (C₃-C₆) cycloalkyl optionally substituted with one or more F or CL.

In some embodiments, the disclosure provides a compound of Formula (I-B) or a pharmaceutically acceptable salt thereof:

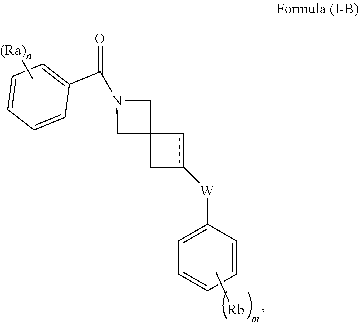

wherein
n is 1, 2, 3, 4 or 5;
each Rₐ is independently halogen, cyano, lower alkyl optionally substituted with one or more halogen, —OR₆, amine, amide, or ester,
R₆ is hydrogen, lower alkyl or cycloalkyl optionally substituted with one or more halogen;
W is A, —C(O)—, or —C(O)N(R₁₀)—;
A is aryl or heteroaryl each optionally substituted with one or more R₃₀;
each R₃₀ is independently lower alkyl optionally substituted with one or more halogen;
R₁₀ is hydrogen or lower alkyl;
m is 1, 2, 3, 4 or 5; and
each R_{b} is independently halogen, or lower alkyl optionally substituted with one or more halogen.

In some embodiments, compounds can be a compound of Formula (I-B), wherein n is 1, 2 or 3 and each Ra is independently halogen, cyano, lower alkyl optionally substituted with one or more halogen, or —OR₆; R₆ is hydrogen, lower alkyl or cycloalkyl optionally substituted with one or more halogen. In compounds of Formula (I-B), n is 1, 2 or 3 and each Ra is independently F or Cl, cyano, (C₁-C₄)alkyl optionally substituted with one or more F, or —OR₆; R₆ is hydrogen, (C₁-C₄)alkyl or cyclopropyl optionally substituted with one or more F.

In some embodiments, the disclosure provides a compound of Formula (I-B) or a pharmaceutically acceptable salt thereof, wherein:
n is 1, 2, or 3;
each Ra is independently halogen, cyano, lower alkyl optionally substituted with one or more halogen, or —OR₆;
R₆ is hydrogen, lower alkyl or lower cycloalkyl optionally substituted with one or more halogen;
W is A, —C(O)—, —C(O)-A-, or —C(O)N(R₁₀)—;
R₁₀ is hydrogen or lower alkyl;
A is a 5-member heteroaryl ring optionally substituted with one or more R₃₀;
R₃₀ is lower alkyl;
m is 1, or 2; and
each R_{b} is independently halogen, or lower alkyl optionally substituted with one or more halogen.

In compounds of Formula (I-B), W is a 5-member heteroaryl ring comprising at least one nitrogen, such as a triazole, imidazole, pyrazole, or an oxadiazole. In some embodiments, one or more of the lower alkyl groups in Ra, Rv, R₆, R₁₀, R₃₀ and R_{b} can independently be methyl. In some embodiments, a compound is a compound of Formula (I-B), wherein each Ra is independently Cl, F, CN, cyano, methyl, or —OR₆; R₆ is hydrogen, (C₁-C₄)alkyl optionally substituted with one or more F or cyclopropyl; W is -A-, —C(O)—, or —C(O)N(R₁₀)—; R₁₀ is hydrogen or methyl; A is a 5-member heteroaryl ring optionally substituted with one or more R₃₀; R₃₀ is (C₁-C₄)alkyl; m is 1, or 2; and each R_{b} is independently halogen, or (C₁-C₄)alkyl optionally substituted with one or more F. In some embodiments, a compound is a compound of Formula (I-B) wherein R₃₀ is methyl; and each R_{b} is independently halogen, or methyl optionally substituted with one or more F. In some embodiments, a compound is a compound of Formula (I-B) wherein A is pyrazole, imidazole, or triazole, each optionally substituted with one methyl. In some embodiments, a compound is a compound of Formula (I-B) wherein A is oxadiazole. In some embodiments, a compound is a compound of Formula (I-B), wherein A is pyrazole substituted with one methyl. In some embodiments, a compound is a compound of Formula (I-B), wherein one Ra is —OR₆.

In some embodiments, compounds of Formula (I-A) of Formula (I-B) can be a compound of Formula (I-B-1), or pharmaceutically acceptable salt thereof:

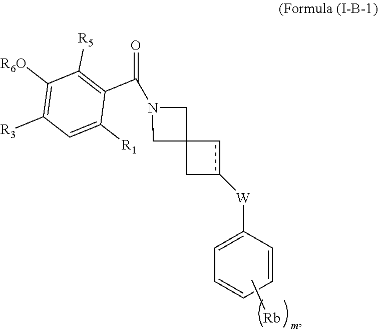

wherein
   R₁ is hydrogen or halogen;
   R₃ is hydrogen, halogen, or lower alkyl optionally substituted with one or more halogen;
   R₅ is hydrogen, halogen, lower alkoxy or lower alkyl each optionally substituted with one or more halogen;
   R₆ is hydrogen, lower alkyl or cycloalkyl optionally substituted with one or more halogen; and
   W is A, —C(O)—, or —C(O)N(R₁₀)—;
   A is aryl or heteroaryl each optionally substituted with one or more R₃₀;
   each R₃₀ is independently lower alkyl optionally substituted with one or more halogen;
   R₁₀ is hydrogen or lower alkyl; and
   m is 1, 2, 3, 4 or 5.

In some embodiments, a compound is a compound of Formula (I-B-1) wherein R₁ is hydrogen, —CN, Cl or F. In some embodiments, R₆ is hydrogen, (C₁-C₄)alkyl or cyclopropyl optionally substituted with one or more F. In some embodiments, a compound is a compound of Formula (I-B-1) wherein R₁ is hydrogen, —CN, Cl or F and R₆ is hydrogen, (C₁-C₄)alkyl or cyclopropyl optionally substituted with one or more F. In some embodiments, R₃ is hydrogen, F, or methyl optionally substituted with one or more halogen. In some embodiments, a compound is a compound of Formula (I-B-1) wherein R₁ is hydrogen, —CN, Cl or F; R₆ is hydrogen, (C₁-C₄)alkyl or cyclopropyl optionally substituted with one or more F; and R₃ is hydrogen, F, or methyl optionally substituted with one or more halogen. In some embodiments, a compound is a compound of Formula (I-B-1) wherein R₁ is hydrogen, —CN, Cl or F; R₆ is hydrogen, (C₁-C₄)alkyl or cyclopropyl optionally substituted with one or more F; R₃ is hydrogen, F, or methyl optionally substituted with one or more halogen; and R₅ is hydrogen.

In some embodiments, a compound is a compound of Formula (I-B-1) wherein R₁ is Cl, F or —CN and R₆ is hydrogen. In some embodiments, a compound is a compound of Formula (I-B-1) wherein R₁ is F. In some embodiments, a compound is a compound of Formula (I-B-1) wherein R₁ is Cl, F or —CN and R₃, R₅ and R₆ are each hydrogen. In some embodiments, a compound is a compound of Formula (I-B-1) wherein R₁ is Cl, F or —CN; R₃ is hydrogen, methyl or F; R₅ is hydrogen, methyl or F; and R₆ is hydrogen. In some embodiments, a compound is a compound of Formula (I-B-1) wherein R₁ is F; R₃ is hydrogen, methyl or F; R₅ is hydrogen; and R₆ is hydrogen. In some embodiments, a compound is a compound of Formula (I-B-1) wherein R₁ is F; R₃ is hydrogen; R₅ is hydrogen or F; and R₆ is hydrogen. In some embodiments, a compound is a compound of Formula (I-B-1) wherein R₁ is hydrogen; R₃ is hydrogen or F; R₅ is hydrogen; and R₆ is hydrogen. In some embodiments, a compound is a compound of Formula (I-B-1) wherein R₁ is F or —CN; R₃ is hydrogen or F; R₅ is hydrogen; and R₆ is hydrogen. In some embodiments, a compound is a compound of Formula (I-B-1) wherein R₁ and R₆ are each hydrogen.

In some embodiments, a compound is a compound of Formula (I-B-1) wherein R₁ is F and R₆ is hydrogen. In some embodiments, a compound is a compound of Formula (I-B-1) wherein R₁ is F, R₆ is hydrogen and at least one of R₃ and R₅ is hydrogen. In some embodiments, a compound is a compound of Formula (I-B-1) wherein R₁ is F, R₆ is hydrogen, and R₃ and R₅ are each hydrogen. In some embodiments, a compound is a compound of Formula (I-B-1) wherein R₁ is F, R₆ is hydrogen and at least one of R₃ and R₅ is hydrogen and at least one of R₃ and R₅ is F or methyl.

In some embodiments, a compound is a compound of Formula (I-B-1) wherein W is A and A is 5-member aryl or 5-member heteroaryl wherein each A is optionally substituted with one or more R₃₀; and each R₃₀ is independently lower alkyl optionally substituted with one or more halogen. In some embodiments, a compound is a compound of Formula (I-B-1) wherein W is A and A is 5-member heteroaryl comprising at least one nitrogen heteroatom wherein each A is optionally substituted with one or more R₃₀; and each R₃₀ is independently (C₁-C₄) alkyl optionally substituted with one or more halogen. In some embodiments, a compound is a compound of Formula (I-B-1) wherein W is A and A is 5-member heteroaryl comprising at least one nitrogen heteroatom wherein each A is optionally substituted with one or more R₃₀; and each R₃₀ is independently F or methyl optionally substituted with one or more F.

In some embodiments, compounds of Formula (I-A) or Formula (I-B) can be a compound of Formula (I-B-2), or pharmaceutically acceptable salt thereof:

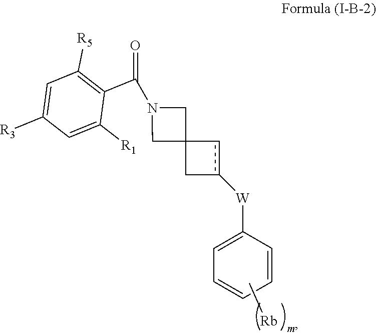

wherein
R₃ is hydrogen or halogen;
R₅ is —O—R₅₂;
R₅₂ is lower alkyl or cycloalkyl, each optionally substituted with halogen,
W is A, —C(O)—, or —C(O)N(R₁₀)—;
A is aryl or heteroaryl each optionally substituted with one or more R₃₀;
each R₃₀ is independently lower alkyl optionally substituted with one or more halogen;
R₁₀ is hydrogen or lower alkyl; and
m is 1, 2, 3, 4 or 5.

In some embodiments, a compound is a compound of Formula (I-B-2) wherein R₃ is hydrogen, Cl or F. In some embodiments, a compound is a compound of Formula (I-B-2) wherein R₃ is F. In some embodiments, a compound is a compound of Formula (I-B-2) wherein R₅₂ is (C₁-C₄)alkyl optionally substituted with one or more halogen, or cyclopropyl. In some embodiments, a compound is a compound of Formula (I-B-2) wherein R₃ is F and R₅₂ is (C₁-C₄)alkyl optionally substituted with one or more halogen, or cyclopropyl. In some embodiments, a compound is a compound of Formula (I-B-2) wherein R₅₂ is (C₁-C₄)alkyl optionally substituted with one or more F, or cyclopropyl. In some embodiments, a compound is a compound of Formula (I-B-2) wherein R₃ is F and R₅₂ is (C₁-C₄)alkyl optionally substituted with one or more F, or cyclopropyl.

In some embodiments, a compound is a compound of Formula (I-B-2) wherein R₅₂ is selected from the group consisting of: methyl, ethyl, propyl optionally substituted with one or more F. In some embodiments, a compound is a compound of Formula (I-B-2) wherein R₃ is F and R₅₂ is selected from the group consisting of: methyl, ethyl, propyl optionally substituted with one or more F. In some embodiments, a compound is a compound of Formula (I-B-2) wherein R₅₂ is selected from the group consisting of: methyl, ethyl, isopropyl, —CH₂—CF₃ and cyclopropyl. In some embodiments, a compound is a compound of Formula (I-B-2) wherein R₃ is F and R₅₂ is selected from the group consisting of: methyl, ethyl, isopropyl, —CH₂—CF₃ and cyclopropyl.

In some embodiments, a compound is a compound of Formula (I-B-2) wherein W is A and A is 5-member aryl or 5-member heteroaryl wherein each A is optionally substituted with one or more R₃₀; and each R₃₀ is independently lower alkyl optionally substituted with one or more halogen. In some embodiments, a compound is a compound of Formula (I-B-2) wherein W is A and A is 5-member heteroaryl comprising at least one nitrogen heteroatom wherein each A is optionally substituted with one or more R₃₀; and each R₃₀ is independently (C₁-C₄) alkyl optionally substituted with one or more halogen. In some embodiments, a compound is a compound of Formula (I-B-2) wherein W is A and A is 5-member heteroaryl comprising at least one nitrogen heteroatom wherein each A is optionally substituted with one or more R₃₀; and each R₃₀ is independently F or methyl optionally substituted with one or more F.

In some embodiments, a compound is a compound of Formula (I-A), Formula (I-B), Formula (I-B-1) or Formula (I-B-2), wherein W is A, and A is an aryl or heteroaryl optionally substituted with one or more R₃₀, and R₃₀ is (C₁-C₄)alkyl optionally substituted with one or more halogen. In some embodiments, a compound is a compound of Formula (I-A), Formula (I-B), Formula (I-B-1) or Formula (I-B-2), wherein A is a 5-member heteroaryl optionally substituted with one or more (C₁-C₄)alkyl optionally substituted with one or more halogen. In some embodiments, a compound is a compound of Formula (I-A), Formula (I-B), Formula (I-B-1) or Formula (I-B-2), wherein A is a 5-member heteroaryl comprising one or more nitrogen heteroatoms and optionally substituted with one or more methyl.

In some embodiments, a compound is a compound of Formula (I-A), Formula (I-B), Formula (I-B-1) or Formula (I-B-2), wherein W is selected from the group consisting of: imidazole, pyrazole, triazole and oxadiazole each optionally substituted with one or more lower alkyl. In some embodiments, a compound is a compound of Formula (I-A), Formula (I-B), Formula (I-B-1) or Formula (I-B-2), wherein W is selected from the group consisting of: imidazole, pyrazole, triazole and oxadiazole each optionally substituted with one or more methyl. In some embodiments, a compound is a compound of Formula (I-A), Formula (I-B), Formula (I-B-1) or Formula (I-B-2), wherein W is selected from the group consisting of A1, A2, A3, A4, A5, A6 and A7 as shown below, wherein R₃₀, R₃₂, R₃₃, R₃₄, R₃₆, R₃₇, R₃₈ and R₃₉ are each independently hydrogen or lower alkyl:

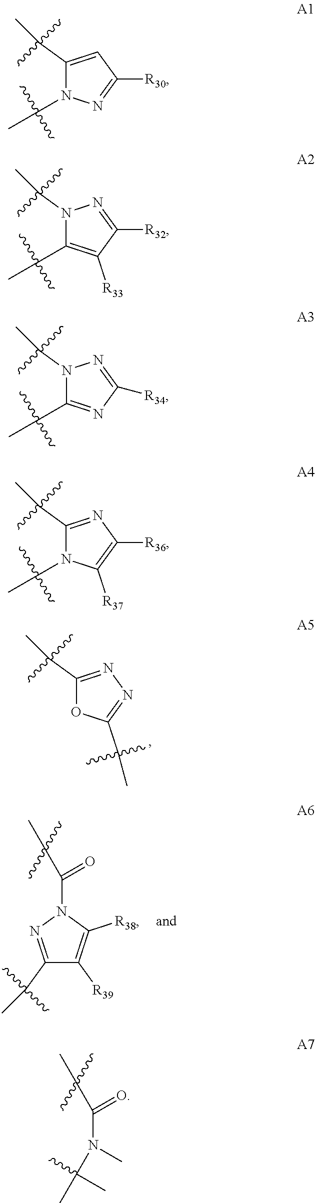

In some embodiments, a compound is a compound of Formula (I-A), Formula (I-B), Formula (I-B-1) or Formula (I-B-2), wherein W is selected from the group consisting of A1, A2, A3, A4, A5, and A6 as shown above, wherein R₃₀, R₃₂, R₃₃, R₃₄, R₃₆, R₃₇, R₃₈ and R₃₉ are each independently hydrogen or methyl. In some embodiments, a compound is a compound of Formula (I-A), Formula (I-B), Formula (I-B-1) or Formula (I-B-2), wherein W is selected from the group consisting of A1, A2, A3, A4, A5, and A6 as shown above, wherein R₃₀, R₃₂, R₃₃, R₃₄, R₃₆, R₃₇, R₃₈ and R₃₉ are each independently hydrogen or methyl.

In some embodiments, a compound is a compound of Formula (I-A), Formula (I-B), Formula (I-B-1) or Formula (I-B-2), wherein W is A1 and R₃₀ is hydrogen or methyl. In some embodiments, a compound is a compound of Formula (I-A), Formula (I-B), Formula (I-B-1) or Formula (I-B-2), wherein W is A1 and R₃₀ is hydrogen. In some embodiments, a compound is a compound of Formula (I-A), Formula (I-B), Formula (I-B-1) or Formula (I-B-2), wherein W is A1 and R₃₀ is methyl. In some embodiments, a compound is a compound of Formula (I-A), Formula (I-B), Formula (I-B-1) or Formula (I-B-2), wherein W is A2 and R₃₂ and R₃₃ is hydrogen or methyl.

In some embodiments, a compound is a compound of Formula (I-A), Formula (I-B), Formula (I-B-1) or Formula (I-B-2), wherein W is A2 and one of R₃₂ and R₃₃ is hydrogen and one of R₃₂ and R₃₃ is methyl. In some embodiments, a compound is a compound of Formula (I-A), Formula (I-B), Formula (I-B-1) or Formula (I-B-2), wherein W is A2 and R₃₂ is methyl and R₃₃ is hydrogen. In some embodiments, a compound is a compound of Formula (I-A), Formula (I-B), Formula (I-B-1) or Formula (I-B-2), wherein W is A2 and R₃₂ is hydrogen and R₃₃ is methyl. In some embodiments, a compound is a compound of Formula (I-A), Formula (I-B), Formula (I-B-1) or Formula (I-B-2), wherein W is A2 and R₃₂ is hydrogen and R₃₃ is hydrogen.

In some embodiments, a compound is a compound of Formula (I-A), Formula (I-B), Formula (I-B-1) or Formula (I-B-2), wherein W is A3 and R₃₄ is hydrogen or methyl. In some embodiments, a compound is a compound of Formula (I-A), Formula (I-B), Formula (I-B-1) or Formula (I-B-2), wherein W is A3 and R₃₄ is hydrogen. In some embodiments, a compound is a compound of Formula (I-A), Formula (I-B), Formula (I-B-1) or Formula (I-B-2), wherein W is A1 and R₃₄ is methyl.

In some embodiments, a compound is a compound of Formula (I-A), Formula (I-B), Formula (I-B-1) or Formula (I-B-2), wherein W is A4 and one of R₃₆ and R₃₇ is hydrogen and one of R₃₆ and R₃₇ is methyl. In some embodiments, a compound is a compound of Formula (I-A), Formula (I-B), Formula (I-B-1) or Formula (I-B-2), wherein W is A4 and R₃₆ is methyl and R₃₇ is hydrogen. In some embodiments, a compound is a compound of Formula (I-A), Formula (I-B), Formula (I-B-1) or Formula (I-B-2), wherein W is A4 and R₃₆ is hydrogen and R₃₇ is methyl. In some embodiments, a compound is a compound of Formula (I-A), Formula (I-B), Formula (I-B-1) or Formula (I-B-2), wherein W is A4 and R₃₆ is hydrogen and R₃₇ is hydrogen.

In some embodiments, a compound is a compound of Formula (I-A), Formula (I-B), Formula (I-B-1) or Formula (I-B-2), wherein W is A5.

In some embodiments, a compound is a compound of Formula (I-A), Formula (I-B), Formula (I-B-1) or Formula (I-B-2), wherein W is A₆ and one of R₃₈ and R₃₉ is hydrogen and one of R₃₈ and R₃₉ is methyl. In some embodiments, a compound is a compound of Formula (I-A), Formula (I-B), Formula (I-B-1) or Formula (I-B-2), wherein W is A6 and R₃₈ is methyl and R₃₉ is hydrogen. In some embodiments, a compound is a compound of Formula (I-A), Formula (I-B), Formula (I-B-1) or Formula (I-B-2), wherein W is A6 and R₃₈ is hydrogen and R₃₉ is methyl. In some embodiments, a compound is a compound of Formula (I-A), Formula (I-B), Formula (I-B-1) or Formula (I-B-2), wherein W is A6 and R₃₈ is hydrogen and R₃₉ is hydrogen.

In some embodiments, a compound is a compound of Formula (I-A), Formula (I-B), Formula (I-B-1) or Formula (I-B-2), wherein at least one of Ra is hydroxyl or (C₁-C₄)alkoxy optionally substituted with one or more halogen, and n is 1, 2 or 3. In some embodiments, a compound is a compound of Formula (I-A), Formula (I-B), Formula (I-B-1) or Formula (I-B-2), wherein at least one of Ra is hydroxyl or (C₁-C₄)alkoxy optionally substituted with one or more F, and n is 1, 2 or 3. In some embodiments, a compound is a compound of Formula (I-A), Formula (I-B), Formula (I-B-1) or Formula (I-B-2), wherein at least one of Rₐ is hydroxyl, (C₁-C₄)alkoxy or —O—(C₁-C₆) cycloalkyl each optionally substituted with one or more F, with the remaining Rₐ selected from the group consisting of halogen, methyl, and cyano; and n is 1, 2 or 3. In some embodiments, a compound is a compound of Formula (I-A), Formula (I-B), Formula (I-B-1) or Formula (I-B-2), wherein at least one of Ra is hydroxyl, (C₁-C₄)alkoxy or —O-(cyclopropyl) each optionally substituted with one or more F, with the remaining Ra selected from the group consisting of halogen, methyl, and cyano; and n is 1, 2 or 3. In some embodiments, a compound is a compound of Formula (I-A), Formula (I-B), Formula (I-B-1) or Formula (I-B-2), wherein at least one of Rₐ is hydroxyl, (C₁-C₄)alkoxy or —O-(cyclopropyl) each optionally substituted with one or more F, with the remaining Ra selected from the group consisting of Cl, F, methyl, and cyano; and n is 1, 2 or 3.

In some embodiments, a compound is a compound of Formula (I-A), Formula (I-B), Formula (I-B-1) or Formula (I-B-2), wherein W is A7.

In some embodiments, a compound is a compound of Formula (I-A), Formula (I-B), Formula (I-B-1) or Formula (I-B-2), wherein W is —C(O)—.

In some embodiments, a compound is a compound of Formula (I-A), Formula (I-B), Formula (I-B-1) or Formula (I-B-2), wherein W is —C(O)N(R₁₀)—, and —R₁₀ is hydrogen. In some embodiments, a compound is a compound of Formula (I-A), Formula (I-B), Formula (I-B-1) or Formula (I-B-2), wherein W is —C(O)N(R₁₀)—, and —R₁₀ is methyl. In some embodiments, a compound is a compound of Formula (I-A), Formula (I-B), Formula (I-B-1) or Formula (I-B-2), wherein W is —C(O)N(R₁₀)—, and —R₁₀ is (C₁-C₄) alkyl optionally substituted with F. In some embodiments, a compound is a compound of Formula (I-A), Formula (I-B), Formula (I-B-1) or Formula (I-B-2), wherein W is —C(O)N(R₁₀)—, and —R₁₀ is lower alkyl optionally substituted with halogen.

In some embodiments, compounds of Formula (I-A) or Formula (I-B) can be a compound of Formula (I-C), or pharmaceutically acceptable salt thereof:

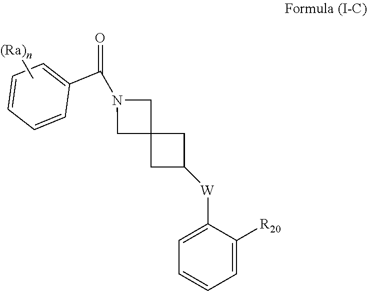

wherein:
   R₂₀ is lower alkyl;
   n is 1, 2, or 3;
   each Rₐ is independently halogen, cyano, lower alkyl optionally substituted with one or more halogen, or —OR₆;
   W is as defined with respect to Formula (I-B); and
   R₆ is hydrogen, lower alkyl or lower cycloalkyl optionally substituted with one or more halogen.

In some embodiments, a compound is a compound of Formula (I-C), wherein R₂₀ is (C₁-C₄)alkyl. In some embodiments, a compound is a compound of Formula (I-C), wherein R₂₀ is methyl. In some embodiments, a compound is a compound of Formula (I-C), wherein W is A7

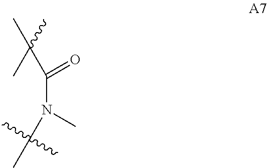

and R₂₀ is methyl, each Ra is independently F, Cl, —CN, (C₁-C₄)alkyl optionally substituted with one or more F or CI, or —OR₆ and R₆ is hydrogen, (C₁-C₄)alkyl or cyclopropyl optionally substituted with one or more F or CL. In some embodiments, a compound is a compound of Formula (I-C), wherein n is 1, 2 or 3, W is A7 and R₂₀ is methyl, each Ra is independently F, Cl, —CN, methyl, or —OH. In some embodiments, a compound is a compound of Formula (I-C), wherein n is 2, W is A7 and R₂₀ is methyl, each Ra is independently F or —OH.

In some embodiments, a compound is a compound of Formula (I-C), wherein at least one of Ra is hydroxyl or (C₁-C₄)alkoxy optionally substituted with one or more halogen, and n is 1, 2 or 3. In some embodiments, a compound is a compound of Formula (I-C), wherein at least one of Ra is hydroxyl or (C₁-C₄)alkoxy optionally substituted with one or more F, and n is 1, 2 or 3. In some embodiments, a compound is a compound of Formula (I-C), wherein at least one of Ra is hydroxyl, (C₁-C₄)alkoxy or —O—(C₁-C₆) cycloalkyl each optionally substituted with one or more F, with the remaining Rₐ selected from the group consisting of halogen, methyl, and cyano; and n is 1, 2 or 3. In some embodiments, a compound is a compound of Formula (I-C), wherein at least one of Rₐ is hydroxyl, (C₁-C₄)alkoxy or —O-(cyclopropyl) each optionally substituted with one or more F, with the remaining Ra selected from the group consisting of halogen, methyl, and cyano; and n is 1, 2 or 3. In some embodiments, a compound is a compound of Formula (I-C), wherein at least one of Ra is hydroxyl, (C₁-C₄)alkoxy or —O-(cyclopropyl) each optionally substituted with one or more F, with the remaining Ra selected from the group consisting of Cl, F, methyl, and cyano; and n is 2 or 3. In some embodiments, a compound is a compound of Formula (I-C), wherein at least one of Ra is hydroxyl, with the remaining Ra selected from the group consisting of F, methyl, and cyano; and n is 2 or 3.

In some embodiments, the disclosure provides a compound of Formula (I-A) or Formula (I-B) that are also compounds of Formula (II) or a pharmaceutically acceptable salt thereof:

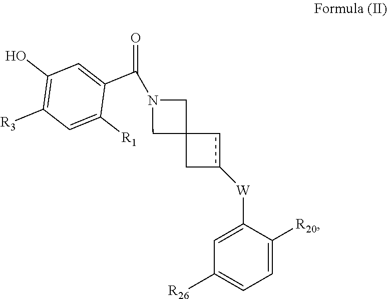

wherein
R₁ is halogen or cyano;
R₃ is hydrogen, lower alkyl, or halogen;
W is A, —C(O)—, or —C(O)N(R₁₀)—;
R₁₀ is hydrogen or lower alkyl;
A is a S-member heteroaryl ring optionally substituted with one or more R₃₀;
R₂₀ is halogen or lower alkyl optionally substituted with one or more halogen;
R₃₀ is lower alkyl optionally substituted with one or more halogen; and
R₂₆ is halogen or hydrogen.

In some embodiments, a compound of Formula (I-A) or Formula (I-B) can be a compound of Formula (II) wherein R₁ is Cl, F or cyano; R₃ is hydrogen, F or methyl; R₁₀ is hydrogen or methyl; R₂₀ and R₃₀ are each independently methyl; and R₂₆ is F or hydrogen. In some embodiments, a compound of Formula (I) can be a compound of Formula (II) wherein R₁ is Cl, F or cyano; R₃ is hydrogen, F or methyl; R₁₀ is hydrogen or methyl; R₂₀ and R₃₀ are each independently methyl; and R₂₆ is hydrogen. In some embodiments, a compound of Formula (I) can be a compound of Formula (II) wherein W is —C(O)N(R₁₀)— wherein R₁₀ is hydrogen or methyl and A is a 5-member heteroaryl ring comprising at least one nitrogen optionally substituted with one or more lower alkyl, the lower alkyl being optionally substituted with one or more halogen; and R₂₆ is hydrogen. In some embodiments, a compound of Formula (I) can be a compound of Formula (II) wherein R₂₀ and R₃₀ are each independently lower alkyl optionally substituted with one or more F and R₂₆ is hydrogen. In some embodiments, a compound of Formula (I) can be a compound of Formula (II) wherein R₂₀ is halogen or lower alkyl optionally substituted with one or more F; R₃₀ is lower alkyl optionally substituted with one or more F; and R₂₆ is hydrogen. In some embodiments, a compound of Formula (I) can be a compound of Formula (II) wherein R₂₀ is Cl, F or methyl optionally substituted with one or more F; R₃₀ is methyl optionally substituted with one or more F; and R₂₆ is hydrogen.

In some embodiments, a compound of Formula (I-A) or Formula (I-B) can be a compound of Formula (II) wherein W is an amide optionally substituted with lower alkyl, carboxyl or 5-member heteroaryl ring comprising at least one nitrogen, such as a pyrazole, imidazole, triazole or oxadiazole, each optionally substituted with lower alkyl. In some embodiments, R₁ is Cl, F or CN in Formula (II); and R₂₆ is hydrogen. In some embodiments, R₃ is methyl; and R₂₆ is hydrogen. In some embodiments, R₃ is hydrogen; and R₂₆ is hydrogen. In some embodiments, R₃ is F; and R₂₆ is hydrogen. In some embodiments, a compound can be a compound of Formula (II) wherein W is pyrazole, imidazole, triazole or oxadiazole, each optionally substituted with methyl; R₁ is Cl, F or CN; R₃ is hydrogen, methyl or F; and R₂₆ is hydrogen.

In some embodiments, a compound of Formula (I-A) or Formula (I-B) can be a compound of Formula (II) wherein the lower alkyl in R₁, R₃, and R₂₀ is methyl; and R₂₆ is hydrogen. In some embodiments, a compound of Formula (I) can be a compound of Formula (II) wherein R₁₀ is methyl; A is pyrazole, imidazole, triazole or oxadiazole each optionally substituted with methyl; and R₂₀ is methyl; and R₂₆ is hydrogen. In some embodiments, a compound of Formula (I) can be a compound of Formula (II) wherein W is pyrazole optionally substituted with one or more methyl; R₁ is the F; R₃ is H or F and R₂₀ is methyl; and R₂₆ is hydrogen. In some embodiments, a compound of Formula (I) can be a compound of Formula (II) wherein W is pyrazole optionally substituted with one or more methyl, R₁ is the F, R₃ is H and R₂₀ is methyl and R₂₆ is hydrogen.

In some embodiments, a compound of Formula (I-A) or Formula (I-B) can be a compound of Formula (II) wherein the lower alkyl in R₁, R₃, and R₂₀ is methyl; and R₂₆ is F. In some embodiments, a compound of Formula (I) can be a compound of Formula (II) wherein R₁₀ is methyl; A is pyrazole, imidazole, triazole or oxadiazole each optionally substituted with methyl; and R₂₀ is methyl; and R₂₆ is F. In some embodiments, a compound of Formula (I) can be a compound of Formula (II) wherein W is pyrazole optionally substituted with one or more methyl; R₁ is the F; R₃ is H or F and R₂₀ is methyl; and R₂₆ is F. In some embodiments, a compound of Formula (I) can be a compound of Formula (II) wherein W is pyrazole optionally substituted with one or more methyl, R₁ is the F, R₃ is H and R₂₀ is methyl and R₂₆ is F.

In compounds of Formula (II), W is a 5-member heteroaryl ring comprising at least one nitrogen, such as a pyrazole. In some embodiments, Riis Cl, F or CN. In some embodiments, R₃ is H. In some embodiments, R₃ is F. In some embodiments, the lower alkyl in each of Ra, R₆, R₁₀, R₃₀ and R_{b} can independently be methyl. In some embodiments, W is pyrazole optionally substituted with one or more methyl, R₁ is the F, R₃ is H or F and each of Ra, R₆, R₁₀, R₃₀ and Rb is methyl in a compound of Formula (II). In some embodiments, W is pyrazole optionally substituted with one or more methyl, R₁ is the F, R₃ is H and each of Ra, R₆, R₁₀, R₃₀ and Rb is methyl in a compound of Formula (II).

In some embodiments, the compound of Formula (II) is selected from the group consisting of:

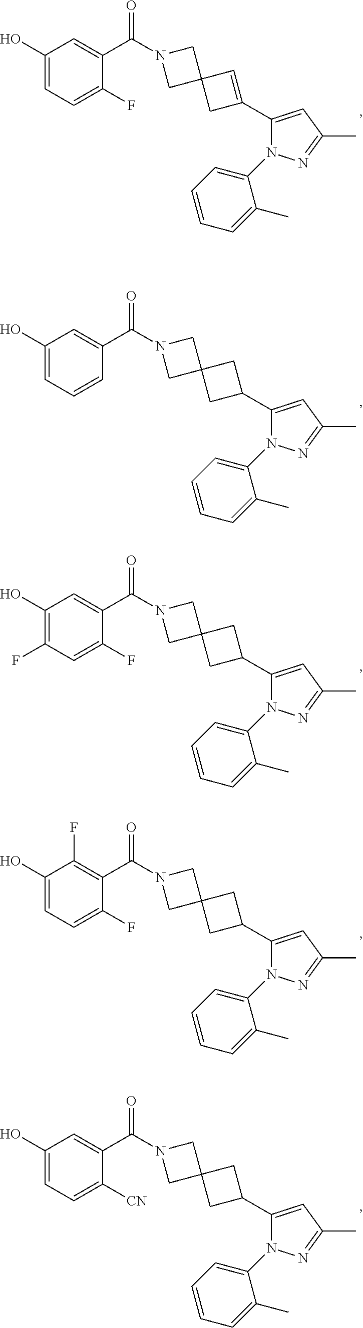 [SMILES: Cc1cc(C2=CC3(C2)CN(C(=O)c2cc(O)ccc2F)C3)n(-c2ccccc2C)n1 ; Cc1cc(C2CC3(C2)CN(C(=O)c2cccc(O)c2)C3)n(-c2ccccc2C)n1 ; Cc1cc(C2CC3(C2)CN(C(=O)c2cc(O)c(F)cc2F)C3)n(-c2ccccc2C)n1 ; Cc1cc(C2CC3(C2)CN(C(=O)c2c(F)ccc(O)c2F)C3)n(-c2ccccc2C)n1 ; Cc1cc(C2CC3(C2)CN(C(=O)c2cc(O)ccc2C#N)C3)n(-c2ccccc2C)n1]

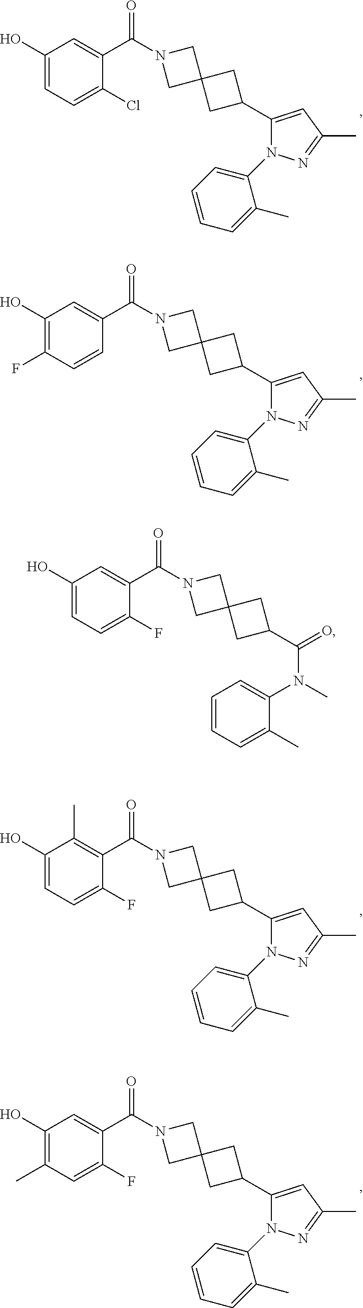 [SMILES: Cc1cc(C2CC3(C2)CN(C(=O)c2cc(O)ccc2Cl)C3)n(-c2ccccc2C)n1 ; Cc1cc(C2CC3(C2)CN(C(=O)c2ccc(F)c(O)c2)C3)n(-c2ccccc2C)n1 ; Cc1ccccc1N(C)C(=O)C1CC2(C1)CN(C(=O)c1cc(O)ccc1F)C2 ; Cc1cc(C2CC3(C2)CN(C(=O)c2c(F)ccc(O)c2C)C3)n(-c2ccccc2C)n1 ; Cc1cc(C2CC3(C2)CN(C(=O)c2cc(O)c(C)cc2F)C3)n(-c2ccccc2C)n1]

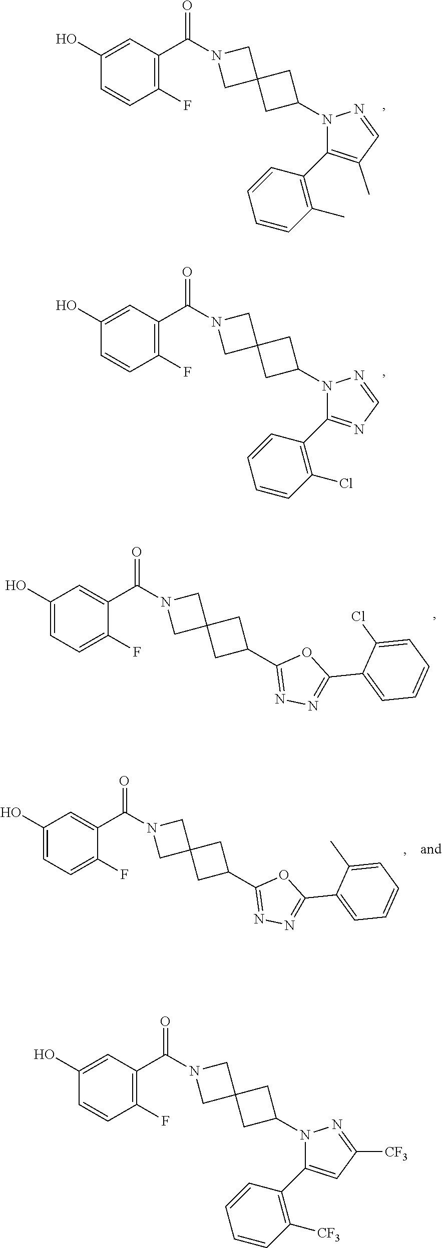 [SMILES: Cc1ccccc1-c1c(C)cnn1C1CC2(C1)CN(C(=O)c1cc(O)ccc1F)C2 ; O=C(c1cc(O)ccc1F)N1CC2(CC(n3ncnc3-c3ccccc3Cl)C2)C1 ; O=C(c1cc(O)ccc1F)N1CC2(CC(c3nnc(-c4ccccc4Cl)o3)C2)C1 ; Cc1ccccc1-c1nnc(C2CC3(C2)CN(C(=O)c2cc(O)ccc2F)C3)o1 ; O=C(c1cc(O)ccc1F)N1CC2(CC(n3nc(C(F)(F)F)cc3-c3ccccc3C(F)(F)F)C2)C1]

or a pharmaceutically acceptable salt thereof.

In some embodiments, the compound of Formula (II) is selected from the group consisting of:

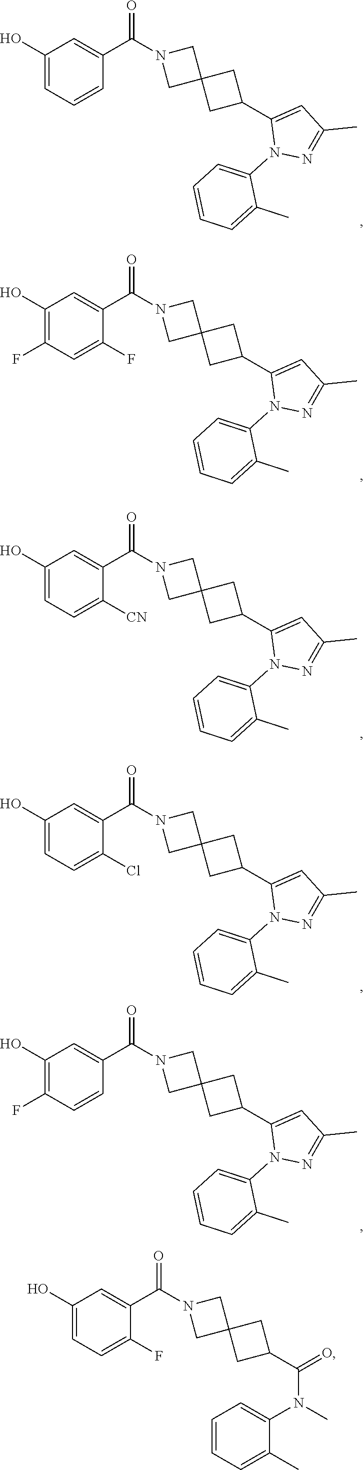 [SMILES: Cc1cc(C2CC3(C2)CN(C(=O)c2cccc(O)c2)C3)n(-c2ccccc2C)n1 ; Cc1cc(C2CC3(C2)CN(C(=O)c2cc(O)c(F)cc2F)C3)n(-c2ccccc2C)n1 ; Cc1cc(C2CC3(C2)CN(C(=O)c2cc(O)ccc2C#N)C3)n(-c2ccccc2C)n1 ; Cc1cc(C2CC3(C2)CN(C(=O)c2cc(O)ccc2Cl)C3)n(-c2ccccc2C)n1 ; Cc1cc(C2CC3(C2)CN(C(=O)c2ccc(F)c(O)c2)C3)n(-c2ccccc2C)n1 ; Cc1ccccc1N(C)C(=O)C1CC2(C1)CN(C(=O)c1cc(O)ccc1F)C2]

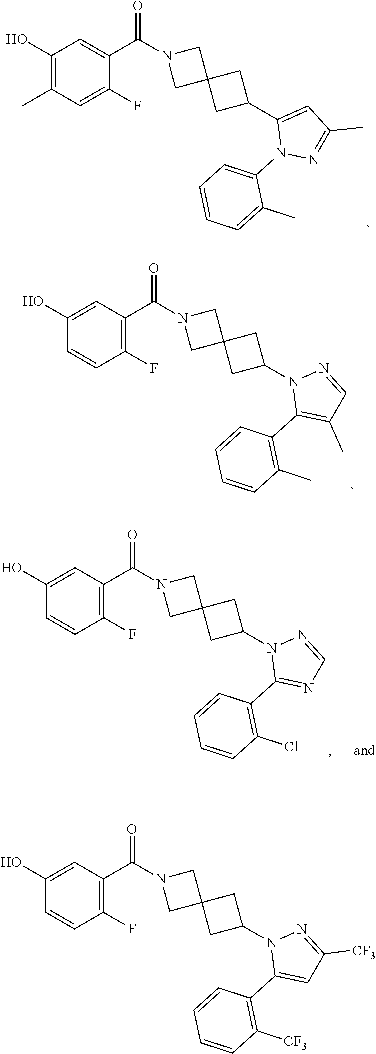 [SMILES: Cc1cc(C2CC3(C2)CN(C(=O)c2cc(O)c(C)cc2F)C3)n(-c2ccccc2C)n1 ; Cc1ccccc1-c1c(C)cnn1C1CC2(C1)CN(C(=O)c1cc(O)ccc1F)C2 ; O=C(c1cc(O)ccc1F)N1CC2(CC(n3ncnc3-c3ccccc3Cl)C2)C1 ; O=C(c1cc(O)ccc1F)N1CC2(CC(n3nc(C(F)(F)F)cc3-c3ccccc3C(F)(F)F)C2)C1]

or a pharmaceutically acceptable salt thereof.

### General Procedure for Acid Amine Coupling for Preparing Compounds of Formula (II)

To a stirred solution of 6-(3-methyl-1-(o-tolyl)-1H-pyrazol-5-yl)-2-azaspiro[3.3]heptane 2,2,2-trifluoroacetate (1 eq.) and 2-fluoro-benzoic acid (1.5 eq.) in DMF (10 V) at 0° C., was added DIPEA (5 eq.) and stirred for 15 min. To this reaction mixture T3P (50% solution in ethyl acetate) (1.5 eq.) was added. The resulting mixture was stirred at room temperature for 2 h. The reaction mixture was quenched by the addition of ice cold water (10 mL) and extracted by ethyl acetate (3×25 mL). The organic layer was dried over sodium sulphate and concentrated under reduced pressure. The crude material was purified by column chromatography using 5% MeOH in DCM.

TABLE 1

| Structure | Description |
|---|---|
| 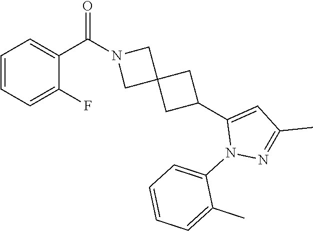 [SMILES: Cc1cc(C2CC3(C2)CN(C(=O)c2ccccc2F)C3)n(-c2ccccc2C)n1] | Compound 122 was prepared by followed the general procedure given above from 70 mg of 6-(3-methyl-1-(o-tolyl)-1H-pyrazol- 5-y1)-2-azaspiro[3.3]heptane 2,2,2-trifluoroacetate (1 eq.) to obtain (2-fluorophenyl)(6-(3-methyl-1-(o-tolyl)-1H-pyrazol-5- yl)-2-azaspiro[3.3]heptan-2-yl)methanone (20 mg, 28%) as a white solid. LCMS: 390.4 m/z [M + H]⁺; ¹H NMR (400 MHz, DMSO-d₆) δ 7.58-7.32 (m, 4H), 7.36-7.21 (m, 3H), 7.16 (t, J = 7.0 Hz, 1H), 6.14 (d, J = 22.8 Hz, 1H), 3.96 (dd, J = 23.5, 17.7 Hz, 4H), 3.00 (m, 1H), 2.39-2.21 (m, 4H), 2.18 (d, J = 10.8 Hz, 3H), 1.93 (s, 3H). |
| PSY-05-00122 | |
| 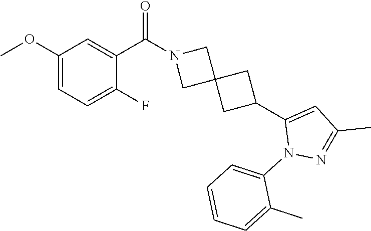 | Compound-123 was prepared by followed the general procedure given above from 70 mg of 6-(3-methyl-1-(o-tolyl)-1H-pyrazol- 5-y1)-2-azaspiro[3.3]heptane 2,2,2-trifluoroacetate (1 eq.) to obtain (2-fluoro-5-methoxyphenyl)(6-(3-methyl-1-(o-tolyl)-1H- pyrazol-5-yl)-2-azaspiro[3.3]heptan-2-yl)methanone (25 mg, 15%) as a white solid. LCMS: 420.1 m/z [M + H]⁺. ¹H NMR (400 MHz, Methanol-d₄) δ 7.48-7.29 (m, 3H), 7.20 (ddd, J = 7.3, 5.6, 1.3 Hz, 1H), 7.17-7.09 (m, IH), 7.09-7.01 (m, IH), 6.97 (dt, J = 5.1, 3.5 Hz, IH), 4.08 (dd, J = 22.9, 17.5 Hz, 4H), 3.80 (d, J = 4.8 Hz, 3H), 3.09 (dt, J = 24.3, 8.6 Hz, 1H), 2.48-2.31 (m, 4H), 2.28 (d, J = 9.8 Hz, 3H), 2.00 (d, J = 2.5 Hz, 3H). |
| PSY-05-00123 | |
| 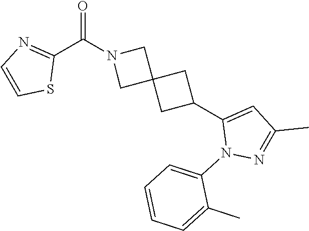 [SMILES: Cc1cc(C2CC3(C2)CN(C(=O)c2nccs2)C3)n(-c2ccccc2C)n1] | Compound-124 was prepared by followed the general procedure given above from 100 mg of 6-(3-methyl-1-(o-tolyl)-1H- pyrazol-5-yl)-2-azaspiro[3.3]heptane 2,2,2-trifluoroacetate (1 eq.) to obtain (6-(3-methyl-1-(o-tolyl)-1H-pyrazol-5-yl)-2- azaspiro[3.3]heptan-2-yl)(thiazol-2-yl)methanone) (25 mg, 25%) as a white solid. LCMS: 379.6 m/z [M + H]⁺; ¹H NMR (400 MHz, Methanol-d₄) δ 7.95 (dd, J = 10.9, 3.1 Hz, 1H), 7.79 (dd, J = 4.8, 3.1 Hz, 1H), 7.46-7.36 (m, 2H), 7.36-7.31 (m, 1H), 7.20 (dd, J = 7.8, 1.3 Hz, 1H), 6.21 (d, J = 2.1 Hz, 1H), 4.67 (d, J = 1.5 Hz, IH), 4.62 (d. J = 1.2 Hz, 1H), 4.15 (d, J = 1.5 Hz, IH), 4.12-4.08 (m, 1H), 3.11 (q, J = 8.5 Hz, IH), 2.49-2.41 (m, 2H), 2.40-2.33 (m, 2H), 2.28 (s, 3H), 2.00 (s, 3H). |
| PSY-05-00124 | |
| 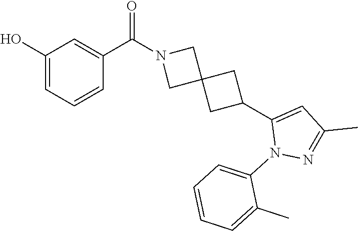 [SMILES: Cc1cc(C2CC3(C2)CN(C(=O)c2cccc(O)c2)C3)n(-c2ccccc2C)n1] | Compound-125 was prepared by followed the general procedure given above from 70 mg of 6-(3-methyl-1-(o-tolyl)-1H-pyrazol- 5-y1)-2-azaspiro[3.3]heptane 2,2,2-trifluoroacetate (1 eq.) to obtain (3-hydroxyphenyl)(6-(3-methyl-1-(o-tolyl)-1H-pyrazol- 5-yl)-2-azaspiro[3.3]heptan-2-yl)methanone)(15 mg, 21%) as a white solid. LCMS: 388.4 m/z [M + H]⁺; ¹H NMR (400 MHz, DMSO-d₆) δ 9.63 (d, J = 13.1 Hz, 1H), 7.44-7.26 (m, 3H), 7.17 (dd, J = 19.2, 8.3 Hz, 2H), 6.96 (d, J = 7.6 Hz, 2H), 6.86 (s, IH), 6.14 (d, J = 12.2 Hz, 1H), 4.19 (d, J = 25.2 Hz, 2H), 3.94 (d, J = 23.0 Hz, 2H), 3.03-2.94 (m, 1H), 2.33 (s, 3H), 2.27-2.12 (m, 5H), 1.93 (s, 3H). |
| PSY-05-00125 | |
| 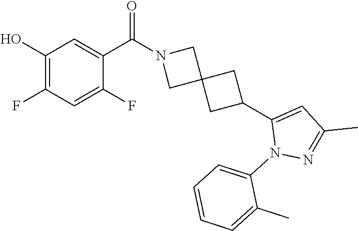 [SMILES: Cc1cc(C2CC3(C2)CN(C(=O)c2cc(O)c(F)cc2F)C3)n(-c2ccccc2C)n1] | Compound-126 was prepared by followed the general procedure given above from 100 mg of 6-(3-methyl-1-(o-tolyl)-1H- pyrazol-5-yl)-2-azaspiro[3.3]heptane 2,2,2-trifluoroacetate (1 eq.) to obtain (2,4-difluoro-5-hydroxyphenyl)(6-(3-methyl-1-(0- tolyl)-1H-pyrazol-5-yl)-2-azaspiro[3.3]heptan-2-yl)methanone (50 mg, 21%) as a white solid. LCMS: 424.4 m/z [M + H]⁺. ¹H NMR (400 MHz, Methanol-d₄) δ 7.34 (dt, J = 25.4, 8.9 Hz, 3H), 7.18 (t, J = 6.3 Hz, 1H), 7.07-6.91 (m, 2H), 6.18 (d, J = 18.4 Hz, IH), 4.05 (dd, J = 22.8, 7.5 Hz, 4H), 3.13-3.02 (m, 1H), 2.36 (td, ) = 16.6, 14.9, 9.8 Hz, 4H), 2.26 (d, J = 8.4 Hz, 3H), 1.98 (d, J = 2.3 Hz, 3H). |
| PSY-05-00126 | |
| 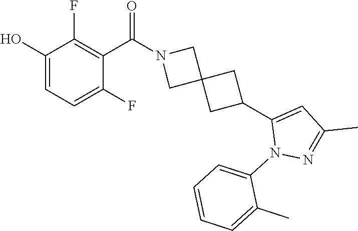 [SMILES: Cc1cc(C2CC3(C2)CN(C(=O)c2c(F)ccc(O)c2F)C3)n(-c2ccccc2C)n1] | Compound-00127 was prepared by followed the general procedure given above from 100 mg of 6-(3-methyl-1-(o-tolyl)- 1H-pyrazol-5-yl)-2-azaspiro[3.3]heptane 2,2,2-trifluoroacetate (1 eq.) to obtain (2,6-difluoro-3-hydroxyphenyl)(6-(3-methyl-1- (o-tolyl)-1H-pyrazol-5-yl)-2-azaspiro[3.3]heptan-2- yl)methanone) (20 mg, 18%) as a white solid. LCMS: 424.7 m/z [M + H]⁺; ¹H NMR (400 MHz, Methanol-d₄) δ 7.50-7.25 (m, 3H), 7.18 (dd, J = 7.6, 5.5 Hz, 1H), 6.91-6.81 (m, 1H), 6.18 (d, J = 21.0 Hz, 1H), 4.10 (d, J = 22.2 Hz, 2H), 3.96 (d, J = 23.3 Hz, 2H), 3.15-3.02 (m, 1H), 2.49-2.29 (m, 4H), 2.26 (d, J = 10.5 Hz, 3H), 1.98 (d, J = 2.8 Hz, 3H). |
| PSY-05-00127 | |

In some embodiments, the disclosure provides methods of using a compound of Formula (III) or a pharmaceutically acceptable salt thereof:

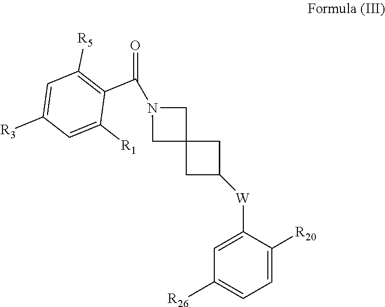

wherein
R₃ is halogen;
R₅ is —O—R₅₂;
R₅₂ is lower alkyl or cycloalkyl, each optionally substituted with halogen,
W is a 5-member heteroaryl ring optionally substituted with one or more R₃₀;
R₃₀ is lower alkyl;
R₂₀ is lower alkyl; and
R₂₆ is hydrogen or halogen.

In some embodiments, R₂₀ is methyl and R₂₆ is hydrogen or F in compounds of Formula (III). In some embodiments, R₂₀ is methyl and R₂₆ is hydrogen in compounds of Formula (III). In some embodiments, R₂₀ is methyl and R₂₆ is F.

In compounds of Formula (III), W is a 5-member heteroaryl ring comprising at least one nitrogen, such as a pyrazole. In some embodiments, R₃ is F. In some embodiments, R₆₂ is lower alkyl or cycloalkyl. In some embodiments, the lower alkyl in R₂₀, R₃₀ and R₆₂ can be methyl. In some embodiments, W is pyrazole optionally substituted with one or more methyl, and R₃ is the F. In some embodiments, W is pyrazole optionally substituted with one or more methyl, and R₃ is the F, R₆₂ is methyl, ethyl, propyl or cyclopropyl each optionally substituted with one or more F, and R₂₀ is methyl in a compound of Formula (III).

In some embodiments, the compound of Formula (III) is selected from the group consisting of:

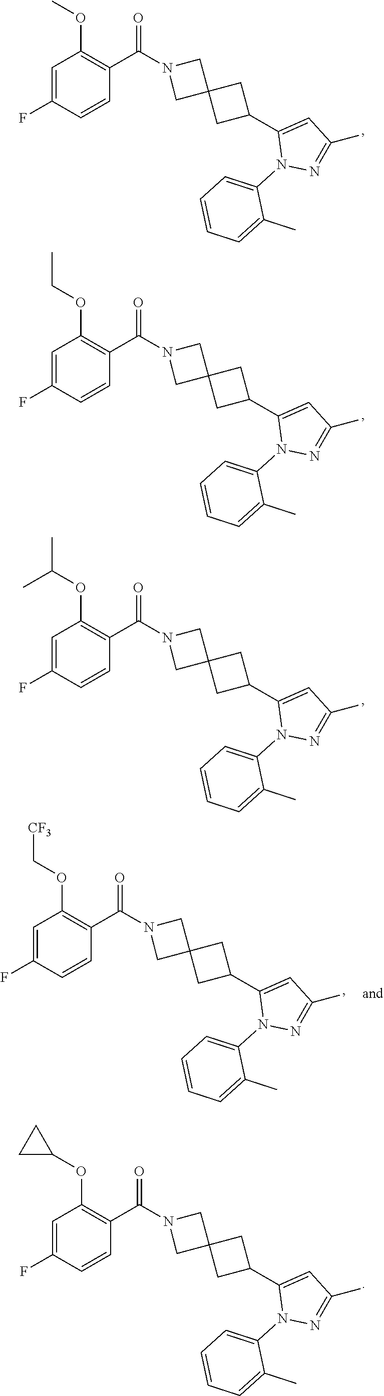

In some embodiments, the compound of Formula (III) is selected from the group consisting of:

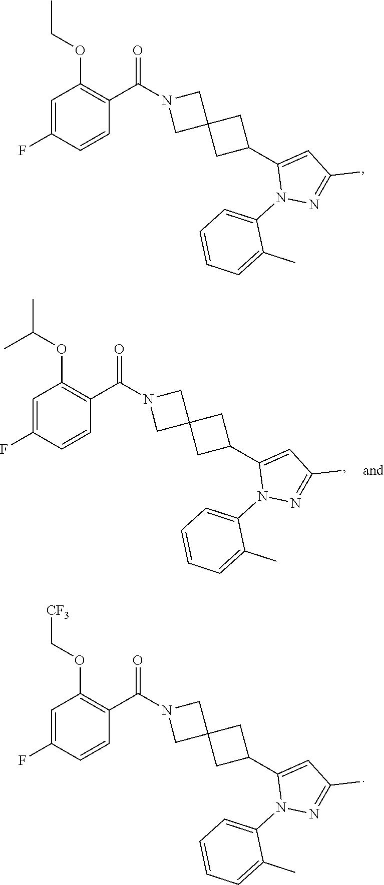

In some embodiments, methods comprise use of a compound selected from the group consisting of:

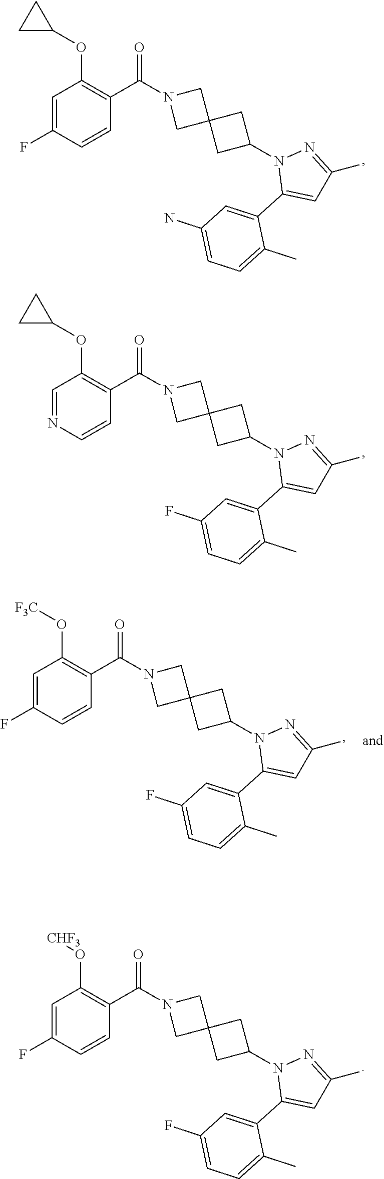

In some embodiments, the disclosure provides methods of using a compound of Formula (III) or a pharmaceutically acceptable salt thereof:

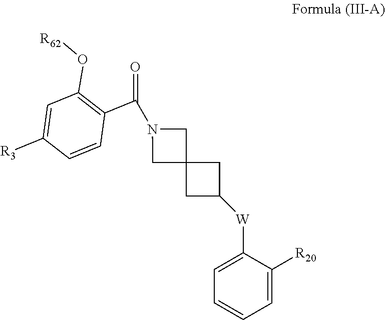

wherein
R₃ is halogen;
R₆₂ is lower alkyl or cycloalkyl, each optionally substituted with halogen,
W is a 5-member heteroaryl ring optionally substituted with one or more R₃₀;
R₃₀ is lower alkyl; and
R₂₀ is lower alkyl.

In compounds of Formula (III-a), W is a 5-member heteroaryl ring comprising at least one nitrogen, such as a pyrazole. In some embodiments, R₃ is F. In some embodiments, Roz is lower alkyl or cycloalkyl. In some embodiments, the lower alkyl in R₂₀, R₃₀ and R₆₂ can be methyl. In some embodiments, W is pyrazole optionally substituted with one or more methyl, and R₃ is the F. In some embodiments, W is pyrazole optionally substituted with one or more methyl, and R₃ is the F, R₆₂ is methyl, ethyl, propyl or cyclopropyl each optionally substituted with one or more F, and R₂₀ is methyl in a compound of Formula (III-A).

### General Procedure for Acid Amine Coupling for Preparing Compounds of Formula (III)

To a stirred solution of 6-(3-methyl-1-(o-tolyl)-1H-pyrazol-5-yl)-2-azaspiro[3.3] heptane 2,2,2-trifluoroacetate (1 eq.) and 4-fluoro-2-methoxybenzoic acid (1.2 eq.) in DMF (10 V) at 0° C., was added DIPEA (3 eq.) and stirred for 15 min. To this reaction mixture T3P (50% solution in ethyl acetate) (1.3 eq.) was added. The resulting mixture was stirred at room temperature for 2 h. The reaction mixture was quenched by the addition of ice cold water (10 mL) and extracted by ethyl acetate (3×25 mL). The organic layer was dried over sodium sulphate and concentrated under reduced pressure. The crude material was purified by column chromatography using 5% MeOH in DCM.

TABLE 2

| Structure | Description |
|---|---|
| 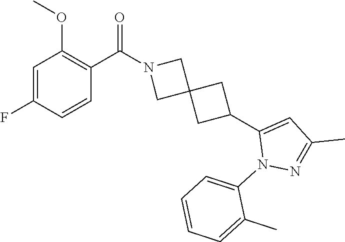 [SMILES: COc1cc(F)ccc1C(=O)N1CC2(CC(c3cc(C)nn3-c3ccccc3C)C2)C1] | Compound 00142-001 was prepared by followed the general procedure given above from 120 mg of 6-(3-methyl-1-(o-tolyl)- 1H-pyrazol-5-yl)-2-azaspiro [3.3]heptane 2,2,2-trifluoroacetate (1 eq.) to obtain (4-fluoro-2-methoxyphenyl)(6-(3-methyl-1-(o- tolyl)-1H-pyrazol-5-yl)-2-azaspiro[3.3] heptan-2-yl)methanone (35 mg, 19.44%) as a white solid. LCMS: 420.41 m/z [M + H]+; 1H NMR (400 MHz, DMSO-d6) δ 7.44-7.25 (m, 4H), 7.16 (t, J = 6.5 Hz, 1H), 6.98 (s, 1H), 6.85-6.74 (m, 1H), 6.17 (s, 1H), 3.95 (s, 1H), 3.89 (s, 1H), 3.86-3.75 (m, 5H), 2.99 (dt, J = 25.9, 8.5 Hz, 1H), 2.26-2.43 (m, 3H), 2.26-2.14 (m, 4H), 1.94 (d. J = 2.2 Hz, 3H). |
| PSY-05-00142-001 | |
| 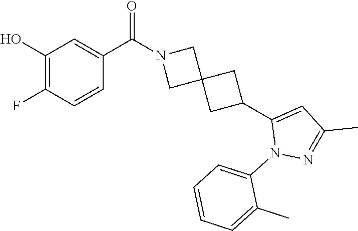 [SMILES: Cc1cc(C2CC3(C2)CN(C(=O)c2ccc(F)c(O)c2)C3)n(-c2ccccc2C)n1] | Compound-00141-001 was prepared by followed the general procedure given above from 120 mg of 6-(3-methyl-1-(o-tolyl)- IH-pyrazol-5-y1)-2-azaspiro [3.3] heptane 2,2,2-trifluoroacetate (1 eq.) to obtain (4-fluoro-3-hydroxyphenyl)(6-(3-methyl-1-(o- tolyl)-1H-pyrazol-5-yl)-2-azaspiro[3.3]heptan-2-yl)methanone (19 mg, 100.49%) as a white solid. LCMS: 406.3 m/z [M + H]+; 1H NMR (400 MHz, DMSO-d6) δ 10.16 (s, 1H), 7.38 (s, 2H), 7.30 (d, J = 5.9 Hz, IH), 7.18 (dd, J = 13.5, 8.4 Hz, 3H), 7.02 (s, 1H), 6.15 (d, J = 10.7 Hz, IH), 4.25 (s, 1H), 4.19 (s, 1H), 3.97 (s, IH), 3.92 (s, IH), 3.04-2.94 (m, 1H), 2.23 (d, J = 9.5 Hz, 2H), 2.19 (s, 5H), 1.93 (s, 3H). |
| PSY-05-00141-001 | |
| 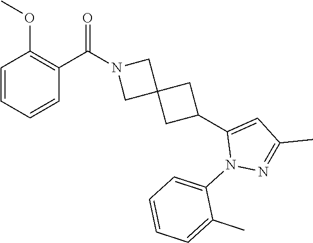 [SMILES: COc1ccccc1C(=O)N1CC2(CC(c3cc(C)nn3-c3ccccc3C)C2)C1] | Compound-00143-001 was prepared by followed the general procedure given above from 80 mg of 6-(3-methyl-1-(o-tolyl)- 1H-pyrazol-5-yl)-2-azaspiro[3.3]heptane 2,2,2-trifluoroacetate (1 eq.) to obtain (2-methoxyphenyl)(6-(3-methyl-1-(o-tolyl)-1H- pyrazol-5-yl)-2-azaspiro[3.3] heptan-2-yl)methanone (25 mg, 20.83%) as a white solid. LCMS: 401.8 m/z [M]+. 1H NMR (400 MHz, Methanol-d4) δ 7.50-7.17 (m, 5H), 7.17-6.96 (m, 3H), 6.23-6.17 (s, IH), 4.11 (s, 1H), 4.05 (s, 1H), 3.97 (s, 1H), 3.93- 3.84 (m, 4H), 3.08 (dq, J = 29.0, 8.5 Hz, IH), 2.48-2.35 (m, 3H), 2.32 (dd, J = 27.0, 12.1 Hz, 4H), 2.01 (d, J = 3.1 Hz, 3H). |
| PSY-05-00143-001 | |
| 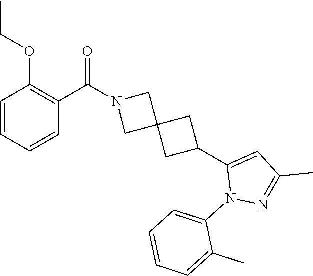 [SMILES: CCOc1ccccc1C(=O)N1CC2(CC(c3cc(C)nn3-c3ccccc3C)C2)C1] | Compound-00145-001 was prepared by followed the general procedure given above from 80 mg of 6-(3-methyl-1-(o-tolyl)- 1H-pyrazol-5-y1)-2-azaspiro[3.3]heptane 2,2,2-trifluoroacetate (1 eq.) to obtain (2-ethoxyphenyl)(6-(3-methyl-1-(o-tolyl)-1H- pyrazol-5-yl)-2-azaspiro[3.3]heptan-2-yl)methanone (30 mg, 24.19%) as a white solid. LCMS: 416.74 m/z [M + H]+; 1H NMR (400 MHz, DMSO-d6) δ 7.45-7.24 (m, 3H), 7.20 (d, J = 7.5 Hz, 1H), 7.14 (q, J = 8.1, 7.2 Hz, IH), 7.08 - 6.89 (m, 2H), 6.17- 6.07 (s, 1H), 4.06 (dq, J = 9.9, 6.9 Hz, 2H), 3.94 (s, IH), 3.88 (s, 1H), 3.83 (s, 1H), 3.77 (s, 1H), 2.98 (dq, J = 25.0, 8.4 Hz, IH), 2.39-2.14 (m, 7H), 1.93 (d, J = 1.9 Hz, 3H), 1.30 (dt, J = 9.8, 6.9 Hz, 3H). |
| PSY-05-00145-001 | |

In some embodiments, V in Formula (I-A) is

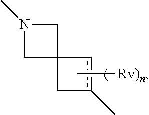

where n and each Rv is as defined above, and the dashed line represents an optional double bond. In some embodiments, in Formula (I-B) the dashed line represents an optional double bond. In some embodiments, in Formula (I-B-1) the dashed line represents an optional double bond. In some embodiments, in Formula (I-B-2) the dashed line represents an optional double bond. In some embodiments, in Formula (II) the dashed line represents an optional double bond.

The compounds described herein can exist as salts, such as with pharmaceutically acceptable acids. Accordingly, such salts of the compounds described herein are included. The term “pharmaceutically acceptable salt” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

### Pharmaceutical Compositions

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. The pharmaceutical composition can be a medicament.

Pharmaceutically acceptable carriers include those known in the art. The choice of a pharmaceutically acceptable carrier can depend, for example, on the desired route of administration of the composition. A pharmaceutical composition (preparation) can be administered to a subject by any of a number of routes of administration including, for example, parenteral administration (e.g. intravenously, subcutaneously, or intramuscularly), oral administration (for example, tablets, and capsules); absorption through the oral mucosa (e.g., sublingually) or transdermally (for example as a patch applied to the skin) or topically (for example, as a cream, ointment or spray applied to the skin).

In some embodiments, pharmaceutical compositions comprising compounds of Formula (I-A), Formula (I-B), Formula (I-B-1), Formula (I-B-2), Formula (I-C), Formula (II), Formula (III) or pharmaceutically acceptable salts thereof can be formulated for oral administration. For example, a compound provided herein can be combined with suitable compendial excipients to form an oral unit dosage form, such as a capsule or tablet, containing a target dose of a compound of Formula (I-A), Formula (I-B), Formula (I-B-1), Formula (I-B-2), Formula (II), Formula (III). The drug product can be prepared by first manufacturing the compound of Formula (I-A), Formula (I-B), Formula (I-B-1), Formula (I-B-2), Formula (I-C), Formula (II), Formula (III) as an active pharmaceutical ingredient (API), followed by roller compaction/milling with intragranular excipients and blending with extra granular excipients. A Drug Product can contain the selected compound of Formula (I-A), Formula (I-B), Formula (I-B-1), Formula (I-B-2), Formula (I-C), Formula (II), Formula (III) as the API and excipient components in a tablet in a desired dosage strength of Compound 1. The blended material can be compressed to form tablets and then film coated. The excipients can be selected from materials appropriate for inclusion in a pharmaceutical composition for an intended purpose and route of delivery including providing a desired manufacturing and stability properties and/or desired in vivo characteristics or other properties to the pharmaceutical composition. In some embodiments, the pharmaceutical composition can include a compound of Formula (I-A), Formula (I-B), Formula (I-B-1), Formula (I-B-2), Formula (I-C), Formula (II), Formula (III) as the API in combination with a filler (e.g., a form of microcrystalline cellulose), a dry binder or disintegrant (e.g., a cross-linked polymer), a glidant (e.g., colloidal silicon dioxide) and/or a lubricant (e.g., magnesium stearate). In some embodiments, the pharmaceutical composition can comprise a material such as an extended release or disintegrant involved in carrying or transporting the API pharmaceutical agent from one organ, or portion of the body, to another organ, or portion of the body of a subject, including materials to desirable control the absorption of the API in the intestine.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

To prepare solid dosage forms for oral administration, the active ingredient is mixed with one or more pharmaceutically acceptable carriers, such as sodium citrate or dicalcium phosphate, and/or any of the following: (1) fillers or extenders, (2) binders, (3) humectants, (4) disintegrating agents, (5) solution retarding agents, (6) absorption accelerators, (7) wetting agents, (8) absorbents, (9) lubricants, (10) complexing agents, and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using suitable excipients. The pharmaceutical compositions according to the present invention may contain conventional pharmaceutical carriers and/or auxiliary agents. In some embodiments, the pharmaceutical compositions according to the present invention may contain conventional carrier agents including a binder, a lubricant and/or a glidant selected from those products and materials generally used in pharmaceutical industry for preparation of pharmaceutical compositions for an intended route of administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

Liquid dosage forms useful for oral administration include pharmaceutically acceptable carriers and the active ingredient provided as a solid form for reconstitution prior to administration or as a liquid (e.g., solutions, suspensions, or emulsions). In addition to the active ingredient, a liquid dosage forms may contain inert diluents commonly used in the art. For example, formulations of pharmaceutically acceptable compositions for injection can include aqueous solutions such as water or physiologically buffered saline or other solvents or vehicles suitable for the intended route of administration. In some embodiments, the pharmaceutical composition is formulated for parenteral administration.

The therapeutically effective amount of a pharmaceutical composition can be determined by human clinical trials to determine the safe and effective dose for a patient with a relevant diagnosis. It is generally understood that the effective amount of the compound may vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the pharmaceutical composition at a dose and dose interval determined to be safe and effective for the patient.

The present disclosure includes the use of pharmaceutically acceptable salts of compounds of the invention in the compositions and methods of the present invention. Pharmaceutically-acceptable salts include, for example, acid-addition salts and base-addition salts. The acid that is added to a compound to form an acid-addition salt can be an organic acid or an inorganic acid. A base that is added to a compound to form a base-addition salt can be an organic base or an inorganic base. In some embodiments, a pharmaceutically-acceptable salt is a metal salt, in some embodiments, a pharmaceutically-acceptable salt is an ammonium salt. For example, a pharmaceutically acceptable acid addition salt can exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

### Methods of Use

The inventors have discovered inter alia compounds of Formula (I-A), Formula (I-B), Formula (II) and Formula (III) can modulate, e.g., inhibit monoacylglycerol lipase. Accordingly, in another aspect, the disclosure provides a method for inhibiting monoacylglycerol lipase (MAGL). Generally, the method comprises administering a compound of Formula (I-A), Formula (I-B), Formula (II) and Formula (III) to a cell, e.g., a cell expressing MAGL. Without limitations, administering to the cell can be in vitro or in vivo. For example, an effective amount of a compound of Formula (I-A), Formula (I-B), Formula (II) and Formula (III) can be administered to a subject for inhibiting MAGL.

In some embodiments, methods of treatment are provided. In some embodiments, a method of treating management of anxiety disorders or for the short-term relief of the symptoms of anxiety or anxiety associated with depressive symptoms comprises administering a therapeutically effective amount of a compound that is a Reversible MAGL Inhibitor and a Selective MAGL Inhibitor to a patient in need thereof. In some embodiments, methods of treatment are provided. In some embodiments, a method of treating management of anxiety disorders or for the short-term relief of the symptoms of anxiety or anxiety associated with depressive symptoms comprises administering a therapeutically effective amount of a compound of Formula (I-A) to a patient in need thereof.

In some embodiments, methods of treatment are provided. In some embodiments, methods of treatment comprise the administration of a reversible MAGL inhibitor compound for the treatment of anxiety or stress and trauma related disorders.

Anxiety is generally defined as an emotional state characterized by maladaptive and excessive emotional responsiveness to potentially dangerous circumstances. The pathological expression of anxiety leads to enduring emotional perturbations with a consistent apprehension towards the possibility of future, vaguely defined negative events (See Nutt D, Allgulander C, Lecrubier Y, Peters T, Wittchen U. Establishing non-inferiority in treatment trials in psychiatry: guidelines from an Expert Consensus Meeting. J Psychopharmacol. 2008; 22 (4): 409-16). According to the current classification of anxiety disorders in the fourth edition of the Diagnostic and Statistical Manual of Mental Disorders (DSM-IV) (Sugiura T, Waku K. 2-Arachidonoylglycerol and the cannabinoid receptors. Chem Phys Lipids. 2000; 108 (1-2): 89-106), the main diagnostic entities in this category are:
generalized anxiety disorder (GAD), featuring general irritability, anxiety attacks, chronic apprehension/anxious expectation and secondary phobic avoidance.
panic disorder, characterized by brief (2-10 min) spells of overwhelming anxiety or fear, accompanied by somatic and cognitive symptoms;
social anxiety disorder (or social phobia), defined as extreme agitation in social contexts and avoidance of social situations;
obsessive-compulsive disorder (OCD), characterized by recurrent and intrusive anxiogenic thoughts (obsessions), and stereotyped behaviors (compulsions) aimed at the reduction of the distress caused by the obsessions.
post-traumatic stress disorder (PTSD), in which a prior intense trauma results in a long-lasting anxious response, with re-experiencing/flashback phenomena, avoidance and emotional numbing

(Tambaro S, Bortolato M. Cannabinoid-related agents in the treatment of anxiety disorders: current knowledge and future perspectives. Recent Pat CNS Drug Discov. 2012; 7 (1): 25-40. doi: 10.2174/157488912798842269).

Anxiety symptoms are highly prevalent in Posttraumatic stress disorder (PTSD), and up until the latest version of the DSM, when it was moved to its own category of trauma- and stress-related disorder, PTSD was categorized as an anxiety disorder (Hill M N, Campolongo P, Yehuda R, Patel S. Integrating Endocannabinoid Signaling and Cannabinoids into the Biology and Treatment of Posttraumatic Stress Disorder. Neuropsychopharmacology. 2018; 43 (1): 80-102. doi: 10.1038/npp.2017.162). PTSD, while once characterized as a variant of an anxiety disorder, is now explicitly viewed as a separate entity and categorized as a trauma- or stressor-related disorder (APA Diagnostic and Statistical Manualof Mental Disorders, 5th edn. American Psychiatric Association: Washington, DC, USA, 2013). PTSD represents a pathological condition that emerges, sometimes after a period of incubation, following either direct or indirect exposure to a trauma. In addition to trauma exposure, a diagnosis to PTSD requires presence of symptoms in four distinct clusters; intrusion, avoidance, arousal/reactivity, and negative cognitions/mood (Yehuda R, Hoge C W, McFarlane A C, Vermetten E, Lan ius RA, Nievergelt C M et al (2015. b). Post-traumatic stress disorder. Nat Rev Dis Primers 1:15057).

In some embodiments, a method of management of anxiety disorders or for the short-term relief of the symptoms of anxiety or anxiety associated with depressive symptoms comprises administering a therapeutically effective amount of a compound that is a Reversible MAGL Inhibitor and a Selective MAGL Inhibitor to a patient in need thereof. In some embodiments, methods of treatment are provided. In some embodiments, a method of treating management of anxiety disorders or for the short-term relief of the symptoms of anxiety or anxiety associated with depressive symptoms comprises administering a therapeutically effective amount of a compound of Formula (I-A) to a patient in need thereof.

In some embodiments, a method of treating a patient diagnosed with a disorder are provided, the method comprising administering a therapeutically effective amount of a compound that is a Reversible MAGL Inhibitor and a Selective MAGL Inhibitor, wherein disorder is selected from the group consisting of: Major Depressive Disorder (MDD), Obsessive Compulsive Disorder (OCD), Panic Disorder (PD), Social Anxiety Disorder (SAD), Generalized Anxiety Disorder (GAD), Posttraumatic Stress Disorder (PTSD). In some embodiments, a method of treating a patient diagnosed with a disorder are provided, the method comprising administering a therapeutically effective amount of a compound that is a Reversible MAGL Inhibitor and a Selective MAGL Inhibitor, wherein disorder is selected from the group consisting of: Major depressive disorder (MDD), Obsessive-compulsive disorder (OCD), Panic disorder (PD), Post-traumatic stress disorder (PTSD), Social anxiety disorder (SAD), and Premenstrual dysphoric disorder (PMDD). In some embodiments, a method of treating a patient diagnosed with a disorder are provided, the method comprising administering a therapeutically effective amount of a compound that is a Reversible MAGL Inhibitor and a Selective MAGL Inhibitor, wherein disorder is selected from the group consisting of: Major depressive disorder (MDD), Obsessive-compulsive disorder (OCD), Panic disorder (PD), Acute and maintenance treatment of Major Depressive Disorder (MDD), Acute and maintenance treatment of Obsessive Compulsive Disorder (OCD), Acute and maintenance treatment of Bulimia Nervosa, and Acute treatment of Panic Disorder, with or without agoraphobia.

In some embodiments, a method of treating MDD comprises administering a therapeutically effective amount of a compound of Formula (I-A) to a patient in need thereof. In some embodiments, a method of treating OCD comprises administering a therapeutically effective amount of a compound of Formula (I-A) to a patient in need thereof. In some embodiments, a method of treating PD comprises administering a therapeutically effective amount of a compound of Formula (I-A) to a patient in need thereof. In some embodiments, a method of treating SAD comprises administering a therapeutically effective amount of a compound of Formula (I-A) to a patient in need thereof. In some embodiments, a method of treating GAD comprises administering a therapeutically effective amount of a compound of Formula (I-A) to a patient in need thereof. In some embodiments, a method of treating PTSD comprises administering a therapeutically effective amount of a compound of Formula (I-A) to a patient in need thereof. In some embodiments, a method of treating PMDD comprises administering a therapeutically effective amount of a compound of Formula (I-A) to a patient in need thereof.

In some embodiments, a compound provided herein can be administered to the cell, e.g. cell expressing MAGL in vitro or ex vivo. As used herein, administering the compound to the cell means contacting the cell with the compound so that the compound is taken up by the cell. Generally, the cell can be contacted with the compound in a cell culture e.g., in vitro or ex vivo, or the compound can be administrated to a subject, e.g., in vivo. The term “contacting” or “contact” as used herein in connection with contacting a cell includes subjecting the cells to an appropriate culture media, which comprises a compound of Formula (I-A), Formula (I-B), Formula (I-B-1), Formula (I-B-2), Formula (II), Formula (III). Where the cell is in vivo, “contacting” or “contact” includes administering the compound, e.g., in a pharmaceutical composition to a subject via an appropriate administration route such that the compound contacts the cell in vivo.

As described herein, the compound of Formula (I-A), Formula (I-B), Formula (I-B-1), Formula (I-B-2), Formula (I-C), Formula (II), Formula (III) can be administered to a cell in vivo for modulating MAGL, e.g., inhibiting MAGL. Accordingly, in some embodiments, a therapeutically effective amount of a compound of Formula (I-A), Formula (I-B), Formula (I-B-1), Formula (I-B-2), Formula (I-C), Formula (II), Formula (III) can be administered to a subject for inhibiting monoacylglycerol lipase. For example, a therapeutically effective amount of a compound of Formula (I-A), Formula (I-B), Formula (I-B-1), Formula (I-B-2), Formula (I-C), Formula (II), Formula (III) can be administrated to a subject for treating a monoglycerol lipase mediated disease or disorder. By a MAGL-mediated disease or disorder is meant a disease or disorder wherein activity of MAGL is a cause of the disease or disorder. (See, e.g., Zanfirescu (Molecules 2021), Deng (Acta Pharm Sinica B, 2020), and Mulvihill (NIH Life Sci 2013)).

In some embodiments, a method of treating anxiety, PTSD and related conditions comprises administering a therapeutically effective amount of a reversible MAGL inhibitor to a patient in need thereof. In some embodiments, a method of treating General Anxiety Disorder (GAD) comprises administering a therapeutically effective amount of a reversible MAGL inhibitor to a patient in need thereof. In some embodiments, a method of treating anxiety, PTSD and related conditions comprises administering a therapeutically effective amount of a compound of Formula (I-A) to a patient in need thereof.

In some embodiments, the disclosure provides methods of using including methods of treatment comprising the use of certain compounds that are both a Selective MAGL Inhibitor Compound and a Reversible MAGL Inhibitor Compound as defined herein, or a pharmaceutically acceptable salt thereof.

A subject can be one who has been previously diagnosed with or identified as suffering from or having a condition in need of treatment a MAGL-mediated disease or disorder or one or more complications related to such a condition, and optionally, have already undergone treatment for such a disease or disorder. Alternatively, a subject can also be one who has not been previously diagnosed as having a MAGL-mediated disease or disorder or one or more complications related to such a disease or disorder. A “subject in need” of treatment for a particular condition can be a subject having that condition, diagnosed as having that condition, or at risk of developing that condition.

In one embodiment, the subject is human. In another embodiment, the subject is an experimental animal or animal substitute as a disease model.

It is noted that the terms “administered” and “subjected” are used interchangeably in the context of treatment of a disease or disorder. In jurisdictions that forbid the patenting of methods that are practiced on the human body, the meaning of “administering” of a composition to a human subject shall be restricted to prescribing a controlled substance that a human subject will be administer to the subject by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, administration will generally be local rather than systemic.

In preferred embodiments, the compositions are orally administered. Without limitations, oral administration can be in the form of solutions, suspensions, tablets, pills, capsules, sustained-release formulations, oral rinses, powders and the like.

The phrase “therapeutically-effective amount” as used herein means that amount of a compound, material, or composition comprising a compound described herein which is effective for producing some desired therapeutic effect in at least a sub-population of cells, e.g., modulate or inhibit activity of MAGL in a subject at a reasonable benefit/risk ratio applicable to any medical treatment. Thus, “therapeutically effective amount” means that amount which, when administered to a subject for treating a disease, is sufficient to affect such treatment for the disease.

Depending on the route of administration, effective doses can be calculated according to the body weight, body surface area, or organ size of the subject to be treated. Optimization of the appropriate dosages can readily be made by one skilled in the art in light of pharmacokinetic data observed in human clinical trials. Alternatively, or additionally, the dosage to be administered can be determined from studies using animal models for the particular type of condition to be treated, and/or from animal or human data obtained from agents which are known to exhibit similar pharmacological activities. The final dosage regimen will be determined by the attending surgeon or physician, considering various factors which modify the action of active agent, e.g., the agent's specific activity, the agent's specific half-life in vivo, the severity of the condition and the responsiveness of the patient, the age, condition, body weight, sex and diet of the patient, the severity of any present infection, time of administration, the use (or not) of other concomitant therapies, and other clinical factors.

The data obtained from the cell culture assays and animal studies can be used in formulating a range of dosage for use in humans. The dosage of such compounds lies preferably within a range of circulating concentrations that include the IC₅₀ with little or no toxicity. The dosage may vary within this range depending upon the dosage form employed and the route of use or administration utilized. The effective dose can be estimated initially from cell culture assays. A dose can be formulated in animal models to achieve a circulating plasma concentration range that includes the IC₅₀ (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay.

It will be appreciated that methods of treatment of the present invention can be employed in combination with additional therapies. For example, a treatment according to the present disclosure can be co-administered with one or more desired therapeutics or medical procedures for treating a MAGL-mediated disease or disorder.

### Definitions

Unless otherwise defined herein, scientific and technical terms used in this application shall have the meanings that are commonly understood by those of ordinary skill in the art.

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein, the compound designation terms “Compound #”, “PSY-#” and “PSY-05-#” (where # indicates any number having one or more digits) are synonymous with each other, unless otherwise indicated (e.g., “Compound 1” refers to a compound alternatively designated as “PSY-05-0001” or “PSY-1”).

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

As used herein, the term “Selective MAGL Inhibitor Compound” refers to a compound that selectively inhibits MAGL with an IC₅₀ that is at least 10 times the IC₅₀ for its inhibition of fatty acid amide hydrolase (FAAH), and that has an IC₅₀ of 100 nM or less (according to the MAGL Potency assay of Example 18 and the FAAH potency assay of Example 15).

As used herein, the term “Reversible MAGL Inhibitor Compound” the percent inhibition after dilution to the IC₅₀ concentration is 50+15% in the assay described for “determining MAGL reversible inhibition” section of Example 17 below.

As used herein, the term “Reversible Selective MAGL Inhibitor Compound” refers to a compound that is both a Selective MAGL Inhibitor Compound and a Reversible MAGL Inhibitor Compound, or a pharmaceutically acceptable salt thereof.

As used herein, the term “alkyl” refers to an aliphatic hydrocarbon group which can be straight or branched having 1 to about 10 carbon atoms in the chain, and which preferably have about 1 to about 6 carbons in the chain. “Lower alkyl” refers to an alkyl group having 1 to about 4 carbon atoms. “Higher alkyl” refers to an alkyl group having about 5 to about 10 carbon atoms. The alkyl group can be optionally substituted with one or more alkyl group substituents which can be the same or different, where “alkyl group substituent” includes halo, amino, aryl, hydroxy, alkoxy, aryloxy, alkyloxy, alkylthio, arylthio, aralkyloxy, aralkylthio, carboxy, alkoxycarbonyl, oxo and cycloalkyl. “Branched” refers to an alkyl group in which a lower alkyl group, such as methyl, ethyl or propyl, is attached to a linear alkyl chain. Exemplary alkyl groups include methyl, ethyl, i-propyl, n-butyl, t-butyl, n-pentyl, heptyl, octyl, decyl, dodecyl, tridecyl, tetradecyl, pentadecyl and hexadecyl. Useful alkyl groups include branched or straight chain alkyl groups of 6 to 50 carbon, and also include the lower alkyl groups of 1 to about 4 carbons and the higher alkyl groups of about 12 to about 16 carbons.

As used herein, the term “cycloalkyl” refers to a non-aromatic mono- or multicyclic ring system of about 3 to about 12 carbon atoms. Representative monocyclic cycloalkyl rings include cyclopropyl, cyclobutyl, and cyclohexyl. Useful multicyclic cycloalkyl rings include adamantyl. “Lower cycloalkyl” refers to an alkyl group having 3 to about 6 carbon atoms in the cycloalkyl ring, optionally substituted with halogen, alkyl, alkoxy or other substituents disclosed herein. “Higher alkyl” refers to an alkyl group having about 5 to about 10 carbon atoms.

“Aryl” refers to an aromatic carbocyclic radical containing about 3 to about 10 carbon atoms. The aryl group can be optionally substituted with one or more substituents, which can be the same or different, where “aryl group substituent” includes alkyl, alkenyl, alkynyl, hydroxy, alkoxy, carboxy, halo, nitro, trihalomethyl, cyano, alkoxycarbonyl, aryloxycarbonyl, aralkoxycarbonyl, acyloxy, aroylamino, acylamino, carbamoyl, alkylcarbamoyl, dialkylcarbamoyl, arylthio, alkylthio, and alkylene. Exemplary aryl groups include substituted or unsubstituted phenyl.

“Heterocyclyl” refers to a nonaromatic 3-8 membered monocyclic, or 8-12 membered bicyclic ring systems having 1-3 heteroatoms if monocyclic, or 1-6 heteroatoms if bicyclic, said heteroatoms selected from O, N, or S (e.g., carbon atoms and 1-3, or 1-6 heteroatoms of N, O, or S if monocyclic, or bicyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like.

“Heteroaryl” refers to an aromatic 3-8 membered monocyclic, or 8-12 membered fused bicyclic ring system having 1-3 heteroatoms if monocyclic, or 1-6 heteroatoms if bicyclic, said heteroatoms selected from O, N, or S (e.g., carbon atoms and 1-3, 1-6, or 1-9 heteroatoms of N, O, or S if monocyclic, bicyclic, or tricyclic, respectively.

Exemplary aryls and heteroaryls include, but are not limited to, phenyl, pyridinyl, pyrimidinyl, furanyl, thienyl, imidazolyl, thiazolyl, pyrazolyl, pyridazinyl, pyrazinyl, triazinyl, tetrazolyl, indolyl, benzyl, naphthyl, anthracenyl, azulenyl, fluorenyl, indanyl, indenyl, naphthyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl or xanthenyl, each of which can be optionally substituted.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application.

The term “haloalkyl” as used herein refers to an alkyl structure with at least one substituent of fluorine, chorine, bromine or iodine, or with combinations thereof. Exemplary halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (CF₃), perfluoroethyl, 2,2,2-trifluoroethyl, 2,2,2-trifluoro-1,1-dichloroethyl, and the like).

As used herein, the term “amino” means —NH₂ or —NH₃⁺ where one or more hydrogens are optionally substituted with alkyl, and the alkyl is optionally further substituted with one or more halogen or other substituents disclosed herein. The term “alkylamino” means a nitrogen moiety having one straight or branched unsaturated aliphatic, cyclyl, or heterocyclyl radicals attached to the nitrogen, e.g., —NH(alkyl). The term “dialkylamino” means a nitrogen moiety having at two straight or branched unsaturated aliphatic, cyclyl, or heterocyclyl radicals attached to the nitrogen, e.g., —N(alkyl)(alkyl). The term “alkylamino” includes “alkenylamino,” “alkynylamino,” “cyclylamino,” and “heterocyclylamino.” The term “arylamino” means a nitrogen moiety having at least one aryl radical attached to the nitrogen. For example, —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like. Exemplary alkylamino includes, but is not limited to, NH(C₁-C₁₀alkyl), such as —NHCH₃, —NHCH₂CH₃, —NHCH₂CH₂CH₃, and —NHCH(CH₃)₂. Exemplary dialkylamino includes, but is not limited to, —N(C₁-C₁₀alkyl)₂, such as N(CH₃)₂, —N(CH₂CH₃)₂, —N(CH₂CH₂CH₃)₂, and —N(CH(CH₃)₂)₂.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The terms “hydroxy” and “hydroxyl” mean the radical —OH.

The terms “alkoxyl” or “alkoxy” as used herein refers to an alkyl group, as defined above, having an oxygen radical attached thereto, and can be represented by one of —O-alkyl, —O-alkenyl, and —O-alkynyl. Aroxy can be represented by —O-aryl or O-heteroaryl, wherein aryl and heteroaryl are as defined herein. The alkoxy and aroxy groups can be substituted as described above for alkyl. Exemplary alkoxy groups include, but are not limited to O-methyl, O-ethyl, O-n-propyl, O-isopropyl, O-n-butyl, O-isobutyl, O-sec-butyl, O-tert-butyl, O-pentyl, O-hexyl, O-cyclopropyl, O-cyclobutyl, O-cyclopentyl, O-cyclohexyl and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. As used herein, a carboxy group includes —COOH, i.e., carboxyl group.

The term “cyano” means the radical —CN.

The term “nitro” means the radical —NO₂.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)—, —OS(O)₂—, and —SS—, wherein RN is H or a further substituent.

“Acyl” refers to an alkyl-CO— group, wherein alkyl is as previously described. Exemplary acyl groups comprise alkyl of 1 to about 30 carbon atoms. Exemplary acyl groups also include acetyl, propanoyl, 2-methylpropanoyl, butanoyl and palmitoyl.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Carbamoyl” refers to an H₂N—CO— group.

“Alkylcarbamoyl” refers to a R′RN—CO— group, wherein one of R and R′ is hydrogen and the other of R and R′ is alkyl as previously described.

“Dialkylcarbamoyl” refers to R′RN—CO— group, wherein each of R and R′ is independently alkyl as previously described.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

For example, any alkyl, alkenyl, cycloalkyl, heterocyclyl, heteroaryl or aryl is optionally substituted with 1, 2, 3, 4 or 5 groups selected from OH, CN, SH, SO₂NH₂, SO₂ (C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O) NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)—C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; or wherein any alkyl, cycloalkyl or heterocyclyl is optionally substituted with oxo; “m” and “p” are independently 1, 2, 3, 4, 5 or 6.

In some embodiments, an optionally substituted group is substituted with 1 substituent. In some other embodiments, an optionally substituted group is substituted with 2 independently selected substituents, which can be same or different. In some other embodiments, an optionally substituted group is substituted with 3 independently selected substituents, which can be same, different or any combination of same and different. In still some other embodiments, an optionally substituted group is substituted with 4 independently selected substituents, which can be same, different or any combination of same and different. In yet some other embodiments, an optionally substituted group is substituted with 5 independently selected substituents, which can be same, different or any combination of same and different.

As used herein, the compound designation terms “Compound #”, “PSY-#” and “PSY-05-#” (where # indicates any number having one or more digits) are synonymous with each other, unless otherwise indicated (e.g., “Compound 1” refers to a compound alternatively designated as “PSY-05-0001” or “PSY-1”).

As used herein, Compound (PSY-05-00074) (alternatively designated as Compound 74) is:

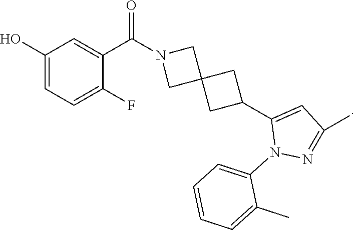

Compound (PSY-05-00074) is referred to by name as (2-fluoro-5-hydroxyphenyl) (6-(3-methyl-1-(o-tolyl)-1H-pyrazol-5-yl)-2-azaspiro[3.3]heptan-2-yl) methanone, of (2-fluoro-5-hydroxyphenyl) {6-[3-methyl-1-(o-tolyl)-5-pyrazolyl]-2-aza-2-spiro[3.3]heptyl}methanone. Either name may be used interchangeably herein to refer to Compound (PSY-05-00074).

### Additional Embodiments

1. A method comprising the administration of a Reversible and Selective MAGL Inhibitor Compound of Formula (I) or a pharmaceutically acceptable salt thereof,

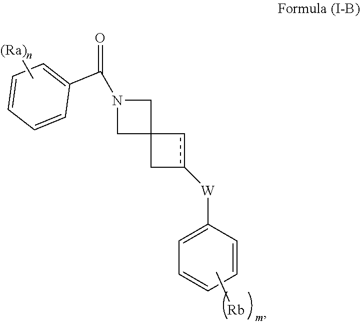

wherein
   n is 1, 2, or 3;
   each Ra is independently halogen, cyano, lower alkyl optionally substituted with one or more halogen, or —OR₆;
   R₆ is hydrogen, lower alkyl or lower cycloalkyl optionally substituted with one or more halogen,
   W is A, —C(O)-A, —C(O)— or —C(O)N(R₁₀)—;
   R₁₀ is hydrogen or lower alkyl;
   A is a 5-member heteroaryl ring optionally substituted with one or more R₃₀;
   R₃₀ is lower alkyl;
   m is 1, or 2; and
   each R_{b} is independently halogen, or lower alkyl optionally substituted with one of more halogen.
2. The method of embodiment 1, wherein
   a. each Rₐ is independently Cl, F, CN, cyano, methyl, or —OR₆;
   b. R₆ is hydrogen, (C₁-C₄)alkyl optionally substituted with one or more F or cyclopropyl;
   c. W is A, —C(O)—, —C(O)-A, —C(O)N(R₁₀)—;
   d. R₁₀ is hydrogen or methyl;
   e. A is a S-member heteroaryl ring optionally substituted with one or more R₃₀;
   f. R₃₀ is (C₁-C₄)alkyl;
   g. m is 1, or 2; and
   h. each R_{b} is independently halogen, or (C₁-C₄)alkyl optionally substituted with one or more F.
3. The method of embodiment 2, wherein R₃₀ is methyl; and each R_{b} is independently halogen, or methyl optionally substituted with one or more F.
4. The compound of embodiment 3, wherein A is selected from the group consisting of
   a. pyrazole, imidazole, or triazole, each optionally substituted with one methyl; and
   b. oxadiazole.
5. The method of embodiment 4, wherein A is pyrazole substituted with one methyl.
6. The method of any one of embodiments 1-5, wherein one Ra is —OR₆.
7. The method of embodiment 6, wherein one R_{b} is methyl.
8. The method of embodiment 1, of Formula (I-A):

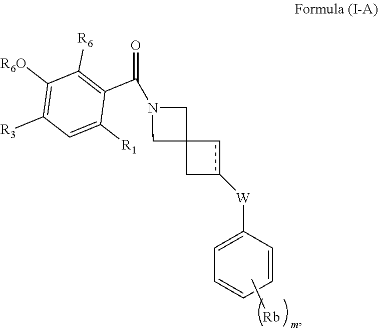

wherein
   R₁ is hydrogen or halogen;
   R₃ is hydrogen, halogen, or lower alkyl optionally substituted with one or more halogen;
   R₅ is hydrogen, halogen, lower alkoxy or lower alkyl each optionally substituted with one or more halogen; and
   R₆ is hydrogen, lower alkyl or cycloalkyl optionally substituted with one or more halogen.
9. The method of embodiment 1, of Formula (I-B),

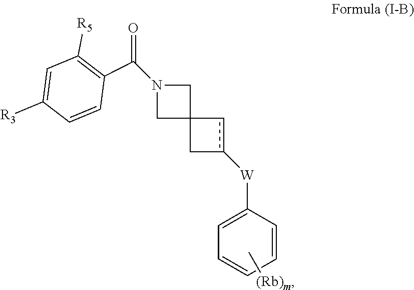

wherein
   R₃ is hydrogen or halogen;
   R₅ is —O—R₅₂; and
   R₅₂ is lower alkyl or cycloalkyl, each optionally substituted with halogen.
10. The method of any one of embodiments 1, 8 or 9, wherein W is selected from the group consisting of A1, A2, A3, A4, A5, A6 and A7,

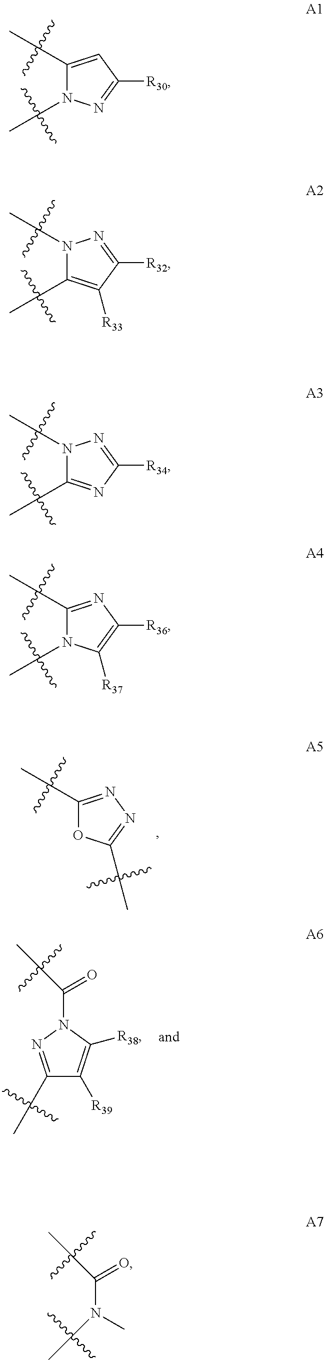

and R₃₀, R₃₂, R₃₃, R₃₄, R₃₆, R₃₇, R₃₈ and R₃₉ are each independently hydrogen or lower alkyl.
11. The method of embodiment 10, wherein R₃₀, R₃₂, R₃₃, R₃₄, R₃₆, R₃₇, R₃₈ and R₃₉ are each independently hydrogen or methyl.
12. The method of embodiment 10, wherein W is A1 and R₃₀ is methyl.
13. The method of embodiment 10, wherein W is A2 and R₃₂ is hydrogen and R₃₃ is methyl.
14. The method of embodiment 10, wherein W is A7 and Rb is methyl.
15. The method of embodiment 1, of Formula (I-C),

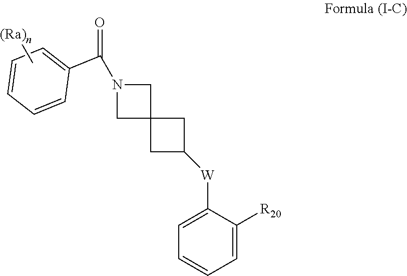

wherein R₂₀ is lower alkyl; and Ra, n, and W are as defined above with respect to Formula (I-C).
16. The method of embodiment 15, wherein R₂₀ is methyl optionally substituted with one or more F.
17. A method comprising the administration of a Reversible and Selective MAGL Inhibitor Compound of Formula (II), or a pharmaceutically acceptable salt thereof,

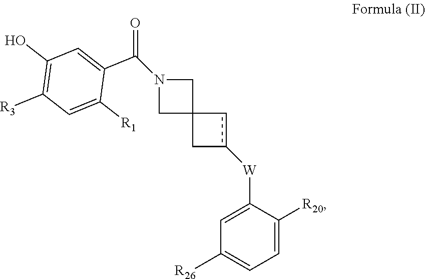

wherein
   R₁ is halogen or cyano;
   R₃ is hydrogen or halogen;
   W is A, —C(O)—, —C(O)N(R₁₀)—;
   R₁₀ is hydrogen or lower alkyl;
   A is a 5-member heteroaryl ring optionally substituted with one or more R₃₀; and
   R₂₀ and R₃₀ are each independently lower alkyl.
18. A method comprising the administration of a Reversible and Selective MAGL Inhibitor Compound of Formula (III), or a pharmaceutically acceptable salt thereof,

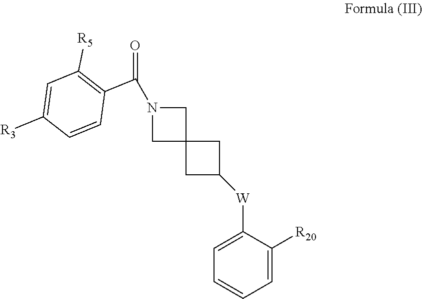

wherein
   R₃ is halogen;
   R₆ is —O—R₆₂;
   R₆₂ is lower alkyl or cycloalkyl, each optionally substituted with halogen,
   W is a 5-member heteroaryl ring optionally substituted with one or more R₃₀;
   R₃₀ is lower alkyl; and
   R₂₀ is lower alkyl.
19. The method of embodiment 18, wherein W is a 5-member heteroaryl ring comprising at least one nitrogen heteroatom optionally substituted with one methyl, the methyl optionally substituted with one or more F.
20. A method comprising the administration of a compound selected from the group consisting of:

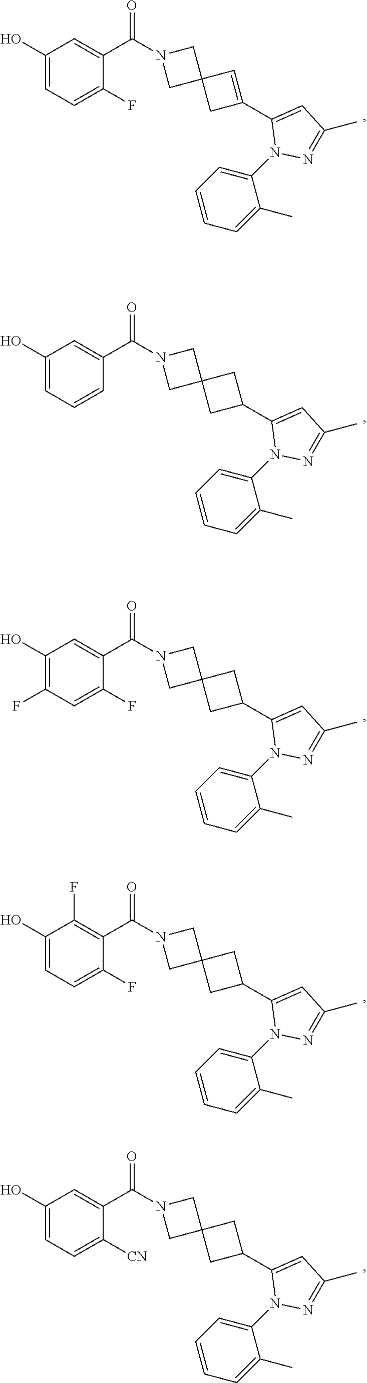 [SMILES: Cc1cc(C2=CC3(C2)CN(C(=O)c2cc(O)ccc2F)C3)n(-c2ccccc2C)n1 ; Cc1cc(C2CC3(C2)CN(C(=O)c2cccc(O)c2)C3)n(-c2ccccc2C)n1 ; Cc1cc(C2CC3(C2)CN(C(=O)c2cc(O)c(F)cc2F)C3)n(-c2ccccc2C)n1 ; Cc1cc(C2CC3(C2)CN(C(=O)c2c(F)ccc(O)c2F)C3)n(-c2ccccc2C)n1 ; Cc1cc(C2CC3(C2)CN(C(=O)c2cc(O)ccc2C#N)C3)n(-c2ccccc2C)n1]

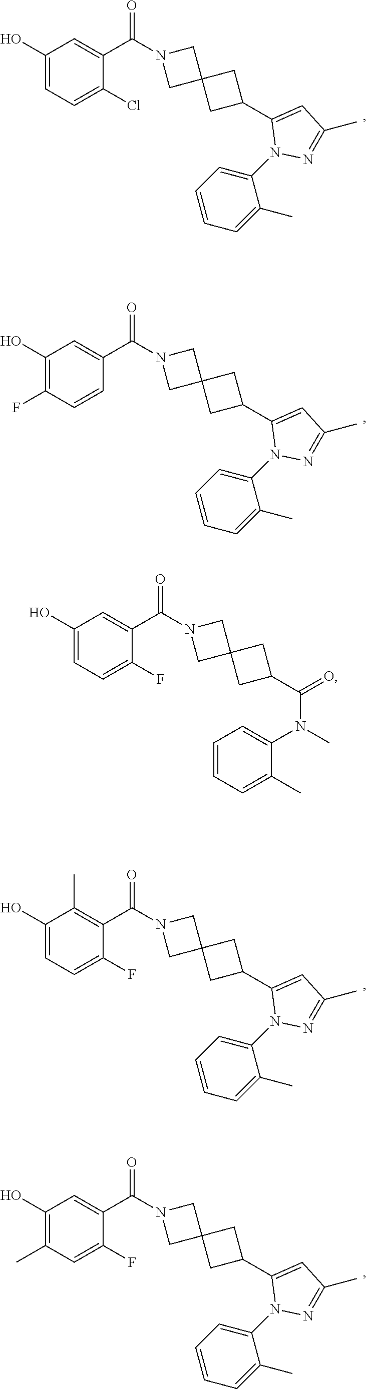 [SMILES: Cc1cc(C2CC3(C2)CN(C(=O)c2cc(O)ccc2Cl)C3)n(-c2ccccc2C)n1 ; Cc1cc(C2CC3(C2)CN(C(=O)c2ccc(F)c(O)c2)C3)n(-c2ccccc2C)n1 ; Cc1ccccc1N(C)C(=O)C1CC2(C1)CN(C(=O)c1cc(O)ccc1F)C2 ; Cc1cc(C2CC3(C2)CN(C(=O)c2c(F)ccc(O)c2C)C3)n(-c2ccccc2C)n1 ; Cc1cc(C2CC3(C2)CN(C(=O)c2cc(O)c(C)cc2F)C3)n(-c2ccccc2C)n1]

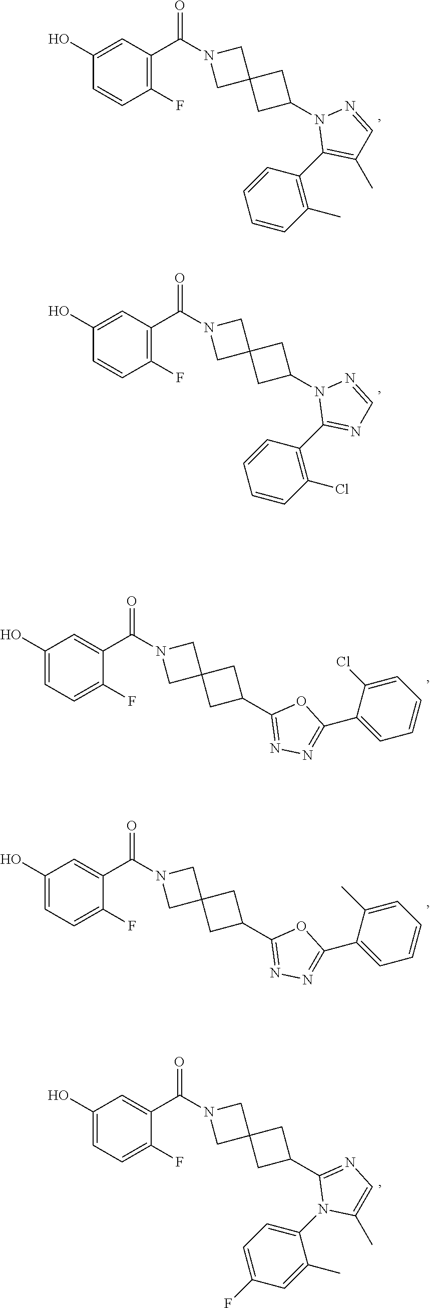 [SMILES: Cc1ccccc1-c1c(C)cnn1C1CC2(C1)CN(C(=O)c1cc(O)ccc1F)C2 ; O=C(c1cc(O)ccc1F)N1CC2(CC(n3ncnc3-c3ccccc3Cl)C2)C1 ; O=C(c1cc(O)ccc1F)N1CC2(CC(c3nnc(-c4ccccc4Cl)o3)C2)C1 ; Cc1ccccc1-c1nnc(C2CC3(C2)CN(C(=O)c2cc(O)ccc2F)C3)o1 ; Cc1cc(F)ccc1-n1c(C)cnc1C1CC2(C1)CN(C(=O)c1cc(O)ccc1F)C2]

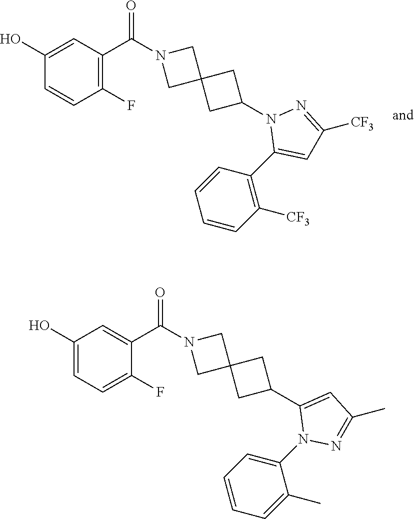 [SMILES: O=C(c1cc(O)ccc1F)N1CC2(CC(n3nc(C(F)(F)F)cc3-c3ccccc3C(F)(F)F)C2)C1 ; Cc1cc(C2CC3(C2)CN(C(=O)c2cc(O)ccc2F)C3)n(-c2ccccc2C)n1]

or a pharmaceutically acceptable salt thereof.
21. A method comprising the administration of a compound selected from the group consisting of:

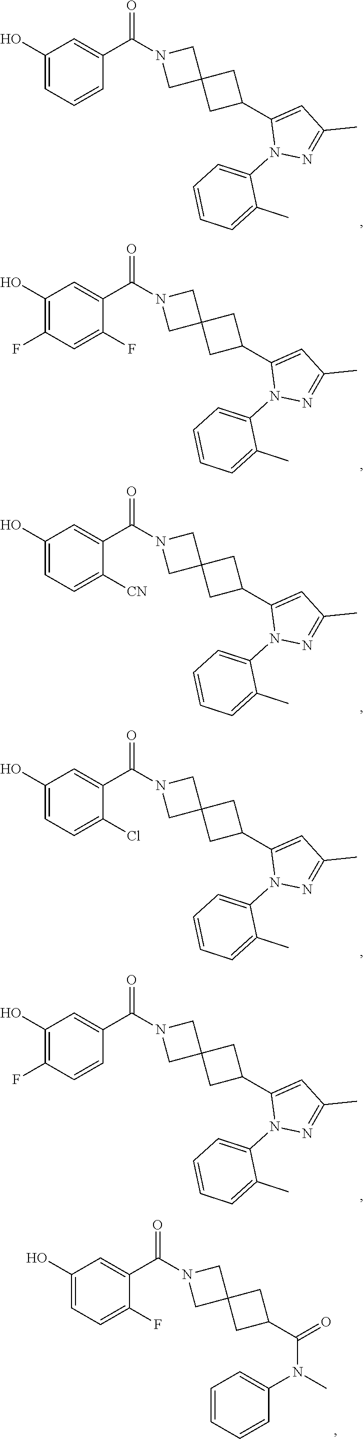 [SMILES: Cc1cc(C2CC3(C2)CN(C(=O)c2cccc(O)c2)C3)n(-c2ccccc2C)n1 ; Cc1cc(C2CC3(C2)CN(C(=O)c2cc(O)c(F)cc2F)C3)n(-c2ccccc2C)n1 ; Cc1cc(C2CC3(C2)CN(C(=O)c2cc(O)ccc2C#N)C3)n(-c2ccccc2C)n1 ; Cc1cc(C2CC3(C2)CN(C(=O)c2cc(O)ccc2Cl)C3)n(-c2ccccc2C)n1 ; Cc1cc(C2CC3(C2)CN(C(=O)c2ccc(F)c(O)c2)C3)n(-c2ccccc2C)n1 ; CN(C(=O)C1CC2(C1)CN(C(=O)c1cc(O)ccc1F)C2)c1ccccc1]

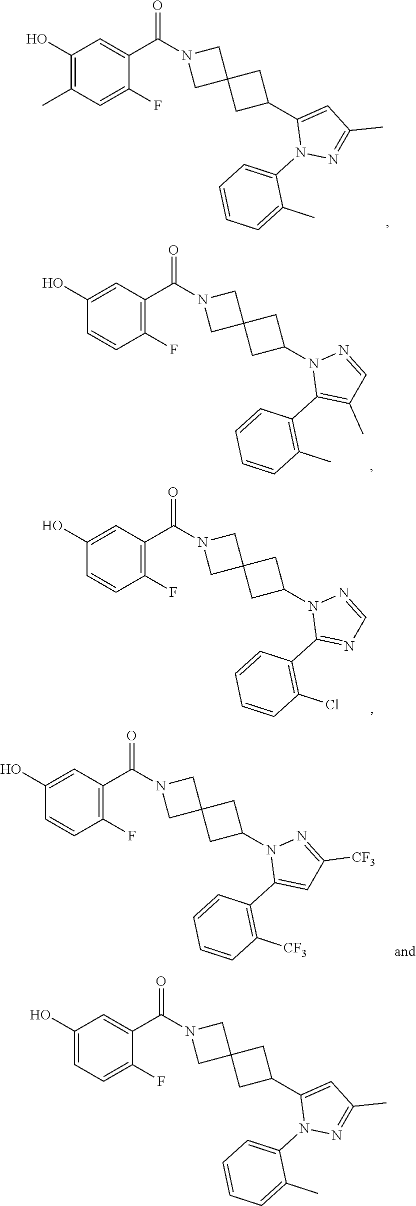 [SMILES: Cc1cc(C2CC3(C2)CN(C(=O)c2cc(O)c(C)cc2F)C3)n(-c2ccccc2C)n1 ; Cc1ccccc1-c1c(C)cnn1C1CC2(C1)CN(C(=O)c1cc(O)ccc1F)C2 ; O=C(c1cc(O)ccc1F)N1CC2(CC(n3ncnc3-c3ccccc3Cl)C2)C1 ; O=C(c1cc(O)ccc1F)N1CC2(CC(n3nc(C(F)(F)F)cc3-c3ccccc3C(F)(F)F)C2)C1 ; Cc1cc(C2CC3(C2)CN(C(=O)c2cc(O)ccc2F)C3)n(-c2ccccc2C)n1]

or a pharmaceutically acceptable salt thereof.
22. A method comprising the administration of a compound selected from the group consisting of:

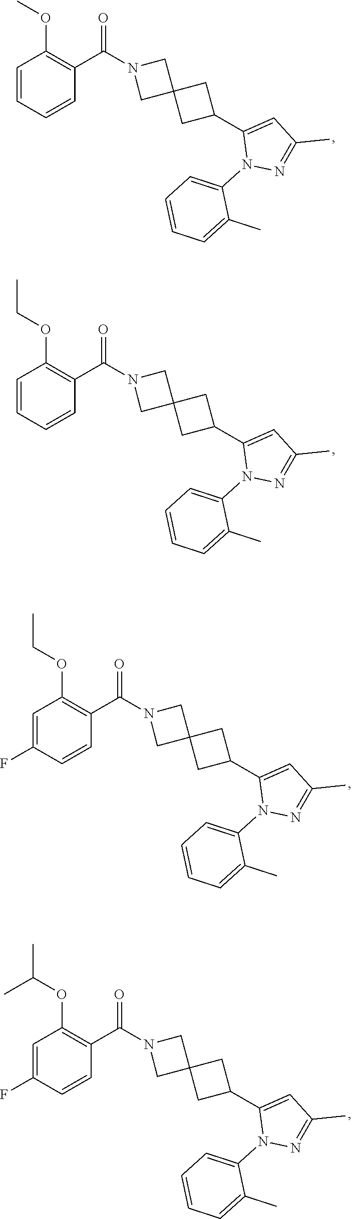 [SMILES: COc1ccccc1C(=O)N1CC2(CC(c3cc(C)nn3-c3ccccc3C)C2)C1 ; CCOc1ccccc1C(=O)N1CC2(CC(c3cc(C)nn3-c3ccccc3C)C2)C1 ; CCOc1cc(F)ccc1C(=O)N1CC2(CC(c3cc(C)nn3-c3ccccc3C)C2)C1 ; Cc1cc(C2CC3(C2)CN(C(=O)c2ccc(F)cc2OC(C)C)C3)n(-c2ccccc2C)n1]

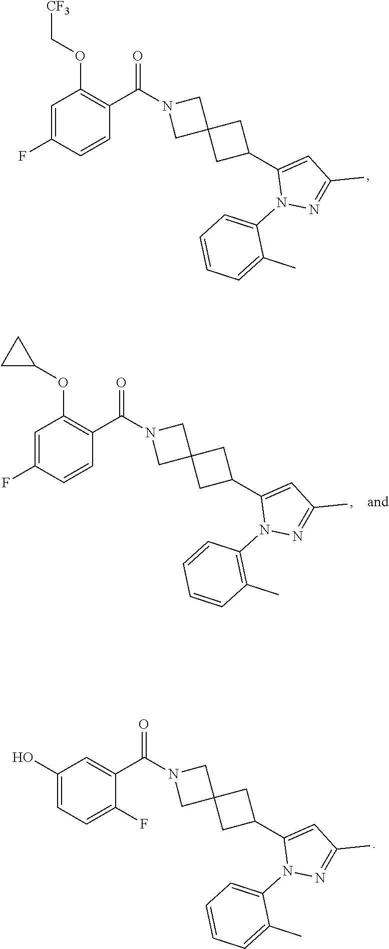 [SMILES: Cc1cc(C2CC3(C2)CN(C(=O)c2ccc(F)cc2OCC(F)(F)F)C3)n(-c2ccccc2C)n1 ; Cc1cc(C2CC3(C2)CN(C(=O)c2ccc(F)cc2OC2CC2)C3)n(-c2ccccc2C)n1 ; Cc1cc(C2CC3(C2)CN(C(=O)c2cc(O)ccc2F)C3)n(-c2ccccc2C)n1]

23. A method comprising the administration of a compound selected from the group consisting of:

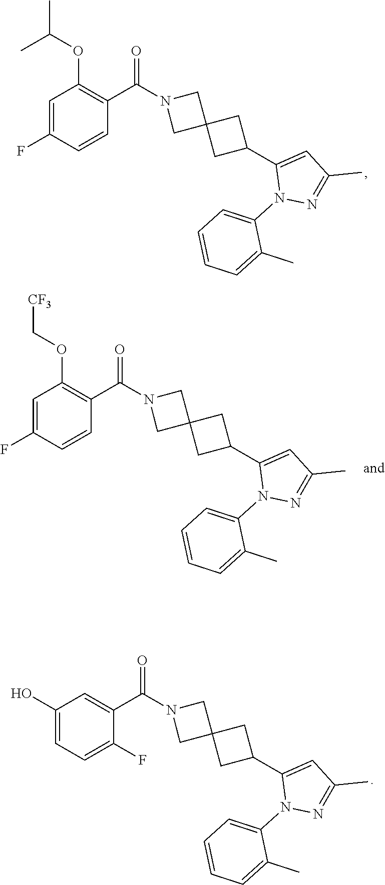 [SMILES: Cc1cc(C2CC3(C2)CN(C(=O)c2ccc(F)cc2OC(C)C)C3)n(-c2ccccc2C)n1 ; Cc1cc(C2CC3(C2)CN(C(=O)c2ccc(F)cc2OCC(F)(F)F)C3)n(-c2ccccc2C)n1 ; Cc1cc(C2CC3(C2)CN(C(=O)c2cc(O)ccc2F)C3)n(-c2ccccc2C)n1]

24. A method comprising the administration of a compound

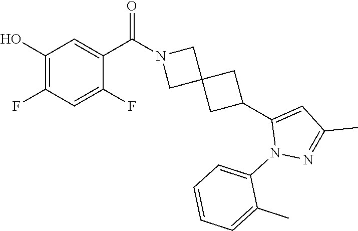 [SMILES: Cc1cc(C2CC3(C2)CN(C(=O)c2cc(O)c(F)cc2F)C3)n(-c2ccccc2C)n1]

or a pharmaceutically acceptable salt thereof.
25. A method comprising the administration of a compound

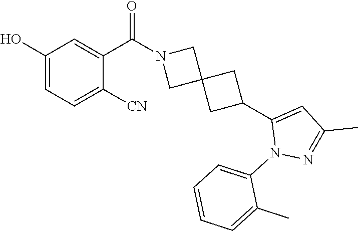 [SMILES: Cc1cc(C2CC3(C2)CN(C(=O)c2cc(O)ccc2C#N)C3)n(-c2ccccc2C)n1]

or a pharmaceutically acceptable salt thereof.
26. A method comprising the administration of a compound

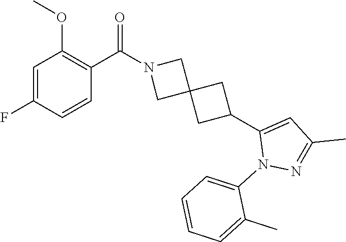 [SMILES: COc1cc(F)ccc1C(=O)N1CC2(CC(c3cc(C)nn3-c3ccccc3C)C2)C1]

or a pharmaceutically acceptable salt thereof.
27. A method comprising the administration of a compound

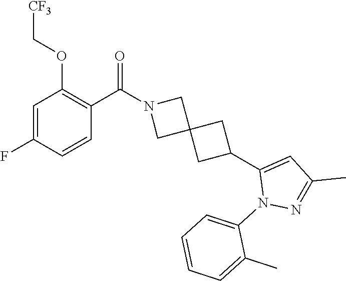 [SMILES: Cc1cc(C2CC3(C2)CN(C(=O)c2ccc(F)cc2OCC(F)(F)F)C3)n(-c2ccccc2C)n1]

or a pharmaceutically acceptable salt thereof.
28. A method comprising the administration of a compound

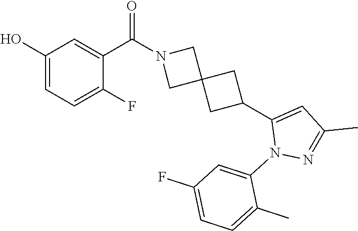 [SMILES: Cc1cc(C2CC3(C2)CN(C(=O)c2cc(O)ccc2F)C3)n(-c2cc(F)ccc2C)n1]

or a pharmaceutically acceptable salt thereof.
29. A method comprising the administration of a compound

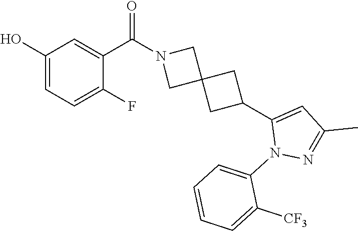 [SMILES: Cc1cc(C2CC3(C2)CN(C(=O)c2cc(O)ccc2F)C3)n(-c2ccccc2C(F)(F)F)n1]

or a pharmaceutically acceptable salt thereof.
30. A method comprising the administration of a compound

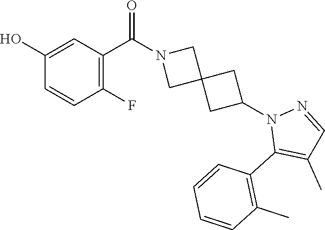 [SMILES: Cc1ccccc1-c1c(C)cnn1C1CC2(C1)CN(C(=O)c1cc(O)ccc1F)C2]

or a pharmaceutically acceptable salt thereof.
31. A method comprising the administration of a compound

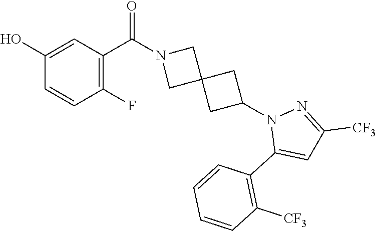 [SMILES: O=C(c1cc(O)ccc1F)N1CC2(CC(n3nc(C(F)(F)F)cc3-c3ccccc3C(F)(F)F)C2)C1]

or a pharmaceutically acceptable salt thereof.
32. A method comprising the administration of a compound

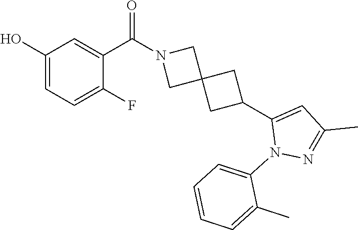 [SMILES: Cc1cc(C2CC3(C2)CN(C(=O)c2cc(O)ccc2F)C3)n(-c2ccccc2C)n1]

or a pharmaceutically acceptable salt thereof.
33. A method comprising the administration of (2-fluoro-5-hydroxyphenyl) (6-(3-methyl-1-(o-tolyl)-1H-pyrazol-5-yl)-2-azaspiro[3.3]heptan-2-yl) methanone or (2-ethoxy-4-fluorophenyl) (6-(3-methyl-1-(o-tolyl)-1H-pyrazol-5-yl)-2-azaspiro[3.3]heptan-2-yl) methanone.
34. A method comprising the administration of a Reversible Selective MAGL Inhibitor Compound.
35. The method of any one of embodiments 1-34, wherein the method comprises the administration of a Reversible Selective MAGL Inhibitor Compound to a subject in an amount effective to transiently increase the level of 2-AG in the brain of the subject.
36. The method of any one of embodiments 1-34, wherein the method comprises the administration of a Reversible Selective MAGL Inhibitor Compound to a subject in an amount effective to transiently increase the level of 2-AG in the brain of the subject within about 30 minutes after administration of the Reversible Selective MAGL Inhibitor Compound.
37. The method of any one of embodiments 1-34, wherein the method comprises the oral administration of a Reversible Selective MAGL Inhibitor Compound to a subject in an amount effective to transiently increase the level of 2-AG in the brain of the subject after administration of the Reversible Selective MAGL Inhibitor Compound.
38. The method of any one of embodiments 1-34, wherein the method comprises the oral administration of a Reversible Selective MAGL Inhibitor Compound to a subject in an amount effective to transiently increase the level of 2-AG in the brain of the subject after administration of the Reversible Selective MAGL Inhibitor Compound.
39. The method of any one of embodiments 1-38, wherein the administration of the Reversible Selective MAGL Inhibitor Compound increases the level of 2-AG in the brain of the subject characterized by a half-life of the increase in the level of 2-AG in the brain of the subject is less than twice the half-life of the Reversible Selective MAGL Inhibitor Compound in the blood plasma of the subject.
40. A method of transiently increasing 2-AG in the brain of a subject, comprising the administration of a Reversible Selective MAGL Inhibitor Compound to the subject.
41. The method of embodiment 40, wherein the half-life of the transient increase of the 2-AG in the brain is less than twice the half-life of the Reversible Selective MAGL Inhibitor Compound in the blood plasma of the subject.
42. The method of any one of embodiments 40 or 41, wherein the subject is human.
43. The method of embodiment 42, wherein the human subject is diagnosed with generalized anxiety disorder or post traumatic stress disorder prior to the administration of the Reversible Selective MAGL Inhibitor Compound.
44. A method of treating generalized anxiety disorder (GAD) in a human subject comprising the oral administration of a therapeutically effective amount of a Reversible Selective MAGL Inhibitor Compound to the subject in need thereof.
45. The method of embodiment 44, wherein the Reversible Selective MAGL Inhibitor Compound is a compound administered in the method of any one of embodiments 1-32, or a pharmaceutically acceptable salt thereof.
46. A method of treating post traumatic stress disorder (PTSD) in a human subject comprising the oral administration of a therapeutically effective amount of a Reversible Selective MAGL Inhibitor Compound to the subject in need thereof.
47. The method of embodiment 44, wherein the Reversible Selective MAGL Inhibitor Compound is a compound administered in the method of any one of embodiments 1-32, or a pharmaceutically acceptable salt thereof.

In some embodiments, the compound is selected from the group consisting of:

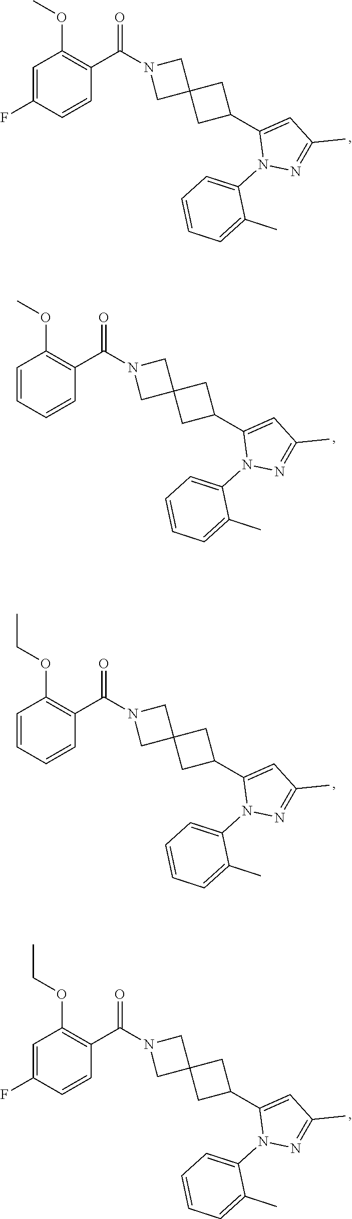 [SMILES: COc1cc(F)ccc1C(=O)N1CC2(CC(c3cc(C)nn3-c3ccccc3C)C2)C1 ; COc1ccccc1C(=O)N1CC2(CC(c3cc(C)nn3-c3ccccc3C)C2)C1 ; CCOc1ccccc1C(=O)N1CC2(CC(c3cc(C)nn3-c3ccccc3C)C2)C1 ; CCOc1cc(F)ccc1C(=O)N1CC2(CC(c3cc(C)nn3-c3ccccc3C)C2)C1]

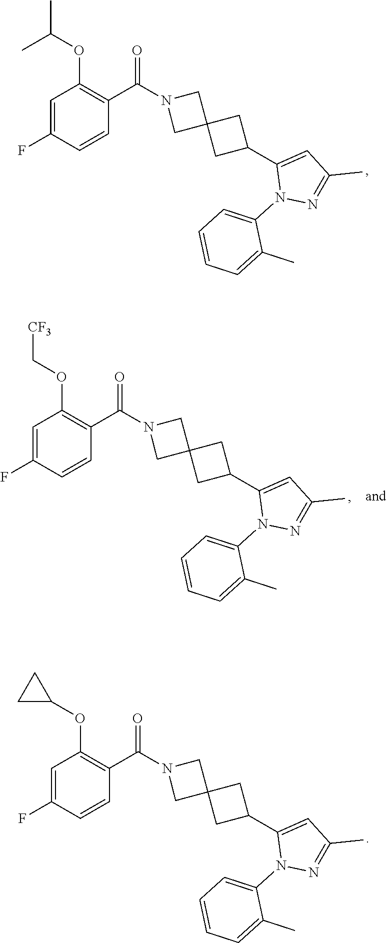 [SMILES: Cc1cc(C2CC3(C2)CN(C(=O)c2ccc(F)cc2OC(C)C)C3)n(-c2ccccc2C)n1 ; Cc1cc(C2CC3(C2)CN(C(=O)c2ccc(F)cc2OCC(F)(F)F)C3)n(-c2ccccc2C)n1 ; Cc1cc(C2CC3(C2)CN(C(=O)c2ccc(F)cc2OC2CC2)C3)n(-c2ccccc2C)n1]

In some embodiments, the compound is selected from the group consisting of:

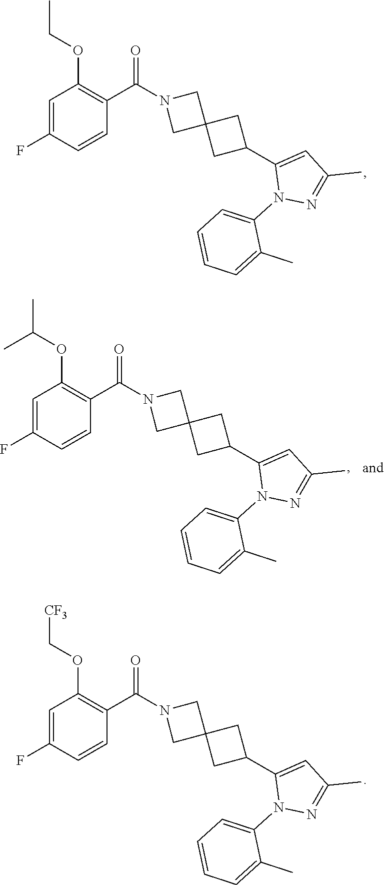 [SMILES: CCOc1cc(F)ccc1C(=O)N1CC2(CC(c3cc(C)nn3-c3ccccc3C)C2)C1 ; Cc1cc(C2CC3(C2)CN(C(=O)c2ccc(F)cc2OC(C)C)C3)n(-c2ccccc2C)n1 ; Cc1cc(C2CC3(C2)CN(C(=O)c2ccc(F)cc2OCC(F)(F)F)C3)n(-c2ccccc2C)n1]

In some embodiments, the compound is selected from the group consisting of:

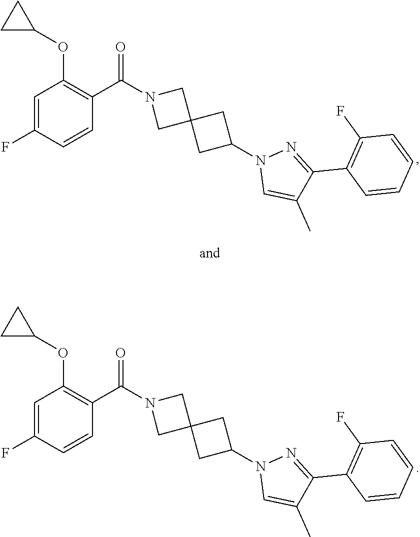 [SMILES: Cc1cn(C2CC3(C2)CN(C(=O)c2ccc(F)cc2OC2CC2)C3)nc1-c1ccccc1F ; Cc1cn(C2CC3(C2)CN(C(=O)c2ccc(F)cc2OC2CC2)C3)nc1-c1ccccc1F]

Other compounds include the compounds shown below that are Selective MAGL Inhibitors and Reversible MAGL Inhibitors:

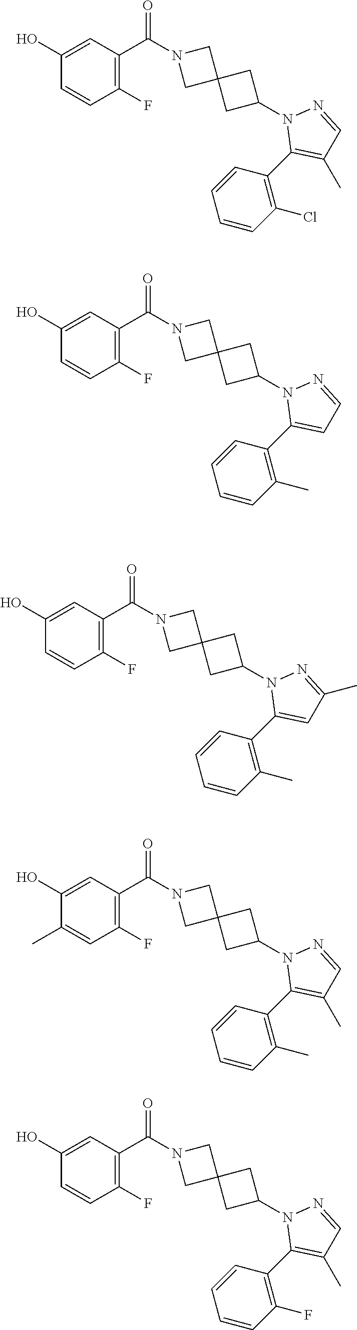 [SMILES: Cc1cnn(C2CC3(C2)CN(C(=O)c2cc(O)ccc2F)C3)c1-c1ccccc1Cl ; Cc1ccccc1-c1ccnn1C1CC2(C1)CN(C(=O)c1cc(O)ccc1F)C2 ; Cc1cc(-c2ccccc2C)n(C2CC3(C2)CN(C(=O)c2cc(O)ccc2F)C3)n1 ; Cc1cc(F)c(C(=O)N2CC3(CC(n4ncc(C)c4-c4ccccc4C)C3)C2)cc1O ; Cc1cnn(C2CC3(C2)CN(C(=O)c2cc(O)ccc2F)C3)c1-c1ccccc1F]

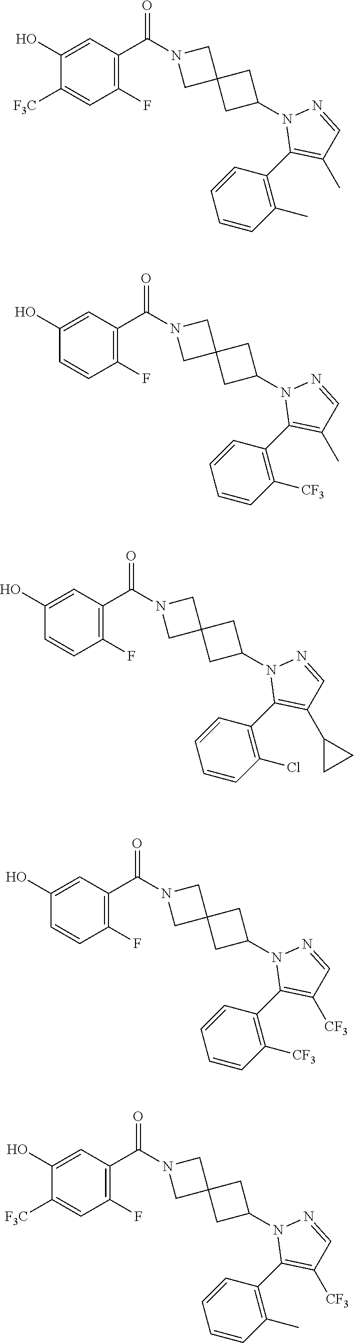 [SMILES: Cc1ccccc1-c1c(C)cnn1C1CC2(C1)CN(C(=O)c1cc(O)c(C(F)(F)F)cc1F)C2 ; Cc1cnn(C2CC3(C2)CN(C(=O)c2cc(O)ccc2F)C3)c1-c1ccccc1C(F)(F)F ; O=C(c1cc(O)ccc1F)N1CC2(CC(n3ncc(C4CC4)c3-c3ccccc3Cl)C2)C1 ; O=C(c1cc(O)ccc1F)N1CC2(CC(n3ncc(C(F)(F)F)c3-c3ccccc3C(F)(F)F)C2)C1 ; Cc1ccccc1-c1c(C(F)(F)F)cnn1C1CC2(C1)CN(C(=O)c1cc(O)c(C(F)(F)F)cc1F)C2]

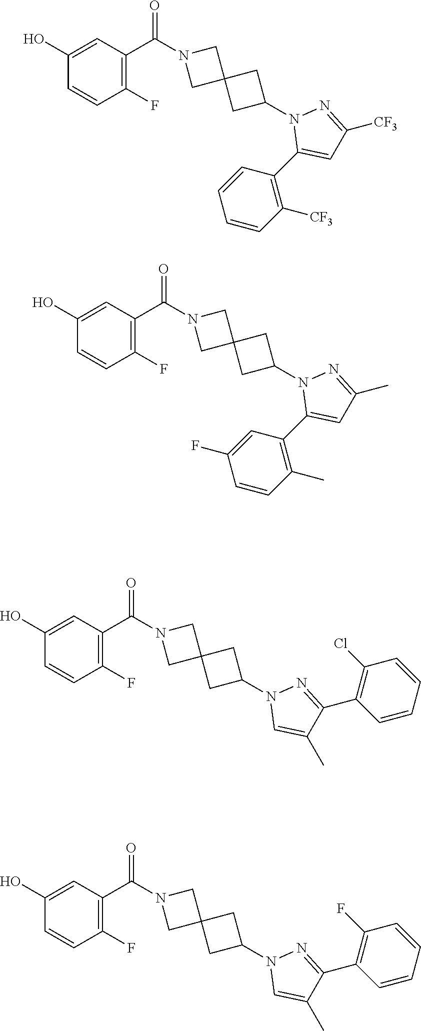 [SMILES: O=C(c1cc(O)ccc1F)N1CC2(CC(n3nc(C(F)(F)F)cc3-c3ccccc3C(F)(F)F)C2)C1 ; Cc1cc(-c2cc(F)ccc2C)n(C2CC3(C2)CN(C(=O)c2cc(O)ccc2F)C3)n1 ; Cc1cn(C2CC3(C2)CN(C(=O)c2cc(O)ccc2F)C3)nc1-c1ccccc1Cl ; Cc1cn(C2CC3(C2)CN(C(=O)c2cc(O)ccc2F)C3)nc1-c1ccccc1F]

Other compounds include compounds shown below:

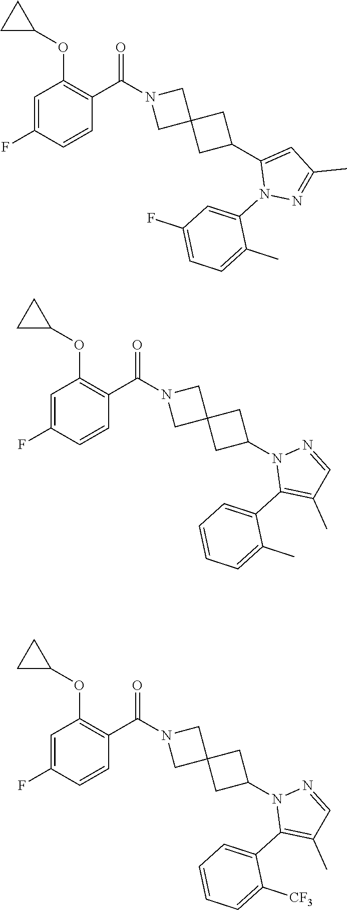 [SMILES: Cc1cc(C2CC3(C2)CN(C(=O)c2ccc(F)cc2OC2CC2)C3)n(-c2cc(F)ccc2C)n1 ; Cc1ccccc1-c1c(C)cnn1C1CC2(C1)CN(C(=O)c1ccc(F)cc1OC1CC1)C2 ; Cc1cnn(C2CC3(C2)CN(C(=O)c2ccc(F)cc2OC2CC2)C3)c1-c1ccccc1C(F)(F)F]

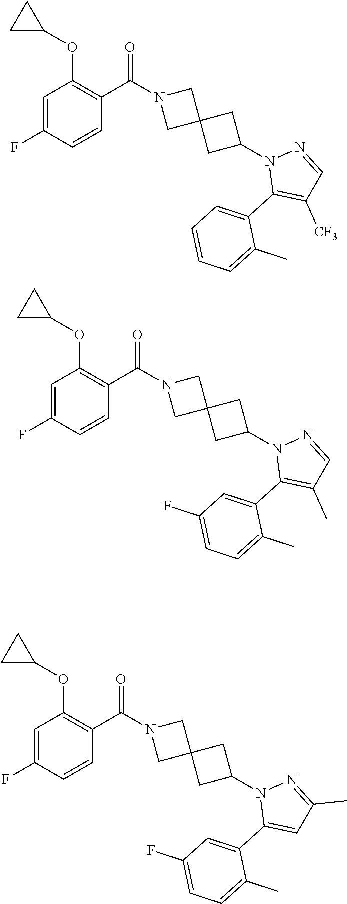 [SMILES: Cc1ccccc1-c1c(C(F)(F)F)cnn1C1CC2(C1)CN(C(=O)c1ccc(F)cc1OC1CC1)C2 ; Cc1ccc(F)cc1-c1c(C)cnn1C1CC2(C1)CN(C(=O)c1ccc(F)cc1OC1CC1)C2 ; Cc1cc(-c2cc(F)ccc2C)n(C2CC3(C2)CN(C(=O)c2ccc(F)cc2OC2CC2)C3)n1]

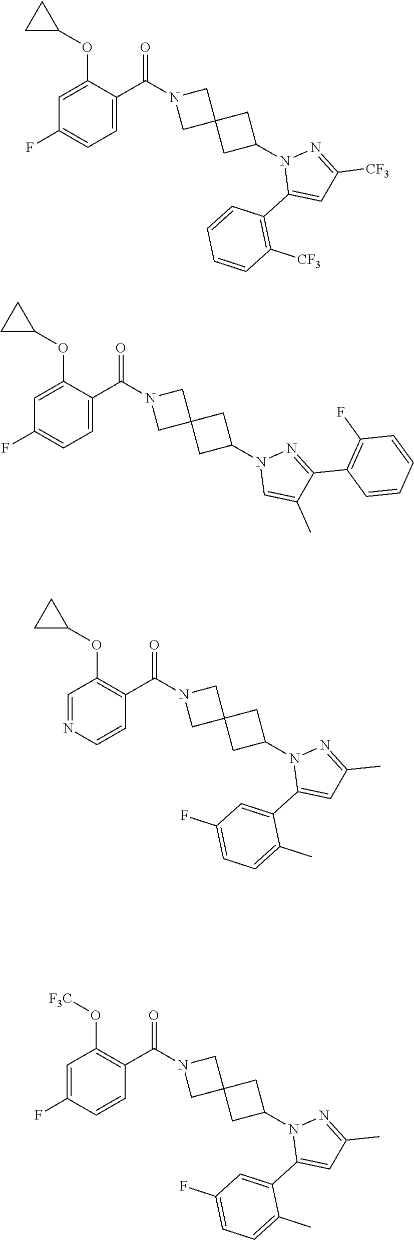 [SMILES: O=C(c1ccc(F)cc1OC1CC1)N1CC2(CC(n3nc(C(F)(F)F)cc3-c3ccccc3C(F)(F)F)C2)C1 ; Cc1cn(C2CC3(C2)CN(C(=O)c2ccc(F)cc2OC2CC2)C3)nc1-c1ccccc1F ; Cc1cc(-c2cc(F)ccc2C)n(C2CC3(C2)CN(C(=O)c2ccncc2OC2CC2)C3)n1 ; Cc1cc(-c2cc(F)ccc2C)n(C2CC3(C2)CN(C(=O)c2ccc(F)cc2OC(F)(F)F)C3)n1]

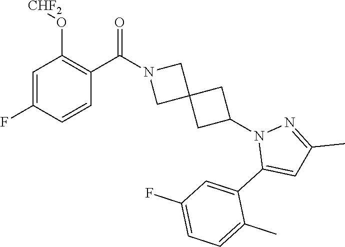 [SMILES: Cc1cc(-c2cc(F)ccc2C)n(C2CC3(C2)CN(C(=O)c2ccc(F)cc2OC(F)F)C3)n1]

### Additional Embodiments

1. A method of transiently increasing 2-AG in the brain of a subject, the method comprising the administration of a Reversible Selective MAGL Inhibitor Compound.
2. The method of embodiment 1, wherein the half-life of the transient increase of 2-AG in the brain of the subject is less than twice the blood plasma half-life of the Reversible Selective MAGL Inhibitor Compound.
3. The method of any one of embodiments 1-2, wherein the Reversible Selective MAGL Inhibitor Compound is orally administered to the subject.
4. The method of embodiment 3, wherein the maximum increase of 2-AG is observed in the brain of the subject 30 minutes after administration of the Reversible Selective MAGL Inhibitor Compound.
5. The method of embodiment 3, wherein the a therapeutically effective increase of 2-AG is observed in the brain of the subject 30 minutes after administration of the Reversible Selective MAGL Inhibitor Compound.
6. A method of treating anxiety or PTSD in a human subject comprising the administration of a therapeutically effective amount of a Reversible Selective MAGL Inhibitor Compound to the subject in need thereof.
7. A method of treating post-traumatic stress disorder in a human subject comprising the oral administration of a therapeutically effective amount of a Reversible Selective MAGL Inhibitor Compound to the subject in need thereof.
8. The method of any one of any one of embodiments 1-7, wherein the Reversible and Selective MAGL Inhibitor Compound of Formula (I-B) or a pharmaceutically acceptable salt thereof,

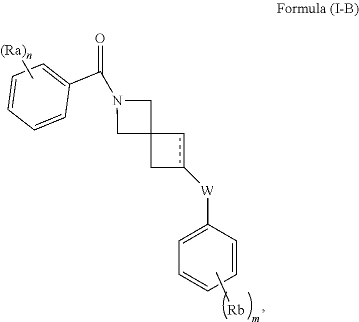

wherein
   n is 1, 2, or 3;
   each Ra is independently halogen, cyano, lower alkyl optionally substituted with one or more halogen, or —OR₆;
   R₆ is hydrogen, lower alkyl or lower cycloalkyl optionally substituted with one or more halogen;
   W is A, C(O)—, —C(O)-A, or —C(O)N(R₁₀)—;
   R₁₀ is hydrogen or lower alkyl;
   A is a 5-member heteroaryl ring optionally substituted with one or more R₃₀;
   R₃₀ is lower alkyl;
   m is 1, or 2; and
   each R_{b} is independently halogen, or lower alkyl optionally substituted with one or more halogen.
9. The method of embodiment 8, wherein
   a. each Ra is independently Cl, F, CN, cyano, methyl, or —OR₆;
   b. R₆ is hydrogen, (C₁-C₄)alkyl optionally substituted with one or more F or cyclopropyl;
   c. W is A;
   d. R₁₀ is hydrogen or methyl;
   e. A is a S-member heteroaryl ring optionally substituted with one or more R₃₀;
   f. R₃₀ is (C₁-C₄)alkyl;
   g. m is 1, or 2; and
   h. each Rₐ is independently halogen, or (C₁-C₄)alkyl optionally substituted with one or more F.
10. The method of embodiment 9, wherein R₃₀ is methyl; and each R_{b} is independently halogen, or methyl optionally substituted with one or more F.
11. The method of embodiment 10, wherein A is selected from the group consisting of
   a. pyrazole, imidazole, or triazole, each optionally substituted with one methyl; and
   b. oxadiazole.
12. The method of embodiment 11, wherein A is pyrazole substituted with one methyl.
13. The method of any one of embodiments 8-12, wherein one Ra is —OR₆.
14. The method of embodiment 12, wherein one Rb is methyl.
15. The method of any one of any one of embodiments 1-7, wherein the Reversible and Selective MAGL Inhibitor Compound of Formula (I-B-1) or a pharmaceutically acceptable salt thereof:

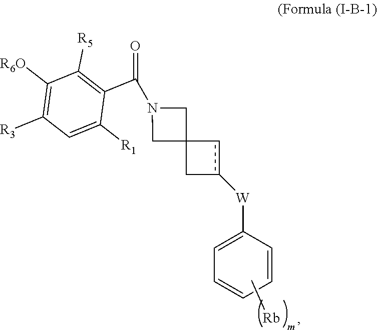

wherein
   R₁ is hydrogen or halogen;
   R₃ is hydrogen, halogen, or lower alkyl optionally substituted with one or more halogen;
   R₅ is hydrogen, halogen, lower alkoxy or lower alkyl each optionally substituted with one or more halogen; and
   R₆ is hydrogen, lower alkyl or cycloalkyl optionally substituted with one or more halogen.
16. The method of embodiment 15, of Formula (I-B-2),

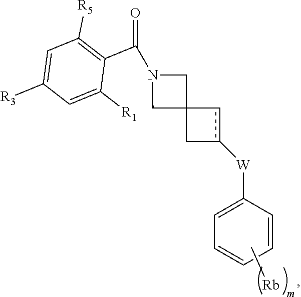

wherein
   R₃ is hydrogen or halogen;
   R₅ is —O—R₅₂; and
   R₅₂ is lower alkyl or cycloalkyl, each optionally substituted with halogen.
17. The method of any one of embodiments 8-16, wherein W is selected from the group consisting of A1, A2, A3, A4, A5, A6 and A7,

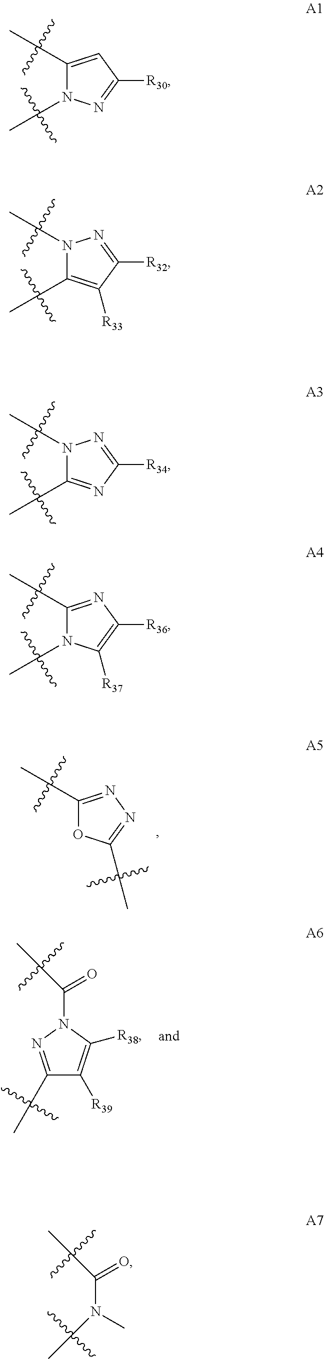

and R₃₀, R₃₂, R₃₃, R₃₄, R₃₆, R₃₇, R₃₈ and R₃₉ are each independently hydrogen or lower alkyl.
18. The method of embodiment 17, wherein R₃₀, R₃₂, R₃₃, R₃₄, R₃₆, R₃₇, R₃₈ and R₃₉ are each independently hydrogen or methyl.
19. The method of embodiment 17, wherein W is A1 and R₃₀ is methyl.
20. The method of embodiment 17, wherein W is A2 and R₃₂ is hydrogen and R₃₃ is methyl.
21. The method of embodiment 17, wherein W is A7 and Rb is methyl.
22. The method of embodiment 17, wherein the Reversible and Selective MAGL Inhibitor Compound is a compound of Formula (I-C),

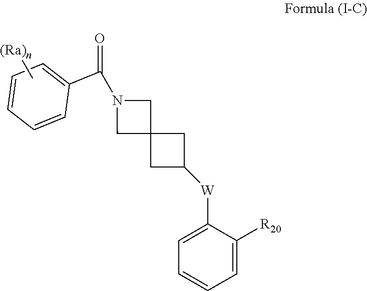

wherein R₂₀ is lower alkyl; and Ra, n, and W are as defined above with respect to Formula (I-B).
23. The method of embodiment 22, wherein R₂₀ is methyl optionally substituted with one or more F.
24. A method comprising the administration of a Reversible and Selective MAGL Inhibitor Compound of Formula (II), or a pharmaceutically acceptable salt thereof,

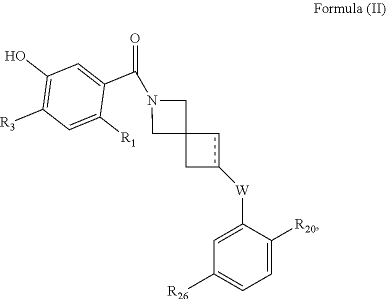

wherein
   R₁ is halogen or cyano;
   R₃ is hydrogen or halogen;
   W is A;
   R₁₀ is hydrogen or lower alkyl;
   A is a 5-member heteroaryl ring optionally substituted with one or more R₃₀; and
   R₂₀ and R₃₀ are each independently lower alkyl.
25. A method comprising the administration of a Reversible and Selective MAGL Inhibitor Compound of Formula (III), or a pharmaceutically acceptable salt thereof,

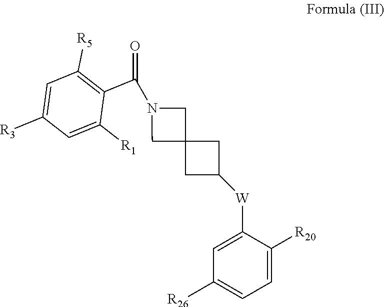

wherein
   R₃ is halogen;
   R₆ is —O—R₆₂;
   R₆₂ is lower alkyl or cycloalkyl, each optionally substituted with halogen,
   W is a S-member heteroaryl ring optionally substituted with one or more R₃₀;
   R₃₀ is lower alkyl; and
   R₂₀ is lower alkyl.
26. The method of embodiment 25, wherein W is a 5-member heteroaryl ring comprising at least one nitrogen heteroatom and optionally substituted with one methyl.
27. A method comprising the administration of a compound selected from the group consisting of:

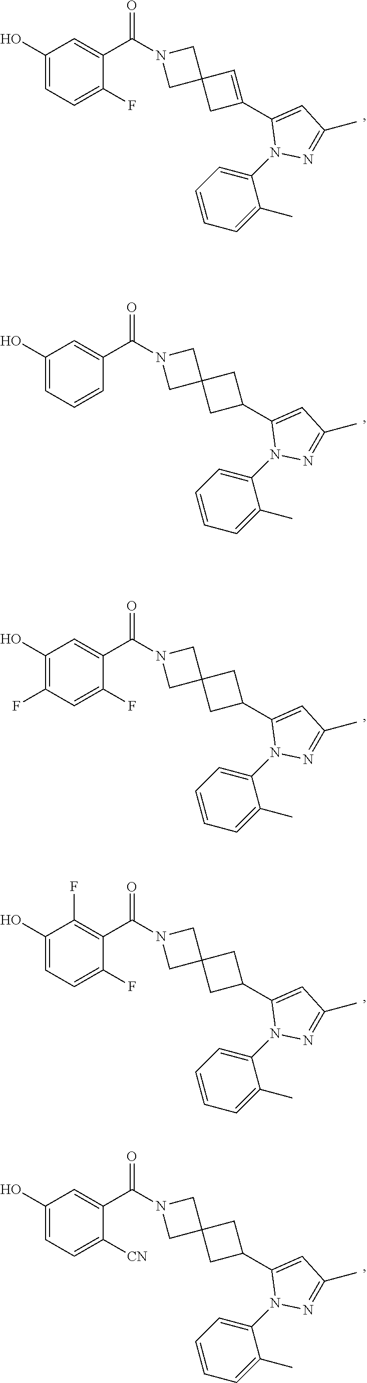 [SMILES: Cc1cc(C2=CC3(C2)CN(C(=O)c2cc(O)ccc2F)C3)n(-c2ccccc2C)n1 ; Cc1cc(C2CC3(C2)CN(C(=O)c2cccc(O)c2)C3)n(-c2ccccc2C)n1 ; Cc1cc(C2CC3(C2)CN(C(=O)c2cc(O)c(F)cc2F)C3)n(-c2ccccc2C)n1 ; Cc1cc(C2CC3(C2)CN(C(=O)c2c(F)ccc(O)c2F)C3)n(-c2ccccc2C)n1 ; Cc1cc(C2CC3(C2)CN(C(=O)c2cc(O)ccc2C#N)C3)n(-c2ccccc2C)n1]

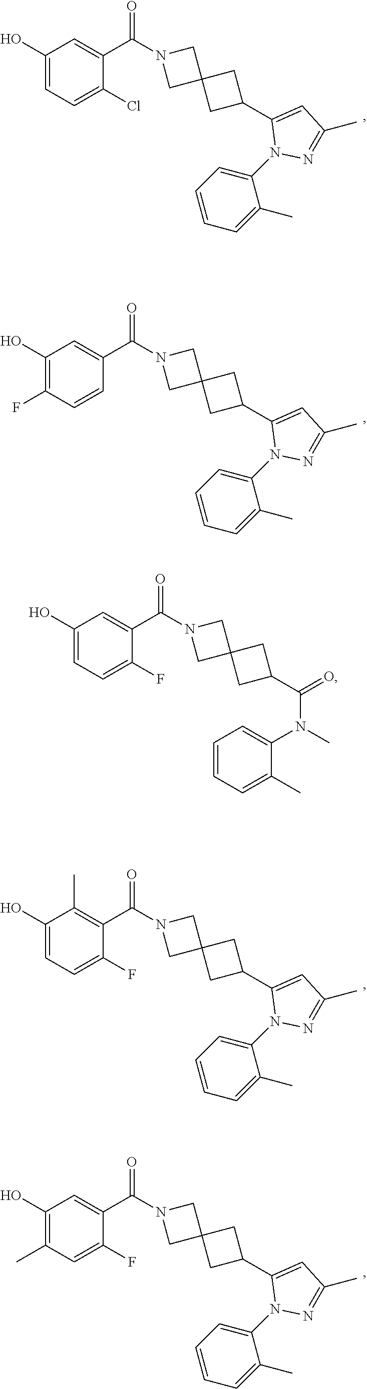 [SMILES: Cc1cc(C2CC3(C2)CN(C(=O)c2cc(O)ccc2Cl)C3)n(-c2ccccc2C)n1 ; Cc1cc(C2CC3(C2)CN(C(=O)c2ccc(F)c(O)c2)C3)n(-c2ccccc2C)n1 ; Cc1ccccc1N(C)C(=O)C1CC2(C1)CN(C(=O)c1cc(O)ccc1F)C2 ; Cc1cc(C2CC3(C2)CN(C(=O)c2c(F)ccc(O)c2C)C3)n(-c2ccccc2C)n1 ; Cc1cc(C2CC3(C2)CN(C(=O)c2cc(O)c(C)cc2F)C3)n(-c2ccccc2C)n1]

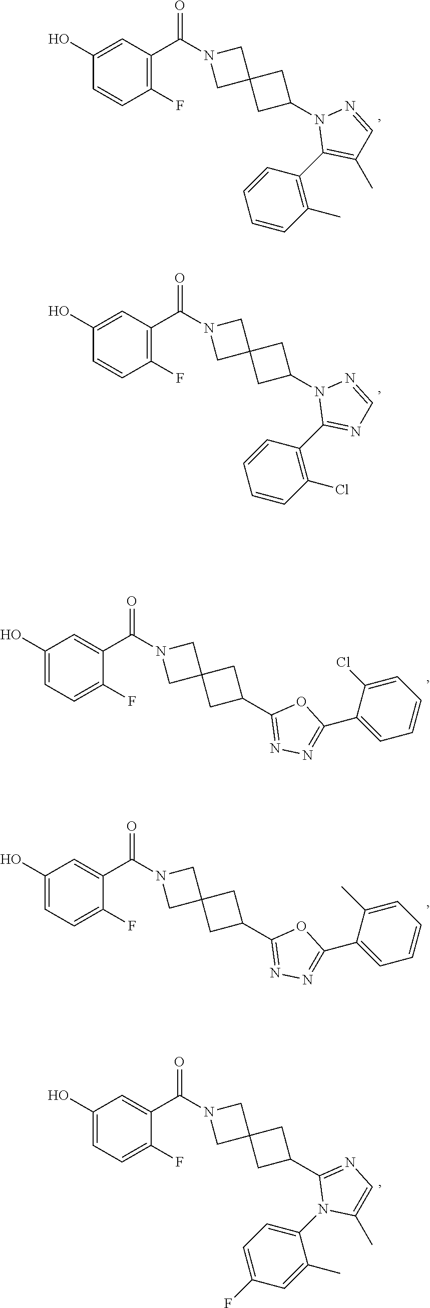 [SMILES: Cc1ccccc1-c1c(C)cnn1C1CC2(C1)CN(C(=O)c1cc(O)ccc1F)C2 ; O=C(c1cc(O)ccc1F)N1CC2(CC(n3ncnc3-c3ccccc3Cl)C2)C1 ; O=C(c1cc(O)ccc1F)N1CC2(CC(c3nnc(-c4ccccc4Cl)o3)C2)C1 ; Cc1ccccc1-c1nnc(C2CC3(C2)CN(C(=O)c2cc(O)ccc2F)C3)o1 ; Cc1cc(F)ccc1-n1c(C)cnc1C1CC2(C1)CN(C(=O)c1cc(O)ccc1F)C2]

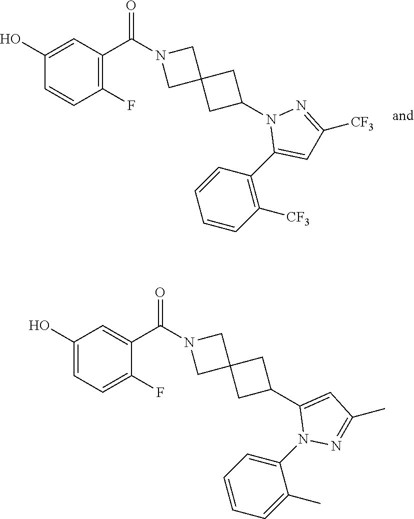 [SMILES: O=C(c1cc(O)ccc1F)N1CC2(CC(n3nc(C(F)(F)F)cc3-c3ccccc3C(F)(F)F)C2)C1 ; Cc1cc(C2CC3(C2)CN(C(=O)c2cc(O)ccc2F)C3)n(-c2ccccc2C)n1]

or a pharmaceutically acceptable salt thereof.
28. A method comprising the administration of a compound selected from the group consisting of:

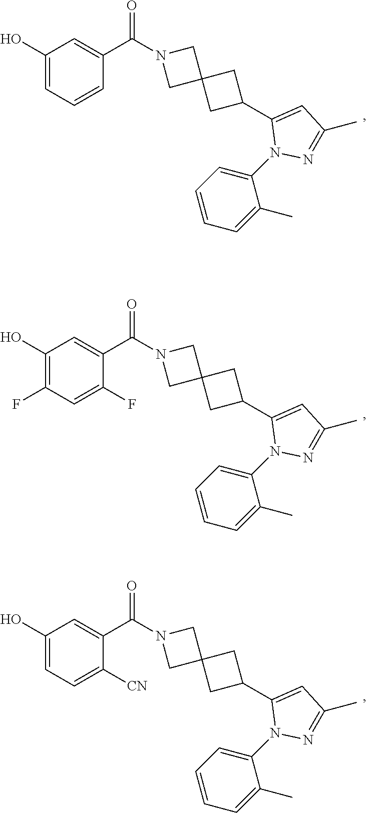 [SMILES: Cc1cc(C2CC3(C2)CN(C(=O)c2cccc(O)c2)C3)n(-c2ccccc2C)n1 ; Cc1cc(C2CC3(C2)CN(C(=O)c2cc(O)c(F)cc2F)C3)n(-c2ccccc2C)n1 ; Cc1cc(C2CC3(C2)CN(C(=O)c2cc(O)ccc2C#N)C3)n(-c2ccccc2C)n1]

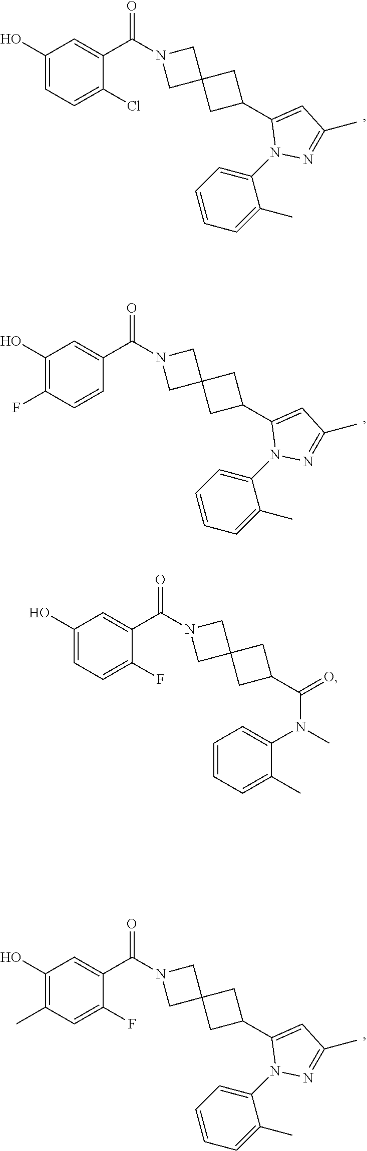 [SMILES: Cc1cc(C2CC3(C2)CN(C(=O)c2cc(O)ccc2Cl)C3)n(-c2ccccc2C)n1 ; Cc1cc(C2CC3(C2)CN(C(=O)c2ccc(F)c(O)c2)C3)n(-c2ccccc2C)n1 ; Cc1ccccc1N(C)C(=O)C1CC2(C1)CN(C(=O)c1cc(O)ccc1F)C2 ; Cc1cc(C2CC3(C2)CN(C(=O)c2cc(O)c(C)cc2F)C3)n(-c2ccccc2C)n1]

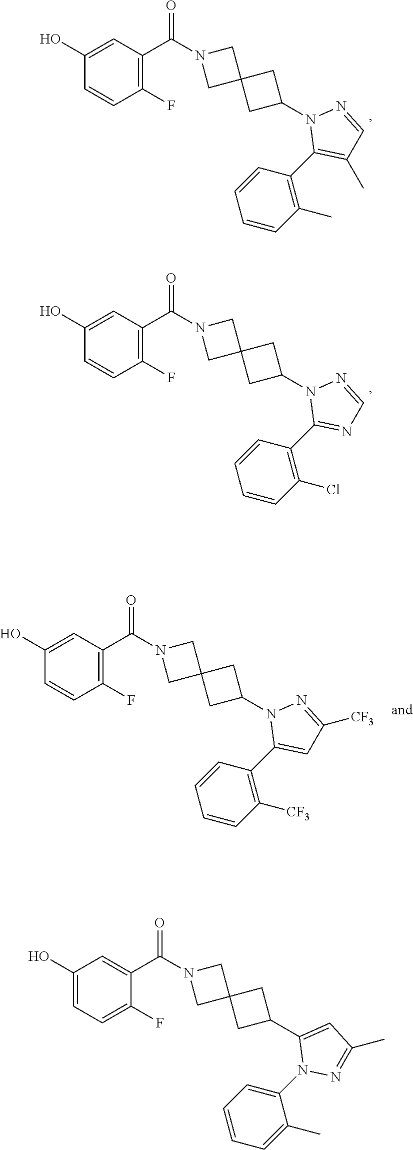 [SMILES: Cc1ccccc1-c1c(C)cnn1C1CC2(C1)CN(C(=O)c1cc(O)ccc1F)C2 ; O=C(c1cc(O)ccc1F)N1CC2(CC(n3ncnc3-c3ccccc3Cl)C2)C1 ; O=C(c1cc(O)ccc1F)N1CC2(CC(n3nc(C(F)(F)F)cc3-c3ccccc3C(F)(F)F)C2)C1 ; Cc1cc(C2CC3(C2)CN(C(=O)c2cc(O)ccc2F)C3)n(-c2ccccc2C)n1]

or a pharmaceutically acceptable salt thereof.
29. A method comprising the administration of a compound selected from the group consisting of:

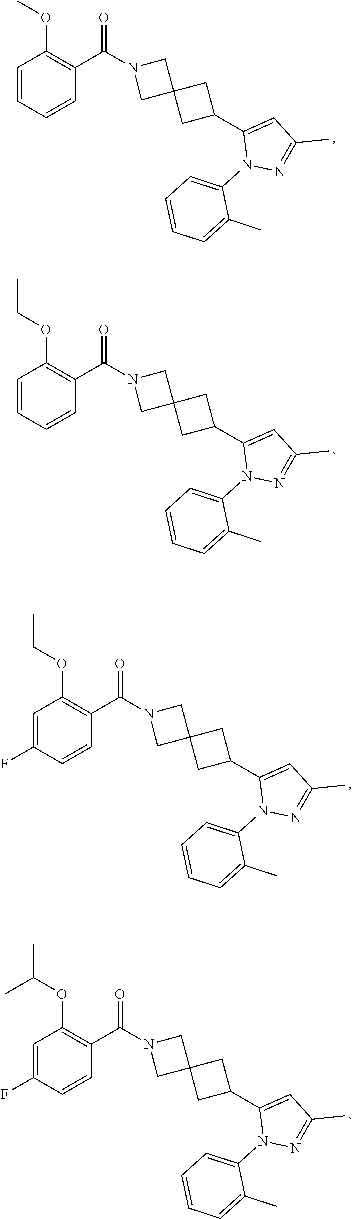 [SMILES: COc1ccccc1C(=O)N1CC2(CC(c3cc(C)nn3-c3ccccc3C)C2)C1 ; CCOc1ccccc1C(=O)N1CC2(CC(c3cc(C)nn3-c3ccccc3C)C2)C1 ; CCOc1cc(F)ccc1C(=O)N1CC2(CC(c3cc(C)nn3-c3ccccc3C)C2)C1 ; Cc1cc(C2CC3(C2)CN(C(=O)c2ccc(F)cc2OC(C)C)C3)n(-c2ccccc2C)n1]

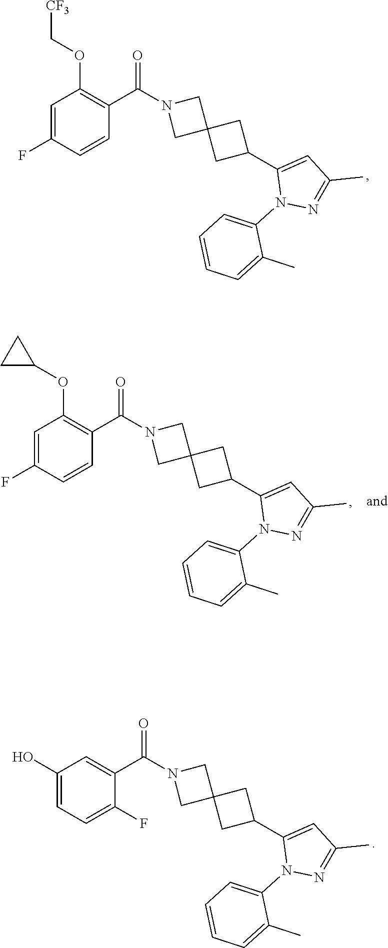 [SMILES: Cc1cc(C2CC3(C2)CN(C(=O)c2ccc(F)cc2OCC(F)(F)F)C3)n(-c2ccccc2C)n1 ; Cc1cc(C2CC3(C2)CN(C(=O)c2ccc(F)cc2OC2CC2)C3)n(-c2ccccc2C)n1 ; Cc1cc(C2CC3(C2)CN(C(=O)c2cc(O)ccc2F)C3)n(-c2ccccc2C)n1]

30. A method comprising the administration of a compound selected from the group consisting of:

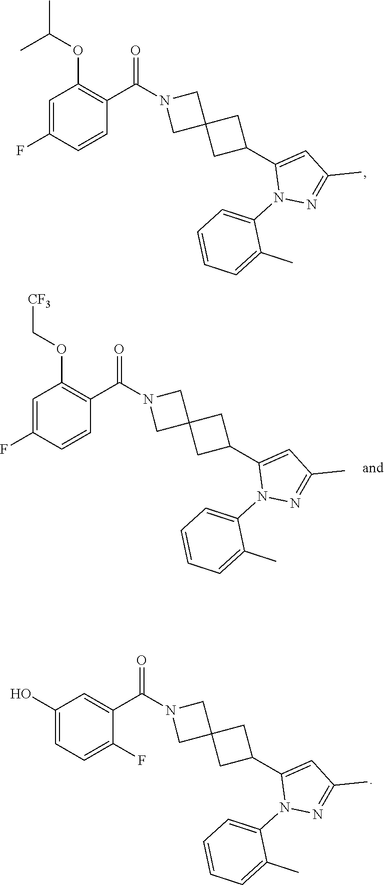 [SMILES: Cc1cc(C2CC3(C2)CN(C(=O)c2ccc(F)cc2OC(C)C)C3)n(-c2ccccc2C)n1 ; Cc1cc(C2CC3(C2)CN(C(=O)c2ccc(F)cc2OCC(F)(F)F)C3)n(-c2ccccc2C)n1 ; Cc1cc(C2CC3(C2)CN(C(=O)c2cc(O)ccc2F)C3)n(-c2ccccc2C)n1]

31. A method comprising the administration of a compound

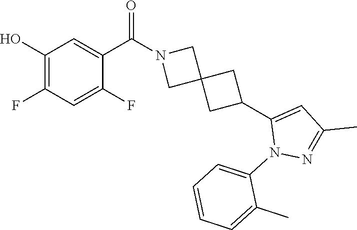 [SMILES: Cc1cc(C2CC3(C2)CN(C(=O)c2cc(O)c(F)cc2F)C3)n(-c2ccccc2C)n1]

or a pharmaceutically acceptable salt thereof.
32. A method comprising the administration of a compound

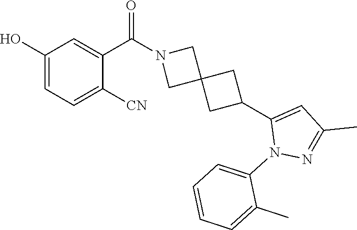 [SMILES: Cc1cc(C2CC3(C2)CN(C(=O)c2cc(O)ccc2C#N)C3)n(-c2ccccc2C)n1]

or a pharmaceutically acceptable salt thereof.
33. A method comprising the administration of a compound

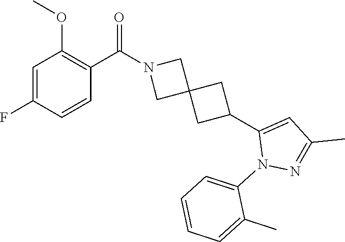 [SMILES: COc1cc(F)ccc1C(=O)N1CC2(CC(c3cc(C)nn3-c3ccccc3C)C2)C1]

or a pharmaceutically acceptable salt thereof.
34. A method comprising the administration of a compound

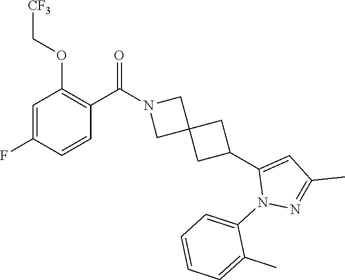 [SMILES: Cc1cc(C2CC3(C2)CN(C(=O)c2ccc(F)cc2OCC(F)(F)F)C3)n(-c2ccccc2C)n1]

or a pharmaceutically acceptable salt thereof.
35. A method comprising the administration of a compound

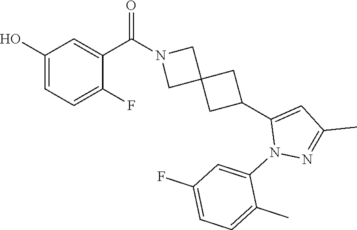 [SMILES: Cc1cc(C2CC3(C2)CN(C(=O)c2cc(O)ccc2F)C3)n(-c2cc(F)ccc2C)n1]

or a pharmaceutically acceptable salt thereof.
36. A method comprising the administration of a compound

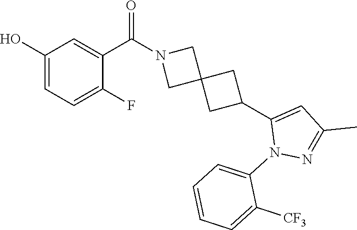 [SMILES: Cc1cc(C2CC3(C2)CN(C(=O)c2cc(O)ccc2F)C3)n(-c2ccccc2C(F)(F)F)n1]

or a pharmaceutically acceptable salt thereof.
37. A method comprising the administration of a compound

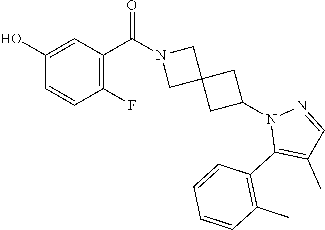 [SMILES: Cc1ccccc1-c1c(C)cnn1C1CC2(C1)CN(C(=O)c1cc(O)ccc1F)C2]

or a pharmaceutically acceptable salt thereof.
38. A method comprising the administration of a compound

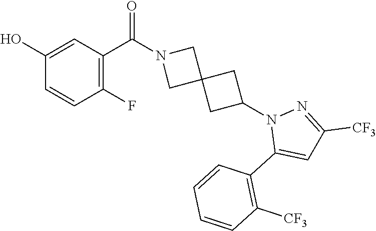 [SMILES: O=C(c1cc(O)ccc1F)N1CC2(CC(n3nc(C(F)(F)F)cc3-c3ccccc3C(F)(F)F)C2)C1]

or a pharmaceutically acceptable salt thereof.
39. A method comprising the administration of a compound

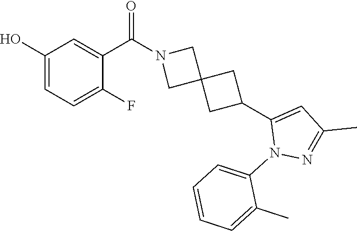 [SMILES: Cc1cc(C2CC3(C2)CN(C(=O)c2cc(O)ccc2F)C3)n(-c2ccccc2C)n1]

or a pharmaceutically acceptable salt thereof.
40. A method of transiently increasing 2-AG in the brain of a subject, comprising the administration of a compound according to the method of any one of embodiments 24-39.
41. The method of embodiment 40, wherein the compound is orally administered to the subject.
42. The method of embodiment 41, wherein the half-life of the transient increase of 2-AG in the brain of the subject is less than twice the blood plasma half-life of the compound.
43. The method of embodiment 42, wherein the maximum increase of 2-AG is observed in the brain of the subject 30 minutes after administration of the Reversible Selective MAGL Inhibitor Compound.
44. The method of any one of embodiments 40-43, wherein the a therapeutically effective increase of 2-AG is observed in the brain of the subject 30 minutes after administration of the compound.
45. A method of treating generalized anxiety disorder in a human subject comprising the oral administration of a therapeutically effective amount of a compound according to the methods of any one of embodiments 24-48 to the subject in need thereof.
46. A method of treating post-traumatic stress disorder in a human subject comprising the oral administration of a therapeutically effective amount of a compound according to the methods of any one of embodiments 24-45 to the subject in need thereof.
47. A method of transiently increasing 2-AG in the brain of a subject, comprising the administration of (2-fluoro-5-hydroxyphenyl) (6-(3-methyl-1-(o-tolyl)-1H-pyrazol-5-yl)-2-azaspiro[3.3]heptan-2-yl) methanone or a pharmaceutically acceptable salt thereof.
48. The method of embodiment 45, wherein the compound is orally administered to the subject.
49. The method of any one of embodiments 1-46, wherein the half-life of the transient increase of 2-AG in the brain of the subject is less than twice the blood plasma half-life of the compound.
50. The method of any one of embodiments 1-49, wherein the maximum increase of 2-AG is observed in the brain of the subject 30 minutes after administration of the compound.
51. The method of any one of embodiments 1-50, wherein a therapeutically effective increase of 2-AG is observed in the brain of the subject 30 minutes after administration of the compound.
52. A method of treating or managing anxiety comprising the step of administering to a subject in need thereof a therapeutically effective amount of a compound of Formula (II-A), or a pharmaceutically acceptable salt thereof, to treat or manage anxiety of the subject:

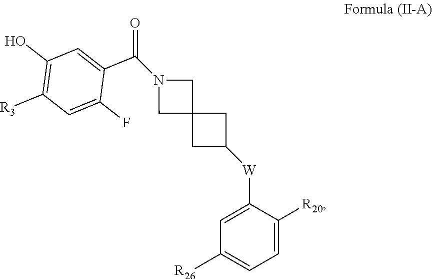

wherein
R₃ is hydrogen, methyl optionally substituted with one or more F, or F;
W is a 5-member heteroaryl ring comprising at least one nitrogen heteroatom optionally substituted with one methyl optionally substituted with one or more F; or cyclopropyl;
R₂₀ is methyl optionally substituted with one or more F, Cl or F;
R₂₆ is hydrogen or F.
53. A method of treating or managing anxiety comprising the step of administering to a subject in need thereof a therapeutically effective amount of a compound of Formula (III-A), or a pharmaceutically acceptable salt thereof, to treat or manage the anxiety of the subject:

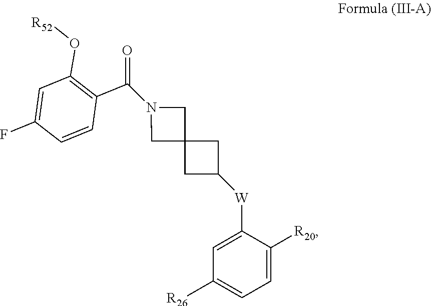

wherein
   R₃₂ is cyclopropyl or (C₁-C₄)alkyl optionally substituted with one or more F;
   W is C(O)N(R₁₀)— and R₁₀ is methyl; or W is a 5-member heteroaryl ring comprising at least one nitrogen heteroatom and optionally substituted with one methyl;
   R₂₀ is (C₁-C₄)alkyl; and
   R₂₆ is hydrogen or F.
54. The method of any one of embodiments 1-53, wherein the method is a method for treating PTSD.
55. A method of treating anxiety to a subject in need thereof, comprising the step of administering a therapeutically effective amount of a compound according to a method of any one of embodiments 1-54.
56. A method of treating anxiety in a subject, comprising the administration to the subject in need thereof: the compound (2-fluoro-5-hydroxyphenyl) (6-(3-methyl-1-(o-tolyl)-1H-pyrazol-5-yl)-2-azaspiro[3.3]heptan-2-yl) methanone or a pharmaceutically acceptable salt thereof.
57. A method of treating PTSD in a subject, comprising the administration to the subject in need thereof: the compound (2-fluoro-5-hydroxyphenyl) (6-(3-methyl-1-(o-tolyl)-1H-pyrazol-5-yl)-2-azaspiro[3.3]heptan-2-yl) methanone or a pharmaceutically acceptable salt thereof.
58. The method of any one of embodiments 8-10, 13-20, 22-26, 40-46 and 52-53 wherein W in the compound is selected from the group consisting of:

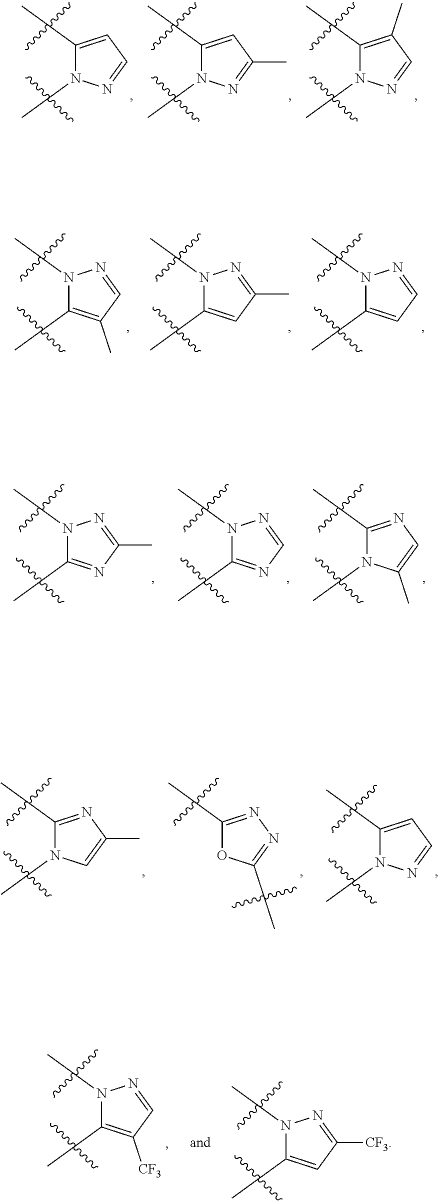

59. The method of embodiment 58, wherein W is

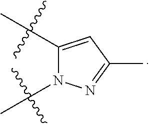 [SMILES: *c1cc(C)nn1*]

60. The method of embodiment 58, wherein W is

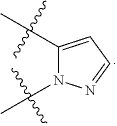 [SMILES: *c1ccnn1*]

61. The method of embodiment 58, wherein W is

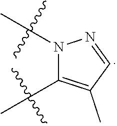 [SMILES: *c1c(C)cnn1*]

62. The method of embodiment 58, wherein W is

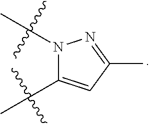 [SMILES: *c1cc(C)nn1*]

63. The method of embodiment 58, wherein W is

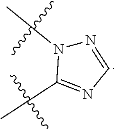 [SMILES: *c1ncnn1*]

64. The method of embodiment 58, wherein W is

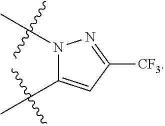 [SMILES: *c1cc(C(F)(F)F)nn1*]

65. The method of embodiment 58, wherein W is

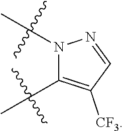 [SMILES: *c1c(C(F)(F)F)cnn1*]

66. A method of treating a disorder selected from the group consisting of: generalized anxiety disorder (GAD), panic disorder, social anxiety disorder, obsessive-compulsive disorder (OCD), and post-traumatic stress disorder (PTSD), comprising the step of administering a therapeutically effective amount of a Reversible Selective MAGL Inhibitor Compound to the patient in need thereof.
67. The method of embodiment 66, wherein the Reversible Selective MAGL Inhibitor Compound is a compound of Formula (I-A) or a pharmaceutically acceptable salt thereof:

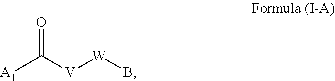

wherein
   A₁ is an aryl or heteroaryl optionally substituted with one or more Ra;
   each Ra is independently halogen, cyano, lower alkyl optionally substituted with one or more halogen, cycloalkyl, aminoalkyl, carboxy, carboxamide, or —OR₆;
   R₆ is hydrogen, lower alkyl or lower cycloalkyl optionally substituted with one or more halogen;
   V is selected from

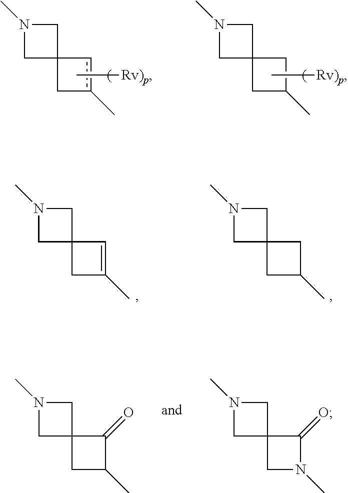

each p is independently 0, 1, 2, 3 or 4;
each Rv is independently hydrogen, halogen, or alkyl optionally substituted with one or more halogen;
W is -A₂-, —C(O)—, C(O)-A₂-, —C(O)N(R₁₀)— and —C(O)N(R₁₀)-A₂-;
A₂ is a 5-member heteroaryl ring optionally substituted with one or more R₃₀;
each R₃₀ is lower alkyl;
R₁₀ is hydrogen or lower alkyl;
B is 5- or 6-member aryl or heteroaryl optionally substituted with one or more R_{b} or or —OR_{b}; and
each R_{b} is independently halogen, cyano, lower alkyl optionally substituted with one or more halogen, cycloalkyl, aminoalkyl, carboxy, or carboxamide.
68. The method of embodiment 66, wherein the Reversible Selective MAGL Inhibitor Compound is a compound of Formula (I-B) or a pharmaceutically acceptable salt thereof:

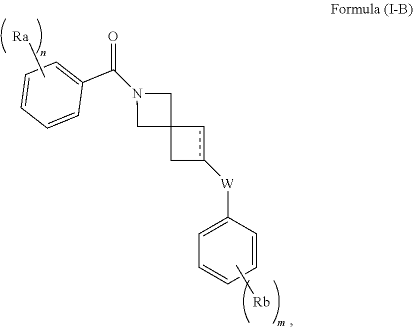

wherein
   n is 1, 2, or 3;
   each Ra is independently halogen, cyano, lower alkyl optionally substituted with one or more halogen, or —OR₆;
   R₆ is hydrogen, lower alkyl or lower cycloalkyl optionally substituted with one or more halogen;
   W is A, —C(O)—, —C(O)-A-, or —C(O)N(R₁₀)—;
   R₁₀ is hydrogen or lower alkyl;
   A is a 5-member heteroaryl ring optionally substituted with one or more R₃₀,
   R₃₀ is lower alkyl;
   m is 1, or 2; and
   each R_{b} is independently halogen, or lower alkyl optionally substituted with one or more halogen.
69. The method of embodiment 66, wherein the Reversible Selective MAGL Inhibitor Compound is a compound of Formula (I-B-1) or a pharmaceutically acceptable salt thereof:

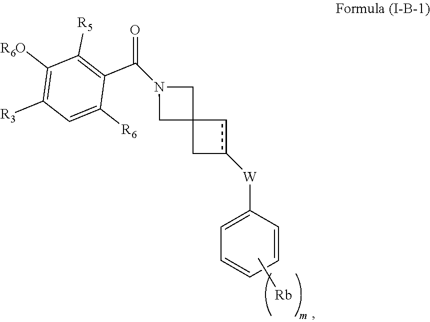

wherein
   R₁ is hydrogen or halogen;
   R₃ is hydrogen, halogen, or lower alkyl optionally substituted with one or more halogen;
   R₅ is hydrogen, halogen, lower alkoxy or lower alkyl each optionally substituted with one or more halogen;
   R₆ is hydrogen, lower alkyl or cycloalkyl optionally substituted with one or more halogen, and
   W is A, —C(O)—, or —C(O)N(R₁₀)—;
   A is aryl or heteroaryl each optionally substituted with one or more R₃₀,
   each R₃₀ is independently lower alkyl optionally substituted with one or more halogen;
   R₁₀ is hydrogen or lower alkyl; and
   m is 1, 2, 3, 4 or 5.
70. The method of embodiment 66, wherein the Reversible Selective MAGL Inhibitor Compound is a compound of Formula (I-B-2) or a pharmaceutically acceptable salt thereof:

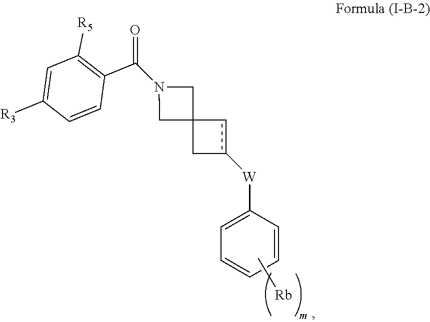

wherein
   R₅ is hydrogen or halogen;
   R₅ is —O—R₅₂;
   R₅₂ is lower alkyl or cycloalkyl, each optionally substituted with halogen,
   W is A, —C(O)—, or —C(O)N(R₁₀)—;
   A is aryl or heteroaryl each optionally substituted with one or more R₃₀;
   each R₃₀ is independently lower alkyl optionally substituted with one or more halogen,
   R₁₀ is hydrogen or lower alkyl; and
   m is 1, 2, 3, 4 or 5.
71. The method of embodiment 66, wherein the Reversible Selective MAGL Inhibitor Compound is a compound of Formula (I-C) or a pharmaceutically acceptable salt thereof:

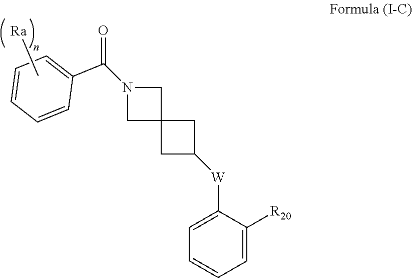

wherein:
   R₂₀ is lower alkyl;
   n is 1, 2, or 3;
   each Ra is independently halogen, cyano, lower alkyl optionally substituted with one or more halogen, or —OR₆;
   W is as defined with respect to Formula (I-B); and
   R₆ is hydrogen, lower alkyl or lower cycloalkyl optionally substituted with one or more halogen.
72. The method of embodiment 66, wherein the Reversible Selective MAGL Inhibitor Compound is a compound of Formula (II) or a pharmaceutically acceptable salt thereof:

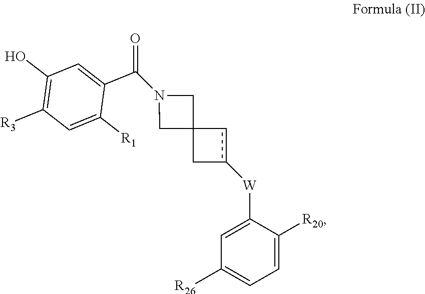

wherein
   R₁ is halogen or cyano;
   R₃ is hydrogen or halogen;
   W is A, —C(O)—, or —C(O)N(R₁₀)—;
   R₁₀ is hydrogen or lower alkyl;
   A is a 5-member heteroaryl ring optionally substituted with one or more R₃₀; and
   R₂₀ and R₃₀ are each independently lower alkyl.
73. The method of embodiment 66, wherein the Reversible Selective MAGL Inhibitor Compound is a compound of Formula (II-A) or a pharmaceutically acceptable salt thereof:

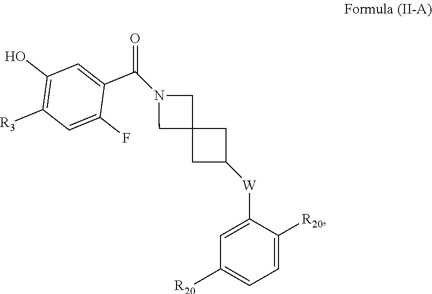

wherein
   R₃ is hydrogen, methyl optionally substituted with one or more F, or F;
   W is a 5-member heteroaryl ring comprising at least one nitrogen heteroatom optionally substituted with one methyl optionally substituted with one or more F; or cyclopropyl;
   R₂₀ is methyl optionally substituted with one or more F, Cl or F;
   R₂₆ is hydrogen or F.
74. The method of embodiment 66, wherein the Reversible Selective MAGL Inhibitor Compound is a compound of Formula (III) or a pharmaceutically acceptable salt thereof:

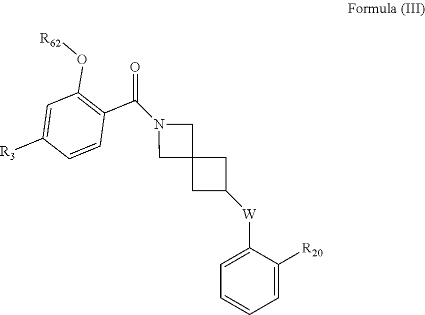

wherein
   R₃ is halogen;
   R₆₂ is lower alkyl or cycloalkyl, each optionally substituted with halogen,
   W is a 5-member heteroaryl ring optionally substituted with one or more R₃₀;
   R₃₀ is lower alkyl; and
   R₂₀ is lower alkyl.
75. The method of embodiment 66, wherein the Reversible Selective MAGL Inhibitor Compound is a compound of Formula (III-A) or a pharmaceutically acceptable salt thereof:

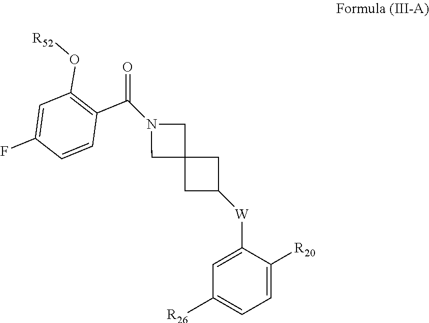

wherein
   R₅₂ is cyclopropyl or (C₁-C₄) alkyl optionally substituted with one or more F;
   W is C(O)N(R₁₀)— and R₁₀ is methyl; or W is a 5-member heteroaryl ring comprising at least one nitrogen heteroatom and optionally substituted with one methyl;
   R₂₀ is (C₁-C₄) alkyl; and
   R₂₆ is hydrogen or F.
76. The method of any one of embodiments 67-75, wherein W in the compound is selected from the group consisting of:

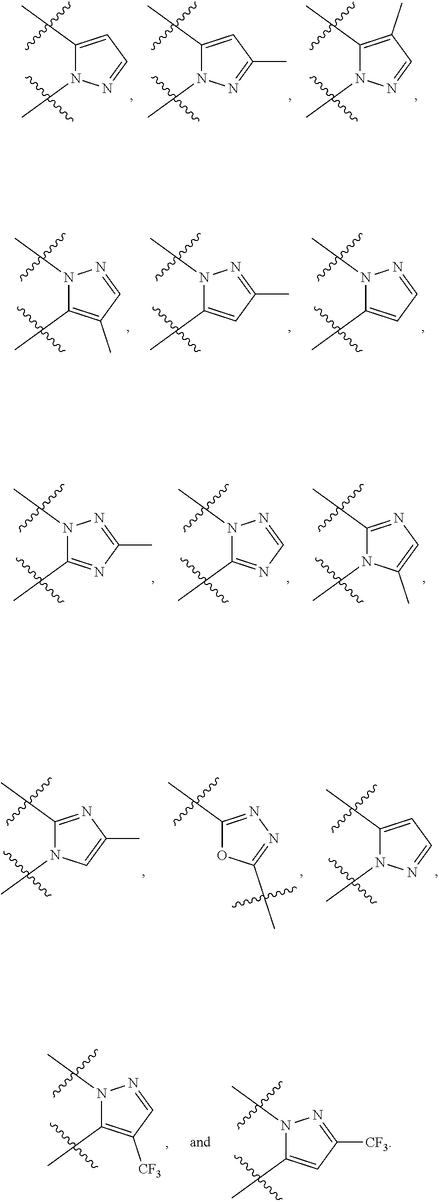

77. The method of embodiment 76, wherein W is

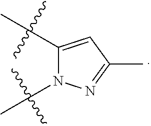 [SMILES: *c1cc(C)nn1*]

78. The method of embodiment 76, wherein W is

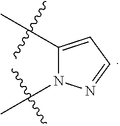 [SMILES: *c1ccnn1*]

79. The method of embodiment 76, wherein W is

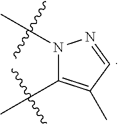 [SMILES: *c1c(C)cnn1*]

80. The method of embodiment 76, wherein W is

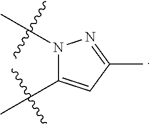 [SMILES: *c1cc(C)nn1*]

81. The method of embodiment 76, wherein W is

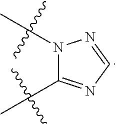 [SMILES: *c1ncnn1*]

82. The method of embodiment 76, wherein W is

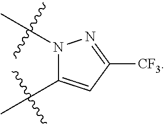 [SMILES: *c1cc(C(F)(F)F)nn1*]

83. The method of embodiment 76, wherein W is

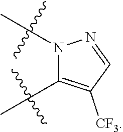 [SMILES: *c1c(C(F)(F)F)cnn1*]

84. The method of embodiment 66, wherein the compound is a compound selected from the group consisting of:

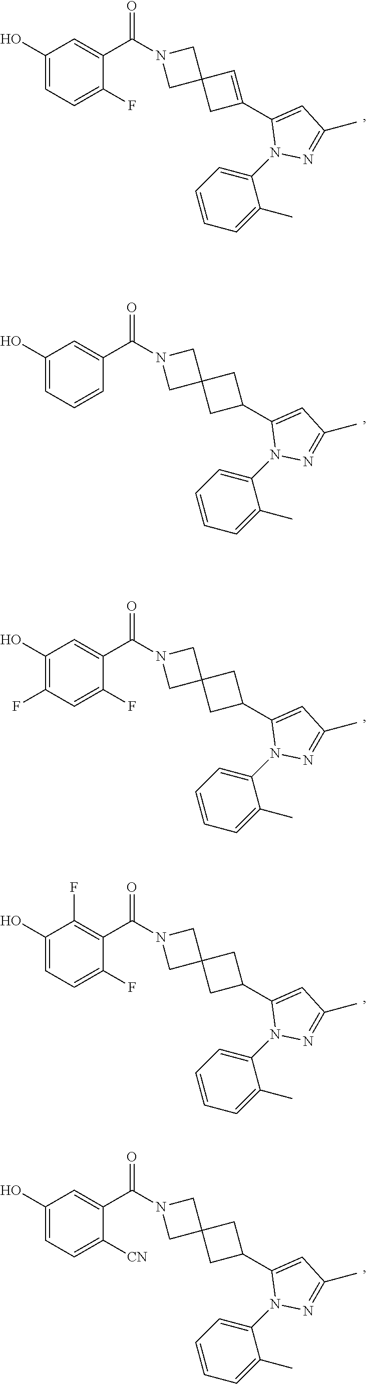 [SMILES: Cc1cc(C2=CC3(C2)CN(C(=O)c2cc(O)ccc2F)C3)n(-c2ccccc2C)n1 ; Cc1cc(C2CC3(C2)CN(C(=O)c2cccc(O)c2)C3)n(-c2ccccc2C)n1 ; Cc1cc(C2CC3(C2)CN(C(=O)c2cc(O)c(F)cc2F)C3)n(-c2ccccc2C)n1 ; Cc1cc(C2CC3(C2)CN(C(=O)c2c(F)ccc(O)c2F)C3)n(-c2ccccc2C)n1 ; Cc1cc(C2CC3(C2)CN(C(=O)c2cc(O)ccc2C#N)C3)n(-c2ccccc2C)n1]

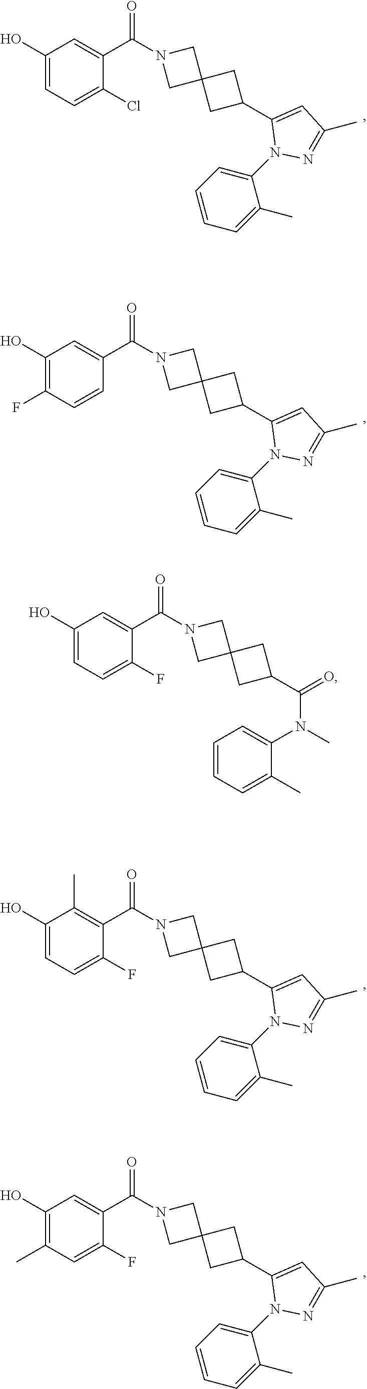 [SMILES: Cc1cc(C2CC3(C2)CN(C(=O)c2cc(O)ccc2Cl)C3)n(-c2ccccc2C)n1 ; Cc1cc(C2CC3(C2)CN(C(=O)c2ccc(F)c(O)c2)C3)n(-c2ccccc2C)n1 ; Cc1ccccc1N(C)C(=O)C1CC2(C1)CN(C(=O)c1cc(O)ccc1F)C2 ; Cc1cc(C2CC3(C2)CN(C(=O)c2c(F)ccc(O)c2C)C3)n(-c2ccccc2C)n1 ; Cc1cc(C2CC3(C2)CN(C(=O)c2cc(O)c(C)cc2F)C3)n(-c2ccccc2C)n1]

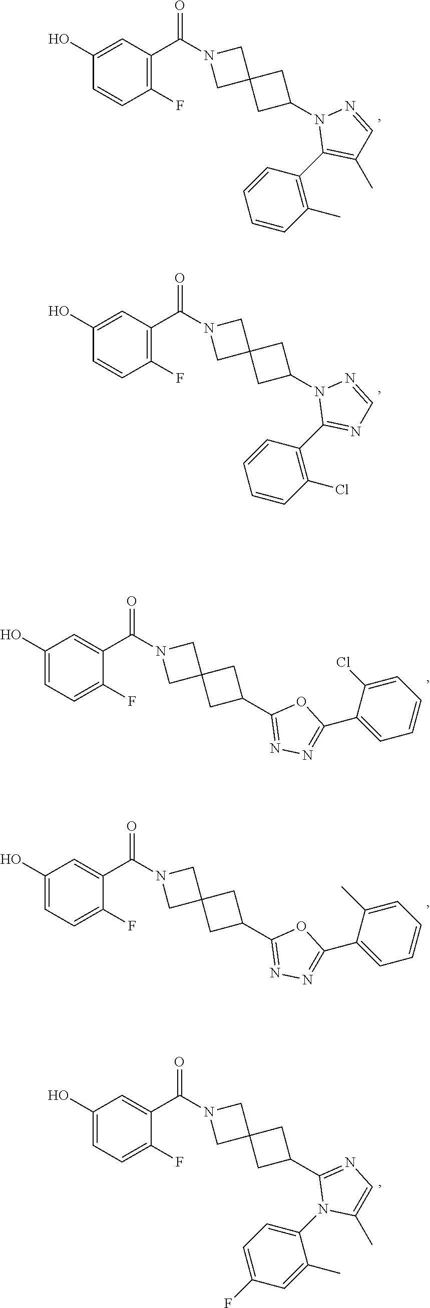 [SMILES: Cc1ccccc1-c1c(C)cnn1C1CC2(C1)CN(C(=O)c1cc(O)ccc1F)C2 ; O=C(c1cc(O)ccc1F)N1CC2(CC(n3ncnc3-c3ccccc3Cl)C2)C1 ; O=C(c1cc(O)ccc1F)N1CC2(CC(c3nnc(-c4ccccc4Cl)o3)C2)C1 ; Cc1ccccc1-c1nnc(C2CC3(C2)CN(C(=O)c2cc(O)ccc2F)C3)o1 ; Cc1cc(F)ccc1-n1c(C)cnc1C1CC2(C1)CN(C(=O)c1cc(O)ccc1F)C2]

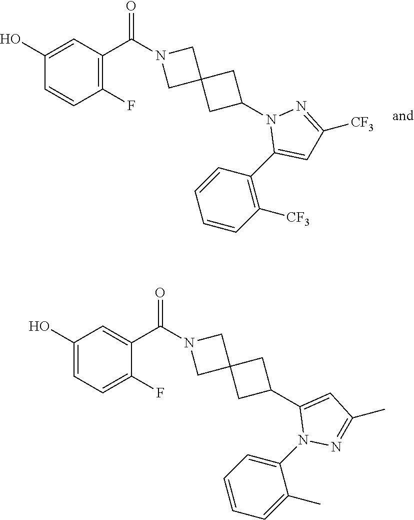 [SMILES: O=C(c1cc(O)ccc1F)N1CC2(CC(n3nc(C(F)(F)F)cc3-c3ccccc3C(F)(F)F)C2)C1 ; Cc1cc(C2CC3(C2)CN(C(=O)c2cc(O)ccc2F)C3)n(-c2ccccc2C)n1]

or a pharmaceutically acceptable salt thereof.
85. The method of embodiment 66, wherein the compound is a compound selected from the group consisting of:

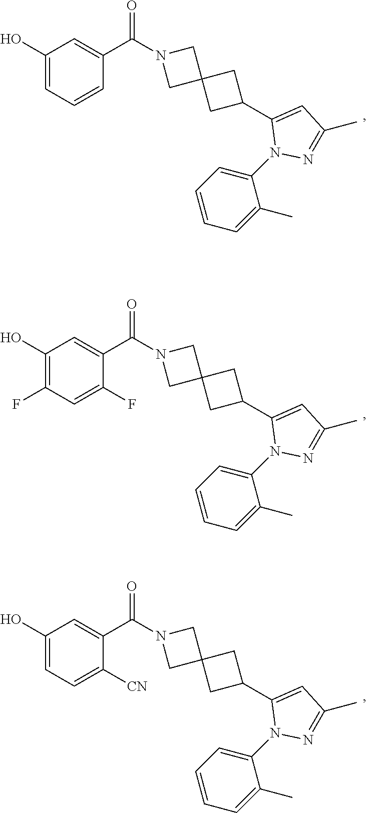 [SMILES: Cc1cc(C2CC3(C2)CN(C(=O)c2cccc(O)c2)C3)n(-c2ccccc2C)n1 ; Cc1cc(C2CC3(C2)CN(C(=O)c2cc(O)c(F)cc2F)C3)n(-c2ccccc2C)n1 ; Cc1cc(C2CC3(C2)CN(C(=O)c2cc(O)ccc2C#N)C3)n(-c2ccccc2C)n1]

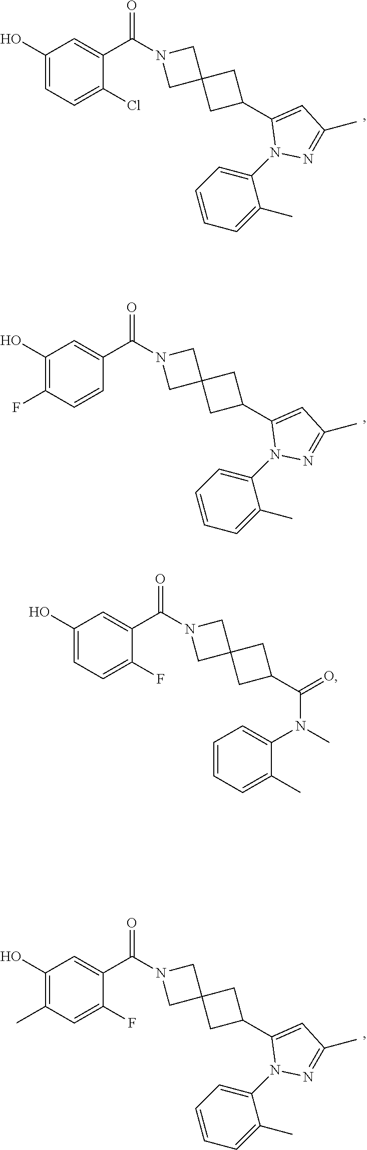 [SMILES: Cc1cc(C2CC3(C2)CN(C(=O)c2cc(O)ccc2Cl)C3)n(-c2ccccc2C)n1 ; Cc1cc(C2CC3(C2)CN(C(=O)c2ccc(F)c(O)c2)C3)n(-c2ccccc2C)n1 ; Cc1ccccc1N(C)C(=O)C1CC2(C1)CN(C(=O)c1cc(O)ccc1F)C2 ; Cc1cc(C2CC3(C2)CN(C(=O)c2cc(O)c(C)cc2F)C3)n(-c2ccccc2C)n1]

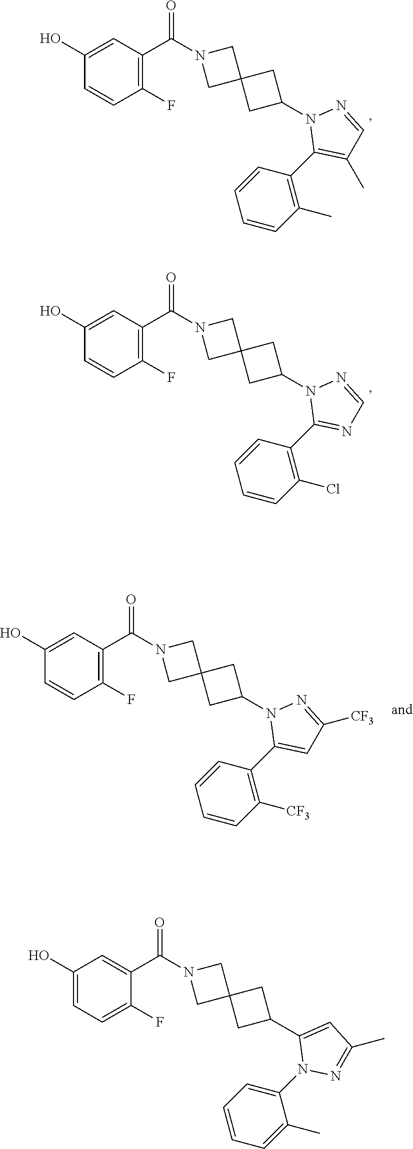 [SMILES: Cc1ccccc1-c1c(C)cnn1C1CC2(C1)CN(C(=O)c1cc(O)ccc1F)C2 ; O=C(c1cc(O)ccc1F)N1CC2(CC(n3ncnc3-c3ccccc3Cl)C2)C1 ; O=C(c1cc(O)ccc1F)N1CC2(CC(n3nc(C(F)(F)F)cc3-c3ccccc3C(F)(F)F)C2)C1 ; Cc1cc(C2CC3(C2)CN(C(=O)c2cc(O)ccc2F)C3)n(-c2ccccc2C)n1]

or a pharmaceutically acceptable salt thereof.
86. The method of embodiment 66, wherein the compound is a compound selected from the group consisting of:

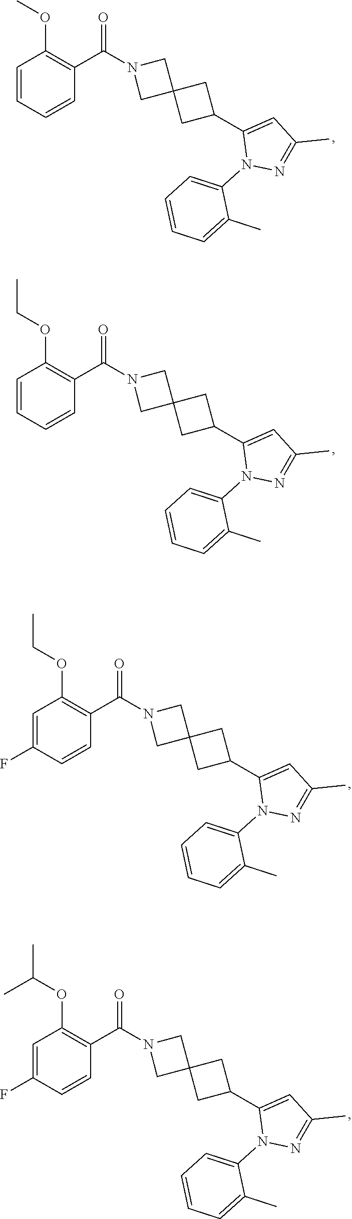 [SMILES: COc1ccccc1C(=O)N1CC2(CC(c3cc(C)nn3-c3ccccc3C)C2)C1 ; CCOc1ccccc1C(=O)N1CC2(CC(c3cc(C)nn3-c3ccccc3C)C2)C1 ; CCOc1cc(F)ccc1C(=O)N1CC2(CC(c3cc(C)nn3-c3ccccc3C)C2)C1 ; Cc1cc(C2CC3(C2)CN(C(=O)c2ccc(F)cc2OC(C)C)C3)n(-c2ccccc2C)n1]

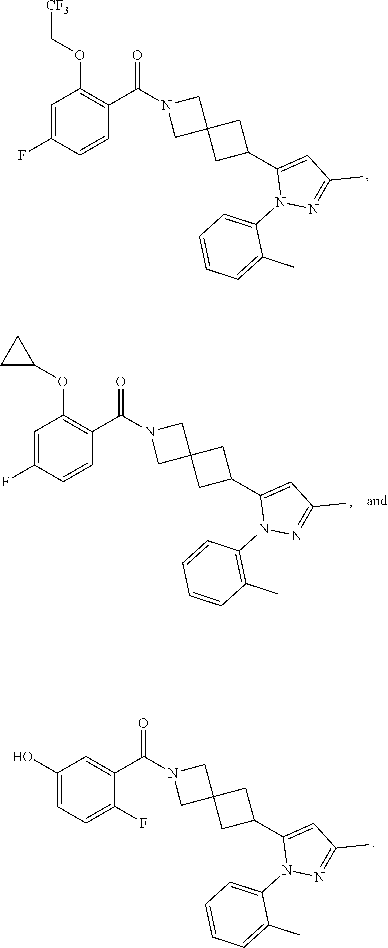 [SMILES: Cc1cc(C2CC3(C2)CN(C(=O)c2ccc(F)cc2OCC(F)(F)F)C3)n(-c2ccccc2C)n1 ; Cc1cc(C2CC3(C2)CN(C(=O)c2ccc(F)cc2OC2CC2)C3)n(-c2ccccc2C)n1 ; Cc1cc(C2CC3(C2)CN(C(=O)c2cc(O)ccc2F)C3)n(-c2ccccc2C)n1]

87. The method of embodiment 66, wherein the compound is a compound selected from the group consisting of:

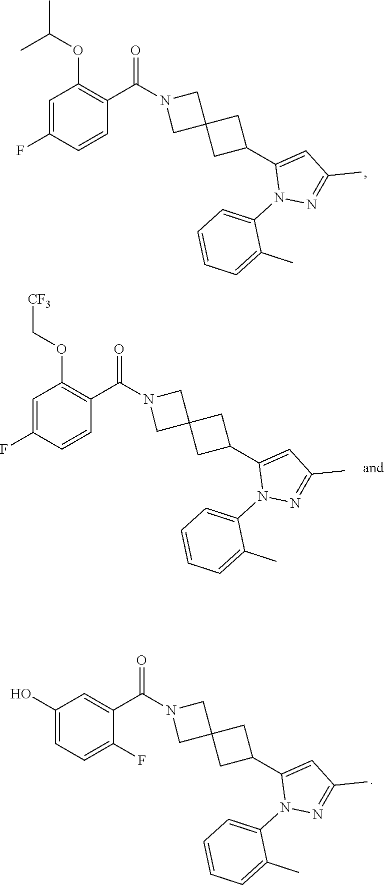 [SMILES: Cc1cc(C2CC3(C2)CN(C(=O)c2ccc(F)cc2OC(C)C)C3)n(-c2ccccc2C)n1 ; Cc1cc(C2CC3(C2)CN(C(=O)c2ccc(F)cc2OCC(F)(F)F)C3)n(-c2ccccc2C)n1 ; Cc1cc(C2CC3(C2)CN(C(=O)c2cc(O)ccc2F)C3)n(-c2ccccc2C)n1]

88. The method of embodiment 66, wherein the compound is a compound

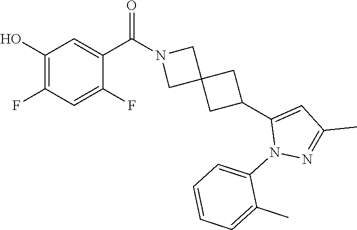 [SMILES: Cc1cc(C2CC3(C2)CN(C(=O)c2cc(O)c(F)cc2F)C3)n(-c2ccccc2C)n1]

or a pharmaceutically acceptable salt thereof.
89. The method of embodiment 66, wherein the compound is a compound

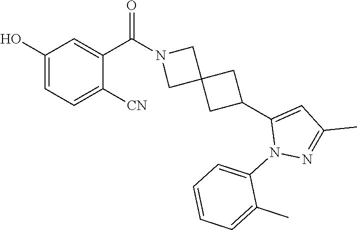 [SMILES: Cc1cc(C2CC3(C2)CN(C(=O)c2cc(O)ccc2C#N)C3)n(-c2ccccc2C)n1]

or a pharmaceutically acceptable salt thereof.
90. The method of embodiment 66, wherein the compound is a compound

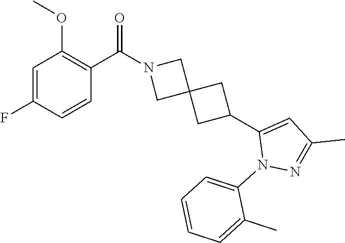 [SMILES: COc1cc(F)ccc1C(=O)N1CC2(CC(c3cc(C)nn3-c3ccccc3C)C2)C1]

or a pharmaceutically acceptable salt thereof.
91. The method of embodiment 66, wherein the compound is a compound

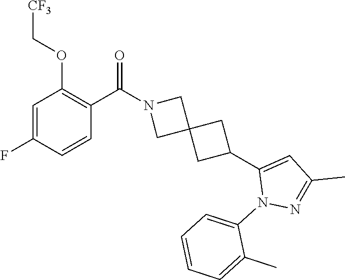 [SMILES: Cc1cc(C2CC3(C2)CN(C(=O)c2ccc(F)cc2OCC(F)(F)F)C3)n(-c2ccccc2C)n1]

or a pharmaceutically acceptable salt thereof.
92. The method of embodiment 66, wherein the compound is a compound

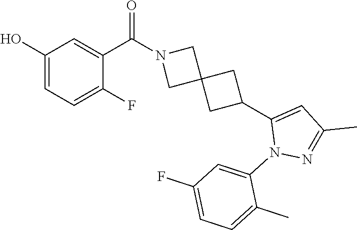 [SMILES: Cc1cc(C2CC3(C2)CN(C(=O)c2cc(O)ccc2F)C3)n(-c2cc(F)ccc2C)n1]

or a pharmaceutically acceptable salt thereof.
93. The method of embodiment 66, wherein the compound is a compound

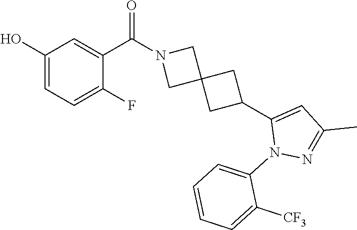 [SMILES: Cc1cc(C2CC3(C2)CN(C(=O)c2cc(O)ccc2F)C3)n(-c2ccccc2C(F)(F)F)n1]

or a pharmaceutically acceptable salt thereof.
94. The method of embodiment 66, wherein the compound is a compound

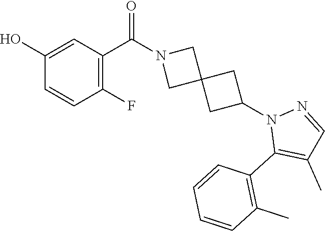 [SMILES: Cc1ccccc1-c1c(C)cnn1C1CC2(C1)CN(C(=O)c1cc(O)ccc1F)C2]

or a pharmaceutically acceptable salt thereof.
95. The method of embodiment 66, wherein the compound is a compound

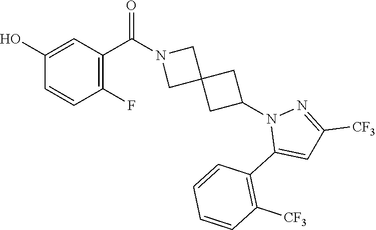 [SMILES: O=C(c1cc(O)ccc1F)N1CC2(CC(n3nc(C(F)(F)F)cc3-c3ccccc3C(F)(F)F)C2)C1]

or a pharmaceutically acceptable salt thereof.
96. The method of embodiment 66, wherein the compound is a compound

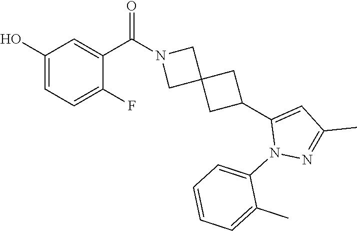 [SMILES: Cc1cc(C2CC3(C2)CN(C(=O)c2cc(O)ccc2F)C3)n(-c2ccccc2C)n1]

or a pharmaceutically acceptable salt thereof.
97. The method of embodiment 66, wherein the compound is selected from the group consisting of:

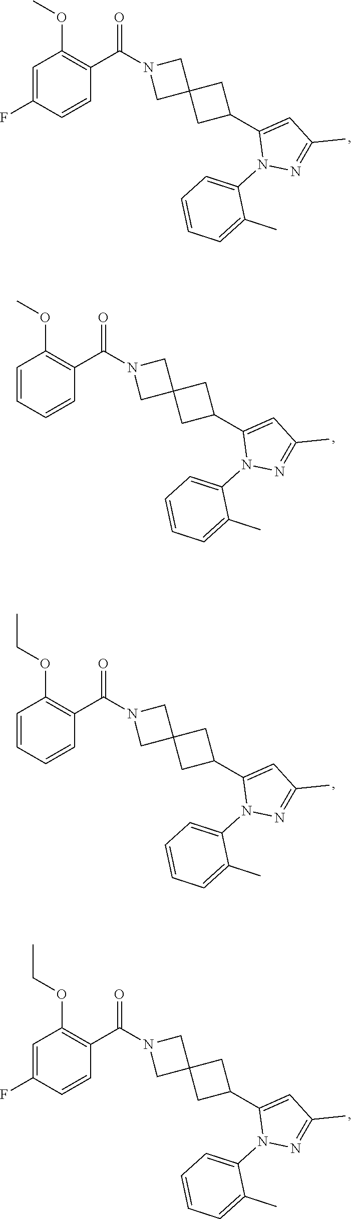 [SMILES: COc1cc(F)ccc1C(=O)N1CC2(CC(c3cc(C)nn3-c3ccccc3C)C2)C1 ; COc1ccccc1C(=O)N1CC2(CC(c3cc(C)nn3-c3ccccc3C)C2)C1 ; CCOc1ccccc1C(=O)N1CC2(CC(c3cc(C)nn3-c3ccccc3C)C2)C1 ; CCOc1cc(F)ccc1C(=O)N1CC2(CC(c3cc(C)nn3-c3ccccc3C)C2)C1]

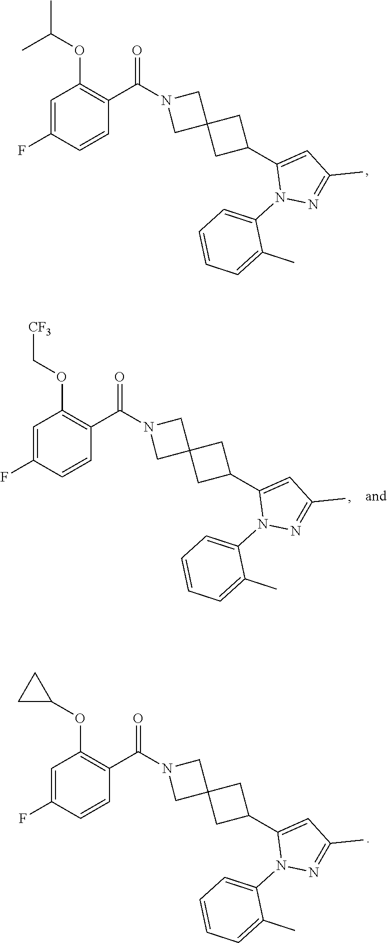 [SMILES: Cc1cc(C2CC3(C2)CN(C(=O)c2ccc(F)cc2OC(C)C)C3)n(-c2ccccc2C)n1 ; Cc1cc(C2CC3(C2)CN(C(=O)c2ccc(F)cc2OCC(F)(F)F)C3)n(-c2ccccc2C)n1 ; Cc1cc(C2CC3(C2)CN(C(=O)c2ccc(F)cc2OC2CC2)C3)n(-c2ccccc2C)n1]

98. The method of embodiment 66, wherein the compound is selected from the group consisting of.

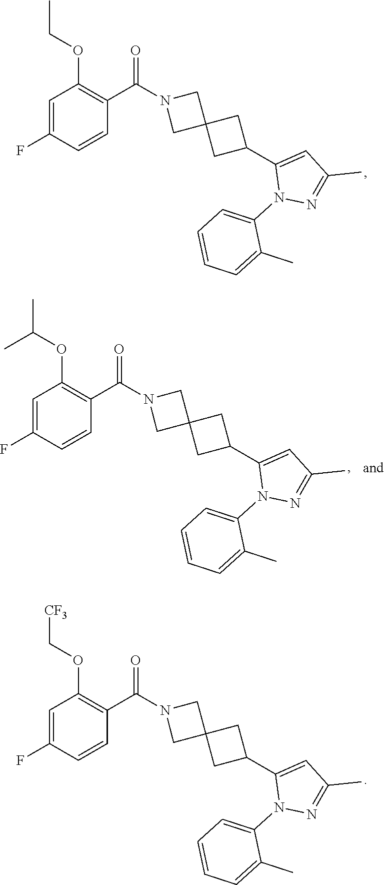 [SMILES: CCOc1cc(F)ccc1C(=O)N1CC2(CC(c3cc(C)nn3-c3ccccc3C)C2)C1 ; Cc1cc(C2CC3(C2)CN(C(=O)c2ccc(F)cc2OC(C)C)C3)n(-c2ccccc2C)n1 ; Cc1cc(C2CC3(C2)CN(C(=O)c2ccc(F)cc2OCC(F)(F)F)C3)n(-c2ccccc2C)n1]

99. The method of embodiment 66, wherein the compound is selected from the group consisting of:

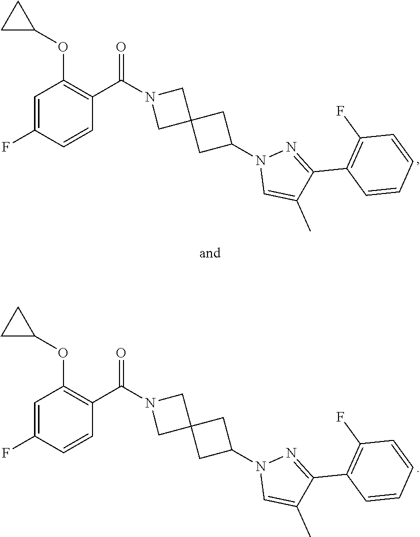 [SMILES: Cc1cn(C2CC3(C2)CN(C(=O)c2ccc(F)cc2OC2CC2)C3)nc1-c1ccccc1F ; Cc1cn(C2CC3(C2)CN(C(=O)c2ccc(F)cc2OC2CC2)C3)nc1-c1ccccc1F]

100. The method of embodiment 66, wherein the compound is a Selective MAGL Inhibitors and Reversible MAGL Inhibitor compound selected from the group consisting of:

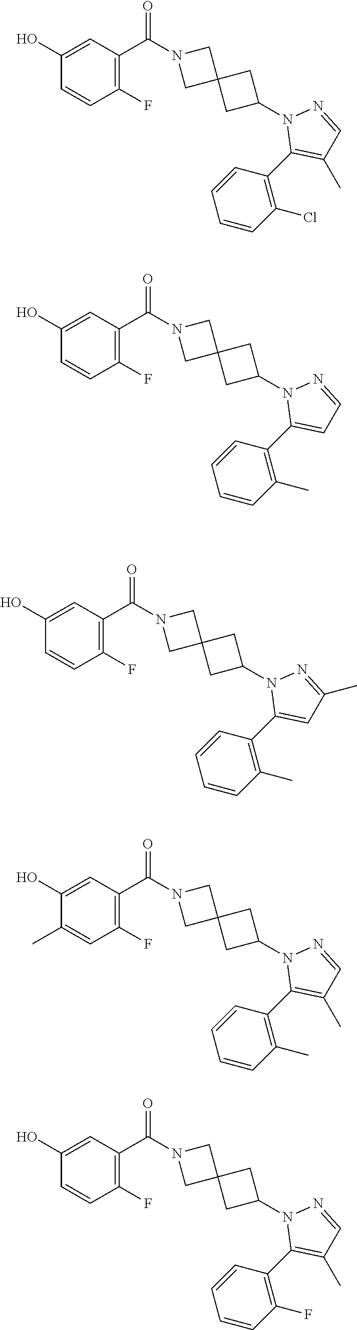 [SMILES: Cc1cnn(C2CC3(C2)CN(C(=O)c2cc(O)ccc2F)C3)c1-c1ccccc1Cl ; Cc1ccccc1-c1ccnn1C1CC2(C1)CN(C(=O)c1cc(O)ccc1F)C2 ; Cc1cc(-c2ccccc2C)n(C2CC3(C2)CN(C(=O)c2cc(O)ccc2F)C3)n1 ; Cc1cc(F)c(C(=O)N2CC3(CC(n4ncc(C)c4-c4ccccc4C)C3)C2)cc1O ; Cc1cnn(C2CC3(C2)CN(C(=O)c2cc(O)ccc2F)C3)c1-c1ccccc1F]

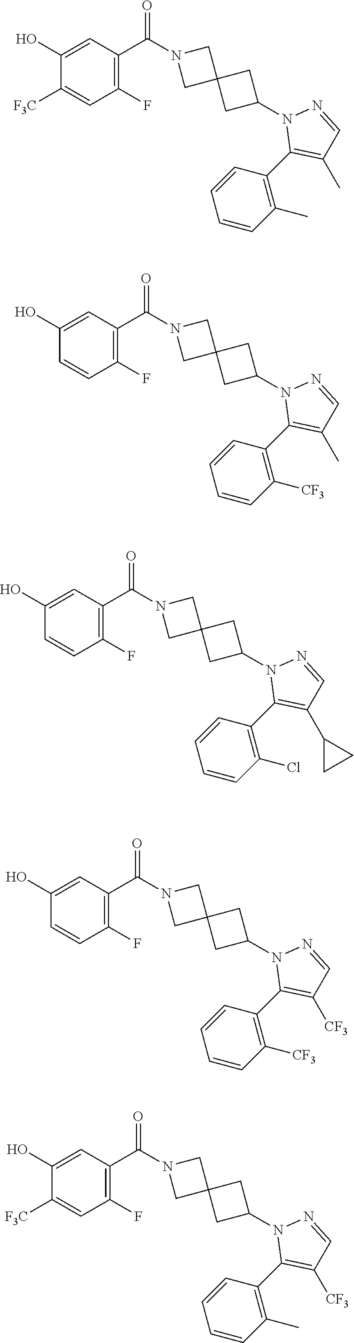 [SMILES: Cc1ccccc1-c1c(C)cnn1C1CC2(C1)CN(C(=O)c1cc(O)c(C(F)(F)F)cc1F)C2 ; Cc1cnn(C2CC3(C2)CN(C(=O)c2cc(O)ccc2F)C3)c1-c1ccccc1C(F)(F)F ; O=C(c1cc(O)ccc1F)N1CC2(CC(n3ncc(C4CC4)c3-c3ccccc3Cl)C2)C1 ; O=C(c1cc(O)ccc1F)N1CC2(CC(n3ncc(C(F)(F)F)c3-c3ccccc3C(F)(F)F)C2)C1 ; Cc1ccccc1-c1c(C(F)(F)F)cnn1C1CC2(C1)CN(C(=O)c1cc(O)c(C(F)(F)F)cc1F)C2]

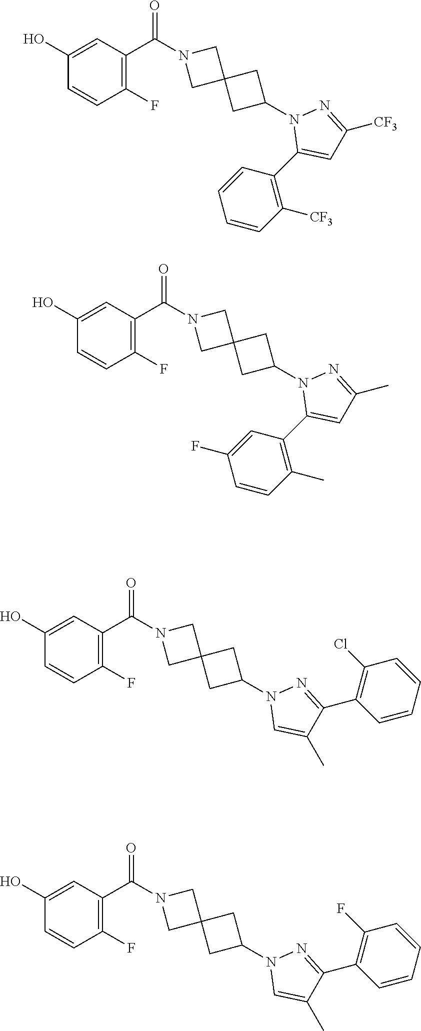 [SMILES: O=C(c1cc(O)ccc1F)N1CC2(CC(n3nc(C(F)(F)F)cc3-c3ccccc3C(F)(F)F)C2)C1 ; Cc1cc(-c2cc(F)ccc2C)n(C2CC3(C2)CN(C(=O)c2cc(O)ccc2F)C3)n1 ; Cc1cn(C2CC3(C2)CN(C(=O)c2cc(O)ccc2F)C3)nc1-c1ccccc1Cl ; Cc1cn(C2CC3(C2)CN(C(=O)c2cc(O)ccc2F)C3)nc1-c1ccccc1F]

101. The method of embodiment 66, wherein the compound is a Selective MAGL Inhibitors and Reversible MAGL Inhibitor compound selected from the group consisting of:

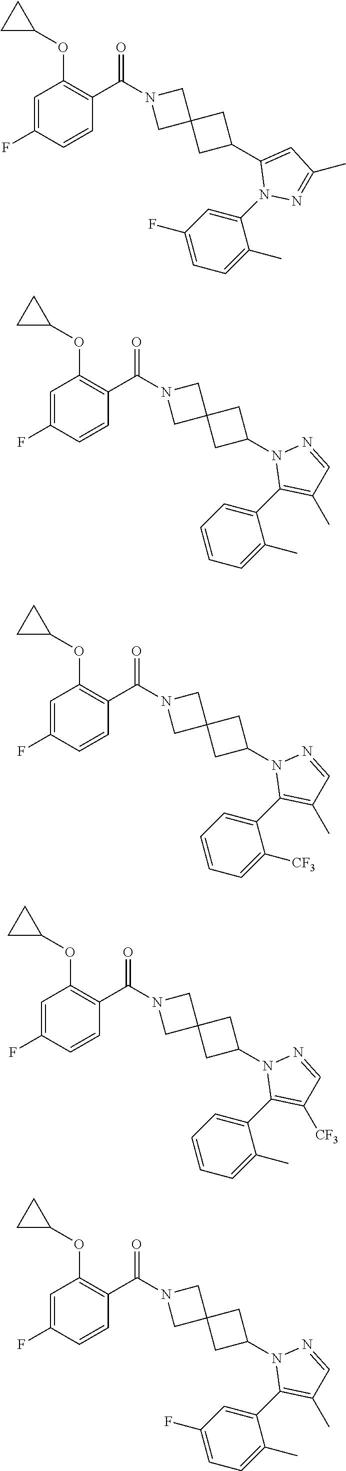 [SMILES: Cc1cc(C2CC3(C2)CN(C(=O)c2ccc(F)cc2OC2CC2)C3)n(-c2cc(F)ccc2C)n1 ; Cc1ccccc1-c1c(C)cnn1C1CC2(C1)CN(C(=O)c1ccc(F)cc1OC1CC1)C2 ; Cc1cnn(C2CC3(C2)CN(C(=O)c2ccc(F)cc2OC2CC2)C3)c1-c1ccccc1C(F)(F)F ; Cc1ccccc1-c1c(C(F)(F)F)cnn1C1CC2(C1)CN(C(=O)c1ccc(F)cc1OC1CC1)C2 ; Cc1ccc(F)cc1-c1c(C)cnn1C1CC2(C1)CN(C(=O)c1ccc(F)cc1OC1CC1)C2]

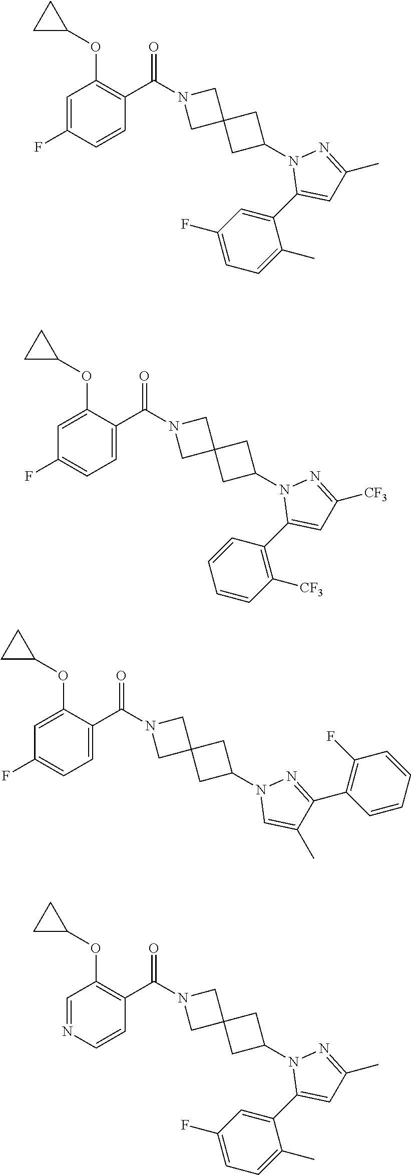 [SMILES: Cc1cc(-c2cc(F)ccc2C)n(C2CC3(C2)CN(C(=O)c2ccc(F)cc2OC2CC2)C3)n1 ; O=C(c1ccc(F)cc1OC1CC1)N1CC2(CC(n3nc(C(F)(F)F)cc3-c3ccccc3C(F)(F)F)C2)C1 ; Cc1cn(C2CC3(C2)CN(C(=O)c2ccc(F)cc2OC2CC2)C3)nc1-c1ccccc1F ; Cc1cc(-c2cc(F)ccc2C)n(C2CC3(C2)CN(C(=O)c2ccncc2OC2CC2)C3)n1]

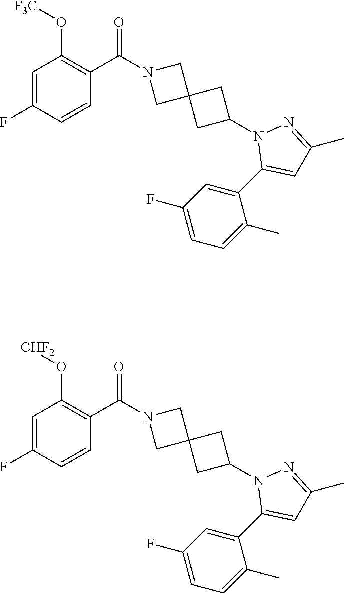 [SMILES: Cc1cc(-c2cc(F)ccc2C)n(C2CC3(C2)CN(C(=O)c2ccc(F)cc2OC(F)(F)F)C3)n1 ; Cc1cc(-c2cc(F)ccc2C)n(C2CC3(C2)CN(C(=O)c2ccc(F)cc2OC(F)F)C3)n1]

102. The method of embodiment 66, wherein the compound is 2-fluoro-5-hydroxyphenyl) (6-(3-methyl-1-(o-tolyl)-1H-pyrazol-5-yl)-2-azaspiro[3.3]heptan-2-yl) methanone.
103. The method of any one of embodiments 67-102, wherein the disorder is generalized anxiety disorder (GAD).
104. The method of any one of embodiments 67-102, wherein the disorder is panic disorder.
105. The method of any one of embodiments 67-102, wherein the disorder is social anxiety disorder.
106. The method of any one of embodiments 67-102, wherein the disorder is obsessive-compulsive disorder (OCD).
107. The method of any one of embodiments 67-102, wherein the disorder is post-traumatic stress disorder (PTSD).

### Additional Embodiments

1. A method of treating an anxiety disorder in a subject in need thereof, the method comprising administering to the subject a therapeutically effective amount of a compound selected from the group consisting of:
(2,4-difluoro-5-hydroxyphenyl) {6-[3-methyl-1-(o-tolyl)-5-pyrazolyl]-2-aza-2-spiro[3.3]heptyl}methanone (Compound 126);
4-hydroxy-2-({6-[3-methyl-1-(o-tolyl)-5-pyrazolyl]-2-aza-2-spiro[3.3]heptyl}carbonyl)benzonitrile (Compound 128);
[4-fluoro-2-(2,2,2-trifluoroethoxy)phenyl]{6-[3-methyl-1-(o-tolyl)-5-pyrazolyl]-2-aza-2-spiro[3.3]heptyl}methanone (Compound 178);
(2-fluoro-5-hydroxyphenyl) {6-[1-(5-fluoro-2-tolyl)-3-methyl-5-pyrazolyl]-2-aza-2-spiro[3.3]heptyl}methanone (Compound 365);
(2-fluoro-5-hydroxyphenyl) (6-{3-methyl-1-[o-(trifluoromethyl)phenyl]-5-pyrazolyl}-2-aza-2-spiro[3.3]heptyl) methanone (Compound 366);
(2-fluoro-5-hydroxyphenyl) (6-{5-(trifluoromethyl)-3-[o-(trifluoromethyl)phenyl]-1-pyrazolyl}-2-aza-2-spiro[3.3]heptyl) methanone (Compound 414);
(2-cyclopropoxy-4-fluorophenyl) (6-{5-(trifluoromethyl)-3-[o-(trifluoromethyl)phenyl]-1-pyrazolyl}-2-aza-2-spiro[3.3]heptyl) methanone (Compound 451);
(2-fluoro-5-hydroxyphenyl) {6-[3-(o-fluorophenyl)-4-(trifluoromethyl)-1-pyrazolyl]-2-aza-2-spiro[3.3]heptyl}methanone (Compound 473);
{6-[5-(2-chloro-5-fluorophenyl)-4-methyl-1-pyrazolyl]-2-aza-2-spiro[3.3]heptyl}(2-fluoro-5-hydroxyphenyl) methanone (Compound 519);
{6-[3-(2-chloro-5-fluorophenyl)-4-methyl-1-pyrazolyl]-2-aza-2-spiro[3.3]heptyl}(2-fluoro-5-hydroxyphenyl) methanone (Compound 520); and
{6-[5-(2,5-difluorophenyl)-4-methyl-1-pyrazolyl]-2-aza-2-spiro[3.3]heptyl}(2-fluoro-5-hydroxyphenyl) methanone (Compound 476), or a pharmaceutically acceptable salt thereof.
   2. The method of embodiment 1, wherein the anxiety disorder is selected from the group consisting of: Generalized Anxiety Disorder (GAD), panic disorder, social anxiety disorder, obsessive-compulsive disorder (OCD), post-traumatic stress disorder (PTSD), separation anxiety disorder, anxious depression, and phobias.
   3. The method of embodiment 1, wherein the anxiety disorder is Generalized Anxiety Disorder (GAD).
   4. The method of embodiment 1, wherein the anxiety disorder is a panic disorder.
   5. The method of embodiment 1, wherein the anxiety disorder is a social anxiety disorder.
   6. The method of embodiment 1, wherein the anxiety disorder is anxious depression.
   7. The method of embodiment 1, wherein the subject is diagnosed with obsessive-compulsive disorder (OCD).
   8 The method of embodiment 1, wherein the subject is diagnosed with post-traumatic stress disorder (PTSD).
   9. The method of any one of embodiments 1-8, wherein the compound is (2,4-difluoro-5-hydroxyphenyl) {6-[3-methyl-1-(o-tolyl)-5-pyrazolyl]-2-aza-2-spiro[3.3]heptyl}methanone (Compound 126), or a pharmaceutically acceptable salt thereof.
   10. The method of any one of embodiments 1-8, wherein the compound is 4-hydroxy-2-({6-[3-methyl-1-(o-tolyl)-5-pyrazolyl]-2-aza-2-spiro[3.3]heptyl}carbonyl)benzonitrile (Compound 128), or a pharmaceutically acceptable salt thereof.
   11. The method of any one of embodiments 1-8, wherein the compound is [4-fluoro-2-(2,2,2-trifluoroethoxy)phenyl]{6-[3-methyl-1-(o-tolyl)-5-pyrazolyl]-2-aza-2-spiro[3.3]heptyl}methanone (Compound 178), or a pharmaceutically acceptable salt thereof.
   12. The method of any one of embodiments 1-8, wherein the compound is (2-fluoro-5-hydroxyphenyl) {6-[1-(5-fluoro-2-tolyl)-3-methyl-5-pyrazolyl]-2-aza-2-spiro[3.3]heptyl}methanone (Compound 365) or a pharmaceutically acceptable salt thereof.
   13. The method of any one of embodiments 1-8, wherein the compound is (2-fluoro-5-hydroxyphenyl) (6-{3-methyl-1-[o-(trifluoromethyl)phenyl]-5-pyrazolyl}-2-aza-2-spiro[3.3]heptyl) methanone (Compound 366), or a pharmaceutically acceptable salt thereof.
   14. The method of any one of embodiments 1-8, wherein the compound is (2-fluoro-5-hydroxyphenyl) (6-{5-(trifluoromethyl)-3-[o-(trifluoromethyl)phenyl]-1-pyrazolyl}-2-aza-2-spiro[3.3]heptyl) methanone (Compound 414), or a pharmaceutically acceptable salt thereof.
   15. The method of any one of embodiments 1-8, wherein the compound is (2-cyclopropoxy-4-fluorophenyl) (6-{5-(trifluoromethyl)-3-[o-(trifluoromethyl)phenyl]-1-pyrazolyl}-2-aza-2-spiro[3.3]heptyl) methanone (Compound 451), or a pharmaceutically acceptable salt thereof.
   16. The method of any one of embodiments 1-8, wherein the compound is (2-fluoro-5-hydroxyphenyl) {6-[3-(o-fluorophenyl)-4-(trifluoromethyl)-1-pyrazolyl]-2-aza-2-spiro[3.3]heptyl}methanone (Compound 473).
   17. The method of any one of embodiments 1-8, wherein the compound is {6-[5-(2-chloro-5-fluorophenyl)-4-methyl-1-pyrazolyl]-2-aza-2-spiro[3.3]heptyl}(2-fluoro-5-hydroxyphenyl) methanone (Compound 519).
   18. The method of any one of embodiments 1-8, wherein the compound is {6-[3-(2-chloro-5-fluorophenyl)-4-methyl-1-pyrazolyl]-2-aza-2-spiro[3.3]heptyl}(2-fluoro-5-hydroxyphenyl) methanone (Compound 520).
   19. The method of any one of embodiments 1-8, wherein the compound is {6-[5-(2,5-difluorophenyl)-4-methyl-1-pyrazolyl]-2-aza-2-spiro[3.3]heptyl}(2-fluoro-5-hydroxyphenyl) methanone (Compound 476), or a pharmaceutically acceptable salt thereof.
   20. A method of reversibly inhibiting MAGL in a subject in need thereof, the method comprising administering to the subject a therapeutically effective amount of a compound selected from the group consisting of:
(2,4-difluoro-5-hydroxyphenyl) {6-[3-methyl-1-(o-tolyl)-5-pyrazolyl]-2-aza-2-spiro[3.3]heptyl}methanone (Compound 126);
4-hydroxy-2-({6-[3-methyl-1-(o-tolyl)-5-pyrazolyl]-2-aza-2-spiro[3.3]heptyl}carbonyl)benzonitrile (Compound 128);
[4-fluoro-2-(2,2,2-trifluoroethoxy)phenyl]{6-[3-methyl-1-(o-tolyl)-5-pyrazolyl]-2-aza-2-spiro[3.3]heptyl}methanone (Compound 178);
(2-fluoro-5-hydroxyphenyl) {6-[1-(5-fluoro-2-tolyl)-3-methyl-5-pyrazolyl]-2-aza-2-spiro[3.3]heptyl}methanone (Compound 365);
(2-fluoro-5-hydroxyphenyl) (6-{3-methyl-1-[o-(trifluoromethyl)phenyl]-5-pyrazolyl}-2-aza-2-spiro[3.3]heptyl) methanone (Compound 366),
(2-fluoro-5-hydroxyphenyl) (6-{5-(trifluoromethyl)-3-[o-(trifluoromethyl)phenyl]-1-pyrazolyl}-2-aza-2-spiro[3.3]heptyl) methanone (Compound 414);
(2-cyclopropoxy-4-fluorophenyl) (6-{5-(trifluoromethyl)-3˜ [o-(trifluoromethyl)phenyl]-1-pyrazolyl}-2-aza-2-spiro[3.3]heptyl) methanone (Compound 451);
(2-fluoro-5-hydroxyphenyl) {6-[3-(o-fluorophenyl)-4-(trifluoromethyl)-1-pyrazolyl]-2-aza-2-spiro[3.3]heptyl}methanone (Compound 473);
{6-[5-(2-chloro-5-fluorophenyl)-4-methyl-1-pyrazolyl]-2-aza-2-spiro[3.3]heptyl}(2-fluoro-5-hydroxyphenyl) methanone (Compound 519);
{6-[3-(2-chloro-5-fluorophenyl)-4-methyl-1-pyrazolyl]-2-aza-2-spiro[3.3]heptyl}(2-fluoro-5-hydroxyphenyl) methanone (Compound 520); and
{6-[5-(2,5-difluorophenyl)-4-methyl-1-pyrazolyl]-2-aza-2-spiro[3.3]heptyl}(2-fluoro-5-hydroxyphenyl) methanone (Compound 476), or a pharmaceutically acceptable salt thereof.
   21. The method of embodiment 20, wherein the compound is (2,4-difluoro-5-hydroxyphenyl) {6-[3-methyl-1-(o-tolyl)-5-pyrazolyl]-2-aza-2-spiro[3.3]heptyl}methanone (Compound 126), or a pharmaceutically acceptable salt thereof.
   22 The method of embodiment 20, wherein the compound is 4-hydroxy-2-({6-[3-methyl-1-(o-tolyl)-5-pyrazolyl]-2-aza-2-spiro[3.3]heptyl}carbonyl)benzonitrile (Compound 128), or a pharmaceutically acceptable salt thereof.
   23. The method of embodiment 20, wherein the compound is [4-fluoro-2-(2,2,2-trifluoroethoxy)phenyl]{6-[3-methyl-1-(o-tolyl)-5-pyrazolyl]-2-aza-2-spiro[3.3]heptyl}methanone (Compound 178), or a pharmaceutically acceptable salt thereof.
   24. The method of embodiment 20, wherein the compound is (2-fluoro-5-hydroxyphenyl) {6-[1-(5-fluoro-2-tolyl)-3-methyl-5-pyrazolyl]-2-aza-2-spiro[3.3]heptyl}methanone (Compound 365), or a pharmaceutically acceptable salt thereof.
   25 The method of embodiment 20, wherein the compound is (2-fluoro-5-hydroxyphenyl) (6-{3-methyl-1-[o-(trifluoromethyl)phenyl]-5-pyrazolyl}-2-aza-2-spiro[3.3]heptyl) methanone (Compound 366), or a pharmaceutically acceptable salt thereof.
   26 The method of embodiment 20, wherein the compound is (2-fluoro-5-hydroxyphenyl) (6-{5-(trifluoromethyl)-3-[o-(trifluoromethyl)phenyl]-1-pyrazolyl}-2-aza-2-spiro[3.3]heptyl) methanone (Compound 414), or a pharmaceutically acceptable salt thereof.
   27 The method of embodiment 20, wherein the compound is (2-cyclopropoxy-4-fluorophenyl) (6-{5-(trifluoromethyl)-3-[o-(trifluoromethyl)phenyl]-1-pyrazolyl}-2-aza-2-spiro[3.3]heptyl) methanone (Compound 451), or a pharmaceutically acceptable salt thereof.
   28. The method of embodiment 20, wherein the compound is (2-fluoro-5-hydroxyphenyl) {6-[3-(o-fluorophenyl)-4-(trifluoromethyl)-1-pyrazolyl]-2-aza-2-spiro[3.3]heptyl}methanone (Compound 473), or a pharmaceutically acceptable salt thereof.
   29. The method of embodiment 20, wherein the compound is {6-[5-(2-chloro-5-fluorophenyl)-4-methyl-1-pyrazolyl]-2-aza-2-spiro[3.3]heptyl}(2-fluoro-5-hydroxyphenyl) methanone (Compound 519), or a pharmaceutically acceptable salt thereof.
   30 The method of embodiment 20, wherein the compound is {6-[3-(2-chloro-5-fluorophenyl)-4-methyl-1-pyrazolyl]-2-aza-2-spiro[3.3]heptyl}(2-fluoro-5-hydroxyphenyl) methanone (Compound 520), or a pharmaceutically acceptable salt thereof.
   31. The method of embodiment 20, wherein the compound is {6-[5-(2,5-difluorophenyl)-4-methyl-1-pyrazolyl]-2-aza-2-spiro[3.3]heptyl}(2-fluoro-S-hydroxyphenyl) methanone (Compound 476), or a pharmaceutically acceptable salt thereof.
   32 The method of any one of embodiments 20-31, wherein the administration of the compound transiently increases a level of 2-AG in a brain of the subject.
   33 The method of embodiment 32, wherein a half-life of the transient increase of the level of 2-AG in the brain of the subject is less than twice the half-life of the compound in a blood plasma of the subject.
   34 The method of any one of embodiments 20-31, wherein the administration is oral administration; and wherein the administration of the compound transiently increases a level of 2-AG in a brain of the subject within about 30 minutes after the oral administration of the compound to the subject.
   35. The method of embodiment 34, wherein a half-life of the transient increase of the level of 2-AG in the brain of the subject is less than twice the half-life of the compound in a blood plasma of the subject.

Further, although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1: Preparation of Synthetic Intermediates

### Intermediate-5:2-(2-ethoxy-4-fluorobenzoyl)-2-azaspiro[3.3]heptane-6-carboxylic Acid

### Synthetic Scheme:

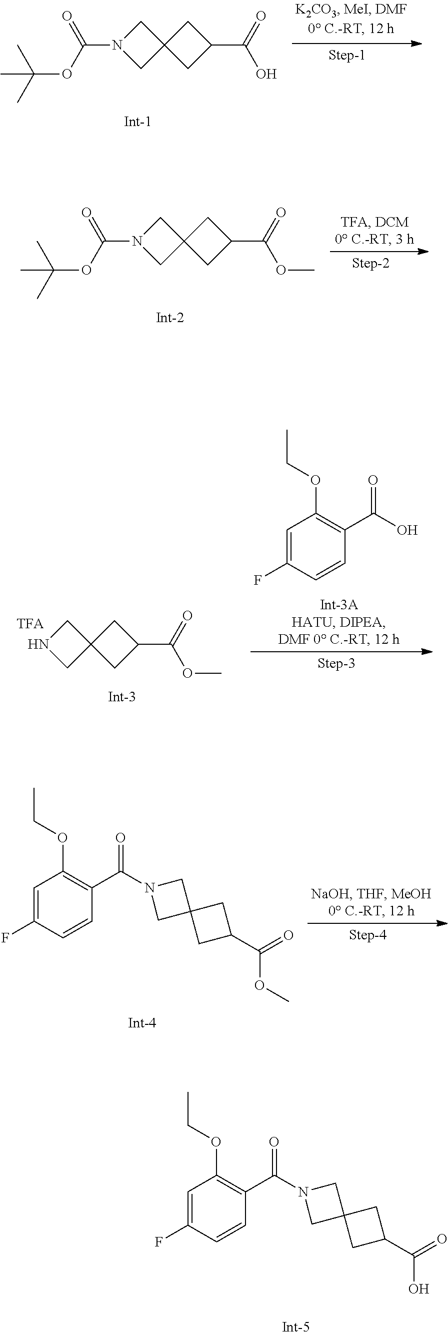 [SMILES: CC(C)(C)OC(=O)N1CC2(CC(C(=O)O)C2)C1 ; COC(=O)C1CC2(C1)CN(C(=O)OC(C)(C)C)C2 ; CCOc1cc(F)ccc1C(=O)O ; COC(=O)C1CC2(CNC2)C1 ; CCOc1cc(F)ccc1C(=O)N1CC2(CC(C(=O)OC)C2)C1 ; CCOc1cc(F)ccc1C(=O)N1CC2(CC(C(=O)O)C2)C1]

### Step-1: Synthesis of 2-(tert-butyl) 6-methyl 2-azaspiro [3.3]heptane-2,6-dicarboxylate (Int-2)

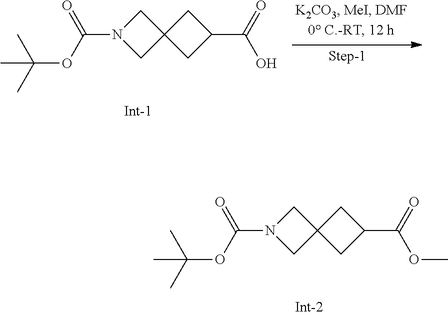 [SMILES: CC(C)(C)OC(=O)N1CC2(CC(C(=O)O)C2)C1 ; COC(=O)C1CC2(C1)CN(C(=O)OC(C)(C)C)C2]

To a stirred solution of 2-(tert-butoxycarbonyl)-2-azaspiro[3.3]heptane-6-carboxylic acid (3.0 gm, 12.44 mmol, 1.0 eq.) in N,N-Dimethyl formamide (30 mL), was added potassium carbonate (2.061 gm, 14.93 mmol, 1.2 eq.) at 0° C. The reaction was stirred at 0° C. for 10 min. Methyl Iodide (2.12 gm, 14.93 mmol, 1.2 eq.) was added dropwise and stirred the reaction mass at room temperature for 12 hrs. After completion of reaction as monitored by TLC, the reaction mixture was diluted with Ice cold water (30 mL) and extracted with Ethyl acetate (3×30 mL). The combined organic layer was washed with brine solution, dried over sodium sulfate and concentrated to obtain crude product, which was purified by combiflash using 30% Ethyl acetate in Hexane as eluent to afford 2-(tert-butyl)₆-methyl 2-azaspiro[3.3]heptane-2,6-dicarboxylate (Int-2) 3.10 gm, (Yield-97.79%). LCMS: 200.2/z [M−56]⁺

### Step-2: Synthesis of methyl 2-azaspiro[3.3]heptane-6-carboxylate (Int-3)

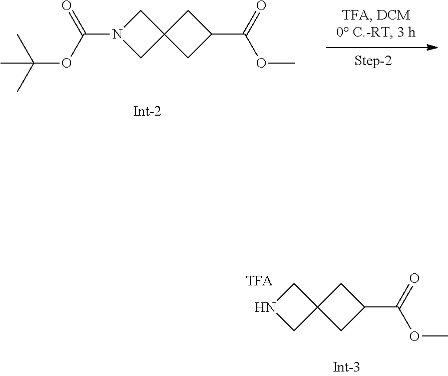 [SMILES: COC(=O)C1CC2(C1)CN(C(=O)OC(C)(C)C)C2 ; COC(=O)C1CC2(CNC2)C1]

To a stirred solution of 2-(tert-butyl)₆-methyl 2-azaspiro[3.3]heptane-2,6-dicarboxylate (Int-2) (3.0 gm, 8.0 mmol, 1.0 eq.) in Dichloromethane (30 mL) was added Trifluoroacetic acid (3.0 mL) at 0° C. and allowed to stirred the reaction at Room temperature for 3 br; the progress of the reaction of the was monitored by TLC. After completion of reaction, the reaction mixture was evaporated under vacuum and basified with bicarbonate solution (15 mL) extracted with Ethyl acetate (3*30 mL) Ethyl acetate layer separated dried over sodium sulfate and concentrated to obtain crude product methyl 2-azaspiro[3.3]heptane-6-carboxylate (Int-3) 2.50 gm (Yield: quantitative); LCMS: 155.90/z [M+1]⁺

### Step-3: Synthesis of methyl 2-(2-ethoxy-4-fluorobenzoyl)-2-azaspiro[3.3]heptane-6-carboxylate Int-4)

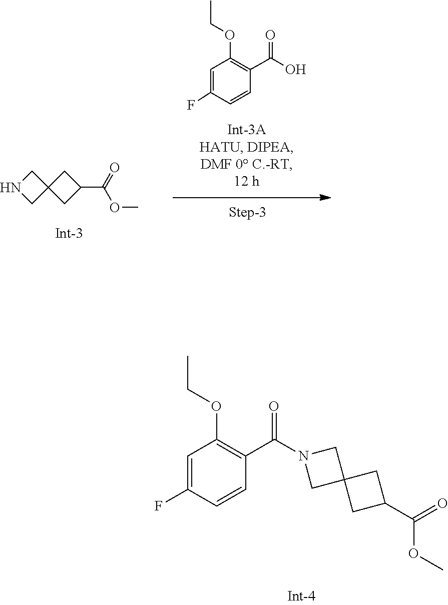 [SMILES: CCOc1cc(F)ccc1CC(=O)O ; COC(=O)C1CC2(CNC2)C1 ; CCOc1cc(F)ccc1C(=O)N1CC2(CC(C(=O)OC)C2)C1]

To a stirred solution of 2-ethoxy-4-fluorobenzoic acid (2.0 gm, 10.86 mmol, 1 eq.) in N,N-Dimethyl formamide (20 mL) were added HATU (6.19 gm, 16.30 mmol, 1.5 eq.) DIPEA (4.20 gm, 32.6 mmol, 3.0 eq.) followed by methyl 2-azaspiro[3.3]heptane-6-carboxylate (Int-3) (1.33 gm, 10.86 mmol, 1.0 eq.) at 0° C. and allowed to stirred the reaction at room temperature for 12 hr; the progress of the reaction of was monitored by TLC. After completion of reaction, the reaction mixture was diluted with water (20 mL) and extracted with ethyl acetate (3×30 mL), washed with brine. The organic layer was dried over sodium sulphate and concentrated under vacuum to get crude material; which was purified by Combi-flash by using 70% Ethyl acetate in n Hexane as mobile phase to give desired product 2-(2-ethoxy-4-fluorobenzoyl)-2-azaspiro[3.3]heptane-6-carboxylic acid (Int-4) 2.4 gm (Yield: 73.52%); LCMS: 322.3 m/z [M+1]⁺

### Step-4: Synthesis of 2-(2-ethoxy-4-fluorobenzoyl)-2-azaspiro[3.3]heptane-6-carboxylic acid (Int-5)

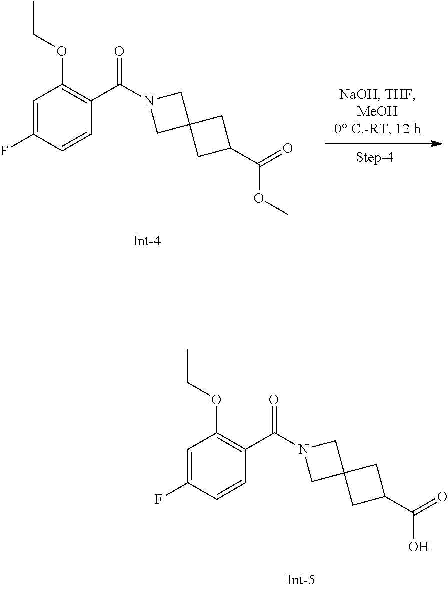 [SMILES: CCOc1cc(F)ccc1C(=O)N1CC2(CC(C(=O)OC)C2)C1 ; CCOc1cc(F)ccc1C(=O)N1CC2(CC(C(=O)O)C2)C1]

To a stirred solution of methyl 2-(2-ethoxy-4-fluorobenzoyl)-2-azaspiro[3.3]heptane-6-carboxylate (Int-4) (1.0 gm, 3.11 mmol, 1.0 eq.) in Tetrahydrofuran (10 mL), Methanol (5 mL), Water (10 mL) was added Sodium Hydroxide [NaOH] (0.13 gm, 6.23 mmol, 2.0 eq.) at 0° C. The reaction mixture was stirred at Room temperature for next 12 hr. The progress of the reaction was monitored by TLC; after completion of reaction, the reaction mixture was evaporated under vacuum. The crude product was acidified with 2N HCL (PH˜4) The white solid was precipitated out which was filtered through Buchner funnel and dried under vacuum to give 2-(2-ethoxy-4-fluorobenzoyl)-2-azaspiro[3.3]heptane-6-carboxylic acid (Int-5), 0.75 gm (Yield: 72.11%); LCMS: 308.3 m/z [M+1]⁺

### Example 2: Synthesis of ((2-fluoro-5-hydroxyphenyl) (6-(3-methyl-1-(o-tolyl)-1H-pyrazol-5-yl)-2-azaspiro[3.3]heptan-2-yl) methanone.) [Compound 74]

### Synthetic Scheme:

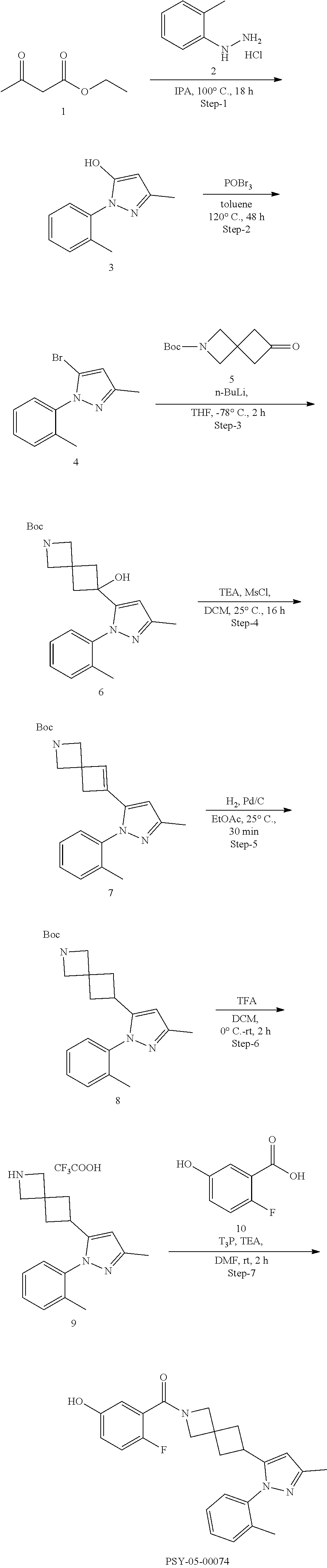

### Step-1: 3-methyl-1-(o-tolyl)-1H-pyrazol-5-ol

To a stirred solution of ethyl 3-oxobutanoate (15 g, 0.115 mol) in IPA (30 ml) was added o-tolylhydrazine hydrochloride (18.28 g, 0.115 mol) the reaction mass was heated at 100° C. for 16 h. After completion of reaction as monitored by TLC, the reaction mixture was concentrated to get residue. The compound was dissolved in methanol (10 ml) and added diethyl ether (100 ml). The mixture was filtered residue was washed with THF (50 ml). The solid was dried under vacuum to get pure compound (12 g, 55%). LCMS: 189.9 m/z [M+H]+.

### Step-2: S-bromo-3-methyl-1-(o-tolyl)-1H-pyrazole

To a stirred solution of 3-methyl-1-(o-tolyl)-1H-pyrazol-5-ol (15 g, 79.7 mmol) in toluene (300 ml) was added POBr3 (91.5 g, 319.14 mmol) at rt. The reaction was stirred at 120° C. for 48 h. After completion of reaction as monitored by TLC, the reaction mixture was quenched sat. NaHCO3 (750 ml) and extracted with ethyl acetate (500 ml*3). The organic layer was washed with brine (100 ml), dried over Na2SO4 and concentrated under reduce pressure to get residue. The residue was purified by combiflash using 2-3% ethyl acetate in hexane as eluent to get 5-bromo-3-methyl-1-(o-tolyl)-1H-pyrazole (12 g, 60%). LCMS: 251.3 m/z [M+H]+; 1H NMR (400 MHZ, DMSO-d6) δ: 7.50 (m, 4H), 5.81 (s, 1H), 2.29 (s, 3H), 2.13 (s, 3H).

### Step-3: tert-butyl 6-hydroxy-6-(3-methyl-1-(o-tolyl)-1H-pyrazol-5-yl)-2-azaspiro[3.3]heptane-2-carboxylate

To a stirred solution of tert-butyl 6-oxo-2-azaspiro[3.3]heptane-2-carboxylate (6.5 g, 0.0306 mol) and 5-bromo-3-methyl-1-(o-tolyl)-1H-pyrazole (11.5 g, 0.459 mol) in THF (130 ml) was added 2M n-BuLi in hexane (24.5 ml, 0.049 mol) at −78° C., 2 h. After completion of reaction as monitored by TLC, the reaction mixture was poured in ice cold water (100 ml) and extracted with ethyl acetate (150 ml*3). The organic layer was washed with brine (100 ml), dried over Na2SO4 and concentrated to get crude (15 g, crude) which was used as such for the next step without further purification. LCMS: 384.0 m/z [M+H]+.

### Step-4: tert-butyl 6-(3-methyl-1-(o-tolyl)-1H-pyrazol-5-yl)-2-azaspiro[3.3]hept-5-ene-2-carboxylate

To a stirred solution of tert-butyl 6-hydroxy-6-(3-methyl-1-(o-tolyl)-1H-pyrazol-5-yl)-2-azaspiro[3.3]heptane-2-carboxylate (15 g, 0.039 mol) in DCM (300 mL) was added TEA (11.9 g, 0.117 mol) followed by addition of mesyl chloride (6.71 g, 0.0585 mol) at 0° C. The reaction was stirred at rt for 16 h. The reaction mixture was concentrated under reduce pressure to get residue. The residue was purified by combiflash using 30% ethyl acetate in hexane as eluent to get tert-butyl 6-(3-methyl-1-(o-tolyl)-1H-pyrazol-5-yl)-2-azaspiro[3.3]hept-5-ene-2-carboxylate (2.9 g, 25% over two steps). LCMS: 366.5 m/z [M+H]+. 1H NMR (400 MHZ, Methanol-d4) δ: 7.49 (m, 1H), 7.44 (m, 2H), 7.29 (d, J=7.6 Hz, 1H), 6.35 (s, 1H), 5.36 (s, 1H), 4.02 (d, J=8.4 Hz, 2H), 3.93 (d, J=8.4 Hz, 2H), 2.74 (s, 2H), 2.37 (s, 3H), 2.04 (s, 3H), 1.43 (s, 9H).

### Step-5: tert-butyl 6-(3-methyl-1-(o-tolyl)-1H-pyrazol-5-yl)-2-azaspiro[3.3]heptane-2-carboxylate

A solution of tert-butyl 6-(3-methyl-1-(o-tolyl)-1H-pyrazol-5-yl)-2-azaspiro[3.3]hept-5-ene-2-carboxylate (1 g, 2.74 mmol) in ethyl acetate (10 ml) was added dropwise to a suspension of dry 10% Pd/C (0.08 g, 0.55 mmol) in ethyl acetate (10 ml) under N₂ atmosphere. The resulting mixture was stirred under H2 gas balloon pressure for 16b. The reaction mass was filtered through celite bed and washed with methanol (100 ml). The combined filtrate was concentrated to get crude material. The crude material was purified by combiflash using 20% ethyl acetate in hexane as eluent to get tert-butyl 6-(3-methyl-1-(o-tolyl)-1H-pyrazol-5-yl)-2-azaspiro[3.3]heptane-2-carboxylate (0.75 g, 75%). LCMS: 368.4 m/z [M+H]+.

### Step-6: 6-(3-methyl-1-(o-tolyl)-1H-pyrazol-5-yl)-2-azaspiro[3.3]heptane 2,2,2-trifluoroacetate

To a stirred solution of tert-butyl 6-(3-methyl-1-(o-tolyl)-1H-pyrazol-5-yl)-2-azaspiro[3.3]heptane-2-carboxylate in DCM (16 ml) was added trifluroacetic acid (2 ml) at 0° C. The reaction was stirred at room temperature for 2 h. After completion of reaction as monitored by TLC, the reaction mixture was concentrated and triturated with mixture of diethyl ether and hexane (1:1, 10 ml*3) to get 6-(3-methyl-1-(o-tolyl)-1H-pyrazol-5-yl)-2-azaspiro[3.3]heptane 2,2,2-trifluoroacetate (1.6 g, Quantitative). LCMS: 268.4 m/z [M+H]+.

### Step-7: (2-fluoro-5-hydroxyphenyl) (6-(3-methyl-1-(o-tolyl)-1H-pyrazol-5-yl)-2-azaspiro[3.3]heptan-2-yl) methanone (Compound-00074)

To a stirred solution of 6-(3-methyl-1-(o-tolyl)-1H-pyrazol-5-yl)-2-azaspiro[3.3]heptane 2,2,2-trifluoroacetate (1.6 g, 4.19 mmol) in DMF (16 ml) were added 2-fluoro-5-hydroxybenzoic acid (0.786 g, 5.03 mmol), TEA (1.28 g, 12.6 mmol) and T3P (50% in ethyl acetate) (3.2 ml, 5.03 mmol) at 0° C. The resulting reaction mixture was stirred at room temperature for 2 h. After completion of reaction as monitored by TLC, the reaction mixture was diluted with water (100 ml) and extracted with ethyl acetate (3*100 ml). The organic layer was washed with brine (3*50 ml), dried over sodium sulphate and concentrated under vacuum to get crude material which was purified by combiflash using 3% MeOH in DCM as eluent. The compound was further purified by Prep-HPLC purification. The fraction was lyophilized to get PSY-05-00074 as white solid (0.45 g, 26%). LCMS: 406.7 m/z [M+H]+. HPLC: 99.78%; 1H NMR (400 MHZ, Methanol-d4) δ 7.47-7.31 (m, J=3H), 7.21 (t, J=5.6 Hz, 1H), 7.00 (q, J=8.8 Hz, 17.6 Hz, 1H), 6.91-6.85 (m, 1H), 6.82-6.75 (m, 1H), 6.20 (d, J=19.6 Hz, 1H), 4.12-4.028 (dd, J=14.4 Hz, 21.6 Hz, 4H), 3.14-3.04 (m, 1H), 2.46-2.32 (m, 4H), 2.28 (d, J=23.6 Hz 3H), 2.01 (s, 3H).

The potency of Compound 74 for inhibiting MAGL was obtained using the following assay.

The monoacylglycerol lipase inhibitor screening assay kit from Cayman Chemical was used to measure the MAGL potency for the compounds in Table A and Table B below. Cayman's Monoacylglycerol Lipase Inhibitor Screening Assay provides a method for screening human MAGL inhibitors. MAGL hydrolyzes 4-nitrophenylacetate resulting in a yellow product, 4-nitrophenol, with an absorbance of 405-412 nm.

### Example 3: Synthesis of (2-fluoro-5-hydroxyphenyl) (6-(3-methyl-1-(o-tolyl)-1H-pyrazol-5-yl)-2-azaspiro[3.3]hept-5-en-2-yl) methanone. [Compound 120]

### Synthetic Scheme:

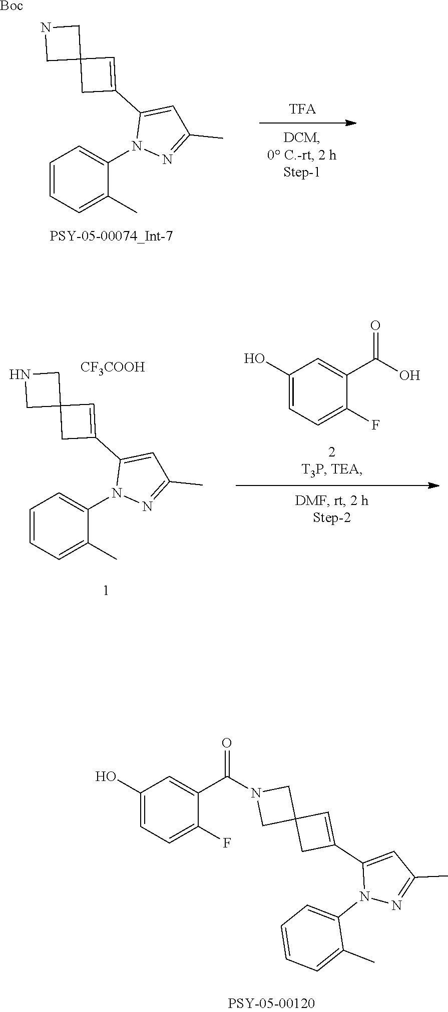 [SMILES: Cc1cc(C2=CC3(CNC3)C2)n(-c2ccccc2C)n1 ; Cc1cc(C2=CC3(CNC3)C2)n(-c2ccccc2C)n1 ; "CF3COOH" ; O=C(O)c1cc(O)ccc1F ; "Boc" ; Cc1cc(C2=CC3(C2)CN(C(=O)c2cc(O)ccc2F)C3)n(-c2ccccc2C)n1]

### Step-1:6-(3-methyl-1-(o-tolyl)-1H-pyrazol-5-yl)-2-azaspiro[3.3]hept-5-ene 2,2,2-trifluoroacetate

To a stirred solution of tert-butyl 6-(3-methyl-1-(o-tolyl)-1H-pyrazol-5-yl)-2-azaspiro[3.3]hept-5-ene-2-carboxylate (100 mg) in DCM (0.5 ml) was added trifluroacetic acid (0.5 ml) at 0° C. The reaction was stirred at room temperature for 1 h. After completion of reaction as monitored by TLC, the reaction mixture was concentrated to get crude material. The crude material was triturated with mixture of diethyl ether and hexane (1:1, 10 mL*3) to get pure desire compound (0.11 g, quantitative) as a white solid. LCMS: 266.3 m/z [M+H]+; ¹H NMR (400 MHZ, Methanol-d4) δ: 7.51-7.47 (m, 1H), 7.44-7.36 (m, 2H), 7.28 (d, J=7.6 Hz, 1H), 6.38 (s, 1H), 5.36 (s, 1H), 4.19-4.13 (m, 4H), 2.88-2.86 (m, 2H), 2.31 (s, 3H), 2.01 (s, 3H).

### Step-2: (2-fluoro-5-hydroxyphenyl) (6-(3-methyl-1-(o-tolyl)-1H-pyrazol-5-yl)-2-azaspiro[3.3]hept-S-en-2-yl) methanone

To a stirred solution of 6-(3-methyl-1-(o-tolyl)-1H-pyrazol-5-yl)-2-azaspiro[3.3]hept-5-ene 2,2,2-trifluoroacetate (0.11 g, 0.421 mmol) in DMF (1 mL) were added 2-fluoro-5-hydroxybenzoic acid (0.054 g, 0.506 mmol), TEA (0.088 g, 1.26 mmol) and T3P (0.111 g, 0.506 mmol) at 0° C. The resulting reaction mixture was stirred at room temperature for 2 h. After completion of reaction as monitored by TLC, the reaction mixture was diluted with water (50 mL) and extracted with ethyl acetate (3*50 mL). The organic layer was washed with brine (3*25 mL), dried over sodium sulphate and concentrated under vacuum to get crude material which was purified by combiflash using 3% MeOH in DCM as eluent to get ((2-fluoro-5-hydroxyphenyl) (6-(3-methyl-1-(o-tolyl)-1H-pyrazol-5-yl)-2-azaspiro[3.3]hept-5-en-2-yl) methanone) PSY-05-00120 (0.03 g, 27%) as white solid. LCMS: 473.7 m/z [M+H]+; 1H NMR (400 MHZ, Methanol-d₄) δ 7.51-7.33 (m, 3H), 7.28 (d, J=7.9 Hz, 1H), 7.00 (t, J=9.2 Hz, 1H), 6.92-6.78 (m, 2H), 6.35 (s, 1H), 5.39 (s, 1H), 4.29-4.09 (m, 4H), 2.78 (d, J=5.6 Hz, 2H), 2.30 (s, 3H), 2.00 (s, 3H).

### Example 4: Synthesis of (4-fluoro-2-isopropoxyphenyl) (6-(3-methyl-1-(o-tolyl)-1H-pyrazol-5-yl)-2-azaspiro[3.3]heptan-2-yl) methanone. [Compound 177]

### Synthetic Scheme:

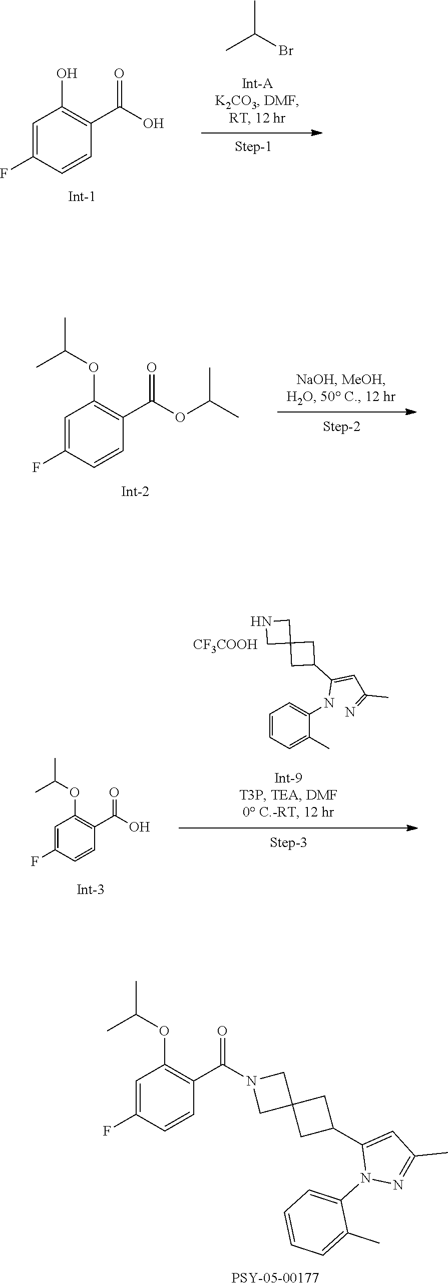

### Step-1: Synthesis of Isopropyl 4-fluoro-2-isopropoxybenzoate (Int-2)

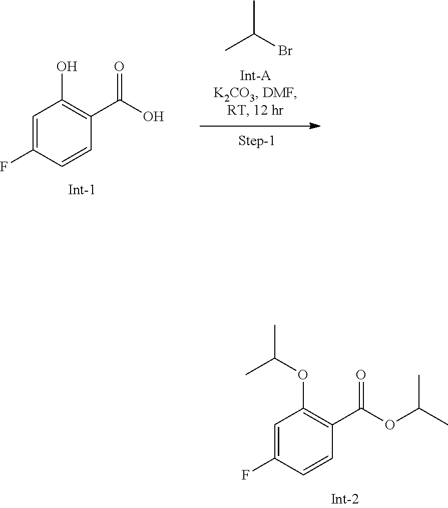

To a stirred solution of 4-fluoro-2-hydroxybenzoic acid (2.0 gm, 12.820 mmol, 1 eq.) in N,N-Dimethyl formamide (20 mL) were added Potassium carbonate (17.07 gm, 51.282 mmol, 4.0 eq.) followed by dropwise addition of Isopropyl iodide (8.88 gm, 51.282 mmol, 4.0 eq.) at 0° C. and allowed to stirred the reaction at room temperature for 12 hr; the progress of the reaction of the was monitored by TLC. After completion of reaction, the reaction mixture was diluted with water (30 mL) and extracted with ethyl acetate (3×50 mL), washed with brine. The organic layer was dried over sodium sulphate and concentrated under vacuum to get crude material; which was purified by combi-flash by using 20% Ethyl acetate in hexane as mobile phase to give desired product 4-fluoro-2-isopropoxybenzoic acid 2.0 gm (Yield: 65.14%); 1H NMR (400 MHz, DMSO-d₆) δ 7.65 (dd, J=8.7, 7.0 Hz, 1H), 7.08 (dd, J=11.8, 2.4 Hz, 1H), 6.82 (td, J=8.4, 2.4 Hz, 1H), 5.09 (hept, J=6.2 Hz, 1H), 4.72 (p, J=6.0 Hz, 1H), 3.18 (d, J=5.2 Hz, 1H), 1.29 (dd, J=6.1, 2.7 Hz, 12H).

### Step-2: Synthesis of 4-fluoro-2-isopropoxybenzoic Acid Carbonate (Int-3)

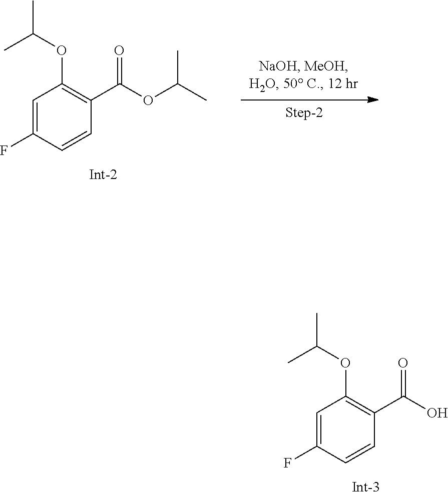 [SMILES: CC(C)OC(=O)c1ccc(F)cc1OC(C)C ; CC(C)Oc1cc(F)ccc1C(=O)O]

To a stirred solution of isopropyl 4-fluoro-2-isopropoxybenzoate (Int-2) (1.60 gm, 6.66 mmol, 1.0 eq.) in Tetrahydrofuran (10 mL), Methanol (10 mL), Water (10 mL) was added Sodium Hydroxide [NaOH] (0.53 gm, 13.33 mmol, 2.0 eq.) at 0° C. The reaction mixture was stirred at 50° C. for next 12 hr. The progress of the reaction was monitored by TLC; after completion of reaction, the reaction mixture was evaporated under vacuum. The crude product was acidified with 2N HCL (PH˜4) The white solid was precipitated out which was filtered through Buchner funnel and dried under vacuum to give isopropyl 4-fluoro-2-isopropoxybenzoic acid (Int-3) 1.20 gm (Yield: 91%); ¹H NMR (400 MHZ, DMSO-d₆) δ 12.53 (s, 1H), 7.69 (t, J=7.9 Hz, 1H), 7.05 (dd, J=11.9, 2.5 Hz, 1H), 6.80 (td, J=8.4, 2.5 Hz, 1H), 4.69 (hept, J=6.0 Hz, 1H), 1.27 (d, J=6.0 Hz, 6H).

### Step-3: Synthesis of (4-fluoro-2-isopropoxyphenyl) (6-(3-methyl-1-(o-tolyl)-1H-pyrazol-5-yl)-2-azaspiro[3.3]heptan-2-yl) methanone (Compound-00177)

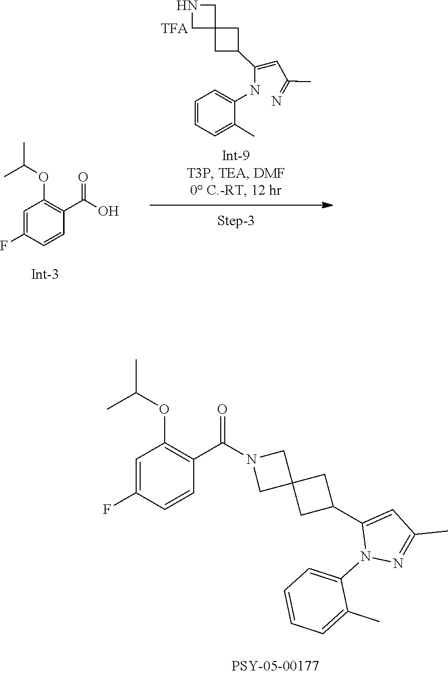

To a stirred solution of 6-(3-methyl-1-(o-tolyl)-1H-pyrazol-5-yl)-2-azaspiro[3.3]heptane 2,2,2-trifluoroacetate (0.150 gm, 0.56 mmol 1.0 eq.) in N,N-Dimethyl formamide (3 mL) were added 4-fluoro-2-isopropoxybenzoic acid (0.133 gm, 0.674 mmol, 1.2 eq.), TEA (0.170 gm, 1.68 mmol, 3.0 eq.) and T3P (50% in ethyl acetate) (0.214 gm, 0.674 mmol, 1.2 eq.) at 0° C. The resulting reaction mixture was stirred at room temperature for 12 h. After completion of reaction as monitored by TLC, the reaction mixture was diluted with water (10 ml) and extracted with ethyl acetate (3*20 ml). The organic layer was washed with brine (10 ml), dried over sodium sulphate and concentrated under vacuum to get crude material which was purified by combiflash using 50% Ethyl acetate in hexane as eluent to get PSY-05-00177 as white solid (0.040. (Yield: 16%); LCMS: 448.5 m/z [M+H]⁺. HPLC: 98.35%; ¹H NMR (400 MHZ, DMSO-d₆) δ 7.38 (t, J=8.3 Hz, 2H), 7.33-7.22 (m, 2H), 7.15 (dd, J=7.9, 4.2 Hz, 1H), 6.97 (dd, J=11.6, 8.5 Hz, 1H), 6.75 (q, J=7.6 Hz, 1H), 6.13 (d, J=17.6 Hz, 1H), 4.70-4.60 (m, 1H), 3.93 (s, 1H), 3.85 (d, J=15.1 Hz, 2H), 3.78 (s, 1H), 3.00 (dt, J=18.8, 8.5 Hz, 1H), 2.34-2.16 (m, 4H), 2.16 (s, 3H), 1.93 (s, 3H), 1.24 (dd, J=11.5, 6.1 Hz, 6H).

### Example 5: Synthesis of (2,4-difluoro-5-hydroxyphenyl) (6-(3-methyl-1-(o-tolyl)-1H-pyrazol-5-yl)-2-azaspiro[3.3]heptan-2-yl) methanone. [Compound 126]

### Synthetic Scheme:

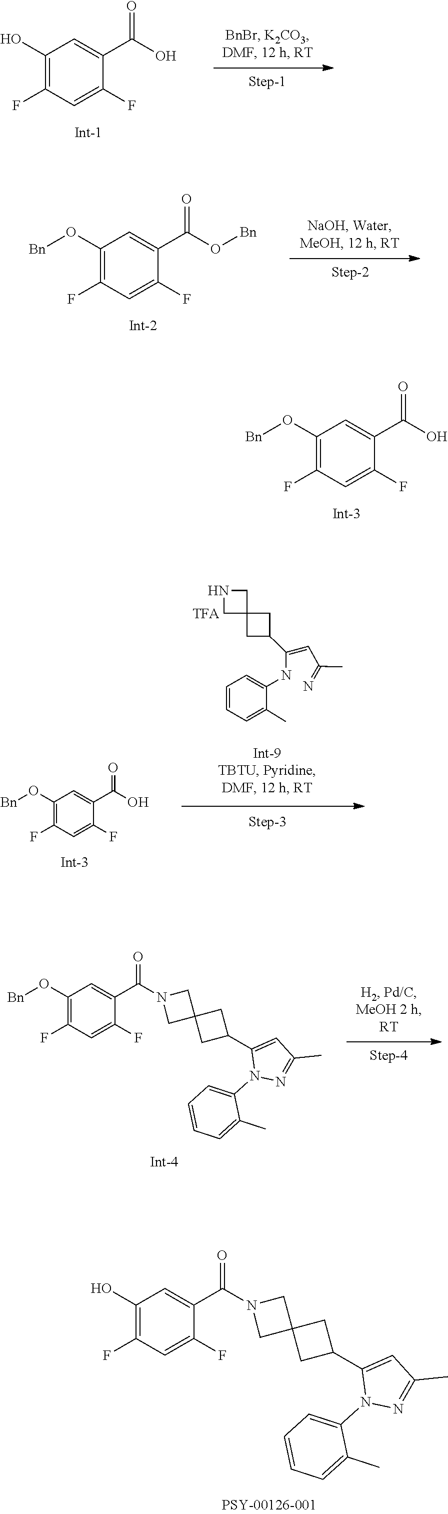

### Step-1: Synthesis of Benzyl 5-(benzyloxy)-2,4-difluorobenzoate (Int-2)

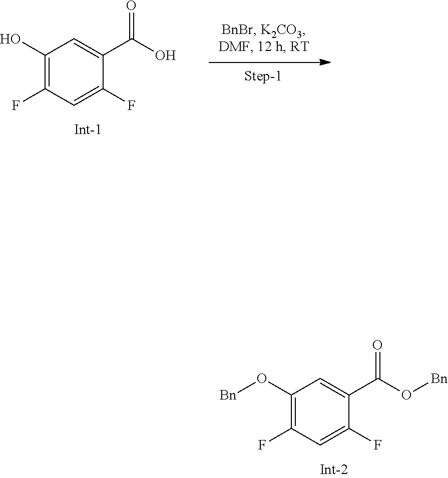 [SMILES: O=C(O)c1cc(O)c(F)cc1F ; O=C(OCc1ccccc1)c1cc(OCc2ccccc2)c(F)cc1F]

To a stirred solution of 2,4-difluoro-5-hydroxybenzoic acid (0.40 gm, 2.13 mmol, 1.0 eq.) in N,N-Dimethyl formamide (5.0 mL) were added Potassium carbonate (0.88 gm, 6.38 mmol, 3.0 eq.) Benzyl bromide (1.09 gm, 6.38 mmol, 3.0 eq.) at 0° C. and allowed to stirred the reaction at room temperature for 12 hr; the progress of the reaction was monitored by TLC. After completion of reaction, the reaction mixture was diluted with water (10 mL) and extracted with ethyl acetate (3×20 mL), washed with brine. The organic layer was dried over sodium sulphate and concentrated under vacuum to get crude material; which was purified by combi-flash by using 35% Ethyl acetate in n-Hexane as mobile phase to give desired product benzyl 5-(benzyloxy)-2,4-difluorobenzoate (Int-2) 0.560 gm (Yield: 69.13%); 1H NMR (400 MHZ, DMSO-d₆) δ 7.68 (dd, J=9.4, 6.8 Hz, 1H), 7.52 (t, J=10.9 Hz, 1H), 7.47 (d, J=1.9 Hz, 2H), 7.47-7.31 (m, 8H), 5.36 (s, 2H), 5.24 (s, 2H).

### Step-2: 5-(benzyloxy)-2,4-difluorobenzoic acid (Int-3)

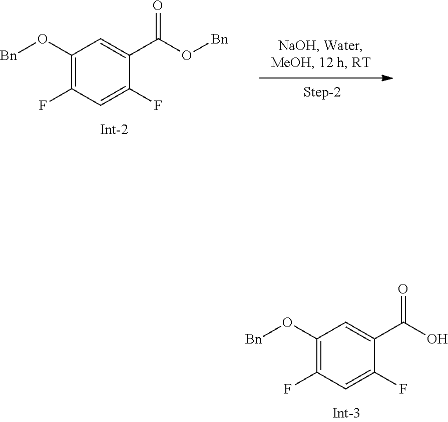 [SMILES: O=C(OCc1ccccc1)c1cc(OCc2ccccc2)c(F)cc1F ; O=C(O)c1cc(OCc2ccccc2)c(F)cc1F]

To a stirred solution of benzyl 5-(benzyloxy)-2,4-difluorobenzoate (Int-2) (0.50 gm, 1.44 mmol, 1.0 eq.) in Tetrahydrofuran (5.0 mL), Methanol (5.0 mL), Water (5.0 mL) was added Sodium Hydroxide [NaOH] (0.11 gm, 2.89 mmol, 2.0 eq.) at 0° C. The reaction mixture was stirred at Room temperature for next 12 hr. The progress of the reaction was monitored by TLC; after completion of reaction, the reaction mixture was evaporated under vacuum. The crude product was acidified with 2N HCL (PH˜4) The white solid was precipitated out which was filtered through Buchner funnel and dried under vacuum to give 5-(benzyloxy)-2,4-difluorobenzoic acid (Int-3), 0.35 gm (Yield: 94.59%); ¹H NMR (400 MHZ, DMSO-d₆) δ 13.42 (s, 1H), 7.65 (dd, J=9.5, 6.9 Hz, 1H), 7.52-7.29 (m, 5H), 5.23 (s, 2H).

### Step-3: Synthesis of (5-(benzyloxy)-2,4-difluorophenyl) (6-(3-methyl-1-(o-tolyl)-1H-pyrazol-5-yl)-2-azaspiro[3.3]heptan-2-yl) methanone (Int-4)

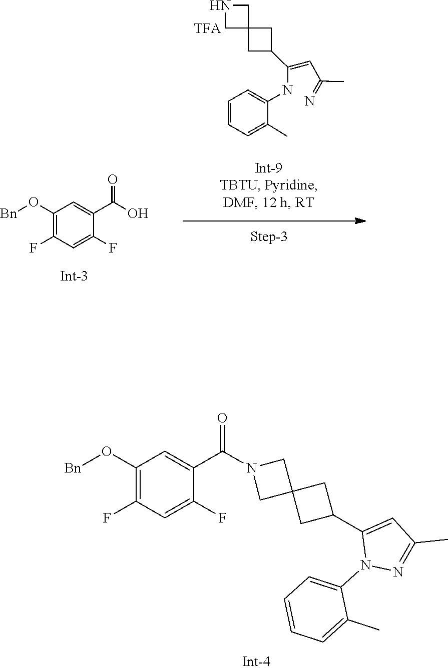

To a stirred solution of 2-(5-(benzyloxy)-2-fluorobenzoyl)-2-azaspiro[3.3]heptane-6-carboxylic acid (0.25 gm, 0.677 mmol, 1.0 eq.) in N,N-Dimethyl formamide (10.0 mL) were added TBTU (0.326 gm, 1.01 mmol, 1.5 eq.) Pyridine (0.15 gm, 2.03 mmol, 3.0 eq.) followed by addition of 5-(benzyloxy)-2,4-difluorobenzoic acid (Int-3) (0.16 gm, 0.677 mmol, 1.0 eq.) at 0° C. and allowed to stirred the reaction at room temperature for 12 hr; the progress of the reaction of the was monitored by TLC. After completion of reaction, the reaction mixture was diluted with water (10 mL) and extracted with ethyl acetate (3×20 mL), washed with brine. The organic layer was dried over sodium sulphate and concentrated under vacuum to get crude material; which was purified by combi-flash by using 80% Ethyl acetate in Hexane as mobile phase to give desired product (5-(benzyloxy)-2,4-difluorophenyl) (6-(3-methyl-1-(o-tolyl)-1H-pyrazol-5-yl)-2-azaspiro[3.3]heptan-2-yl) methanone (Int-4)_{0.25} gm (Yield: 52.08%); LCMS: 514.05 m/z [M⁺].

### Step-4: Synthesis of (2,4-difluoro-5-hydroxyphenyl) (6-(3-methyl-1-(o-tolyl)-1H-pyrazol-5-yl)-2-azaspiro[3.3]heptan-2-yl) methanone (Compound-00126)

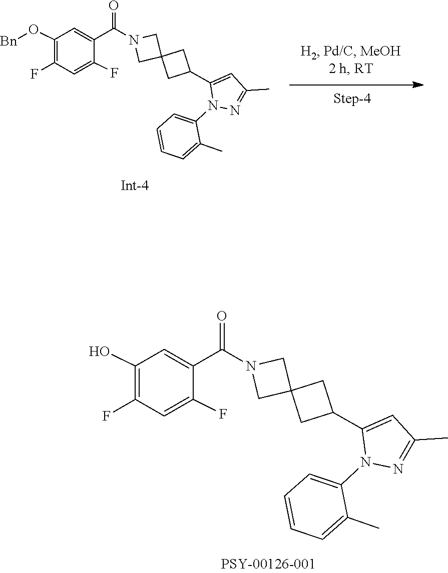 [SMILES: Cc1cc(C2CC3(C2)CN(C(=O)c2cc(OCc4ccccc4)c(F)cc2F)C3)n(-c2ccccc2C)n1 ; Cc1cc(C2CC3(C2)CN(C(=O)c2cc(O)c(F)cc2F)C3)n(-c2ccccc2C)n1]

To a stirred solution (5-(benzyloxy)-2,4-difluorophenyl) (6-(3-methyl-1-(o-tolyl)-1H-pyrazol-5-yl)-2-azaspiro[3.3]heptan-2-yl) methanone (Int-4) (0.20 gm, 0.400 mmol, 1.0 eq.) was dissolved in Methanol (10 mL). 10% Pd/C (with 50% moisture) 0.050 gm was added at Room Temperature and Reaction mixture was allowed to stir for 2 hr. under Hydrogen atmosphere. Reaction was monitored by TLC. After completion of the reaction, Reaction mixture was filtered through celite bed, washed with Methanol (50 mL) and concentrated to get crude compound, which was purified by column chromatography using 60-120 mesh size silica gel and 80% Ethyl acetate in Hexane as mobile phase to give the desired product (2,4-difluoro-5-hydroxyphenyl) (6-(3-methyl-1-(o-tolyl)-1H-pyrazol-5-yl)-2-azaspiro[3.3]heptan-2-yl) methanone (PSY-05-00126) as a white solid, 0.080 gm, (Yield: 48.78%); LCMS: 424.4 m/z [M+1]⁺¹H NMR (400 MHZ, DMSO-d₆) δ 10.30 (s, 1H), 7.38 (t, J=7.7 Hz, 3H), 7.33-7.23 (m, 1H), 7.15 (t, J=6.7 Hz, 1H), 7.02-6.93 (m, 1H), 6.14 (d, J=18.3 Hz, 1H), 3.97 (d, J=3.4 Hz, 2H), 3.91 (s, 2H), 2.98 (dt, J=18.5, 8.8 Hz, 1H), 2.45 (s, 1H), 2.34-2.22 (m, 1H), 2.19 (t, J=11.7 Hz, 3H), 1.93 (s, 3H).

### Example 6: Synthesis of 4-hydroxy-2-(6-(3-methyl-1-(o-tolyl)-1H-pyrazol-5-yl)-2-azaspiro[3.3]heptane-2-carbonyl)benzonitrile [Compound 128]

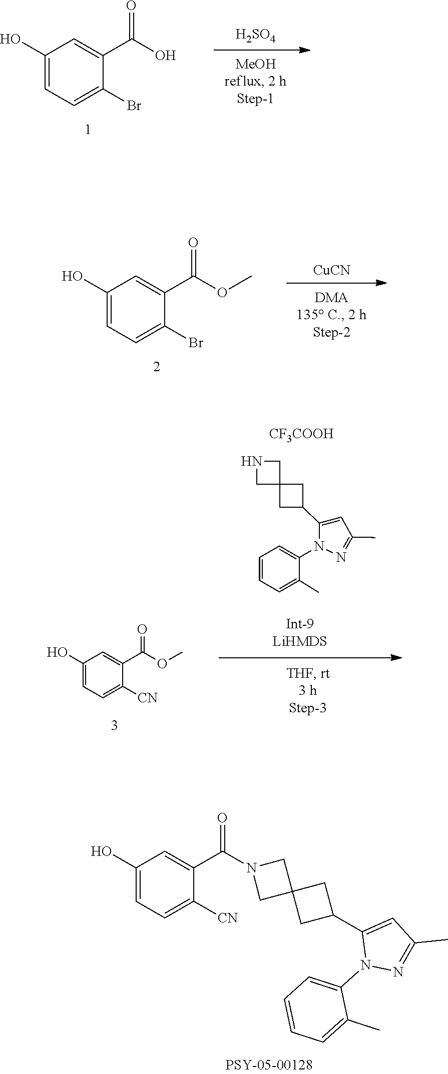

### Step-1: Methyl 2-bromo-S-hydroxybenzoate (2)

To stirred solution of 2-bromo-5-hydroxybenzoic acid (0.5 g, 2.325 mmol), in MeOH, Con. H₂SO₄ (2 mL) was added dropwise. The reaction mixture was heated to 90° C. and stirred at same temperature for 2 h. After completed reaction RM was concentered, diluted with ethylacetate, washed with aq. NaHCO₃ solution (3×15 mL), dried over Na₂SO₄, and evaporated. The crude material was purified by combiflash using 30% ethylacetate in hexane to give methyl 2-bromo-5-hydroxybenzoate (300 mg, 56%) as colorless liquid. LCMS: 229.9 m/z [M+H]⁺.

### Step-2: Methyl 2-cyano-5-hydroxybenzoate (3)

To stirred solution of methyl 2-bromo-5-hydroxybenzoate (0.3 g, 1.31 mmol) in DMA, was added CuCN (0.174 g, 1.89 mmol) and stirred at 135° C. for 2 h. The reaction was quenched by the addition of cold water (50 mL) and extracted with ethyl acetate (3×25 mL). The organic layer was concentrated and purified by combiflash using 30% ethylacetate in hexane to give methyl 2-cyano-5-hydroxybenzoate (140 mg, 61%) as a white solid. ¹H NMR (400 MHZ, CD3Cl) δ 7.71 (d, J=8.4 Hz, 1H), 7.60 (d, J=2 Hz, 1H), 7.13-7.10 (m, 1H), 6.31 (s, 1H), 4.01 (s, 3H).

### Step-3: (4-hydroxy-2-(6-(3-methyl-1-(o-tolyl)-1H-pyrazol-5-yl)-2-azaspiro[3.3]heptane-2-carbonyl)benzonitrile) (Compound-00128)

To stirred solution of 6-(3-methyl-1-(o-tolyl)-1H-pyrazol-5-yl)-2-azaspiro[3.3]heptane 2,2,2-trifluoroacetate (0.120 g, 0.449 mmol) and methyl 2-cyano-5-hydroxybenzoate (0.0795 g, 0.449 mmol) in THF at −78° C., was added LiHMDS (1.35 mL, 1.347 mmol; 1 M solution in THF) for 15 min then it stirred at rt for 3 h. The reaction mass was quenched by the addition of water (50 mL) and extracted by ethyl acetate (3×30 mL). The organic layer was concentrated under reduced pressure and purified by combiflash using 5% MeOH in DCM to give (4-hydroxy-2-(6-(3-methyl-1-(o-tolyl)-1H-pyrazol-5-yl)-2-azaspiro[3.3]heptane-2-carbonyl)benzonitrile) (60 mg 21%) as a white solid. LCMS: 413.4 m/z [M+H]⁺; ¹H NMR (400 MHZ, DMSO-d₆) δ 7.68 (t, J=7.8 Hz, 1H), 7.43-7.26 (m, 3H), 7.15 (dd, J=7.8, 3.9 Hz, 1H), 6.98-6.85 (m, 2H), 6.13 (d, J=19.6 Hz, 1H), 3.99 (dd, J=24.0, 13.9 Hz, 4H), 2.99 (dt, J=22.2, 8.4 Hz, 1H), 2.39-2.28 (m, 2H), 2.23 (dd, J=12.7, 9.0 Hz, 2H), 2.17 (d, J=8.5 Hz, 3H), 1.92 (s, 3H).

### Example 7: Synthesis of 4-fluoro-3-((6-(3-methyl-1-(o-tolyl)-1H-pyrazol-5-yl)-2-azaspiro[3.3]heptan-2-yl)methyl) phenol. [Compound 150]

### Synthetic Scheme:

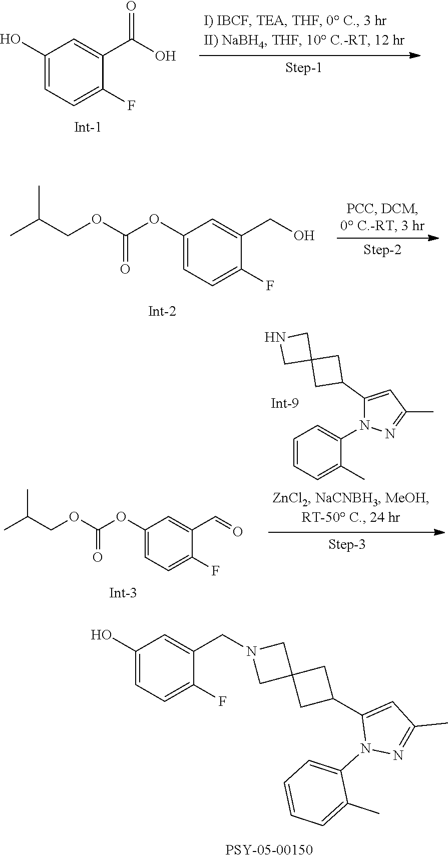 [SMILES: O=C(O)c1cc(O)ccc1F ; CC(C)COC(=O)Oc1ccc(F)c(CO)c1 ; Cc1cc(C2CC3(CNC3)C2)n(-c2ccccc2C)n1 ; CC(C)COC(=O)Oc1ccc(F)c(C=O)c1 ; Cc1cc(C2CC3(C2)CN(Cc2cc(O)ccc2F)C3)n(-c2ccccc2C)n1]

### Step-1: Synthesis of 4-fluoro-3-(hydroxymethyl) phenyl isobutyl carbonate (Int-2)

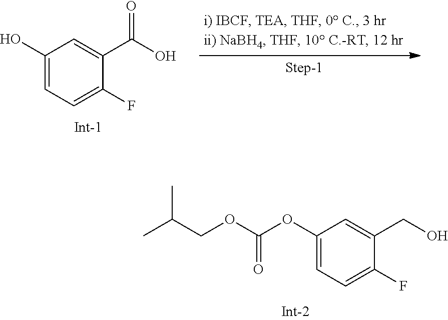 [SMILES: O=C(O)c1cc(O)ccc1F ; CC(C)COC(=O)Oc1ccc(F)c(CO)c1]

To a stir solution of 2-fluoro-S-hydroxybenzoic acid (1 gm, 6.410 mmol, 1 eq.) in THE (10 mL) were added Triethylamine (1.29 gm, 12.820 mmol, 2 eq.) fallowed by dropwise addition of isobutyl chloroformate [IBCF] (1.7 gm, 12.820 mmol, 2 eq.) at 0° C. and allowed to stirred the reaction at room temperature for 3 hr; the progress of the reaction of the was monitored by TLC. After completion of the reaction, the reaction mixture was filtered through Celite pad was washed with THE, filtered obtained was taken in single neck flask under N₂ atm. to that sodium borohydride (0.219 gm, 5.769 mmol, 0.9 eq.) was added at 0° C. and allowed to stirred reaction mixture for next 12 hr. The progress of the reaction was monitored on TLC, after completion of reaction, the reaction mixture was diluted with water (20 mL) and extracted with ethyl acetate (3×50 mL), washed with sat. NaHCO3 and brine. The combined organic layer was dried over anhydrous sodium sulphate, filtered and concentrated to get crude compound; which was purified by Combi-flash by using 30% Ethyl acetate in hexane as mobile phase to give desired product as 4-fluoro-3-(hydroxymethyl)phenyl isobutyl carbonate (Int-2) 0.74 gm (Yield: 48%); 1H NMR (400 MHZ, DMSO-d6) δ 7.30 (dd, J=6.2, 2.9 Hz, 1H), 7.27-7.12 (m, 2H), 5.41 (t, J=5.7 Hz, 1H), 4.55 (d, J=5.7 Hz, 2H), 4.01 (d, J=6.6 Hz, 2H), 1.98 (dp, J=13.4, 6.8 Hz, 1H), 0.94 (d, J=6.7 Hz, 6H).

### Step-2: Synthesis of 4-fluoro-3-formylphenyl isobutyl carbonate (Int-3)

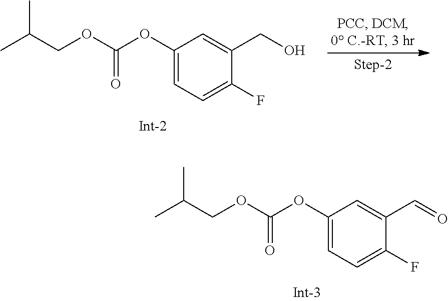 [SMILES: CC(C)COC(=O)Oc1ccc(F)c(CO)c1 ; CC(C)COC(=O)Oc1ccc(F)c(C=O)c1]

To a stir solution of 4-fluoro-3-(hydroxymethyl)phenyl isobutyl carbonate (Int-2) (0.35 gm, 1.446 mmol, 1 eq.) in DCM (10 mL) were added Pyridinium Chlorochromate [PCC] (0.46 gm, 2.16 mmol, 1.5 eq.) at 0° C. The resulting reaction mixture was stirred at room temperature for next 3 hr. The progress of the reaction was monitored by TLC; After completion of reaction, the reaction mixture was diluted with water (10 mL) and extracted with Ethyl acetate (3*20 mL), washed with brine. The organic layer was dried over sodium sulphate and concentrated under vacuum to get crude material; which was purified by Combi-flash by using 20% Ethyl acetate in hexane as mobile phase to give desired product as 4-fluoro-3-formylphenyl isobutyl carbonate (Int-3) 0.30 gm (Yield: 88%); 1H NMR (400 MHz, DMSO-d6) δ 10.20 (s, 1H), 7.70 (ddd, J=12.9, 7.3, 3.6 Hz, 1H), 7.61 (s, 1H), 7.52 (t, J=9.5 Hz, 1H), 4.04 (d, J=6.5 Hz, 2H), 2.00 (dp, J=13.4, 6.7 Hz, 1H), 0.96 (d, J=6.7 Hz, 6H).

### Step-3: Synthesis of 4-fluoro-3-(6-(3-methyl-1-(o-tolyl)-1H-pyrazol-5-yl)-2-azaspiro[3.3] heptan-2-yl)methyl) phenol (Compound-00150)

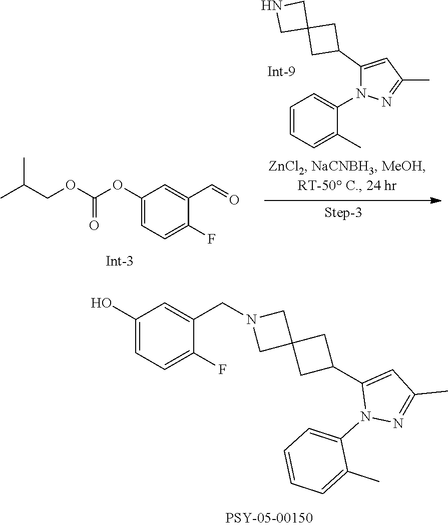 [SMILES: Cc1cc(C2CC3(CNC3)C2)n(-c2ccccc2C)n1 ; CC(C)COC(=O)Oc1ccc(F)c(C=O)c1 ; Cc1cc(C2CC3(C2)CN(Cc2cc(O)ccc2F)C3)n(-c2ccccc2C)n1]

To a stirred solution of 6-(3-methyl-1-(o-tolyl)-1H-pyrazol-5-yl)-2-azaspiro [3.3] heptane (Int-9) (0.20 gm, 0.749 mmol, 1 eq.) in Methanol (10 mL) were added 4-fluoro-3-formylphenyl isobutyl carbonate (Int-3) (0.179 gm, 0.749 mmol, 1 eq.) followed by zinc(II) chloride (0.050 gm, 0.374 mmol, 0.5 eq.) was added to that reaction mixture at Room temperature and stirred the reaction mixture at 50° C. for next 12 hr; the progress of the reaction was monitored by TLC (imine formation). Sodium cyano borohydride (0.094 gm, 1.498 mmol, 2 eq.) was added to that reaction mixture at 0° C. and stirred the reaction mixture for 12 hr, the progress of the reaction was monitored by TLC. After completion of the reaction mixture; the reaction mixture was concentrated under reduced pressure to get crude compound. Crude compound was diluted with water and extracted with ethyl acetate (3×30 mL); The organic layer was washed with brine; dried over sodium sulphate and concentrated under vacuum to get crude material; which was purified by Prep HPLC using 0.1% Formic acid in Water-100% Acetonitrile as mobile phase to give desired compound as 4-fluoro-3-((6-(3-methyl-1-(o-tolyl)-1H-pyrazol-5-yl)-2-azaspiro [3.3]heptan-2-yl)methyl) phenol (PSY-05-00150) 0.035 gm (Yield: 10.93%) m/z 392.0 [M+1]+; 1H NMR (400 MHZ, Methanol-d4) δ 8.510 (s, 1H), 7.48-7.30 (m, 3H), 7.19 (d, J=7.8 Hz, 1H), 7.00 (t, J=9.1 Hz, 1H), 6.80 (d, J=12.4 Hz, 1H), 6.80 (s, 1H), 6.19 (s, 1H), 4.04 (s, 2H), 3.87 (s, 2H), 3.80 (s, 2H), 3.15-3.06 (m, 1H), 2.43 (m, 2H), 2.38 (dt, J=36.4, 11.3 Hz, 2H), 2.27 (s, 3H), 2.00 (s, 3H).

### Example 8: Synthesis of 2-(2-Fluoro-5-hydroxybenzoyl)-N-Methyl-N-(o-tolyl)-2-azaspiro[3.3] heptane-6-carboxamide [Compound 185] and 2-(2-Ethoxy-4-fluorobenzoyl)-N-Methyl-N-(o-tolyl)-2-azaspiro [3.3]heptane-6-carboxamide [Compound 187]

### Reaction Scheme:

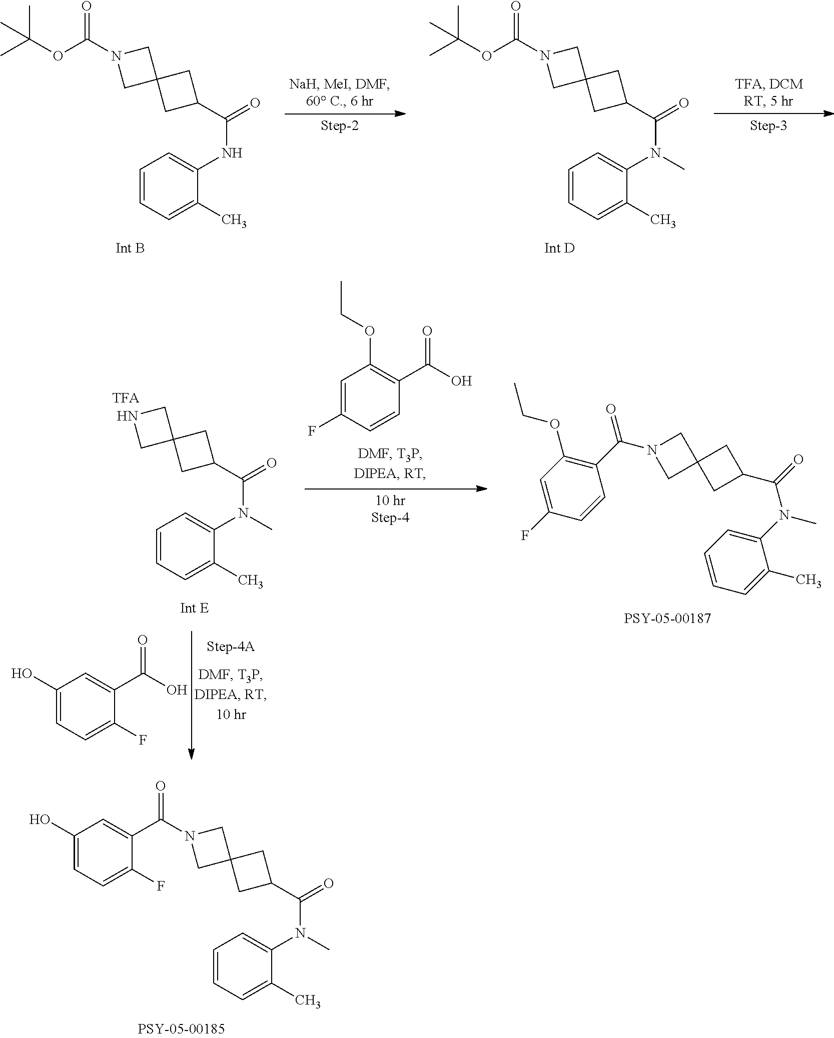

### Step-1:-Synthesis of Tert-butyl 6-(methyl(o-tolyl) carbamoyl)-2-azaspiro[3.3]heptane-2-carboxylate (Int-D)

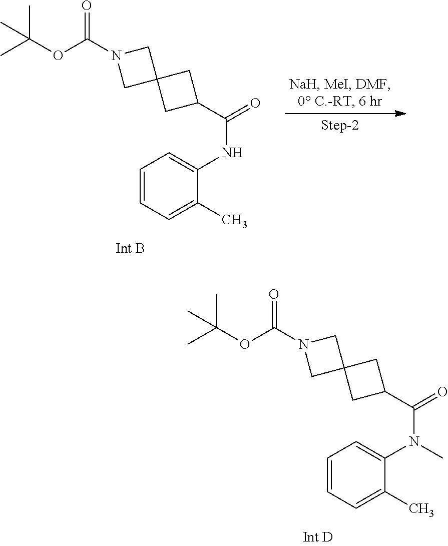 [SMILES: Cc1ccccc1NC(=O)C1CC2(C1)CN(C(=O)OC(C)(C)C)C2 ; Cc1ccccc1N(C)C(=O)C1CC2(C1)CN(C(=O)OC(C)(C)C)C2]

Tert-butyl 6-(o-tolylcarbamoyl)-2-azaspiro[3.3]heptane-2-carboxylate 0.5 g (1.5151 mmol, 1 eq.) dissolved in Dimethylformamide 5 mL. Sodium hydride 0.08 g (1.9696 mmol, 1.3 eq.) was added, stirred reaction mixture at room temperature for 15 min followed by Methyl Iodide 0.25 g (1.818 mmol, 1.2 eq.) added stirred reaction mixture at 60° C. for 6 hr. Reaction monitored on TLC. After completion of the reaction, the reaction mixture diluted with water. Product extracted with Ethyl Acetate (3*25 mL), combined organics concentrated to obtain crude product. Crude purified by column chromatography using Ethyl acetate: Hexanes as solvent system to get desired product. Tert-butyl 6-(methyl(o-tolyl) carbamoyl)-2-azaspiro[3.3]heptane-2-carboxylate. 0.51 g (Solid) (yield-98%) m/z 345.4 [M+1]+1H. NMR (400 MHz, Chloroform-d) δ 7.35-7.20 (m, 3H), 7.05 (d, J=7.6 Hz, 1H), 3.91-3.76 (m, 4H), 3.20 (s, 3H), 2.70 (q, J=8.3 Hz, 1H), 2.57-2.47 (m, 1H), 2.49-2.39 (m, 1H), 2.22 (s, 3H), 2.05-1.95 (m, 2H), 1.43 (s, 9H).

### Step-2: —Synthesis of N-Methyl-N-(o-tolyl)-2-azaspiro[3.3]heptane-6-carboxamide (Int-E)

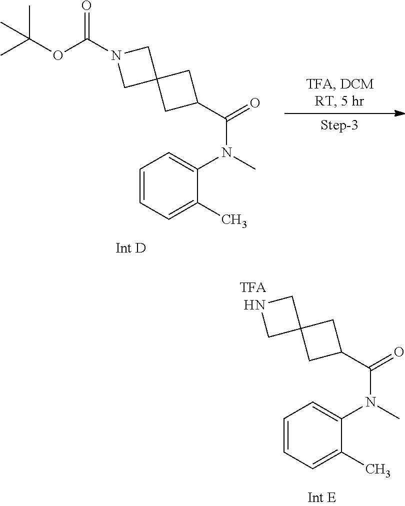 [SMILES: Cc1ccccc1N(C)C(=O)C1CC2(C1)CN(C(=O)OC(C)(C)C)C2 ; Cc1ccccc1N(C)C(=O)C1CC2(CNC2)C1]

Tert-butyl 6-(methyl(o-tolyl) carbamoyl)-2-azaspiro [3.3] heptane-2-carboxylate 0.5 g was taken in Dichloromethane 50 mL, cooled it to 0° C., Trifluoroacetic acid 0.8 mL was added, stirred reaction mixture at room temperature for 5 hr. Reaction was monitored on TLC. After completion of the reaction it was concentrated completed was triturated with diethyl ether to get desired product as N-Methyl-N-(o-tolyl)-2-azaspiro[3.3]heptane-6-carboxamide 0.5 g (Solid) (yield-92%) m/z 245.4 [M+1]+.

### Step-3: —Synthesis of 2-(2-Fluoro-5-hydroxybenzoyl)-N-Methyl-N-(o-tolyl)-2-azaspiro [3.3] heptane-6-carboxamide (Compound-00187)

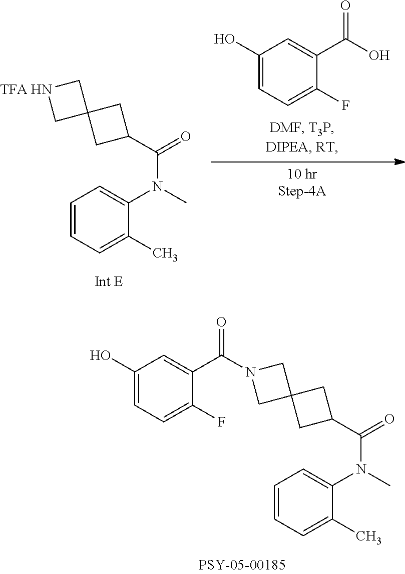

To a solution of 2-Fluoro-4-hydroxy benzoic acid 0.1 g (0.676 mmol, 1.1 eq.) in Dimethylformamide 2 mL. TFA salt of N-Methyl-N-(o-tolyl)-2-azaspiro[3.3]heptane-6-carboxamide 0.15 g (0.6147 mmol, 1 eq.) added and stir for 10 min. DIPEA 0.4 mL (2.459 mmol, 4 eq.), stirred reaction mixture for 10 min. T3P 0.4 mL (0.7377 mmol, 1.2 eq.) was added and stir, reaction mixture at room temperature for 10 hr. Reaction monitored on TLC. After completion of the reaction, it diluted with water. Product extracted with Ethyl acetate (3*15 mL), combined organics washed with water and dried over anhydrous Sodium sulphate. Organic layer concentrated to get crude compound. Crude gum, which purified by column chromatography using Ethyl acetate: Hexanes as solvent system. Desired product obtained as 2-(2-Fluoro-5-hydroxybenzoyl)-N-Methyl-N-(o-tolyl)-2-azaspiro[3.3]heptane-6-carboxamide 0.05 g (Solid). (yield-22.22%) m/z 383.35 [M+1]+1H NMR (400 MHZ, DMSO-d6) ô 9.62 (d, J=13.5 Hz, 1H), 7.40-7.23 (m, 3H), 7.16 (s, 1H), 7.17-6.98 (m, 1H), 6.83 (ddt, J=12.9, 8.7, 3.7 Hz, 1H), 6.78-6.69 (m, 1H), 3.97-3.80 (m, 4H), 3.05 (d, J=6.1 Hz, 3H), 2.62 (td, J=15.6, 7.7 Hz, 1H), 2.39-2.23 (m, 2H), 2.22-2.05 (m, 3H), 2.00-1.90 (m, 2H).

### Step-4:-Synthesis of 2-(2-Ethoxy-4-fluorobenzoyl)-N-Methyl-N-(o-tolyl)-2-azaspiro [3.3] heptane-6-carboxamide (Compound-00187)

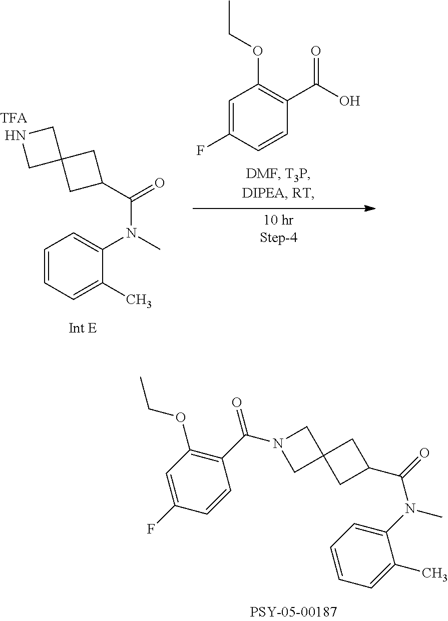 [SMILES: Cc1ccccc1N(C)C(=O)C1CC2(CNC2)C1 ; CCOc1cc(F)ccc1C(=O)O ; CCOc1cc(F)ccc1C(=O)N1CC2(CC(C(=O)N(C)c3ccccc3C)C2)C1]

To a solution of 2-Ethoxy-4-fluorobenzoic acid 0.15 g (0.676 mmol, 1.1 eq.) in Dimethyl formamide 2 mL. TFA salt of N-Methyl-N-(o-tolyl)-2-azaspiro [3.3]heptane-6-carboxamide 0.15 g (0.6147 mmol, 1 eq.) was added followed by Di-isopropyl ethyl amine 0.4 mL (2.459 mmol, 4 eq.), stirred reaction mixture for 10 min. T3P 0.4 mL (0.7377 mmol, 1.2 eq.) was added, stirred reaction mixture at room temperature for 10 hr. Reaction was monitored on TLC. Reaction mixture diluted with water and extracted with Ethyl acetate (3*15 mL). Organic layer combined, concentrated to obtained crude. Crude compound as gum, which purified by column chromatography using EtOAC: Hexanes as solvent system to get desired product. 2-(2-Ethoxy-4-fluorobenzoyl)-N-Methyl-N-(o-tolyl)-2-azaspiro [3.3]heptane-6-carboxamide 0.05 g (Solid) (yield-21.22%) m/z 411.4 [M+1]+ 1H NMR (400 MHZ, Methanol-d4) δ 7.42-7.22 (dq, J=27.9, 7.8 Hz, 4H), 7.14 (d, J=7.5 Hz, 1H), 6.91-6.66 (m, 2H), 4.18-3.89 (m, 5H), 3.87 (s, 1H), 3.17 (d, J=5.4 Hz, 3H), 2.79 (dp, J=24.3, 8.0 Hz, 1H), 2.43 (ddt, J=27.5, 19.5, 9.9 Hz, 2H), 2.20 (s, 3H), 2.17-1.98 (m, 2H), 1.41 (dt, J=18.6, 7.0 Hz, 3H).

### Example 9: Synthesis of (2-fluoro-5-hydroxyphenyl) (6-(1-(o-tolyl)-1H-pyrazol-5-yl)-2-azaspiro [3.3]heptan-2-yl) methanone [Compound 367]

### Synthetic Scheme:

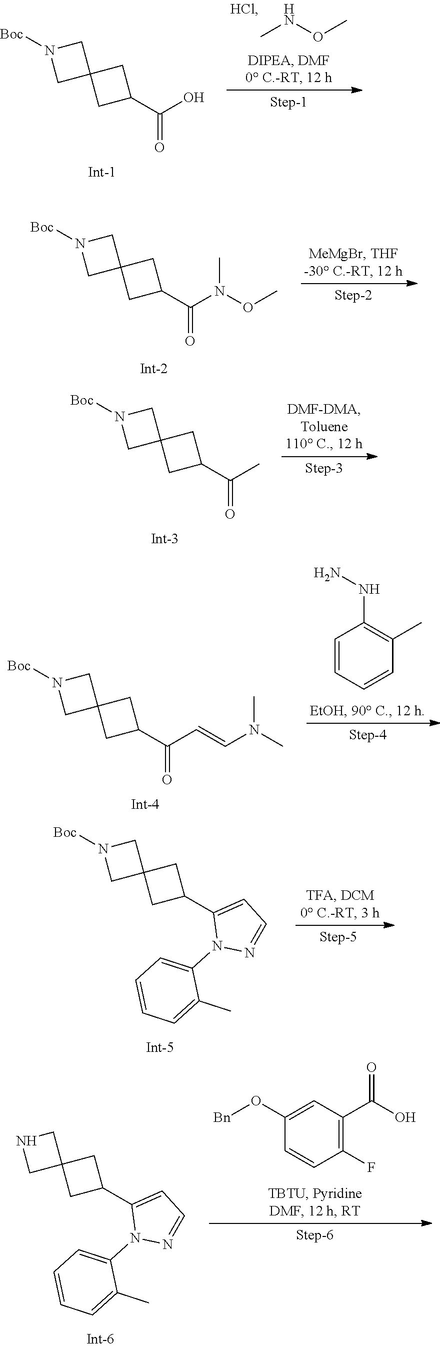

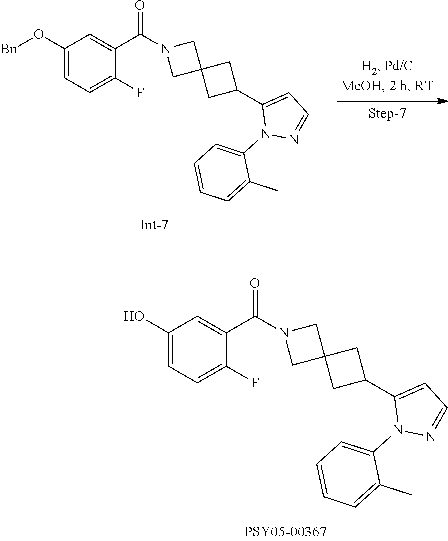 [SMILES: Cc1ccccc1-n1nccc1C1CC2(C1)CN(C(=O)c1cc(OCc3ccccc3)ccc1F)C2 ; Cc1ccccc1-n1nccc1C1CC2(C1)CN(C(=O)c1cc(O)ccc1F)C2]

### Step-1: Synthesis of tert-butyl 6-(methoxy(methyl) carbamoyl)-2-azaspiro [3.3]heptane-2-carboxylate (Int-2)

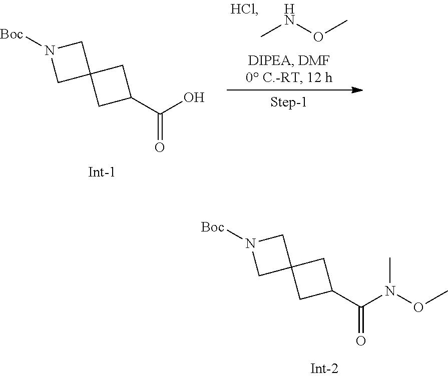

To a stirred solution of 2-(tert-butoxycarbonyl)-2-azaspiro[3.3]heptane-6-carboxylic acid (2.0 gm, 8.298 mmol, 1.0 eq.) in N,N-Dimethyl formamide (20 mL) were added HATU (3.15 gm, 8.29 mmol, 1.5 eq.), DIPEA (3.21 gm, 24.89 mmol, 3.0 eq.) followed by addition of N,O-Dimethylhydroxylamine Hydrochloride (1.00 gm, 10.37 mmol, 1.25 eq.) at 0° C. and allowed to stirred the reaction at room temperature for 12 hr; the progress of the reaction of the was monitored by TLC. After completion of reaction, the reaction mixture was diluted with water (25 mL) and extracted with ethyl acetate (3×30 mL), washed with brine. The organic layer was dried over sodium sulphate and concentrated under vacuum to get crude material; which was purified by combi-flash by using 100% Ethyl acetate as mobile phase to give desired product tert-butyl 6-(methoxy(methyl) carbamoyl)-2-azaspiro[3.3]heptane-2-carboxylate (Int-2) 1.50 gm (Yield: 63.82%), 1H NMR (400 MHZ, DMSO-d6) δ 3.89 (s, 2H), 3.73 (s, 2H), 3.62 (s, 3H), 3.08 (s, 3H), 2.30 (dt, J=8.1, 2.1 Hz, 4H), 1.37 (s, 9H).

### Step-2: Synthesis of tert-butyl 6-acetyl-2-azaspiro[3.3]heptane-2-carboxylate (Int-3)

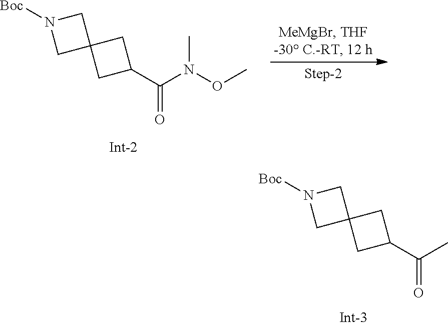 [SMILES: CON(C)C(=O)C1CC2(C1)CN(C(=O)OC(C)(C)C)C2 ; CC(=O)C1CC2(C1)CN(C(=O)OC(C)(C)C)C2]

To a stirred solution of tert-butyl 6-(methoxy(methyl) carbamoyl)-2-azaspiro[3.3] heptane-2-carboxylate (Int-2) (1.7 gm, 5.98 mmol, 1.0 eq.) in THF was added Methyl magnesium bromide (3M in THF) (5.98 mL, 17.95 mmol, 3.0 eq.) under inert atmosphere at −30° C. The reaction mixture was stirred at Room temperature for next 12 hr. The progress of the reaction was monitored by TLC; after completion of reaction, the reaction mixture was cooled to 0° C. and quenched with saturated ammonium chloride solution (10 mL) further diluted with water (30 mL) and extracted with ethyl acetate (3×30 mL), washed with brine. The organic layer was dried over sodium sulphate and concentrated under vacuum to get crude material; which was purified by combi-flash by using 80% Ethyl acetate in Hexane as mobile phase to give desired product tert-butyl 6-acetyl-2-azaspiro[3.3]heptane-2-carboxylate (Int-3) 1.30 gm (Yield: 90.90%); 1H NMR (400 MHz, Chloroform-d) δ 3.98 (s, 2H), 3.85 (s, 2H), 3.16 (p, J=8.3 Hz, 1H), 2.46-2.32 (m, 4H), 2.14 (s, 3H), 1.47 (s, 9H).

### Step-3: Synthesis of tert-butyl (E)-6-(3-(dimethylamino) acryloyl)-2-azaspiro [3.3] heptane-2-carboxylate (Int-4)

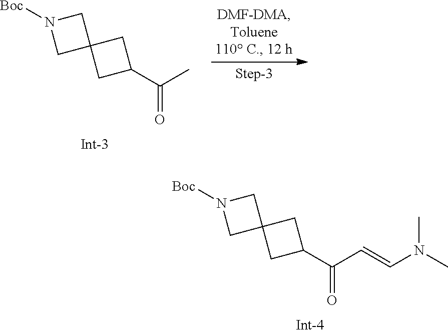 [SMILES: CC(=O)C1CC2(C1)CN(C(=O)OC(C)(C)C)C2 ; CN(C)/C=C/C(=O)C1CC2(C1)CN(C(=O)OC(C)(C)C)C2]

To a stirred solution of tert-butyl 6-acetyl-2-azaspiro[3.3]heptane-2-carboxylate (Int-3) (0.050 gm, 0.209 mmol, 1.0 eq.) in Toluene (1 mL) was added DMF-DMA (0.074 gm, 0.627 mmol, 3.0 eq.) and allowed to stirred the reaction at 100° C. for 12 hr; the progress of the reaction of the was monitored by TLC. After completion of reaction, the reaction mixture was diluted with water (3 mL) and extracted with ethyl acetate (3×5 mL), washed with brine. The organic layer was dried over sodium sulphate and concentrated under vacuum to get crude material; which was purified by combi-flash by using 90% Ethyl acetate in Hexane as mobile phase to give desired product tert-butyl (E)-6-(3 (dimethylamino) acryloyl)-2-azaspiro[3.3]heptane-2-carboxylate (Int-4) 0.040 gm (Yield: 65.04%); LCMS: 295.3 m/z [M+1]+

### Step-4: Synthesis of ter-butyl 6-(1-(o-tolyl)-1H-pyrazol-5-yl)-2-azaspiro[3.3]heptane-2-carboxylate (Int-5)

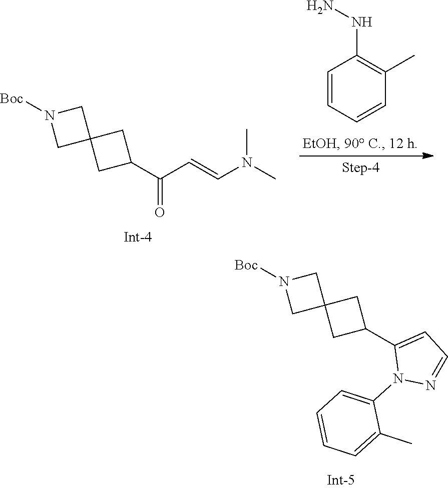

To a stirred solution of tert-butyl (E)-6-(3 (dimethylamino) acryloyl)-2-azaspiro[3.3] heptane-2-carboxylate (Int-4) (0.30 gm, 0.020 mmol, 1.0 eq.) in Ethanol (9 mL) was added o-tolylhydrazine (0.177 gm, 1.122 mmol, 1.1 eq.) and allowed to stirred the reaction at 90° C. for 12 hr; the progress of the reaction of the was monitored by TLC. After completion of reaction, the reaction mixture was evaporated under vacuum to get crude compound which was absorbed on silica gel and purified by combi-flash by using 100% Ethyl acetate as mobile phase to give desired product tert-butyl 6-(1-(o-tolyl)-1H-pyrazol-5-yl)-2-azaspiro[3.3]heptane-2-carboxylate (Int-5) 0.340 gm (Yield: 94.44%); LCMS: 354.3 m/z [M+1]+

### Step-5: Synthesis of 6-(1-(o-tolyl)-1H-pyrazol-5-yl)-2-azaspiro[3.3]heptane (Int-6)

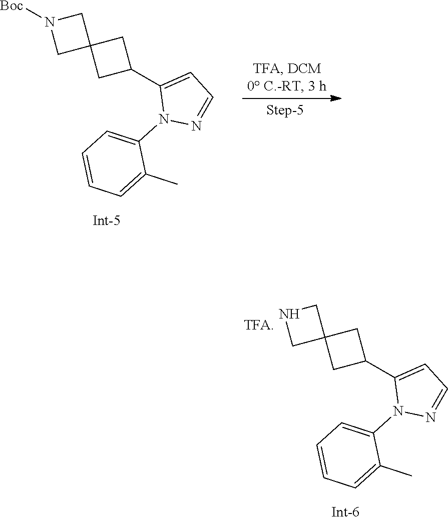 [SMILES: Cc1ccccc1-n1nccc1C1CC2(C1)CN(C(=O)OC(C)(C)C)C2 ; Cc1ccccc1-n1nccc1C1CC2(CNC2)C1]

To a stirred solution of tert-butyl 6-(1-(o-tolyl)-1H-pyrazol-5-yl)-2-azaspiro[3.3] heptane-2-carboxylate (Int-5) (0.30 gm, 0.84 mmol, 1.0 eq.) in Dichloromethane (10 mL) was added Trifluoroacetic acid (1.0 mL) at 0° C. and allowed to stirred the reaction at Room temperature for 3 hr; the progress of the reaction of the was monitored by TLC. After completion of reaction, the reaction mixture was evaporated under vacuum and triturated with pentane to afford crude compound 6-(1-(o-tolyl)-1H-pyrazol-5-yl)-2-azaspiro[3.3]heptane (Int-6) 0.30 gm (Yield: quantitative); LCMS: 254.2 m/z [M+1]+

### Step-6: Synthesis of (5-(benzyloxy)-2-fluorophenyl) (6-(1-(o-tolyl)-1H-pyrazol-5-yl)-2-azaspiro [3.3]heptan-2-yl) methanone (Int-7)

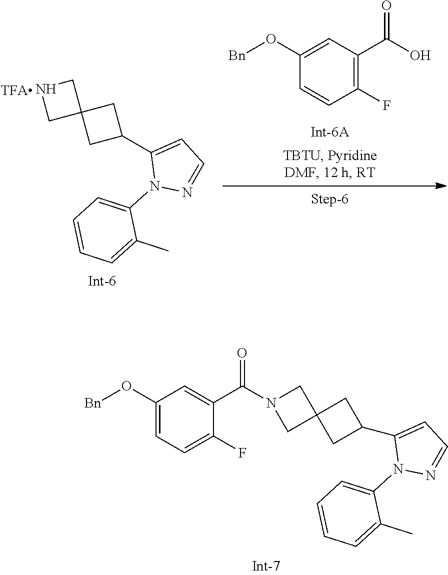 [SMILES: Cc1ccccc1-n1nccc1C1CC2(CNC2)C1 ; O=C(O)c1cc(OCc2ccccc2)ccc1F ; Cc1ccccc1-n1nccc1C1CC2(C1)CN(C(=O)c1cc(OCc3ccccc3)ccc1F)C2]

To a stirred solution of 5-(benzyloxy)-2-fluorobenzoic acid 6-(1-(o-tolyl)-1H-pyrazol-5-yl)-2-azaspiro[3.3]heptane (Int-6A) (0.25 gm, 1.016 mmol, 1.0 eq.) in N,N-Dimethyl formamide (10 mL) were added TBTU (0.58 gm, 1.52 mmol, 1.5 eq.) Pyridine (0.24 gm, 3.04 mmol, 3.0 eq.) followed by addition of 6-(1-(o-tolyl)-1H-pyrazol-5-yl)-2-azaspiro[3.3]heptane (Int-6) (0.30 gm, 1.21 mmol, 1.2 eq.) at 0° C. and allowed to stirred the reaction at room temperature for 12 hr; the progress of the reaction of the was monitored by TLC. After completion of reaction, the reaction mixture was diluted with water (15 mL) and extracted with ethyl acetate (3×20 mL), washed with brine. The organic layer was dried over sodium sulphate and concentrated under vacuum to get crude material; which was purified by combi-flash by using 90% Ethyl acetate in Hexane as mobile phase to give desired product (5-(benzyloxy)-2-fluorophenyl) (6-(1-(o-tolyl)-1H-pyrazol-5-yl)-2-azaspiro[3.3]heptan-2-yl) methanone (Int-7), 0.250 gm (Yield: 51.22%); LCMS: 482.3 m/z [M+1]+

### Step-7: Synthesis of (2-fluoro-5-hydroxyphenyl) (6-(1-(o-tolyl)-1H-pyrazol-5˜yl)-2-azaspiro[3.3] heptan-2-yl) methanone (Compound-00367)

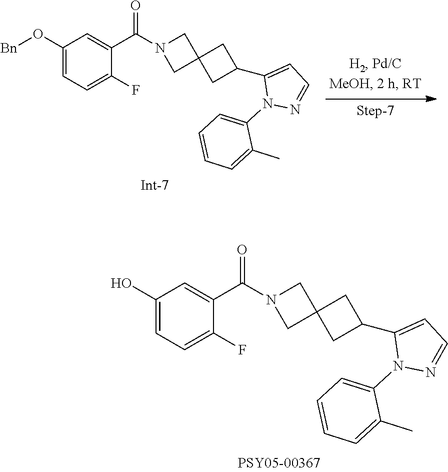 [SMILES: Cc1ccccc1-n1nccc1C1CC2(C1)CN(C(=O)c1cc(OCc3ccccc3)ccc1F)C2 ; Cc1ccccc1-n1nccc1C1CC2(C1)CN(C(=O)c1cc(O)ccc1F)C2]

To a stirred solution of (S-(benzyloxy)-2-fluorophenyl) (6-(1-(o-tolyl)-1H-pyrazol-5-yl)-2-azaspiro[3.3]heptan-2-yl) methanone (Int-7) (0.20 gm, 0.415 mmol, 1.0 eq.) was dissolved in Methanol (10 mL). 10% Pd/C (with 50% moisture) 0.050 gm was added at Room Temperature and Reaction mixture was allowed to stir for 2 hr. under Hydrogen atmosphere. Reaction was monitored by TLC. After completion of the reaction, Reaction mixture was filtered through celite bed, washed with Methanol (50 mL) and concentrated to get crude compound, which was purified by column chromatography using 60-120 mesh size silica gel and 80% Ethyl acetate in Hexane as mobile phase to give the desired product (2-fluoro-5-hydroxyphenyl) (6-(1-(o-tolyl)-1H-pyrazol-5-yl)-2-azaspiro[3.3]heptan-2-yl) methanone (PSY-05-00367) as a white solid, 0.120 gm, (Yield: 74.07%); LCMS: 392.1 m/z [M+] 1H NMR (400 MHZ, DMSO-d6) δ 9.63 (dd, J=8.3, 2.5 Hz, 1H), 7.56 (d, J=9.5 Hz, 1H), 7.41 (t, J=8.5 Hz, 2H), 7.32 (d, J=8.7 Hz, 1H), 7.19 (t, J=7.4 Hz, 1H), 7.05 (tt, J=9.2, 5.3 Hz, 1H), 6.83 (s, 1H), 6.74 (dd, J=5.5, 2.7 Hz, 1H), 6.36 (d, J=18.6 Hz, 1H), 3.94 (dd, J=25.2, 10.5 Hz, 3H), 3.05 (dt, J=18.8, 8.4 Hz, 1H), 2.34 (q, J=12.2, 11.5 Hz, 2H), 2.24 (q, J=10.5, 9.9 Hz, 2H), 1.91 (d, J=2.4 Hz, 3H).

### Example 10: Synthesis of [(2-chloro-5-hydroxyphenyl) (6-(3-methyl-1-(o-tolyl)-1H-pyrazol-5-yl)-2-azaspiro [3.3] heptan-2-yl) methanone] [Compound 140]

### Synthetic Scheme:

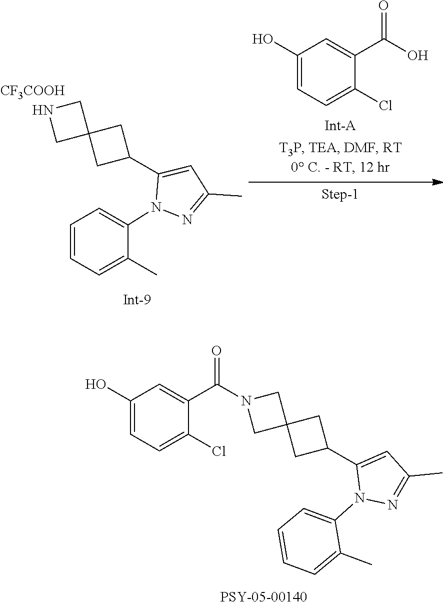 [SMILES: "CF3COOH" ; Cc1cc(C2CC3(CNC3)C2)n(-c2ccccc2C)n1 ; O=C(O)c1cc(O)ccc1Cl ; Cc1cc(C2CC3(C2)CN(C(=O)c2cc(O)ccc2Cl)C3)n(-c2ccccc2C)n1]

### Step-1: Synthesis of [(2-chloro-5-hydroxyphenyl) (6-(3-methyl-1-(o-tolyl)-1H-pyrazol-5-yl)-2-azaspiro [3.3] heptan-2-yl) methanone] [Compound-00140]

To a stirred solution of 6-(3-methyl-1-(o-tolyl)-1H-pyrazol-5-yl)-2-azaspiro [3.3] hept-5-ene 2,2,2-trifluoroacetate (0.100 gm, 0.2624 mmol) in DMF (5.0 mL) were added 2-chloro-5-hydroxybenzoic acid (0.054 gm, 0.314 mmol), Triethylamine (0.106 gm, 1.049 mmol) and Propanephosphonic acid anhydride [T3P, 50 wt. % in ethyl acetate] (0.125 gm, 0.393 mmol) at 0° C. The resulting reaction mixture was stirred at room temperature for next 12 hr. The progress of the reaction was monitored by TLC; After completion of reaction, the reaction mixture was diluted with water (10 mL) and extracted with Ethyl acetate (3*50 mL), The organic layer was washed with brine, dried over sodium sulphate and concentrated under vacuum to get crude material; which was purified by Prep HPLC using 0.1% Formic acid in water-100% Acetonitrile as mobile phase to give desired compound as [(2-chloro-5-hydroxyphenyl) (6-(3-methyl-1-(0-tolyl)-1H-pyrazol-5-yl)-2-azaspiro[3.3]heptan-2-yl) methanone] (PSY-05-0140) 0.028 gm (Yield: 26%) m/z 422.6 [M+1]⁺; ¹H NMR (400 MHZ, DMSO-d6) δ 9.95 (s, 1H), 7.43-7.1 (m, 4H), 7.15 (t, J=7.7 Hz, 1H), 6.80 (t, 1H), 6.67 (d, J=2.8 Hz, 1H), 6.16-6.10 (s, 1H), 3.98 (s, 1H), 3.92 (s, 1H), 3.82 (s, 1H), 3.76 (s, 1H), 2.99 (dt, J=20.8, 8.4 Hz, 1H), 2.32-2.19 (m, 2H), 2.17 (d, J=10.7 Hz, 2H), 2.09 (s, 3H), 1.93 (d, J=2.2 Hz, 3H).

### Example 11: Synthesis of 2-(2-ethoxy-4-fluorobenzoyl)-N-(2-isopropylphenyl)-N-methyl-2-azaspiro [3.3] heptane-6-carboxamide [Compound 205]

### Synthetic Scheme:

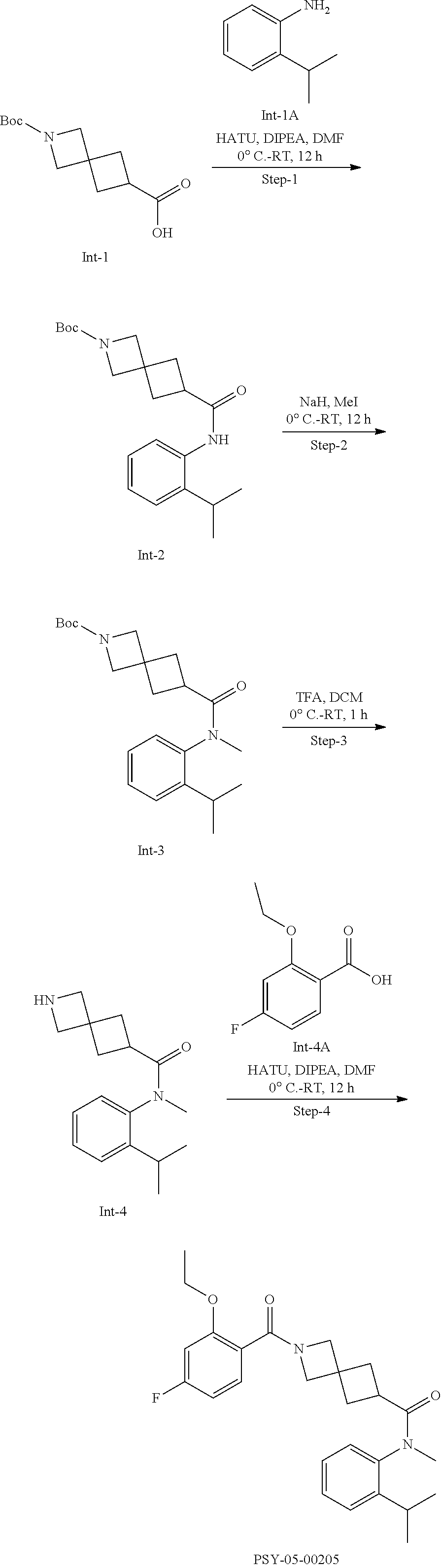

### Step-1: Synthesis of tert-butyl 6-((2-isopropylphenyl) carbamoyl)-2-azaspiro[3.3]heptane-2-carboxylate (Int-2)

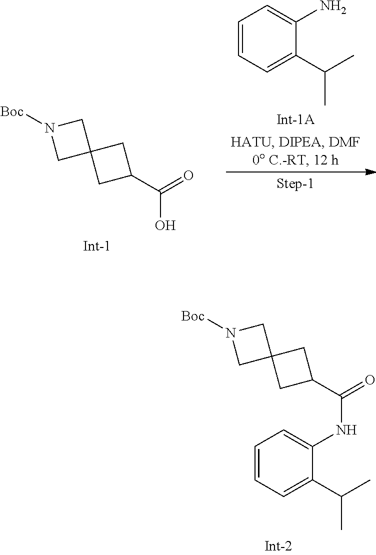

To a stirred solution of 2-(tert-butoxycarbonyl)-2-azaspiro[3.3]heptane-6-carboxylic acid (0.30 gm, 1.24 mmol, 1 eq.) in N,N-Dimethyl formamide (5 mL) were added HATU (0.70 gm, 1.86 mmol, 1.5 eq.) DIPEA (0.802 gm, 6.22 mmol, 5.0 eq.) followed by addition of 2-isopropylaniline (Int-1A) (0.21 gm, 1.24 mmol, 1.0 eq.) at 0° C. and allowed to stirred the reaction at room temperature for 12 hr; the progress of the reaction of was monitored by TLC. After completion of reaction, the reaction mixture was diluted with water (10 mL) and extracted with ethyl acetate (3×20 mL), washed with brine. The organic layer was dried over sodium sulphate and concentrated under vacuum to get crude material; which was purified by combi-flash by using 30% Ethyl acetate in n Hexane as mobile phase to give desired product tert-butyl 6-((2-isopropylphenyl) carbamoyl)-2-azaspiro[3.3]heptane-2-carboxylate (Int-2) 0.280 gm (Yield: 63.63%); LCMS: 359.6 m/z [M+1]+

### Step-2: Synthesis tert-butyl 6-((2-isopropylphenyl)(methyl) carbamoyl)-2-azaspiro[3.3]heptane-2-carboxylate acid (Int-3)

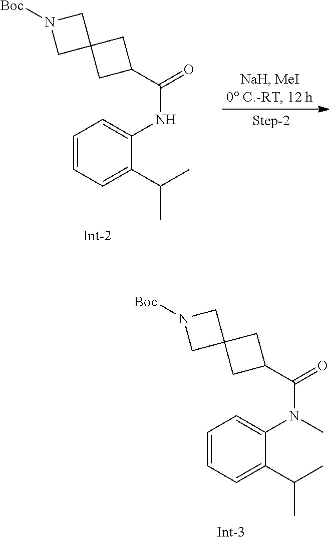 [SMILES: CC(C)c1ccccc1NC(=O)C1CC2(C1)CN(C(=O)OC(C)(C)C)C2 ; CC(C)c1ccccc1N(C)C(=O)C1CC2(C1)CN(C(=O)OC(C)(C)C)C2]

To a stirred solution of tert-butyl 6-((2-isopropylphenyl) carbamoyl)-2-azaspiro[3.3] heptane-2-carboxylate (0.4 gm, 1.11 mmol, 1.0 eq.) in N,N-Dimethyl formamide (5 mL), was added Sodium hydride (0.031 gm, 1.33 mmol, 1.2 eq.) at 0° C. The reaction was stirred at 0° C. for 10 min. Methyl Iodide (0.187 gm, 1.33 mmol, 1.2 eq.) was added dropwise and stirred the reaction mass at room temperature for 12 hrs. After completion of reaction as monitored by TLC, the reaction mixture was diluted with Ice cold water (10 mL) and extracted with Ethyl acetate (2×30 mL). The combined organic layer was washed with brine solution, dried over sodium sulfate and concentrated to obtain crude product, which was purified by combiflash using 30% Ethyl acetate in Hexane as eluent to afford tert-butyl 6-((2-isopropylphenyl)(methyl) carbamoyl)-2-azaspiro[3.3]heptane-2-carboxylate (Int-3)_{0.31} gm, (Yield-75.60%). LCMS: 373.4 m/z [M⁺]

### Step-3: Synthesis of N-(2-isopropylphenyl)-N-methyl-2-azaspiro [3.3] heptane-6-carboxamide (Int-4)

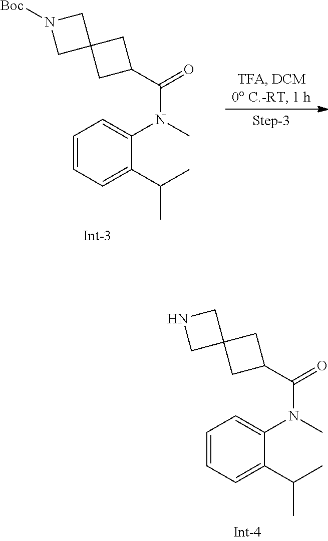 [SMILES: CC(C)c1ccccc1N(C)C(=O)C1CC2(C1)CN(C(=O)OC(C)(C)C)C2 ; CC(C)c1ccccc1N(C)C(=O)C1CC2(CNC2)C1]

To a stirred solution tert-butyl 6-((2-isopropylphenyl)(methyl) carbamoyl)-2-azaspiro [3.3]heptane-2-carboxylate (Int-3) (0.50 gm, 1.33 mmol, 1.0 eq.) in Dichloromethane (10 mL) was added Trifluoroacetic acid (1.0 mL) at 0° C. and allowed to stirred the reaction at Room temperature for 3 hr; the progress of the reaction of the was monitored by TLC. After completion of reaction, the reaction mixture was evaporated under vacuum and basified with bicarbonate solution (5 mL) extracted with Ethyl acetate. Ethyl acetate layer separated dried over sodium sulfate and concentrated to obtain crude product N-(2-isopropylphenyl)-N-methyl-2-azaspiro [3.3] heptane-6-carboxamide (Int-4) 0.350 gm (Yield: quantitative); LCMS: 273.4 m/z [M+1]+

### Step-4: Synthesis of 2-(2-ethoxy-4-fluorobenzoyl)-N-(2-isopropylphenyl)-N-methyl-2-azaspiro[3.3]heptane-6-carboxamide (Compound-00205)

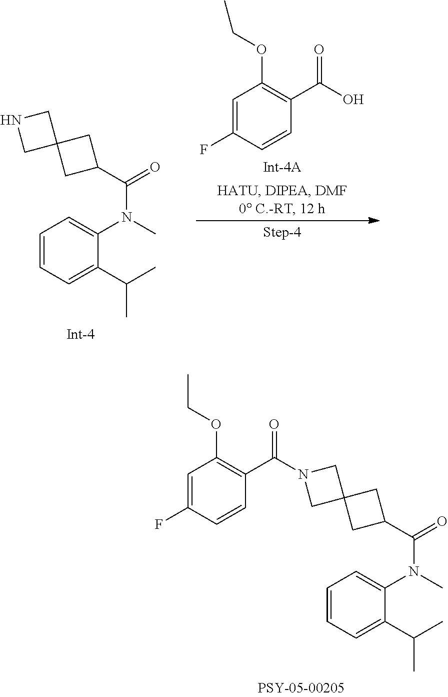 [SMILES: CC(C)c1ccccc1N(C)C(=O)C1CC2(CNC2)C1 ; CCOc1cc(F)ccc1C(=O)O ; CCOc1cc(F)ccc1C(=O)N1CC2(CC(C(=O)N(C)c3ccccc3C(C)C)C2)C1]

To a stirred solution of 2-ethoxy-4-fluorobenzoic acid (Int-4A), (0.150 gm, 0.815 mmol 1.0 eq.) in N,N-Dimethyl formamide (3 ml) were added N-(2-isopropylphenyl)-N-methyl-2-azaspiro [3.3] heptane-6-carboxamide (0.221 gm, 0.815 mmol, 1 eq.), DIPEA (0.31 gm, 2.445 mmol, 3.0 eq.), HATU (0.10 gm, 1.222 mmol, 1.5 eq.) at 0° C. The resulting reaction mixture was stirred at room temperature for 12 h. After completion of reaction as monitored by TLC, the reaction mixture was diluted with water (10 ml) and extracted with ethyl acetate (3*20 ml). The organic layer was washed with brine (10 ml), dried over sodium sulphate and concentrated under vacuum to get crude material which was purified by combi-flash using 10% Methanol in Dichloromethane as eluent to get 2-(2-ethoxy-4-fluorobenzoyl)-N-(2-isopropylphenyl)-N-methyl-2-azaspiro [3.3] heptane-6-carboxamide (PSY-05-00205) as white solid 0.070 gm (Yield: 20%); LCMS: 439.7 m/z 1H NMR (400 MHz, DMSO-d6) δ 7.51-7.38 (m, 1H), 7.42-7.25 (m, 1H), 7.26 (dd, J=7.9, 4.7 Hz, 1H), 6.95 (ddd, J=19.4, 11.6, 2.4 Hz, 1H), 6.84-6.71 (m, 1H), 4.08 (dq, J=21.4, 6.8 Hz, 2H), 3.82 (t, J=4.8 Hz, 3H), 3.75 (dq, J=14.6, 8.9, 6.8 Hz, 1H), 3.16-3.01 (m, 3H), 2.83 (p, J=6.8 Hz, 1H), 2.68-2.53 (m, 1H), 2.35-2.20 (m, 2H), 2.00 (ddd, J=30.9, 12.2, 8.7 Hz, 2H), 1.31 (dt, J=21.4, 7.0 Hz, 3H), 1.21-1.09 (m, 6H).

### Example 12: Synthesis of 2-(2-ethoxy-4-fluorobenzoyl)-N-ethyl-N-(o-tolyl)-2-azaspiro [3.3] heptane-6-carboxamide [Compound 203]

### Synthetic Scheme:

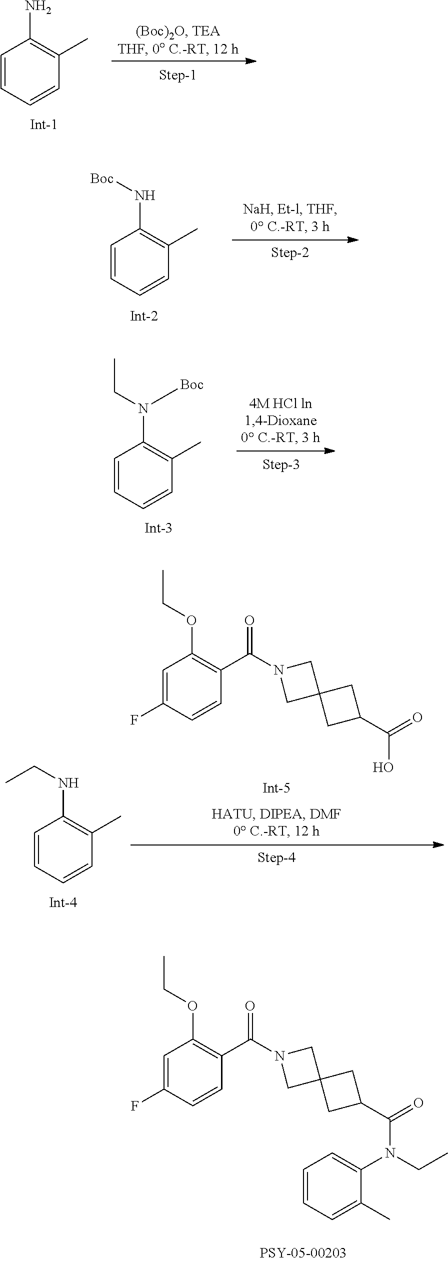

### Step-1: Synthesis of tert-butyl o-tolyl Carbamate (Int-2)

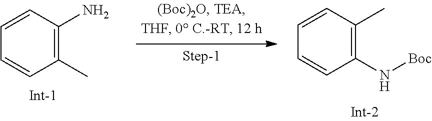

To a stirred solution of o-toluidine (1.0 gm, 9.34 mmol, 1.0 eq.) in N,N-Dimethyl formamide (10 mL) were added TEA (1.88 gm, 18.69 mmol, 2.0 eq.) followed by addition of Boc anhydride (2.24 gm, 10.288 mmol, 1.0 eq.) at 0° C. and allowed to stirred the reaction at room temperature for 12 hr; the progress of the reaction of was monitored by TLC. After completion of reaction, the reaction mixture was diluted with water (10 mL) and extracted with ethyl acetate (3×20 mL), washed with brine. The organic layer was dried over sodium sulphate and concentrated under vacuum to get crude material; which was purified by combi-flash by using 30% Ethyl acetate in n Hexane as mobile phase to give desired product tert-butyl o-tolylcarbamate (Int-2) 1.1 gm (Yield: 56.99%); LCMS: 208.2 m/z [M+1]+

### Step-2: Synthesis of tert-butyl ethyl (o-tolyl) carbamate (Int-3)

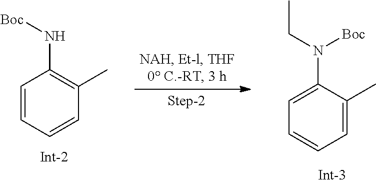 [SMILES: Cc1ccccc1NC(=O)OC(C)(C)C ; CCN(C(=O)OC(C)(C)C)c1ccccc1C]

To a stirred solution of tert-butyl o-tolylcarbamate (0.5 gm, 2.415 mmol, 1.0 eq.) in N,N-Dimethyl formamide (8 mL), was added Sodium hydride (0.069 gm, 2.898 mmol, 1.2 eq.) at 0° C. The reaction was stirred at 0° C. for 10 min. Ethyl Iodide (0.741 gm, 2.898 mmol, 1.2 eq.) was added dropwise and stirred the reaction mass at room temperature for 12 hrs. After completion of reaction as monitored by TLC, the reaction mixture was diluted with Ice cold water (10 mL) and extracted with Ethyl acetate (2×30 mL). The combined organic layer was washed with brine solution, dried over sodium sulfate and concentrated to obtain crude product, which was purified by combiflash using 30% Ethyl acetate in Hexane as eluent to afford tert-butyl ethyl(o-tolyl) carbamate (Int-3) 0.45 gm, (Yield-80.35%). LCMS: 180.1 m/z [M−56]+

### Step-3: Synthesis of N-ethyl-2-methylaniline (Int-4)

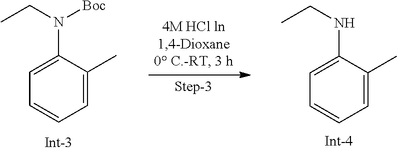

To a stirred solution of tert-butyl ethyl(o-tolyl) carbamate (Int-3) (0.50 gm, 2.127 mmol, 1.0 eq.) in Dichloromethane (10 mL) was added Trifluoroacetic acid (1.0 mL) at 0° C. and allowed to stirred the reaction at Room temperature for 3 hr; the progress of the reaction of the was monitored by TLC. After completion of reaction, the reaction mixture was evaporated under vacuum and basified with bicarbonate solution (5 mL) extracted with Ethyl acetate. Ethyl acetate layer separated dried over sodium sulfate and concentrated to obtain crude product N-ethyl-2-methylaniline (Int-4) 0.350 gm (Yield: quantitative); LCMS: 136.1 m/z [M+1]+

### Step-4: Synthesis of 2-(2-ethoxy-4-fluorobenzoyl)-N-ethyl-N-(o-tolyl)-2-azaspiro[3.3]heptane-6-carboxamide (Compound-00203)

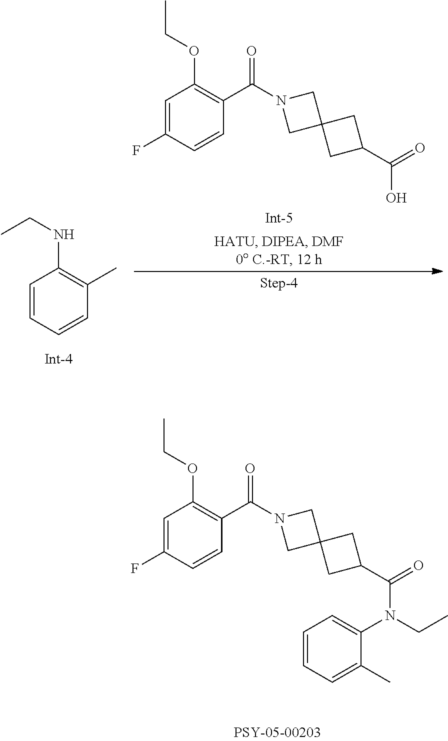

To a stirred solution of 2-(2-ethoxy-4-fluorobenzoyl)-2-azaspiro[3.3]heptane-6-carboxylic acid (Int-5), (0.20 gm, 0.651 mmol 1. eq.) in N,N-Dimethyl formamide (5 ml) were N-ethyl-2-methylaniline (0.096 gm, 0.651 mmol, 1.0 eq.), DIPEA (0.25 gm, 1.954 mmol, 3.0 eq.), HATU (0.371 gm, 0.977 mmol, 1.5 eq.) at 0° C. The resulting reaction mixture was stirred at room temperature for 12 h. After completion of reaction as monitored by TLC, the reaction mixture was diluted with water (10 ml) and extracted with ethyl acetate (3*20 ml). The organic layer was washed with brine (10 ml), dried over sodium sulphate and concentrated under vacuum to get crude material which was purified by combiflash using 10% Methanol in Dichloromethane as eluent to get 2-(2-ethoxy-4-fluorobenzoyl)-N-ethyl-N-(o-tolyl)-2-azaspiro[3.3]heptane-6-carboxamide (PSY-05-00203) as white solid 0.10 gm (Yield: 37.03%); LCMS: 425.7 m/z 1H NMR (400 MHz, DMSO-d6) δ 7.33 (s, 2H), 7.33-7.20 (m, 2H), 7.09 (d, J=7.6 Hz, 1H), 6.94 (ddd, J=17.5, 11.7, 2.4 Hz, 1H), 6.76 (dtd, J=10.8, 8.4, 2.5 Hz, 1H), 4.08 (dt, J=13.8, 6.9 Hz, 3H), 4.06-3.88 (m, 1H), 3.86-3.73 (m, 2H), 3.71 (s, 2H), 3.10 (dt, J=13.6, 6.8 Hz, 1H), 2.33-2.19 (m, 2H), 2.12 (d, J=1.9 Hz, 3H), 1.92 (s, 1H), 1.93-1.82 (m, 1H), 1.30 (dt, J=21.2, 7.0 Hz, 3H), 0.98 (td, J=7.1, 5.2 Hz, 3H).

### Example 13: Synthesis of N-(2-chlorophenyl)-2-(2-ethoxy-4-fluorobenzoyl)-N-methyl-2-azaspiro [3.3] heptane-6-carboxamide [Compound 206]

### Synthetic Scheme:

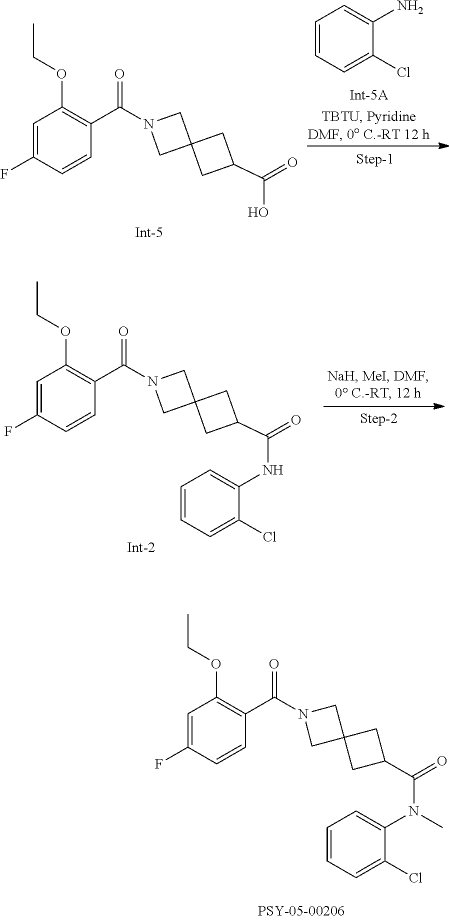 [SMILES: CCOc1cc(F)ccc1C(=O)N1CC2(CC(C(=O)O)C2)C1 ; Nc1ccccc1Cl ; CCOc1cc(F)ccc1C(=O)N1CC2(CC(C(=O)Nc3ccccc3Cl)C2)C1 ; CCOc1cc(F)ccc1C(=O)N1CC2(CC(C(=O)N(C)c3ccccc3Cl)C2)C1]

### Step-1: Synthesis of N-(2-chlorophenyl)-2-(2-ethoxy-4-fluorobenzoyl)-2-azaspiro[3.3]heptane-6-carboxamide (Int-6)

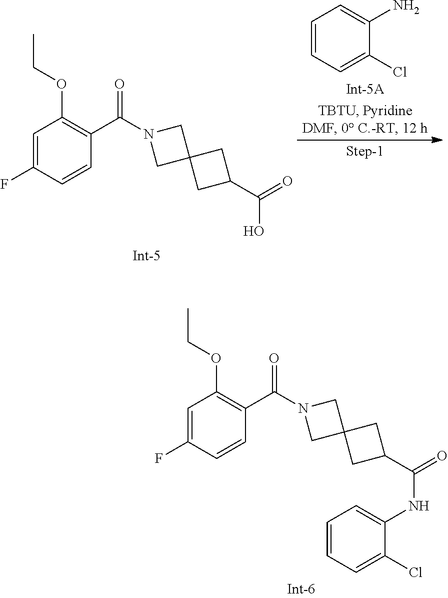 [SMILES: CCOc1cc(F)ccc1C(=O)N1CC2(CC(C(=O)O)C2)C1 ; Nc1ccccc1Cl ; CCOc1cc(F)ccc1C(=O)N1CC2(CC(C(=O)Nc3ccccc3Cl)C2)C1]

To a stirred solution of 2-(2-ethoxy-4-fluorobenzoyl)-2-azaspiro[3.3]heptane-6-carboxylic acid (0.150 gm, 0.488 mmol, 1.0 eq.) in N,N-Dimethyl formamide (5 mL) were added TBTU (0.326 gm, 0.879 mmol, 1.8 eq.) Pyridine (0.154 gm, 1.954 mmol, 5.0 eq.) followed by addition of 2-chloroaniline (Int-5A) (0.082 gm, 0.586 mmol, 1.2 eq.) at 0° C. and allowed to stirred the reaction at room temperature for 12 hr; the progress of the reaction was monitored by TLC. After completion of reaction, the reaction mixture was diluted with water (10 mL) and extracted with ethyl acetate (3×20 mL), washed with brine. The organic layer was dried over sodium sulphate and concentrated under vacuum to get crude material; which was purified by combi-flash by using 5% Methanol in Dichloromethane as mobile phase to give desired product N-(2-chlorophenyl)-2-(2-ethoxy-4-fluorobenzoyl)-2-azaspiro[3.3]heptane-6-carboxamide (Int-6) 0.150 gm (Yield: 75%); LCMS: 417.7 m/z [M+]

### Step-2: Synthesis of N-(2-chlorophenyl)-2-(2-ethoxy-4-fluorobenzoyl)-N-methyl-2-azaspiro [3.3] heptane-6-carboxamide (Compound-00206)

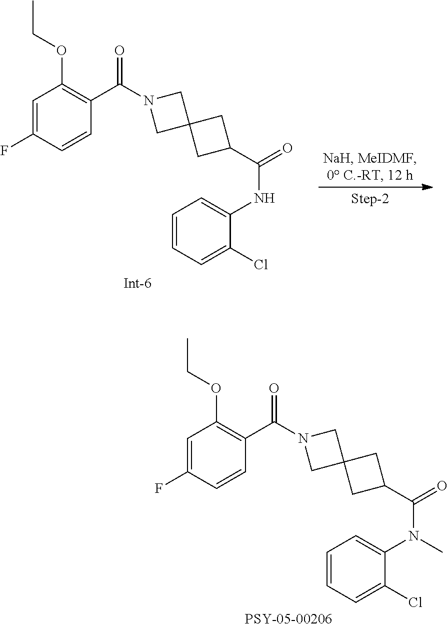 [SMILES: CCOc1cc(F)ccc1C(=O)N1CC2(CC(C(=O)Nc3ccccc3Cl)C2)C1 ; CCOc1cc(F)ccc1C(=O)N1CC2(CC(C(=O)N(C)c3ccccc3Cl)C2)C1]

To a stirred solution of N-(2-chlorophenyl)-2-(2-ethoxy-4-fluorobenzoyl)-2-azaspiro [3.3]heptane-6-carboxamide (0.175 gm, 0.420 mmol, 1 eq.) in N,N-Dimethyl formamide (3 mL), was added Sodium hydride (0.012 gm, 0.504 mmol, 1.2 eq.) at 0° C. The reaction was stirred at 0° C. for 10 min. Methyl Iodide (0.071 gm, 0.504 mmol, 1.2 eq.) was added dropwise and stirred the reaction mass at room temperature for 12 hrs. After completion of reaction as monitored by TLC, the reaction mixture was diluted with Ice cold water (10 mL) and extracted with Ethyl acetate (2×20 mL). The combined organic layer was washed with brine solution, dried over sodium sulfate and concentrated to obtain crude product, which was purified by combiflash using 5% Methanol in Dichloromethane as mobile phase to give desired product N-(2-chlorophenyl)-2-(2-ethoxy-4-fluorobenzoyl)-N-methyl-2-azaspiro[3.3]heptane-6-carboxamide (PSY-05-00206) 0.10 gm, (Yield-55.55%). LCMS: 431.4 m/z [M+] 1H NMR. (400 MHz, DMSO-d6) δ 7.64 (ddd, J=9.3, 6.6, 3.9 Hz, 1H), 7.46 (ddd, J=13.8, 6.0, 3.3 Hz, 3H), 7.38-7.22 (m, 1H), 6.95 (ddd, J=15.6, 11.6, 2.4 Hz, 1H), 6.77 (dtd, J=10.6, 8.4, 2.4 Hz, 1H), 4.11 (q, J=5.9, 5.1 Hz, 1H), 4.06 (q, J=6.9, 5.9 Hz, 1H), 3.84 (d, J=7.1 Hz, 2H), 3.76 (d, J=14.2 Hz, 2H), 3.07 (d, J=6.4 Hz, 3H), 2.68 (hept, J=8.2 Hz, 1H), 2.34-2.24 (m, 2H), 2.08-1.85 (m, 2H), 1.42-1.22 (m, 3H).

### Example 14: Synthesis of (4-fluoro-2-(2,2,2-trifluoroethoxy)phenyl) (6-(3-methyl-1-(o-tolyl)-1H-pyrazol-5-yl)-2-azaspiro[3.3]heptan-2-yl) methanone. [Compound 178]

### Synthetic Scheme:

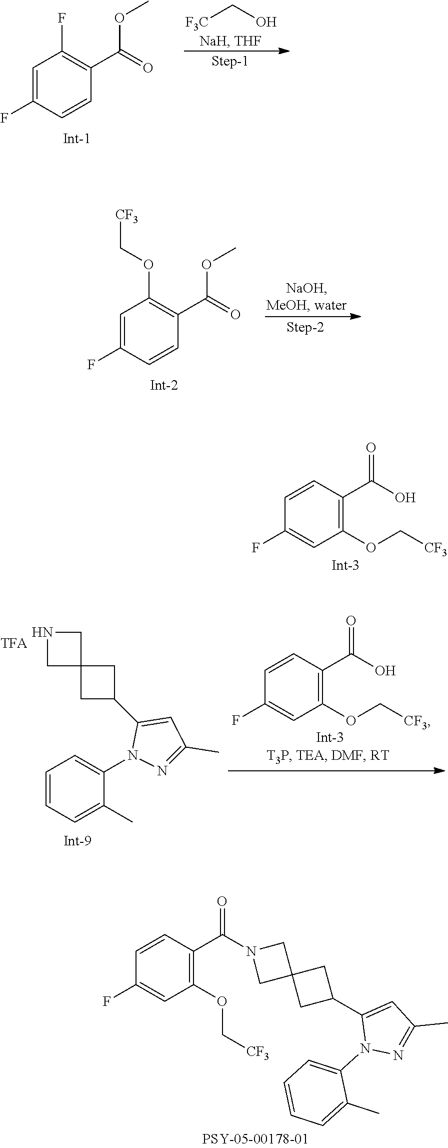

### Step-1: Synthesis of methyl 4-fluoro-2-(2, 2, 2-trifluoroethoxy)benzoate (Int-2)

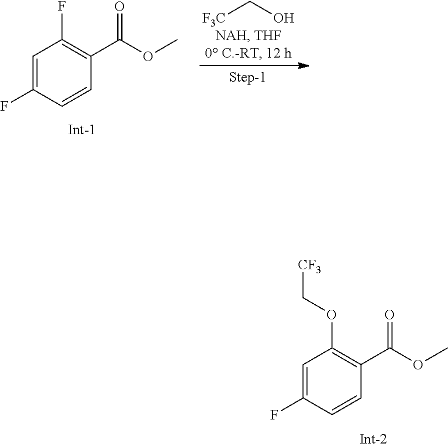 [SMILES: COC(=O)c1ccc(F)cc1F ; OCC(F)(F)F ; COC(=O)c1ccc(F)cc1OCC(F)(F)F]

To a stirred solution of methyl 2,4-difluorobenzoate (0.10 gm, 0.58 mmol, 1.0 eq.) in N,N-Dimethyl formamide (2 mL), was added Sodium hydride (0.016 gm, 0.679 mmol, 1.2 eq.) at 0° C. The reaction was stirred at 0° C. for 10 min. 2, 2, 2-trifluoroethan-1-ol (0.081 gm, 0.813 mmol) was added dropwise and stirred the reaction mass at room temperature for 12 hrs. After completion of reaction as monitored by TLC, the reaction mixture was diluted with Ice cold water (5 mL) and extracted with Ethyl acetate (2×20 mL). The combined organic layer was washed with brine solution, dried over sodium sulfate and concentrated to obtain crude product, which was purified by combiflash using 10% Ethyl acetate in Hexane as eluent to afford methyl 4-fluoro-2-(2,2,2-trifluoroethoxy)benzoate (Int-2) 0.10 gm, (Yield-68%). The compound was containing disubstituted product which was non separable by column chromatography was carried forward as a mixture for next step.

### Step-2: Synthesis of 4-fluoro-2-(2,2,2-trifluoroethoxy)benzoic acid (Int-3)

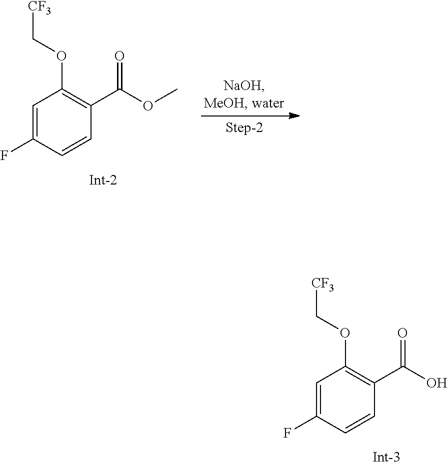 [SMILES: COC(=O)c1ccc(F)cc1OCC(F)(F)F ; O=C(O)c1ccc(F)cc1OCC(F)(F)F]

To a stirred solution of methyl 4-fluoro-2-(2,2,2-trifluoroethoxy)benzoate (Int-2) (1.0 gm, 3.96 mmol, 1 eq.) in THF (10 mL), MeOH (5 mL), Water (5 mL) was added Sodium Hydroxide [NaOH] (0.23 gm, 5.95 mmol, 1.5 eq.) at 0° C. The reaction mixture was stirred at room temperature for next 12 hr. The progress of the reaction was monitored by TLC; after completion of reaction, the reaction mixture was evaporated under vacuum. The crude product was acidified with 2N HCL (PH˜4) The white solid was precipitated out which was filtered through Buchner funnel and dried under vacuum to give 4-fluoro-2-(2,2,2-trifluoroethoxy)benzoic acid (Int-3) 0.50 gm (Yield: 53%); 1H NMR (400 MHZ, DMSO-d6) § 12.83 (s, 1H), 7.77 (dd, J=8.7, 7.0 Hz, 1H), 7.17 (dd, J=11.1, 2.4 Hz, 1H), 6.96 (td, J=8.4, 2.4 Hz, 1H), 4.84 (q, J=8.8 Hz, 2H).

### Step-3: Synthesis of (4-fluoro-2-(2,2,2-trifluoroethoxy)phenyl) (6-(3-methyl-1-(o-tolyl)-1H-pyrazol-5-yl)-2-azaspiro[3.3]heptan-2-yl) methanone (Compound-00178-001)

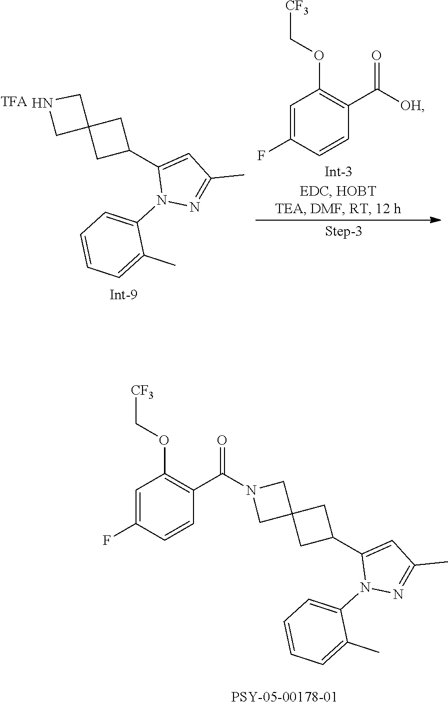

To a stirred solution of 6-(3-methyl-1-(o-tolyl)-1H-pyrazol-S-yl)-2-azaspiro[3.3] heptane 2,2,2-trifluoroacetate (0.10 gm, 0.37 mmol 1 .eq.) in N,N-Dimethyl formamide (3 ml) were added 4-fluoro-2-(2,2,2-trifluoroethoxy)benzoic acid (0.89 gm, 0.37 mmol, 1.0 eq.), TEA (0.11 gm, 1.12 mmol, 3.0 eq.), EDC-HCl (0.10 gm, 0.56 mmol, 1.5 eq.), and HOBT (0.025 gm, 0.18 mmol, 0.5 eq.) at 0° C. The resulting reaction mixture was stirred at room temperature for 12 h. After completion of reaction as monitored by TLC, the reaction mixture was diluted with water (10 ml) and extracted with ethyl acetate (3*20 ml). The organic layer was washed with brine (10 ml), dried over sodium sulphate and concentrated under vacuum to get crude material which was purified by combiflash using 80% Ethyl acetate in hexane as eluent to get PSY-05-00178 as white solid (0.050. (Yield: 28%); LCMS: 488.5 m/z [M+] 1H NMR (400 MHZ, DMSO-d6) δ 7.46-7.36 (m, 2H), 7.40-7.25 (m, 2H), 7.16 (s, 1H), 7.21-7.10 (m, 1H), 6.98-6.87 (m, 1H), 6.17 (s, 1H), 4.93-4.79 (m, 2H), 3.95 (s, 1H), 3.86 (d, J=19.9 Hz, 2H), 3.78 (s, 1H), 3.03-2.92 (m, 1H), 2.33 (t, J=10.4 Hz, 2H), 2.21 (dd, J=23.9, 9.2 Hz, 5H), 1.93 (d, J=2.3 Hz, 3H).

### Example 15: Synthesis of (2-cyclopropoxy-4-fluorophenyl) (6-(3-(trifluoromethyl)-5-(2-(trifluoromethyl)phenyl)-1H-pyrazol-1-yl)-2-azaspiro[3.3]heptan-2-yl) methanone (Compound 424); and (2-cyclopropoxy-4-fluorophenyl) (6-(5-(trifluoromethyl)-3-(2-(trifluoromethyl)phenyl)-1H-pyrazol-1-yl)-2-azaspiro[3.3]heptan-2-yl) methanone (Compound 451)

### Synthetic Scheme:

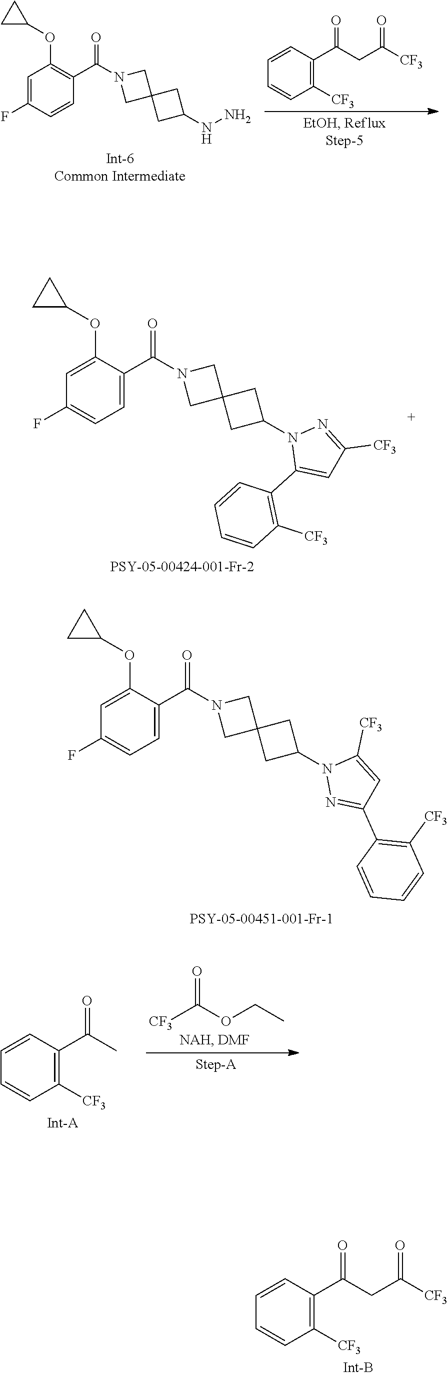

### Step-A: 4,4,4-trifluoro-1-(2-(trifluoromethyl)phenyl) butane-1,3-dione (Int-B)

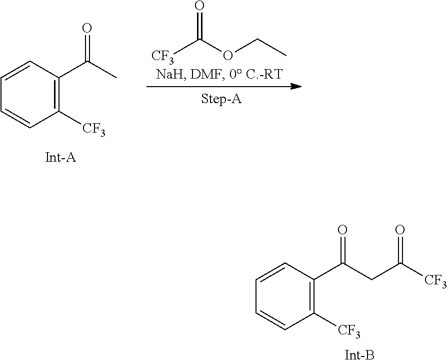

To a stirred solution of 1-(2-(trifluoromethyl)phenyl) ethan-1-one (3.0 g, 1.59 mmol, 1.0 eq.) in N,N-Dimethylformamide at 0° C. under N₂ atmosphere, NaH 60% (0.500 g, 2.393 mmol, 1.5 eq.) was added and allowed to stir for 30 mins at 0° C. Then added Trifluoroethylacetate (3.50 g, 2.388 mmol, 1.5 eq.) to the reaction mass and stirred the reaction at room temperature for 16 hr. After completion of reaction as monitored by TLC, the reaction mixture was poured in ice cold water (100 mL). Acidified with dil. HCl up to PH 4.0 and extracted with ethyl acetate (50 mL*3). The organic layer was dried over Sodium sulfate and concentrated under reduce pressure to get residue. The residue was purified by combiflash using 50% ethyl acetate in n-hexane as eluent to get tert-butyl 6-(methoxy(methyl) carbamoyl)-2-azaspiro[3.3]heptane-2-carboxylate (Int-B) (2.0 g, 44.15%).

### Step-5: (2-cyclopropoxy-4-fluorophenyl) (6-(3-(trifluoromethyl)-5-(2-(trifluoromethyl)phenyl)-1H-pyrazol-1-yl)-2-azaspiro[3.3]heptan-2-yl) methanone (PSY-05-00424-001) and (2-cyclopropoxy-4-fluorophenyl) (6-(5-(trifluoromethyl)-3-(2-(trifluoromethyl)phenyl)-1H-pyrazol-1-yl)-2-azaspiro[3.3]heptan-2-yl) methanone (PSY-05-00451-001)

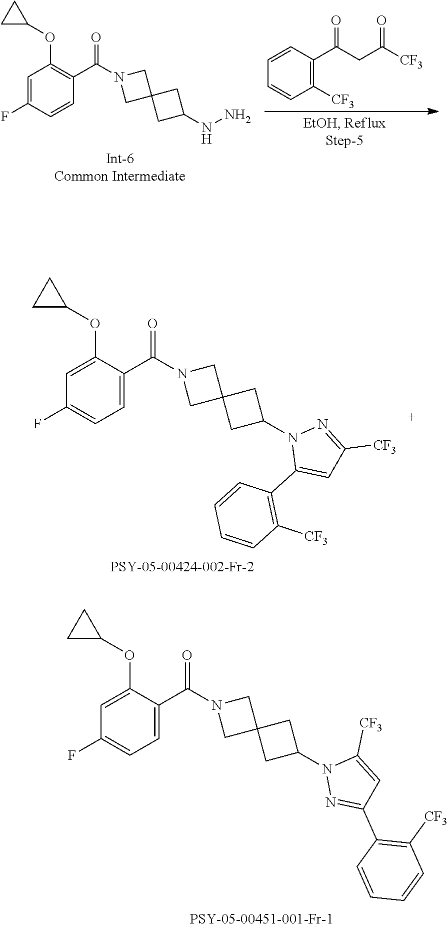 [SMILES: NNC1CC2(C1)CN(C(=O)c1ccc(F)cc1OC1CC1)C2 ; O=C(CC(=O)C(F)(F)F)c1ccccc1C(F)(F)F ; O=C(c1ccc(F)cc1OC1CC1)N1CC2(CC(n3nc(C(F)(F)F)cc3-c3ccccc3C(F)(F)F)C2)C1 ; O=C(c1ccc(F)cc1OC1CC1)N1CC2(CC(n3nc(-c4ccccc4C(F)(F)F)cc3C(F)(F)F)C2)C1]

To a stirred solution of tert-butyl 6-(methoxy(methyl) carbamoyl)-2-azaspiro[3.3] heptane-2-carboxylate (Int-B) (0.3 g, 1.05 mmol, 1.0 eq.) in Ethanol (5 mL) was added (2-cyclopropoxy-4-fluorophenyl) (6-hydrazineyl-2-azaspiro[3.3]heptan-2-yl) methanone (Int-6) (0.62 g, 2.10 mmol, 2.0 eq.) the reaction mass was heated at 80° C. for 16 hr. After completion of reaction as monitored by TLC, the reaction mixture was concentrated to get residue. The residue was purified by combiflash using 80% ethyl acetate in n-hexane as eluent to get mixture of two region isomers. The crude was submitted to prep HPLC for purification. Two Fraction was collected.

Fraction-1: (2-cyclopropoxy-4-fluorophenyl) (6-(5-(trifluoromethyl)-3-(2-(trifluoromethyl)phenyl)-1H-pyrazol-1-yl)-2-azaspiro[3.3]heptan-2-yl) methanone (PSY-05-00451-001) (0.027 g, 4.97%). LCMS: m/z 553.91 [M+1]⁺. NMR: 1H NMR (400 MHZ, DMSO-d₆) δ 8.68 (s, 1H), 8.05-7.83 (m, 3H), 7.63 (d, J=7.5 Hz, 1H), 7.30 (dq, J=27.9, 10.5, 9.1 Hz, 1H), 7.21-7.28 (dq, J=27.9, 10.5, 9.1 Hz, 1H), 6.92 (d, J=13.5 Hz, 2H), 4.46 (s, 1H), 4.00-3.95 (s, 3H), 3.80 (s, 2H), 2.78-2.70 (d, J=18.7 Hz, 4H), 0.87 (d, J=16.1 Hz, 2H), 0.79-0.70 (m, 2H).

Fraction-2: (2-cyclopropoxy-4-fluorophenyl) (6-(3-(trifluoromethyl)-5-(2-(trifluoromethyl)phenyl)-1H-pyrazol-1-yl)-2-azaspiro[3.3]heptan-2-yl) methanone (PSY-05-00424-001) (0.050 g, 9.20%). LCMS: m/z 553.91 [M+1]⁺. NMR: 1H NMR (400 MHZ, DMSO-d6) δ 8.68 (s, 1H), 8.15-8.37 (m, 5H), 7.63 (d, J=7.5 Hz, 1H), 7.40 (dq, J=27.9, 10.5, 9.1 Hz, 1H), 6.92 (d, J=13.5 Hz, 1H), 5.52 (d, J=13.5 Hz, 1H), 4.56 (s, 1H), 4.00 (s, 3H), 3.63 (s, 2H), 2.78-2.70 (d, J=18.7 Hz, 2H), 2.24 (d, J=7.3 Hz, 2H), 0.87 (d, J=16.1 Hz, 2H), 0.79-0.70 (m, 2H), 0.72 (s, 2H).

### Example 16: Synthesis of (2-fluoro-5-hydroxyphenyl) (6-(5-(2-fluorophenyl)-4-(trifluoromethyl)-1H-pyrazol-1-yl)-2-azaspiro[3.3]heptan-2-yl) methanone [Compound 472] and (2-fluoro-5-hydroxyphenyl) (6-(3-(2-fluorophenyl)-4-(trifluoromethyl)-1H-pyrazol-1-yl)-2-azaspiro[3.3]heptan-2-yl) methanone [Compound 473]

### Synthetic Scheme:

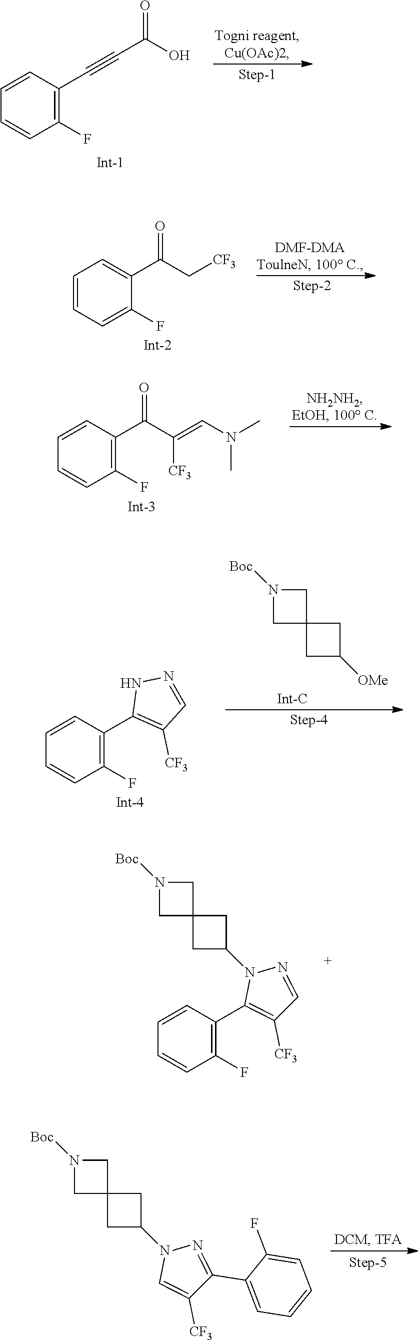

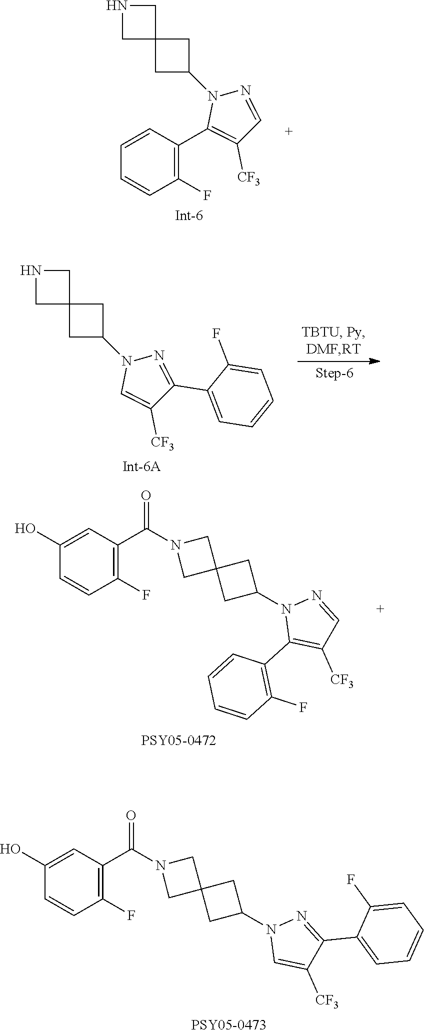 [SMILES: Fc1ccccc1-c1c(C(F)(F)F)cnn1C1CC2(CNC2)C1 ; Fc1ccccc1-c1nn(C2CC3(CNC3)C2)cc1C(F)(F)F ; O=C(c1cc(O)ccc1F)N1CC2(CC(n3ncc(C(F)(F)F)c3-c3ccccc3F)C2)C1 ; O=C(c1cc(O)ccc1F)N1CC2(CC(n3cc(C(F)(F)F)c(-c4ccccc4F)n3)C2)C1]

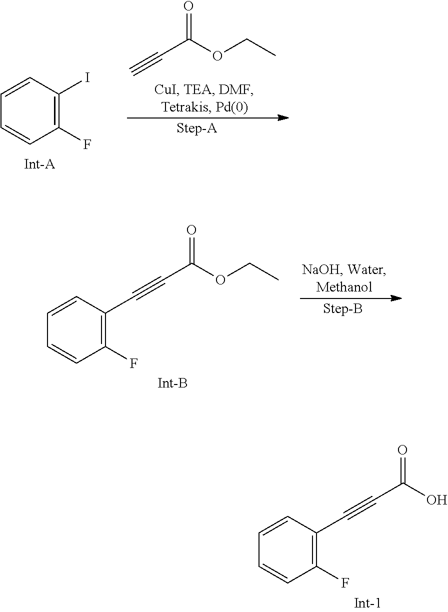

### Step-A: ethyl 3-(2-fluoro-phenyl) propiolate (Int-B)

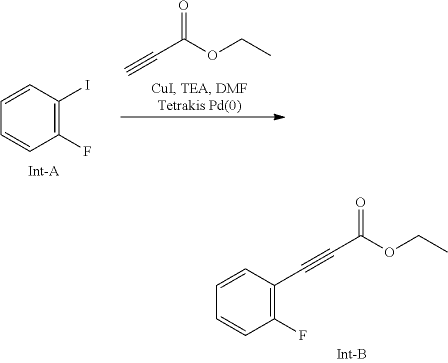

A mixture of the 1-fluoro-2-iodo-benzene (Int-A) (11.3 g, 56.06 mmol, 1.1 eq.), Tetrakis (Triphenyl phosphine) palladium (0) (5.8 g, 5.096 mmol, 0.1 eq.), and CuI (copper (1) iodide) (1.0 g, 5.096 mmol, 0.1 eq.) in Triethylamine (50 ml) was purged with nitrogen and treated with the ethyl propiolate (5.0 g, 50.96 mmol, 1.0 eq.). The resultant mixture was stirred at room temperature overnight. The reaction mixture was poured into sat aq. NaHCO₃ Solⁿ and extracted with ethyl acetate. The organic layer was washed with brine, dried sodium sulphate, decanted, concentrated, and purified by flash chromatography (0-20% ethyl acetate in n-hexane) to get desired product ethyl 3-(2-fluoro-phenyl) propiolate (Int-B) as a clear oil. (2.0 g, 20.44%).

### Step-B: 3-(2-fluoro-phenyl) propiolic acid (Int-1)

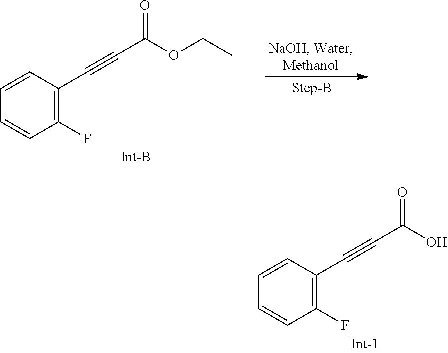

To a solution of the ethyl 3-(2-fluoro-phenyl) propiolate (Int-B) (2.0 g, 10.42 mmol, 1.0 eq.) in Methanol (5 mL) and H₂O (15 mL) was added NaOH (0.916 g, 22.91 mmol, 2.2 eq.). The mixture stirred at room temperature for 16 h, then concentrated in vacuum. The crude was diluted with water (100 mL) and acidified with an aq. solution of 2M HCl. This aq mixture was extracted with ethyl acetate (2×200 mL) and the combined organic extracts were washed with brine (400 mL), dried over sodium sulphate and concentrated in vacuum to provide to product of 3-(2-fluoro-phenyl) propiolic acid (Int-1) as solid. (1.6 g, 93.63%).

### Step-1:3,3,3-trifluoro-1-(2-fluoro-phenyl) propan-1-one (Int-2)

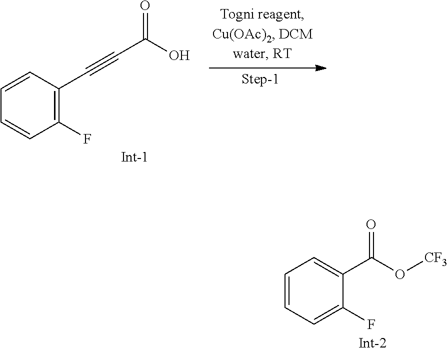 [SMILES: O=C(O)C#Cc1ccccc1F ; O=C(OC(F)(F)F)c1ccccc1F]

A Sealed test tube with a magnetic stirring bar was charged with 3-(2-fluoro-phenyl) propiolic acid (1.6 g, 9.816 mmol, 1.0 eq.) Togni-(II) (6.4 g, 19.632 mmol, 2.0 eq.) Cu(OAc)₂·H₂O (3.56 g, 19.632 mmol, 2.0 eq), TMEDA (3.6 mL, 24.54 mmol, 2.5 eq.), followed by dichloromethane (16 mL) and H₂O (24 mL). The reaction mixture was stirred at room temperature. After stirring for 24 h, the reaction mixture was extracted with dichloromethane (15 mL×3), dried over sodium sulphate, filtered and concentrated. The residue was purified with silica gel chromatography to provide pure product of 3,3,3-trifluoro-1-(2-fluoro-phenyl) propan-1-one (Int-2) as solid (0.200 g, 9.95%).

### Step-2: (Z)-3-(dimethylamino)-1-(2-fluorophenyl)-2-(trifluoromethyl) prop-2-en-1-one (Int-3)

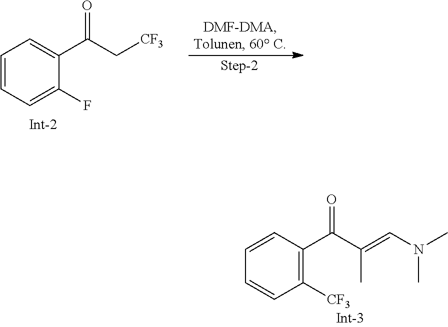 [SMILES: O=C(CC(F)(F)F)c1ccccc1F ; C/C(=C\N(C)C)C(=O)c1ccccc1C(F)(F)F]

To a stirred solution of 3,3,3-trifluoro-1-(2-fluoro-phenyl) propan-1-one (Int-2) (1.5 g, 7.28 mmol, 1.0 eq.) in Toluene (10 mL) was added N,N-Dimethylformamide dimethyl-acetal (2.6 g, 21.84 mmol, 3.0 eq.) the reaction mass was heated at 60° C. for 16 h. After completion of reaction as monitored by TLC, the reaction mixture was poured in ice cold water (100 mL) and extracted with ethyl acetate (50 mL*3). The organic layer was washed with brine (100 mL), dried over sodium sulphate and concentrated under reduce pressure to get residue. The residue was purified by combiflash using 15-20% ethyl acetate in n-hexane as eluent to get (Z)-3-(dimethylamino)-1-(2-fluorophenyl)-2-(trifluoromethyl) prop-2-en-1-one (Int-3) (1.5 g, 78.91%).

### Step-3:5-(2-fluorophenyl)-4-(trifluoromethyl)-1H-pyrazole (Int-4)

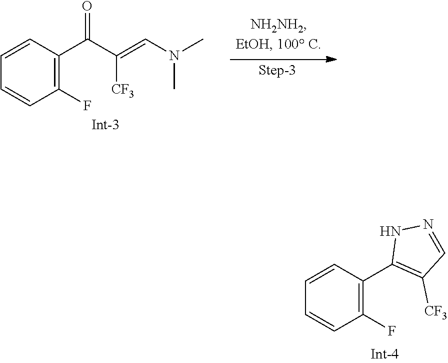 [SMILES: CN(C)/C=C(/C(=O)c1ccccc1F)C(F)(F)F ; Fc1ccccc1-c1[nH]ncc1C(F)(F)F]

To a stirred solution of (Z)-3-(dimethylamino)-1-(2-fluorophenyl)-2-(trifluoromethyl) prop-2-en-1-one (1.5 g, 6.95 mmol, 1.0 eq.) in isopropyl alcohol (10 mL) was added hydrazine hydrate (0.38 g, 7.65 mmol, 1.1 eq.) the reaction mass was heated at 80° C. for 16 hr. After completion of reaction as monitored by TLC, the reaction mixture was concentrated to get residue. The residue was purified by combiflash using 15-20% ethyl acetate in n-hexane as eluent to get 5-(2-fluorophenyl)-4-(trifluoromethyl)-1H-pyrazole (Int-4) (0.90 g, 68.01%).

### Step-4: Tert-butyl 6-(5-(2-fluoro-phenyl)-4-(trifluoromethyl)-1H-pyrazol-1-yl)-2-azaspiro [3.3] heptane-2-carboxylate (Int-5) and tert-butyl6-(3-(2-fluoro-phenyl)-4-(trifluoromethyl)-1H-pyrazol-1-yl)-2-azaspiro [3.3] heptane-2-carboxylate (Int-5A)

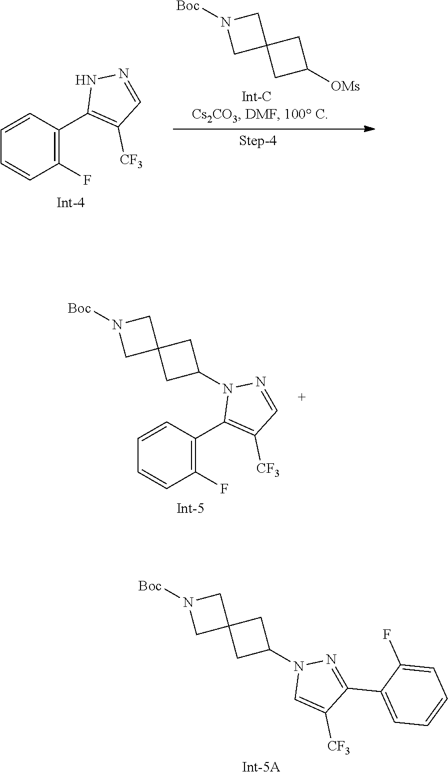 [SMILES: Fc1ccccc1-c1[nH]ncc1C(F)(F)F ; CC(C)(C)OC(=O)N1CC2(CC(OS(C)(=O)=O)C2)C1 ; CC(C)(C)OC(=O)N1CC2(CC(n3ncc(C(F)(F)F)c3-c3ccccc3F)C2)C1 ; CC(C)(C)OC(=O)N1CC2(CC(n3cc(C(F)(F)F)c(-c4ccccc4F)n3)C2)C1]

To a solution of 5-(2-fluoro-5-methylphenyl)-4-(trifluoromethyl)-1H-pyrazole (Int-4) (0.900 g, 3.9121 mmol, 1.0 eq.) in N,N-Dimethylformamide (10 mL), tert-butyl 6-((methyl sulfonyl)oxy)-2-azaspiro[3.3]heptane-2-carboxylate (Int-C) (1.37 g, 4.694 mmol, 1.2 eq.) and cesium carbonate (2.54 g, 7.824 mmol, 2.0 eq.) were added, and the reaction mixture was heated at 100° C. for 16 hr. After cooling to room temperature, the reaction mixture was poured into water (20 mL), and the solution was extracted with ethyl acetate (3×20 mL). The combined organic layers were washed with brine (20 mL), dried over sodium sulfate, filtered and concentrated under reduced pressure. The residue was purified by flash column chromatography on silica gel with ethyl acetate and n-hexane to get two region isomers of tert-butyl6-(5-(2-fluoro-phenyl)-4-(trifluoromethyl)-1H-pyrazol-1-yl)-2-azaspiro[3.3]heptane-2-carboxylate (Int-5) and tert-butyl6-(3-(2-fluoro-phenyl)-4-(trifluoromethyl)-1H-pyrazol-1-yl)-2-azaspiro[3.3]heptane-2-carboxylate (Int-5A) (1.0 g, 60.11%).

### Step-5:6-(5-(2-fluoro-phenyl)-4-(trifluoromethyl)-1H-pyrazol-1-yl)-2-azaspiro[3.3]heptane (Int-6) and 6-(3-(2-fluoro-5phenyl)-4-(trifluoromethyl)-1H-pyrazol-1-yl)-2-azaspiro[3.3]heptane (Int-6A)

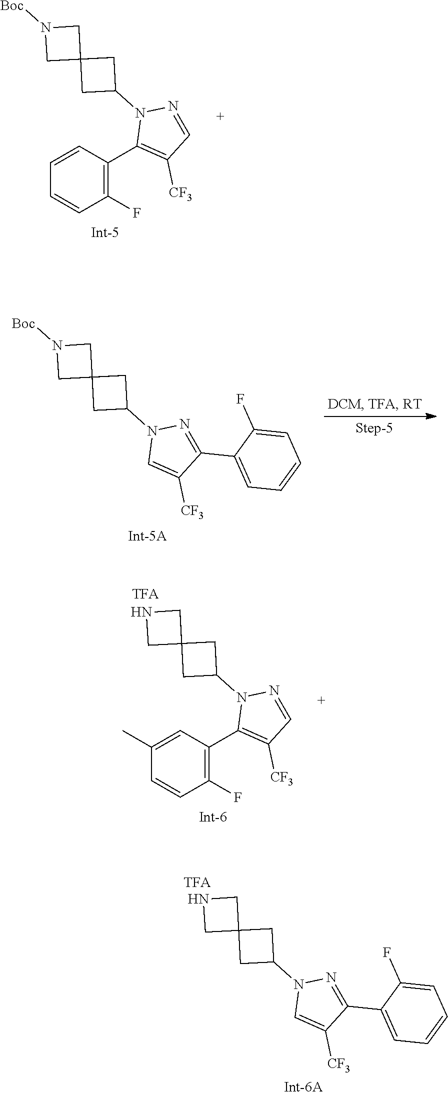 [SMILES: CC(C)(C)OC(=O)N1CC2(CC(n3ncc(C(F)(F)F)c3-c3ccccc3F)C2)C1 ; CC(C)(C)OC(=O)N1CC2(CC(n3cc(C(F)(F)F)c(-c4ccccc4F)n3)C2)C1 ; Cc1ccc(F)c(-c2c(C(F)(F)F)cnn2C2CC3(CNC3)C2)c1 ; Fc1ccccc1-c1nn(C2CC3(CNC3)C2)cc1C(F)(F)F]

To a stirred solution of tert-butyl6-(5-(2-fluoro-phenyl)-4-(trifluoromethyl)-1H-pyrazol-1-yl)-2-azaspiro[3.3]heptane-2-carboxylate (Int-5) and tert-butyl6-(3-(2-fluoro-phenyl)-4-(trifluoromethyl)-1H-pyrazol-1-yl)-2-azaspiro[3.3]heptane-2-carboxylate (Int-5A) (1.0 g, 0.6605 mmol, 1.0 eq.) in dichloromethane (10 ml) was added Trifluroacetic acid (2.5 mL, 2.5 v) at 0° C. The reaction was stirred at room temperature for 16 hr. After completion of reaction as monitored by TLC, the reaction mixture was concentrated and triturated with mixture of diethyl ether and hexane (1:1, 10 ml*3) to get two regio-isomers. 6-(5-(5-fluoro-phenyl)-3-methyl-1H-pyrazol-1-yl)-2-azaspiro [3.3] heptane (Int-6) and 6-(3-(5-fluoro-phenyl)-5-methyl-1H-pyrazol-1-yl)-2-azaspiro [3.3] heptane) (Int-6A) (1.5 g (TFA salt).

### Step-6:2-fluoro-5-hydroxyphenyl) (6-(5-(2-fluorophenyl)-4-(trifluoromethyl)-1H-pyrazol-1-yl)-2-azaspiro[3.3]heptan-2-yl) methanone (PSY-05-00474-001) and 2-fluoro-5-hydroxyphenyl) (6-(3-(2-fluorophenyl)-4-(trifluoromethyl)-1H-pyrazol-1-yl)-2-azaspiro[3.3]heptan-2-yl) methanone (PSY-05-00475-001)

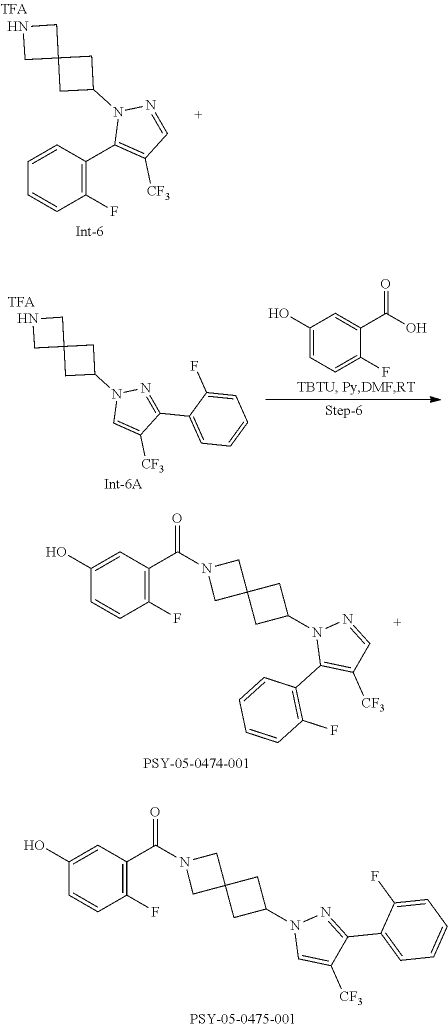 [SMILES: Fc1ccccc1-c1c(C(F)(F)F)cnn1C1CC2(CNC2)C1 ; Fc1ccccc1-c1nn(C2CC3(CNC3)C2)cc1C(F)(F)F ; O=C(O)c1cc(O)ccc1F ; O=C(c1cc(O)ccc1F)N1CC2(CC(n3ncc(C(F)(F)F)c3-c3ccccc3F)C2)C1 ; O=C(c1cc(O)ccc1F)N1CC2(CC(n3cc(C(F)(F)F)c(-c4ccccc4F)n3)C2)C1]

To a stirred solution of 6-(5-(5-fluoro-phenyl)-3-methyl-1H-pyrazol-1-yl)-2-azaspiro[3.3]heptane (INT-5) &6-(3-(5-fluoro-phenyl)-5-methyl-1H-pyrazol-1-yl)-2-azaspiro[3.3]heptane) (Int-5A) (0.500 g, 1.537 mmol, 1.0 eq.) in N,N-Dimethylformamide (5.0 mL) were added, pyridine (1.07 g, 15.37 mmol, 10.0 eq.), TBTU (0.740 g, 0.2.306 mmol, 1.5 eq.) at 0° C. Reaction to stirred for 15 min. then to add a 2-fluoro-5-hydroxybenzoic acid (0.359 g, 2.306 mmol, 1.5 eq.) at 0° C. The resulting reaction mixture was stirred at room temperature for 16 hr. After completion of reaction as monitored by TLC, the reaction mixture was diluted with water (15 ml) and extracted with ethyl acetate (3*20 ml) and again washed with sat. NaHCO₃ solution. The organic layer was dried over sodium sulphate and concentrated under vacuum to get crude material which was purified by using reverse phase Prep-HPLC. To get two fraction.

Fraction-1: (2-fluoro-5-hydroxyphenyl) (6-(3-(2-fluorophenyl)-4-(trifluoromethyl)-1H-pyrazol-1-yl)-2-azaspiro[3.3]heptan-2-yl) methanone (PSY-05-00473-001) (0.012 g 1.68%). LCMS: m/z 464.01 [M+H]⁺. ¹H NMR (400 MHZ, DMSO-d₆) δ 9.68 (s, 1H), 8.12 (d, J=9.7 Hz, 1H), 7.63-7.43 (dq, J=19.3, 9.9, 7.4 Hz, 4H), 7.08 (q, J=9.5 Hz, 1H), 6.85 (s, 1H), 6.77 (s, 1H), 4.50 (dt, J=14.3, 7.5 Hz, 1H), 4.03 (d, J=9.0 Hz, 4H), 2.79-2.63 (m, 4H).

Fraction-2:2-fluoro-5-hydroxyphenyl) (6-(5-(2-fluorophenyl)-4-(trifluoromethyl)-1H-pyrazol-1-yl)-2-azaspiro[3.3]heptan-2-yl) methanone (PSY-05-00472-001) (0.031 g 4.35%). LCMS: m/z 464.41 [M+H]⁺. ¹H NMR (400 MHZ, DMSO-d6) δ 9.69 (d, J=5.5 Hz, 1H), 8.62 (d, J=9.6 Hz, 1H), 7.53 (s, 1H), 7.51-7.42 (m, 1H), 7.39-7.25 (m, 2H), 7.10 (q, J=9.0 Hz, 1H), 6.89-6.84 (m, 1H), 6.81 (s, 1H), 4.90 (dt, J=23.7, 7.9 Hz, 1H), 4.16 (d, J=10.3 Hz, 2H), 4.06 (d, J=8.1 Hz, 2H), 2.74 (dd, J=24.9, 10.1 Hz, 4H).

### Example 17: Synthesis of (6-(5-(2,5-Difluorophenyl)-4-methyl-1H-pyrazol-1-yl)-2-azaspiro[3.3] heptan-2-yl) (2-fluoro-5-hydroxyphenyl) methanone [Compound 476] and (6-13-(2,5-Difluorophenyl)-4-methyl-1H-pyrazol-1-yl)-2-azaspiro[3.3]heptan-2-yl) (2-fluoro-5-hydroxyphenyl) methanone [Compound 477]

### Synthetic Scheme:

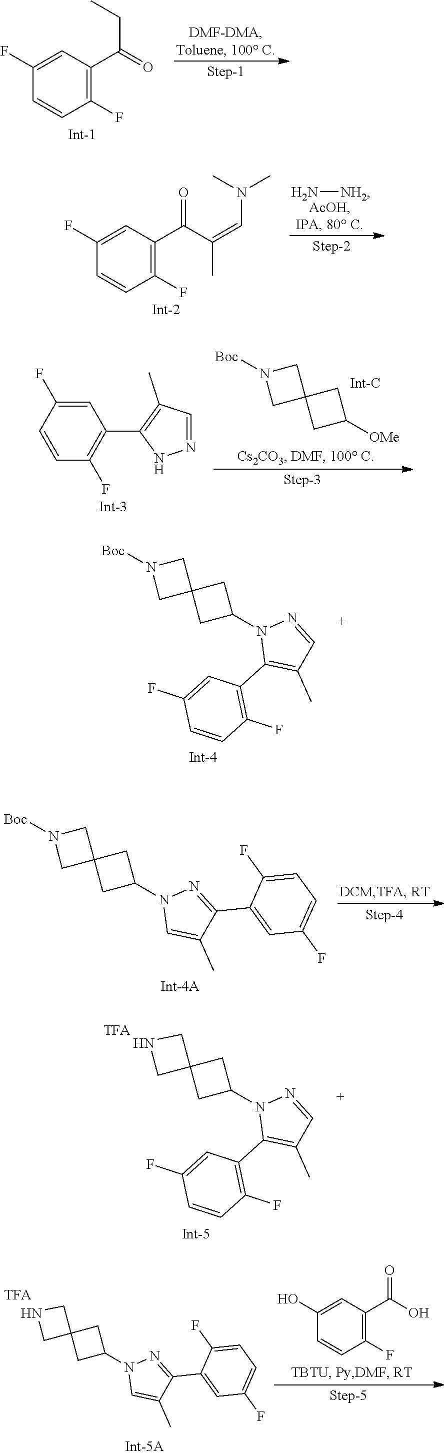

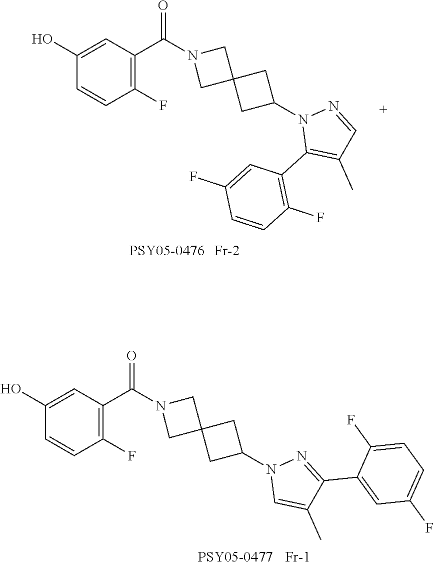

### Step-1: Synthesis of (Z)-1-(2, 5-Difluorophenyl)-3-(dimethyl amino)-2-methylprop-2-en-1-one (Int-2)

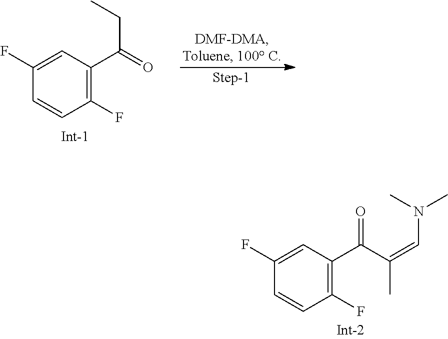 [SMILES: CCC(=O)c1cc(F)ccc1F ; C/C(=C/N(C)C)C(=O)c1cc(F)ccc1F]

To a stirred solution of 1-(2,5-Difluorophenyl) propan-1-one (1.0 g, 5.8768 mmol, 1.0 eq.) in Toluene (10 mL) was added N,N-Dimethylformamide dimethylacetal (3.0 mL, 29.384 mmol, 5.0 eq.) the reaction mass was heated at 100° C. for 16 h. After completion of reaction as monitored by TLC and LCMS. After completion, reaction mixture was poured in ice cold water (50 mL) and extracted with ethyl acetate (20 mL*3). The organic layer was washed with brine (50 mL), dried over Sodium sulphate and concentrated under reduce pressure to get residue. The residue was purified by combi flash using 30-40% ethyl acetate in hexane as eluent to (Z)-1-(2, 5-Difluorophenyl)-3-(dimethyl amino)-2-methylprop-2-en-1-one (Int-2) (1.1 g, 84.61%).

### Step-2: Synthesis of 5-(2,5-Difluorophenyl)-4-methyl-1H-pyrazole (Int-3)

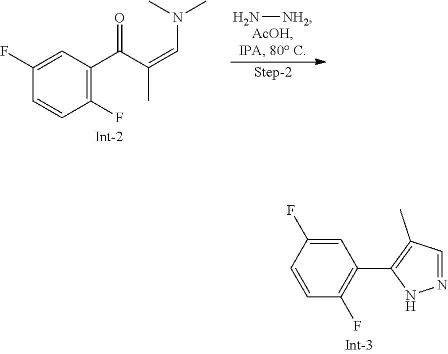

To a stirred solution of (Z)-1-(2,5-Difluorophenyl)-3-(dimethylamino)-2-methylprop-2-en-1-one (1.1 g, 4.88 mmol, 1.0 eq.) in Isopropyl alcohol (20 mL) was added hydrazine hydrate (0.36 mL, 7.33 mmol, 1.5 eq.) the reaction mass was heated at 80° C. for 16 h. After completion of reaction as monitored by TLC and LCMS, the reaction mixture was concentrated to get residue. The residue was purified by combi flash using 20-30% ethyl acetate in hexane as eluent to get 5-(2,5-Difluorophenyl)-4-methyl-1H-pyrazole (Int-3) (0.80 g, 84.38%).

Step-3: Synthesis of Tert-butyl 6-(5-(pyridin-3-yl)-3-(trifluoromethyl)-1H-pyrazol-1-yl)-2-azaspiro[3.3]heptane-2-carboxylate and Tert-butyl 6-(3-(pyridin-3-yl)-5-(trifluoromethyl)-1H-pyrazol-1-yl)-2-azaspiro[3.3]heptane-2-carboxylate (mix of Int-4 and Int-4A)

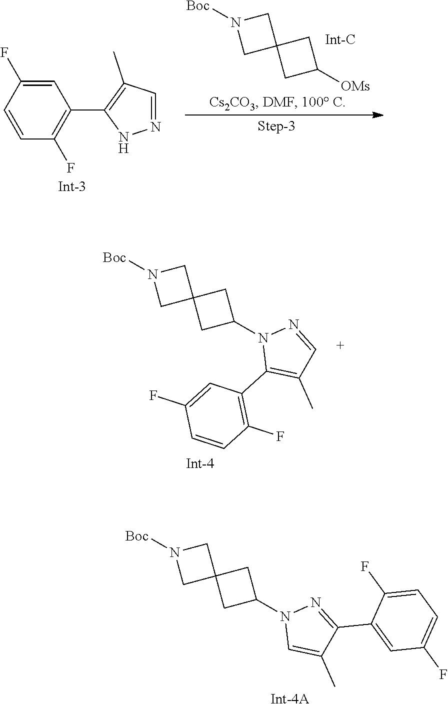 [SMILES: Cc1cn[nH]c1-c1cc(F)ccc1F ; CC(C)(C)OC(=O)N1CC2(CC(OS(C)(=O)=O)C2)C1 ; Cc1cnn(C2CC3(C2)CN(C(=O)OC(C)(C)C)C3)c1-c1cc(F)ccc1F ; Cc1cn(C2CC3(C2)CN(C(=O)OC(C)(C)C)C3)nc1-c1cc(F)ccc1F]

To a well stirred reaction mixture of 3-(3-(Trifluoromethyl)-1H-pyrazol-5-yl) pyridine (0.800 g, 4.12 mmol, 1.0 eq.), CS₂CO₃ (2.01 g, 6.18 mmol, 1.5 eq.) in N,N-di methyl formamide (10 mL) was added Tert-butyl 6-((methyl sulfonyl)oxy)-2-azaspiro[3.3]heptane-2-carboxylate (1.44 g, 4.94 mmol, 1.2 eq.). The reaction was heated at 100° C. for 12 h. After completion of reaction as monitored by TLC, the reaction mixture was poured in water (100 mL) and extracted with ethyl acetate (50 mL*3). The organic layer was washed with brine (100 mL), dried over Sodium sulphate and concentrated under reduce pressure to get residue. The residue was purified by combi flash using 30-50% ethyl acetate in hexane as eluent to get to mixture of Int-4 and Int-4A (1.0 g, 62.5%).

### Step-4: Synthesis of 6-(5-(2,5-difluorophenyl)-4-methyl-1H-pyrazol-1-yl)-2-azaspiro[3.3]heptane TFA salt (Int-5) and 6-(3-(2,5-difluorophenyl)-4-methyl-1H-pyrazol-1-yl)-2-azaspiro[3.3]heptane TFA salt (Int-5A)

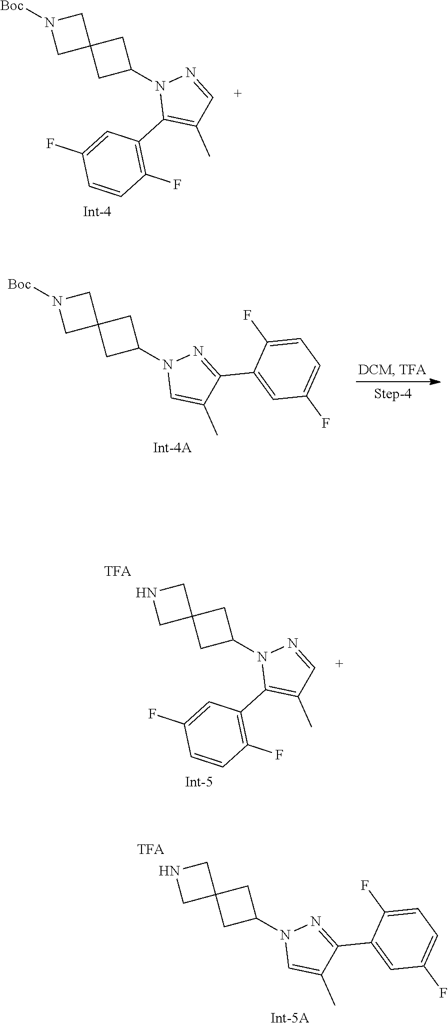 [SMILES: Cc1cnn(C2CC3(C2)CN(C(=O)OC(C)(C)C)C3)c1-c1cc(F)ccc1F ; Cc1cn(C2CC3(C2)CN(C(=O)OC(C)(C)C)C3)nc1-c1cc(F)ccc1F ; Cc1cnn(C2CC3(CNC3)C2)c1-c1cc(F)ccc1F ; "TFA" ; Cc1cn(C2CC3(CNC3)C2)nc1-c1cc(F)ccc1F]

To a well stirred reaction mixture of Int-4 and Int-4A (1.0 g, 2.5706 mmol, 1.0 eq.) in dichloromethane (10 mL) was added TFA (2.0 mL) dropwise at 0° C. After completion of reaction as monitored by TLC and LCMS, the reaction mixture was concentrated to get TFA salt of Int-5 and Int-5A (1.1 g).

### Step-5: Synthesis of (6-(5-(2,5-Difluorophenyl)-4-methyl-1H-pyrazol-1-yl)-2-azaspiro [3.3] heptan-2-yl) (2-fluoro-5-hydroxyphenyl) methanone and (6-(3-(2,5-Difluorophenyl)-4-methyl-1H-pyrazol-1-yl)-2-azaspiro[3.3]heptan-2-yl) (2-fluoro-5-hydroxyphenyl) methanone (PSY-05-00476-001 and PSY-05-00477-001)

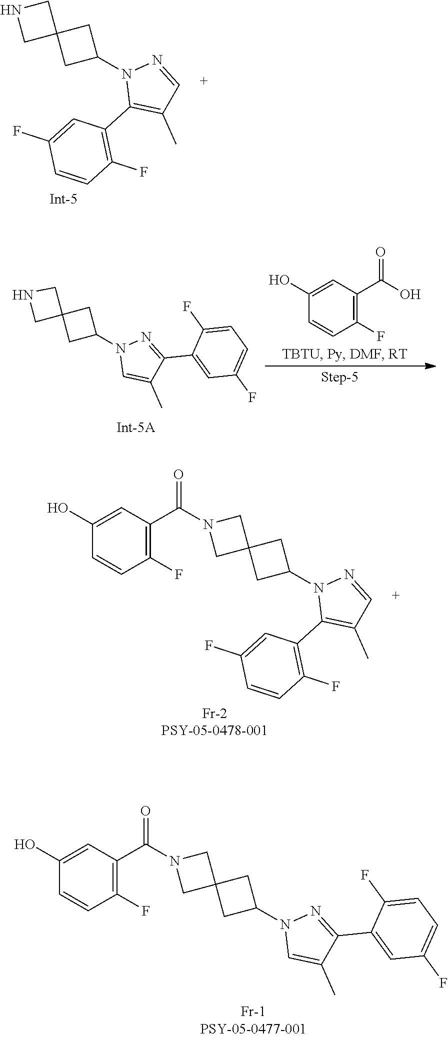 [SMILES: Cc1cnn(C2CC3(CNC3)C2)c1-c1cc(F)ccc1F ; Cc1cn(C2CC3(CNC3)C2)nc1-c1cc(F)ccc1F ; O=C(O)c1cc(O)ccc1F ; Cc1cnn(C2CC3(C2)CN(C(=O)c2cc(O)ccc2F)C3)c1-c1cc(F)ccc1F ; Cc1cn(C2CC3(C2)CN(C(=O)c2cc(O)ccc2F)C3)nc1-c1cc(F)ccc1F]

To a well stirred reaction mixture of Int-5 and Int-5A (1.13 g, 2.9230 mmol, 1.2 eq.), Pyridine (0.98 mL, 12.1794 mmol, 5.0 eq.), 2-Fluoro-5-hydroxybenzoic acid (0.38 g, 2.4358 mmol, 1.0 eq.) in N,N-dimethyl formamide (5 mL) was added TBTU (0.321 g, 3.6538 mmol, 1.5 eq.) at 0° C. The reaction mass was stirred at room temperature for 3-4 hr. After completion of reaction as monitored by TLC and LCMS, the reaction mixture was poured in water (100 mL) and extracted with ethyl acetate (50 mL*3). The organic layer was washed with saturated solution of sodium carbonate (50 mL) and brine (100 mL), dried over sodium sulphate (Na₂SO₄) and concentrated under reduce pressure to get residue. The residue was purified by combi flash using 40-50% ethyl acetate in hexane as eluent. The crude was submitted to prep-HPLC for purification. (PSY-05-00476-001-FR-2:0.308 g, 20.95%) (PSY-05-00477-001-FR-1:0.096 g, 6.53%).

Fraction-1: (6-(3-(2,5-Difluorophenyl)-4-methyl-1H-pyrazol-1-yl)-2-azaspiro [3.3] heptan-2-yl) (2-fluoro-5-hydroxyphenyl) methanone (PSY-05-0477-001 Fr-1). LCMS: m/z 428.03 [M+1]⁺. ¹H NMR (400 MHZ, DMSO-d6) δ 7.52-7.40 (m, 3H), 7.30 (dd, J=5.4, 2.8 Hz, 1H), 7.08 (q, J=8.9 Hz, 1H), 6.85 (d, J=3.9 Hz, 1H), 6.81-6.75 (m, 1H), 4.47 (dt, J=15.6, 7.8 Hz, 1H), 4.07 (dd, J=24, 5, 5.0 Hz, 4H), 2.60 (dd, J=20.2, 8.3 Hz, 4H), 1.90 (s, 3H).

Fraction-2: (6-(5-(2,5-Difluorophenyl)-4-methyl-1H-pyrazol-1-yl)-2-azaspiro [3.3] heptan-2-yl) (2-fluoro-S-hydroxyphenyl) methanone (PSY-05-00476-001 Fr-2). LCMS: m/z 428.3 [M+1]⁺. ¹H NMR (400 MHZ, DMSO-d6) δ 9.69 (s, 1H), 7.72 (d, J=5.9 Hz, 1H), 7.39-7.26 (m, 3H), 7.10 (q, J=9.1 Hz, 1H), 6.82-6.87 (q, J=9.1 Hz, 1H), 6.74-6.79 (q, J=9.1 Hz, 1H), 4.76 (dt, J=23.3, 7.9 Hz, 1H), 4.15 (d, J=11.2 Hz, 2H), 4.05 (d, J=8.7 Hz, 2H), 2.76-2.60 (m, 4H), 1.99 (s, 3H).

### Example 18: Measuring MAGL Inhibition Potency (IC₅₀)

The potency of certain compounds for inhibiting MAGL were obtained using the following assays.

The monoacylglycerol lipase inhibitor screening assay kit from Cayman Chemical was used to measure the MAGL potency for the compounds in Table 3 and Table 4.

Cayman's Monoacylglycerol Lipase Inhibitor Screening Assay provides a method for screening human MAGL inhibitors. MAGL hydrolyzes 4-nitrophenylacetate resulting in a yellow product, 4-nitrophenol, with an absorbance of 405-412 nm.

MAGL Inhibition was measured by the following assay. Monoacylglycerol Lipase (MAGL) inhibition was measured using recombinant MAGL enzyme (aa 2-303 RBC, internal preparation) and the substrate 4-Nitrophenyl acetate (4NPA) (Sigma-Aldrich, N8130). Hydrolysis of the substrate in the presence of the enzyme was measured by absorbance at 405 nm. 10 μL of assay buffer (10 mM Tris pH 7.5, 1 mM EDTA, 0.9% DMSO) was added to a black 384-well non-binding plate with clear bottom (Greiner, 781906) for each reaction. Compounds were dispensed using an acoustic liquid handler (Echo, Beckman) at 45 nL (0.1% DMSO). Test compounds and control for MAGL inhibitor JZL-184 (Caymen Chemical, 13158) were tested in 10-concentration IC50 mode with 3-fold serial dilution at a starting concentration of 10 μM. DMSO control wells were included for reference. A 10.8 nM (1.8×) MAGL mix in assay buffer was prepared, with 25 L added to each reaction well, for a final assay concentration of 6 nM. No enzyme wells received 25 μL of buffer. Plate was incubated at room temperature for 30 minutes. 35 mM solution of 4NPA in methanol was prepared daily. A 4.5×4NPA substrate solution was prepared in assay buffer and 10 μL was added to each reaction well, for a final assay concentration of 0.25 mM. Plate was spun for 1 minute at 1000 rpm before measuring absorbance using a CLARIOstar plate reader (BMG Labtech). A kinetic reading at 405 nm was done every minute for 30 minutes. Data was analyzed using the linear slope of the reaction progress curve and the average of the no-enzyme wells (background) was subtracted from the data. The background-subtracted slope data was converted to % activity using the average of wells with enzyme and DMSO vehicle. IC50s were calculated using GraphPad software (Sigmoidal dose response, variable slope equation).

Table 3 and Table 4 provide exemplary compounds of Formula (I) and their potency for MAGL inhibition measured using the Potency Assay of Example 18 above, with the following modifications described in Table C below. Table 5 provides potency measurements of MAGL inhibition measured using the Potency Assay of Example 18 above, with the following modifications described in Table C.

TABLE C
Methods Used to Measure MAGL Inhibition with 4NPA Substrate

| Method | | |
|---|---|---|
| No. | Name | Modification to Assay of Example 16 |
| 1 | MAGL (6 nM) Reaction | Reaction Biology's internal MAGL enzyme prep was used. |
| | Biology Enzyme | Concentration of MAGL enzyme in the assay was 6 nM. |
| 2 | MAGL (10 nM) Cayman | Cayman Chemical's MAGL enzyme prep was used. |
| | Chemical Enzyme | Concentration of MAGL enzyme in the assay was 10 nM. |
| 3 | MAGL (10 nM) Reaction | Reaction Biology's internal MAGL enzyme prep was used. |
| | Biology Enzyme | Concentration of MAGL enzyme in the assay was 10 nM. |
| 4 | MAGL (15 nM) Cayman | Cayman Chemical's MAGL enzyme prep was used. |
| | Chemical Enzyme | Concentration of MAGL enzyme in the assay was 15 nM. |
| 5 | MAGL (25 nM) Cayman | Cayman Chemical's MAGL enzyme prep was used. |
| | Chemical Enzyme | Concentration of MAGL enzyme in the assay was 25 nM. |
| 6 | MAGL IC50 Enzyme | Reaction Biology's internal MAGL enzyme prep was used. |
| | Conc 6, Reaction Biology | Concentration of MAGL enzyme in the assay was 6 nM. |
| 7 | MAGL IC50 Enzyme | Reaction Biology's internal MAGL enzyme prep was used. |
| | Conc 10, Reaction | Concentration of MAGL enzyme in the assay was 10 nM. |
| | Biology | |
| 8 | MAGL IC50 Enzyme | Cayman Chemical's MAGL enzyme prep was used. |
| | Conc 10, Cayman | Concentration of MAGL enzyme in the assay was 10 nM. |
| | Chemical | |
| 9 | MAGL IC50 Enzyme | Cayman Chemical's MAGL enzyme prep was used. |
| | Conc 15, Cayman | Concentration of MAGL enzyme in the assay was 15 nM. |
| | Chemical | |
| 10 | MAGL IC50 Enzyme | Cayman Chemical's MAGL enzyme prep was used. |
| | Conc 25, Cayman | Concentration of MAGL enzyme in the assay was 25 nM. |
| | Chemical | |
| 11 | MAGL IC50 Enzyme | Reaction Biology's internal MAGL enzyme prep was used. |
| | Conc 20, Reaction | Concentration of MAGL enzyme in the assay was 25 nM. |
| | Biology | |
| 12 | MAGL IC50 Enzyme | Reaction Biology's internal MAGL enzyme prep was used. |
| | Conc 15, Reaction | Concentration of MAGL enzyme in the assay was 15 nM. |
| | Biology | |
| 13 | MAGL IC50 Enzyme | Reaction Biology's internal MAGL enzyme prep was used. |
| | Conc 12, Reaction | Concentration of MAGL enzyme in the assay was 12 nM. |
| | Biology | |
| 14 | MAGL IC50 Enzyme | Reaction Biology's internal MAGL enzyme prep was used. |
| | Conc “0”, Source “0” | Concentration of MAGL enzyme in the assay was 6 nM. |

Results of potency measurements in Table 3, Table 4 and Table 5 are expressed as the following ranges: “A” refers to an IC₅₀ measurement of <50 nM, “B” refers to an IC₅₀ measurement of between 50 nM and 150 nM, “C” refers to an IC₅₀ measurement of greater than 150 nM and less than 500 nM, “D” refers to an IC₅₀ measurement of 500 nM to 1 micromolar, and “E” refers to an IC₅₀ measurement of greater than 1 micromolar up to 5.1 micromolar. The number after each letter for potency measurement value indicates the Method from Table C that was used to obtain that measurement value (e.g., “A(1)” indicates an IC₅₀ measurement of <50 nM obtained from the Method 1 of Example 18 in Table C).

### Example 19: Measuring FAAH Inhibition Potency (IC₅₀)

Comparative compound potency at FAAH can be obtained with the following assay. A “Selective MAGL Inhibitor Compound” refers to a compound that selectively inhibits MAGL with an IC₅₀ that is at least 10× the IC₅₀ for its inhibition of fatty acid amide hydrolase (FAAH), and that has an IC₅₀ of 150 nM or less for MAGL inhibition (according to the MAGL Potency assay of Example 18).

MAGL inhibitor compounds were also counter-screened for FAAH inhibition potency using the following assay. Assessment of FAAH inhibition was performed using Fatty Acid Amide Hydrolase Inhibitor Screening Assay Kit (Cayman Item No. 10005196) following manufacture's instruction with some modifications. The kit utilizes human recombinant FAAH and the fluorescent substrate, AMC Arachidonoyl amide (AAMCA). 5 μL of assay buffer (125 mM Tris, pH 9.0, 1 mM EDTA, i.e. ethylenediaminetetraacetic acid) was added to a 384-well black plate (Corning, 3573). Test compounds and control inhibitor JZL-195 (Cayman Chemical, 13668) were tested in 10-concentration IC50 mode with 3-fold serial dilution at a starting concentration of 100 μM and 10 μM, respectively. 300 nL or 30 nL of test compounds were delivered into a 384-well black plate (Corning, 3573) using a Labcyte Echo, followed by addition of 15 μL of FAAH enzyme (Cayman, 700302) in assay buffer. After a 5-minute pre-incubation at room temperature, 10 μL of AAMCA was added in assay buffer to start the reaction. Final concentration of FAAH enzyme is not specified and AAMCA substrate was used at the 20 μM. After these dilutions, the final concentration of the test compounds ranged from 100 μM to 5.08 nM or 10 μM down to 0.508 nM. The reaction was allowed to progress for 60 minutes, while the plate was read on an Envision plate reader at an Ex/Em of 350/460 nm with readings every minute. The data was analyzed in Microsoft Excel, using the slope between 30 and 59 minutes. The average of the no-enzyme wells (background) was subtracted from the data. The background-subtracted slope data was converted to % activity using the average of wells with enzyme and DMSO vehicle. IC₅₀ values were calculated using GraphPad software (Sigmoidal dose response, variable slope equation).

Compounds listed in Table D2 were tested in the FAAH Counterscreen of Example 19

TABLE D1
Methods Used to Measure FAAH Inhibition

| Method | Modification to Assay of Example 19 |
|---|---|
| A | Starting concentration of 100 μM |
| B | Starting concentration of 10 μM |

TABLE D2
FAAH Counter Screen

| Method | Compound | Structure | FAAH IC₅₀ (nM) |
|---|---|---|---|
| A | Comparator | 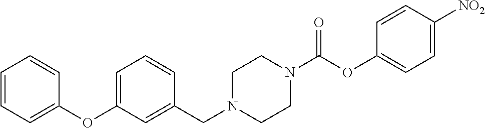 [SMILES: O=C(Oc1ccc([N+](=O)[O-])cc1)N1CCN(Cc2cccc(Oc3ccccc3)c2)CC1] | 459 |
| A | Comparator | 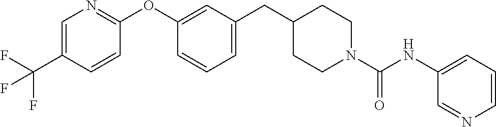 [SMILES: O=C(Nc1cccnc1)N1CCC(Cc2cccc(Oc3ccc(C(F)(F)F)cn3)c2)CC1] | 14 |
| A | 117 | 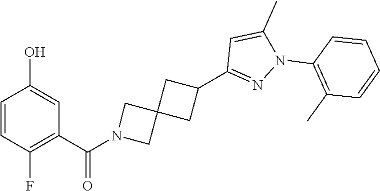 [SMILES: Cc1ccccc1-n1nc(C2CC3(C2)CN(C(=O)c2cc(O)ccc2F)C3)cc1C] | >10,000 |
| A | 120 | 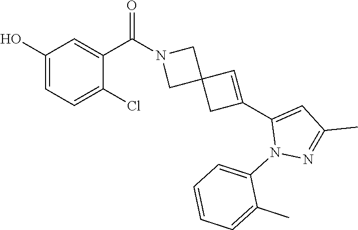 [SMILES: Cc1cc(C2=CC3(C2)CN(C(=O)c2cc(O)ccc2Cl)C3)n(-c2ccccc2C)n1] | >10,000 |
| A | 122 | 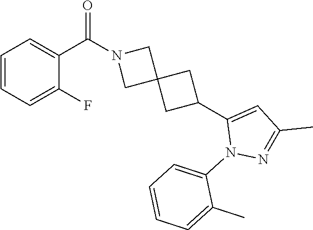 [SMILES: Cc1cc(C2CC3(C2)CN(C(=O)c2ccccc2F)C3)n(-c2ccccc2C)n1] | >10,000 |
| A | 123 | 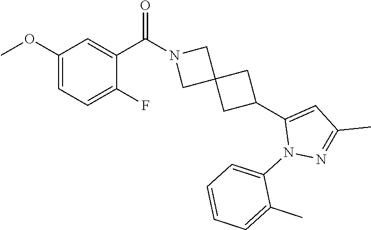 | >10,000 |
| A | 125 | 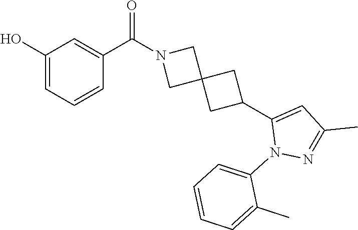 [SMILES: Cc1cc(C2CC3(C2)CN(C(=O)c2cccc(O)c2)C3)n(-c2ccccc2C)n1] | >10,000 |
| A | 127 | 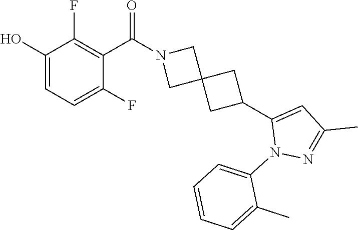 [SMILES: Cc1cc(C2CC3(C2)CN(C(=O)c2c(F)ccc(O)c2F)C3)n(-c2ccccc2C)n1] | >10,000 |
| B | 473 | 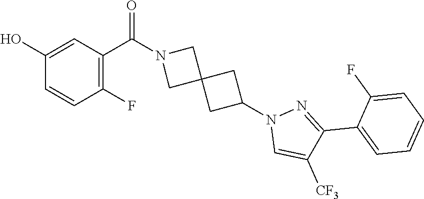 [SMILES: O=C(c1cc(O)ccc1F)N1CC2(CC(n3cc(C(F)(F)F)c(-c4ccccc4F)n3)C2)C1] | >10,000 |
| B | 493 | 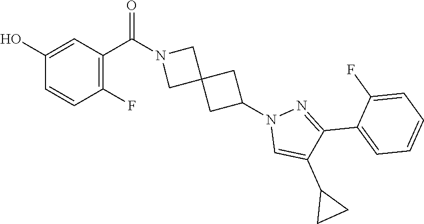 [SMILES: O=C(c1cc(O)ccc1F)N1CC2(CC(n3cc(C4CC4)c(-c4ccccc4F)n3)C2)C1] | >10,000 |

### Example 24: Measuring Reversible MAGL Inhibition (IC₅₀)

The reversible mechanism of MAGL inhibition of a test compound of Formula (I) can be determined. Flag-tagged MAGL enzyme will be immobilized on anti-Flag beads. Immobilized enzyme will be incubated+/−inhibitor at a dose that produces complete inhibition. Colorimetric substrate (4-NPA) will be added and the reaction monitored on a plate reader for 30 minutes to verify complete inhibition. Immobilized enzyme will then be washed thoroughly to remove the inhibitor, and fresh substrate will be added. The reaction will be monitored for an additional 30 minutes; returning enzymatic activity will indicate reversibility of inhibition.

To confirm the hypothesized reversible mechanism of inhibition, the effects of dilution and preincubation on the MAGL inhibitory activity of a compound can be evaluated using methods disclosed in J. Med. Chem. 2019, 62, 1932-1958, 1942. In the presence of an irreversible mechanism of inhibition, the potency should not decrease after dilution, whereas for a reversible inhibition, the potency level should be strongly reduced after dilution. Therefore, the inhibition produced by incubation with a 4000 nM concentration of a test compound can be measured after a 40× dilution and compared to the potency observed by a 4000 and a 100 nM of the test compound. A reversible mechanism of inhibition can be identified when the inhibition produced by 100 nM of the test compound is similar to that obtained after a 40× dilution and was considerably lower than that produced by the same compound at a concentration of 4000 nM.

As a second assay to identify or confirm a reversible MAGL inhibitory activity of a test compound, the inhibition activity of a test compound can be measured at different preincubation times with MAGL. The test compound can be preincubated with the enzyme for 0, 30, and 60 min before adding the substrate to start the enzymatic reaction. An irreversible inhibition should produce a higher potency after longer incubation times, whereas a reversible inhibitor should produce a constant inhibition potency over all the different incubation times.

### Determining MAGL Reversible Inhibition:

MAGL enzyme was incubated for 30 minutes in the presence of 40× the IC50 concentration of inhibitor. Enzyme+inhibitor mix was then diluted 40-fold so that the final concentration of the inhibitor equaled the IC₅₀ concentration. Substrate was added and the reaction was monitored for 30 minutes. For a reversible inhibitor, percent inhibition after dilution to the IC₅₀ concentration should be 50+15%.

The MAGL reversible inhibition assay of Example 24 above was performed to test reversibility of inhibition by compounds depicted in Table E. Column A shows the degree to which MAGL enzymatic activity returned following washout of test compounds (pre-washout compound concentration: 1 μM), reflecting reversibility of inhibition, as compared to the complete lack of return of MAGL enzymatic activity after washout of the irreversible inhibitor comparator compound JZL-184 (pre-washout compound concentration: 1 μM). Column B shows % MAGL activity returning after washout. % Activity returning after washout represents the amount of MAGL enzymatic activity, relative to control (no inhibitor before washout) reaction amounts, that occurred immediately following a 30 minute washout of the test compound. For some test compounds, % activity returning after washout may be less than 100% if complete compound unbinding takes longer than 30 minutes.

TABLE E
Reversible MAGL inhibition

| Compound | Structure | A | B |
|---|---|---|---|
| JZL-184 (Comparator) | 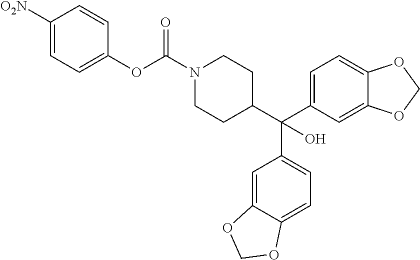 [SMILES: O=C(Oc1ccc([N+](=O)[O-])cc1)N1CCC(C(O)(c2ccc3c(c2)OCO3)c2ccc3c(c2)OCO3)CC1] | | <1% |
| 074 | 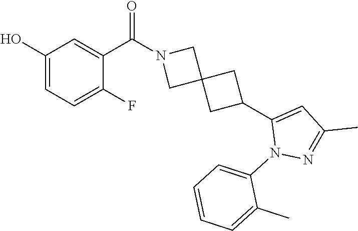 [SMILES: Cc1cc(C2CC3(C2)CN(C(=O)c2cc(O)ccc2F)C3)n(-c2ccccc2C)n1] | 162 | >50% |
| 473 | 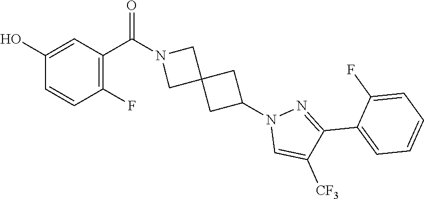 [SMILES: O=C(c1cc(O)ccc1F)N1CC2(CC(n3cc(C(F)(F)F)c(-c4ccccc4F)n3)C2)C1] | 41 | <50% |
| 493 | 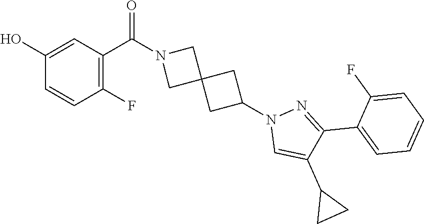 [SMILES: O=C(c1cc(O)ccc1F)N1CC2(CC(n3cc(C4CC4)c(-c4ccccc4F)n3)C2)C1] | 47 | <50% |
| 519 | 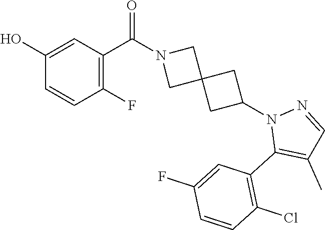 [SMILES: Cc1cnn(C2CC3(C2)CN(C(=O)c2cc(O)ccc2F)C3)c1-c1cc(F)ccc1Cl] | 155 | >50% |
| 520 | 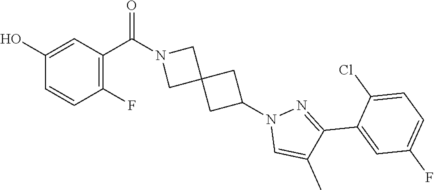 [SMILES: Cc1cn(C2CC3(C2)CN(C(=O)c2cc(O)ccc2F)C3)nc1-c1cc(F)ccc1Cl] | 100 | >50% |

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims.

### Example 18: Pharmacokinetics and Estimation of 2-AG in Male C₅₇BL/6 Mice Brain Following a Single Intraperitoneal Administration of a Reversible MAGL Inhibitor Compound [Compound 74]

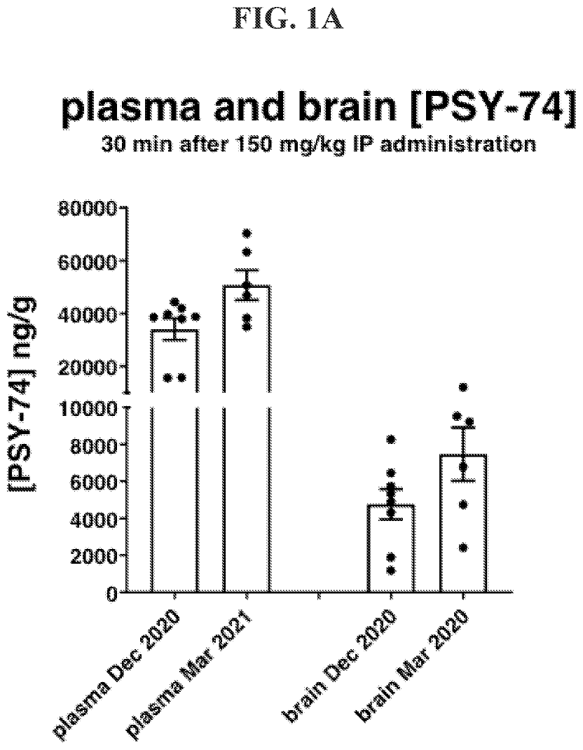

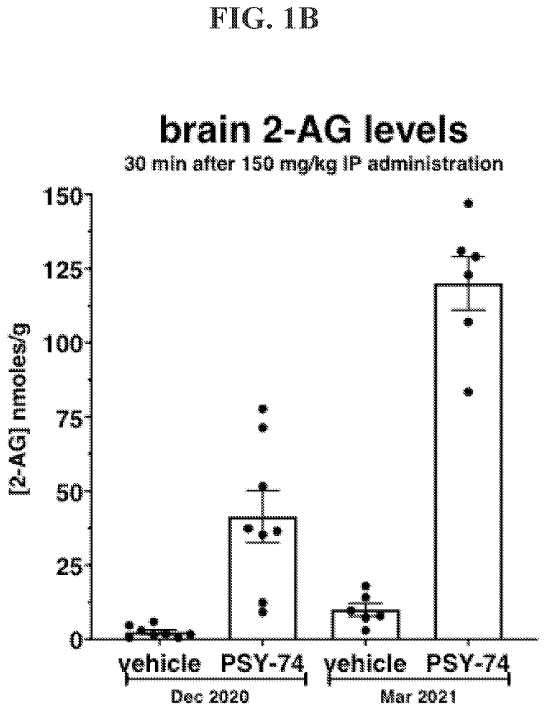

This experiment was performed to determine plasma pharmacokinetics and estimate 2-AG levels in male C57BL/6 mice brain following a single intraperitoneal administration of Compound-74 (Dose: 5, 15, 50 and 150 mg/kg). The experiment was repeated at 150 mg/kg on 3 separate test dates to obtain the data shown in FIG. 1A (plasma and brain concentration of Compound 74 30 minutes after IP injection), and on the first and third test dates to obtain the data shown in FIG. 1B (brain concentration of 2-AG30 minutes after IP injection of Compound 74).

Compound 74, Molecular Wt.: 405.46 (Freebase), % Purity: Considered 100%.

Formulation: DMSO

Dose: 5, 15, 50 and 150 mg/kg

Dose volume: 2 mL/kg

Feeding regimen: freely fed

Groups:

| Group | Dose (mg/kg) |
|---|---|
| Group 1 | 5 |
| Group 2 | 15 |
| Group 3 | 50 |
| Group 4 | 150 |
| Group 5 | Vehicle |

Study Design: A total of thirty male mice were used into the study and divided in to five groups with six mice in each group (n=6 mice/group/treatment design). Animals in Group 1 to Group 4 were administered intraperitoneally with Compound 74 solution formulation at 5, 15, 50 and 150 mg/kg dose, respectively. Animals in Group 5 were administered intraperitoneally with vehicle at 2 mL/kg dose. Blood and brain samples were collected at 0.5 h post dose. Mice were euthanized by cervical dislocation and brain samples were dissected rapidly and snap frozen in liquid nitrogen, weighed and homogenized in 2 mL of acetonitrile containing 2-AGd5. Blood samples were centrifuged at 4000 rpm for 10 minutes at 4° C. Plasma was harvested and separated in pre-labeled tubes. All samples were stored below −70° C. until bioanalysis.

Analysis: Brain samples were processed for 2-AG estimation. 2-AG levels were the detected values. Plasma concentrations were determined by fit for purpose LC-MS/MS method. LLOQ: 5.09 ng/mL (Plasma and brain)

Results: Following a single intraperitoneal administration of Compound 74 in male C57BL/6 mice, levels of 2-AG were elevated slightly at 5 and 15 mg/kg dose, while, significant increase was observed at 50 and 150 mg/kg dose groups compared to baseline (vehicle treatment). At 0.5 h, Brain-Kp were ranged 0.51-0.68 (5 mg/kg), 0.16-0.44 (15 mg/kg), 0.06-0.08 (50 mg/kg) and 0.07-0.20 (150 mg/kg).

TABLE 18A
Individual 2-AG concentration-time data following a single
intraperitoneal administration of PSY-05-00074-003 in
male C57BL/6 mice (Dose: 5, 15, 50 and 150 mg/kg)

| | | Mean 2AG Brain | |
|---|---|---|---|
| | | Concentration | |
| Time (h) | Group/Route/Dose | (nmole/g) | SD |
| 0.5 | Group 1/IP/5 mg/kg | 13.13 | 5.47 |
| | Group 2/IP/15 mg/kg | 13.46 | 7.57 |
| | Group 3/IP/50 mg/kg | 42.35 | 17.82 |
| | Group 4/IP/150 mg/kg | 120.02 | 22.1 |
| | Group 5/IP/Vehicle | 10.01 | 5.34 |

TABLE 18B
Individual plasma concentration-time data following a single
intraperitoneal administration of PSY-05-00074-003 in
male C57BL/6 mice (Dose: 5, 15, 50 and 150 mg/kg)

| | | Plasma | Brain | Brain Plama |
|---|---|---|---|---|
| | | concentration | concentration | Ratio (Kp) |
| Time (h) | Group/Route/Dose | range (ng/mL) | range (ng/g) | range |
| 0.5 | Group 1/IP/5 mg/kg | 12-186 | 16-19 | 0.51-0.68 |
| | Group 2/IP/15 mg/kg | 70-414 | 25-67 | 0.16-0.44 |
| | Group 3/IP/50 mg/kg | 447-14914 | 34-1250 | 0.06-0.08 |
| | Group 4/IP/150 mg/kg | 34920-70256 | 2412-12111 | 0.07-0.20 |

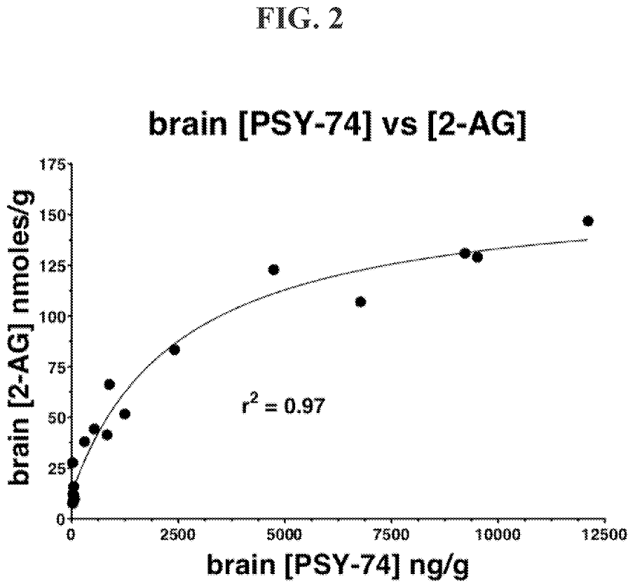

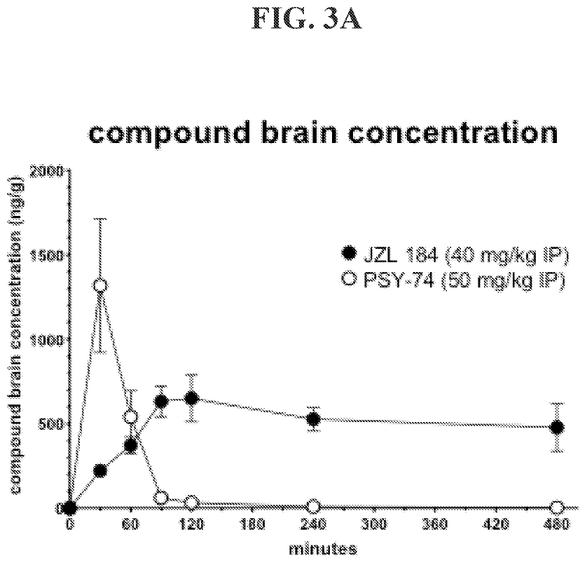

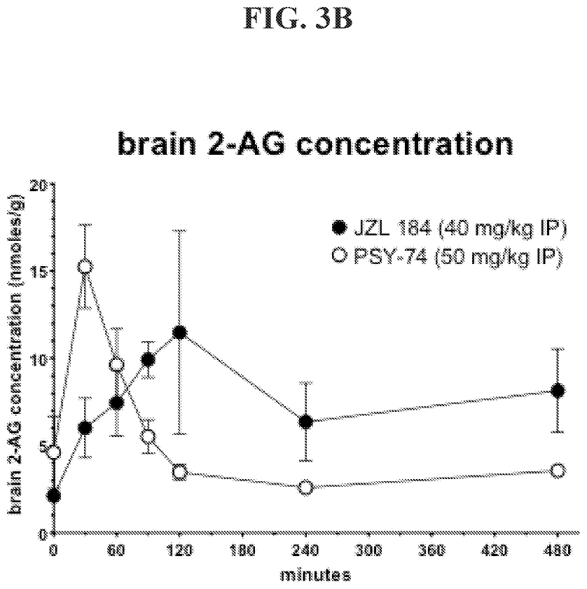

FIG. 1A is a bar graph showing both the plasma and brain concentrations of Compound 74 30 minutes after IP administration of 150 mg/kg of Compound 74 to the mouse model described above. FIG. 1B is a bar graph showing 2-AG measurement in the brain of the mouse model described above observed 30 minutes after the IP administration of 150 mg/kg of Compound 74. FIG. 2 is a scatter plot of brain 2-AG levels vs brain concentration of Compound 74.

### Example 19: Pharmacokinetics and Estimation of 2-AG in Male C57BL/6 Mice Brain Following a Single Intraperitoneal Administration of a Reversible MAGL Inhibitor Compound [Compound 473]

This experiment was performed to determine plasma pharmacokinetics and estimate 2-AG levels in male C₅₇BL/6 mice brain following a single intraperitoneal administration of Compound-473 (Dose: 20 mg/kg).

Compound 473, Molecular Wt.: 463.41 (Freebase), % Purity: >98%.

Formulation: DMSO

Dose: 20 mg/kg

Dose volume: 10 mL/kg

Feeding regimen: Food and water ad libitum

Groups:

| Group | Dose (mg/kg) |
|---|---|
| Group1 | 20 mg/kg |
| Group 2 | Vehicle |

Study Design: A total of twelve C₅₇BL/6 male mice were used in this study. Six animals were administered intraperitoneally with solution formulation of PSY-05-00473-002 at 20 mg/kg dose. Six animals were administered vehicle (5% NMP, 5% Solutol HS15 and 90% Normal saline) intraperitoneally at 10 mL/kg dose volume. Blood samples (approximately 60 μL) were collected under light isoflurane anesthesia from a set of six mice at 0.5 h. Plasma was harvested by centrifugation of blood and stored at −70±10° C. until analysis. After blood collection, mice were euthanized by cervical dislocation and brain samples were dissected rapidly and snapped frozen in liquid nitrogen and stored at −80. To homogenize, removed brain samples from the freezer, were divided into two equal hemispheres and weighed while still in frozen condition, and begun homogenization/extraction procedure without allowing the brain samples to thaw. One part is used for PK estimation and the other part is used for 2-AG estimation.

Analysis: For PK estimation, brain samples were homogenized in 2 volumes of PBS of brain weight. Total homogenate volume was three times of the brain weight. For 2-AG estimation, the frozen brain samples were homogenized in 2 mL acetonitrile containing 2-AGd5 (8.88 nmoles) will be incubated overnight at −20° C. to precipitate proteins. Blood samples were centrifuged at 10000 rpm for 10 minutes at 4° C. All samples were stored below-70° C. until bioanalysis. Plasma and brain homogenate samples were quantified by fit-for-purpose LC-MS/MS method (LLOQ: 1.02 ng/mL for plasma and 2.03 ng/ml for brain).

Results: Following single intraperitoneal dose administration of PSY-05-00473-002 at 20 mg/kg to male C₅₇BL/6 mice, plasma and brain concentrations were quantifiable at 0.5 h. Brain kp was found to be 1.44 following 20 mg/kg intraperitoneal dose administration. Following single intraperitoneal dose administration of PSY-05-00473-002 at 20 mg/kg to male C57BL/6 mice, 2-AG concentrations were found to be 17.38 nmole/gram at 0.5 h. Following only vehicle (5% NMP, 5% Solutol HS15 and 90% Normal saline) administration intraperitoneally at 10 mL/kg dose volume, plasma concentrations were not detected in vehicle group whereas mean 2-AG concentrations in brain were found to be 4.17 nmole/gram at 0.5 h.

TABLE 19A
Individual 2-AG concentration-time data following
a single intraperitoneal administration of PSY-05-
000473-001 in male C57BL/6 mice (Dose: 20 mg/kg)

| | | | Mean 2AG | | |
|---|---|---|---|---|---|
| | | | Concentration | | |
| | | | in Brain | | |
| Route | Dose (mg/kg) | Time (h) | (nmole/g) | SD | % CV |
| IP | 20 mg/kg | 0.5 | 17.38 | 9.19 | 53 |
| IP | Vehicle | 0.5 | 4.17 | 1.61 | 39 |

TABLE 19B
Individual plasma and brain concentration-time data following
a single intraperitoneal administration of PSY-05-00473-
001 in male C57BL/6 mice (Dose: 20 mg/kg)

| | | | Plasma | Brain | |
|---|---|---|---|---|---|
| | Time | | Concentration | Concentration | Brain/Plasma |
| Route | (h) | | (ng/mL) | (ng/g) | Ratio (kp) |
| IP | 0.5 | Mean | 1815 | 2633 | 1.44 |
| | | SD | 329.29 | 582.53 | .12 |
| | | % CV | 18 | 22 | 8 |

### Example 20: Pharmacokinetic Study of Compounds in Mice

Healthy male C57BL/6 mice (8-12 weeks old) weighing between 17 to 35 g were procured from Global, India. Temperature and humidity were maintained at 22±3° C. and 30-70%, respectively and illumination was controlled to give a sequence of 12 hr light and 12 hr dark cycle. Temperature and humidity were recorded by auto-controlled data logger system. All the animals were provided laboratory rodent diet. Reverse osmosis water treated with ultraviolet light was provided ad libitum.

Protocol A: Twenty-four male mice were divided into two groups as; Group 1:30 mg/kg/IP, Plasma and brain; Number of animals=12; Animal #1-12. Group 2:30 mg/kg/PO; Plasma and brain; Number of animals=12; Animal #13-24. Animals in Group 1 were administered intraperitoneally with solution formulation of PSY-05-00074-001 in 5% NMP, 5% Solutol HS-15 and 90% normal saline at 30 mg/kg dose. Animals in Group 2 were administered orally with solution formulation of PSY-05-00074-001 in 5% NMP, 5% Solutol HS-15 and 90% normal saline at 30 mg/kg dose. Blood samples (approximately 60 μL) were collected under light isoflurane anesthesia from retro orbital plexus at 0.25, 0.5, 1, 2, 4 and 8 hr (IP and PO). Plasma samples were separated by centrifugation of whole blood and stored below −70° C. until bioanalysis. Immediately after collection of blood from intraperitoneal and oral group animals, animals were euthanized with excess CO2 and brain samples were collected from set of two mice at each time point. Brain samples were divided into two parts. Half brain samples were homogenized using ice-cold phosphate buffer saline (pH-7.4) and homogenates were stored below −70±10° C. until analysis. Total homogenate volume was three times the brain weight. Other part of brain was stored below −70±10° C. for further analysis. All samples were processed for analysis by protein precipitation using acetonitrile and analyzed with fit-for-purpose LC/MS/MS method (LLOQ-2.02 ng/mL for plasma and 6.06 ng/g for brain). Pharmacokinetic parameters were calculated using the non-compartmental analysis tool of Phoenix WinNonlin®.

Protocol B: Thirty six mice were included into the study and divided in to three groups as Group 1 (n=12), Group 2 (n=12) and Group 3 (n=12) with n=2 mice per time-point design. Animals in Group 1 were administered intravenously with solution formulation of PSY-05-00414-001 at 5 mg/kg dose. Animals in Group 2 were administered orally with solution formulation of PSY-05-00414-001 at a dose of 5 mg/kg. Animals in Group 3 were administered intraperitoneally with solution formulation of PSY-05-00414-001 at 5 mg/kg dose. The formulation vehicle for all the three groups was 5% NMP, 5% Solutol HS-15 and 90% normal saline. Blood samples (approximately 60 μL) were collected under light isoflurane anesthesia from two mice at 0.25, 0.5, 1, 2, 4 and 8 h. Plasma was harvested by centrifugation of blood and stored at −70±10° C. until analysis. After blood collection, brain was perfused and isolated at 0.25, 0.5, 1, 2, 4 and 8 h. Brain samples were dipped thrice in ice-cold phosphate buffer saline, blotted dry and cut in to two equal portion. Half-brain samples from each time-point were weighed and homogenized using ice-cold phosphate buffer saline with twice volume of brain weight making the total homogenate three volumes and stored below −70±10° C. until analysis. Remaining half-portions of brain samples was snap freeze and kept in −70±10° C. until further confirmation from client. Plasma and brain samples were quantified by fit-for-purpose LC-MS/MS method (LLOQ: 1.01 ng/mL for plasma and 2.02 ng/ml for brain).

Protocol C: Fifty four male mice were included in study and divided in to three groups as Group 1 (n=18), Group 2 (n=18) and Group 3 (n=18) with 3 mice/time point design. Animals from Group 1, Group 2 and Group 3 were administered by intravenous, oral and intraperitoneal route with solution formulation of PSY-05-00451-001 at 5 mg/kg dose, respectively. The formulation vehicle used was 5% v/v NMP, 5% v/v Solutol HS-15 and 90% w/v Normal saline. Blood samples (approximately 60 μL) were collected under light isoflurane anesthesia (Surgivet®) from retro orbital plexus from a set of three mice at 0.25, 0.5, 1, 2, 4 and 8 h. Immediately after blood collection, plasma was harvested by centrifugation at 4000 rpm, 10 min at 4° C. and samples were stored at −70±10° C. until bioanalysis. Following blood collection, animals were sacrificed followed by vena-cava was cut open and whole body was perfused from heart using 10 mL of normal saline. Brain samples were collected from set of three mice at 0.25, 0.5, 1, 2, 4 and 8 h. After isolation, brain samples were rinsed three times in ice cold normal saline (for 5-10 seconds/rinsed using ˜5-10 mL normal saline in disposable petri dish for each rinse), dried on blotting paper and cut in to two equal portion. Half-brain was used for PK estimation and half brain was snap freezed and stored below −70±10° C. Half brain (for PK estimation) was weighed and homogenized using ice-cold phosphate buffer saline with twice volume of brain weight making the total homogenate three volumes and stored below −70±10° C. until analysis. All samples were processed for analysis by protein precipitation method and analyzed with fit-for-purpose LC-MS/MS method (LLOQ=2.03 ng/mL for plasma and 1.02 ng/mL for brain). The pharmacokinetic parameters were estimated using non-compartmental analysis tool of Phoenix® WinNonlin software.

Protocol D: Thirty six male mice were included in study and divided into two groups as Group 1 (n=18) and Group 2 (n=18) with 3 mice/time point as sparse design. Animals from Group 1 and Group 2 were administered by intravenous and oral route with solution formulation of PSY-05-00473-001 at 5 mg/kg dose, respectively. The formulation vehicle used was 5% v/v NMP, 5% v/v Solutol HS-15 and 90% v/v Normal saline. Blood samples (approximately 60 μL) were collected under light isoflurane anesthesia (Surgivet®) from retro orbital plexus from a set of three mice at 0.25, 0.5, 1, 2, 4 and 8 h. Immediately after blood collection, plasma was harvested by centrifugation at 4000 rpm, 10 min at 4° C. and samples were stored at −70±10° C. until bioanalysis. Following blood collection, whole body was perfused using 10 mL of normal saline. Brain samples were collected from set of three mice at 0.25, 0.5, 1, 2, 4 and 8 h. After isolation, brain samples were rinsed three times in ice cold normal saline (for 5-10 seconds/rinsed using ˜5-10 mL normal saline in disposable petri dish for each rinse), dried on blotting paper and cut in to two equal portion. Half-brain was used for PK estimation and half brain was snap freezed and stored below −70±10° C. Half brain (for PK estimation) was weighed and homogenized using ice-cold phosphate buffer saline with twice volume of brain weight making the total homogenate to three volumes and stored below −70±10° C. until analysis. All samples were processed for analysis by protein precipitation method and analyzed with fit-for-purpose LC-MS/MS method (LLOQ)=1.01 ng/mL for plasma and 2.01 for brain). The pharmacokinetic parameters were estimated using non-compartmental analysis tool of Phoenix® WinNonlin software.

Protocol E: Total fifty four male mice were included in study and divided in to three groups as Group 1 (n=18), Group 2 (n=18) and Group 3 (n=18) with 3 mice/time point design. Animals from Group 1, Group 2 and Group 3 were administered by intravenous, oral and intraperitoneal route with solution formulation of PSY-05-00476-001 at 5 mg/kg dose, respectively. The formulation vehicle used was 5% v/v NMP, 5% v/v Solutol HS-15 and 90% v/v Normal saline. Blood samples (approximately 60 μL) were collected under light isoflurane anesthesia (Surgivet®) from retro orbital plexus from a set of three mice at 0.25, 0.5, 1, 2, 4 and 8 h. Immediately after blood collection, plasma was harvested by centrifugation at 4000 rpm, 10 min at 4° C. and samples were stored at −70±10° C. until bioanalysis. Following blood collection, animals were sacrificed followed by vena-cava was cut open and whole body was perfused from heart using 10 mL of normal saline. Brain samples were collected from set of three mice at 0.25, 0.5, 1, 2, 4 and 8 h. After isolation, brain samples were rinsed three times in ice cold normal saline (for 5-10 seconds/rinsed using ˜5-10 mL normal saline in disposable petri dish for each rinse), dried on blotting paper and cut in to two equal portion. Half-brain was used for PK estimation and half brain was snap freezed and stored below −70±10° C. Half brain (for PK estimation) was weighed and homogenized using ice-cold phosphate buffer saline with twice volume of brain weight making the total homogenate three volumes and stored below −70±10° C. until analysis. All samples were processed for analysis by protein precipitation method and analyzed with fit-for-purpose LC-MS/MS method (LLOQ=1.01 ng/mL for plasma and brain). The pharmacokinetic parameters were estimated using non-compartmental analysis tool of Phoenix® WinNonlin software.

TABLE F
Data obtained from Pharmacokinetic study of Example 25

| | | | | | | | Brain |
|---|---|---|---|---|---|---|---|
| | | | | Dose | Cₘₐₓ | AUCₗₐₛₜ | KP |
| Protocol | Compound | Matrix | Route | (mg/kg) | (ng/g) | (hr*ng/mL) | (Cₘₐₓ) |
| A | Compound 74 | Brain | Oral | 30 | 101.76 | 116.52 | .42 |
| B | Compound 414 | Brain | Oral | 5 | 135.83 | 774.91 | 1.19 |
| C | Compound 451 | Brain | Oral | 5 | 271.39 | 1204.22 | .32 |
| D | Compound 473 | Brain | Oral | 5 | 171.59 | 464.42 | 1.74 |
| E | Compound 476 | Brain | Oral | 5 | 72 | 223.64 | .72 |

Referring to the data in Table F, the Cₘₐₓ is the peak concentration of a drug observed over time after a dose of the drug has been administered. Data presented in the table above demonstrate the ability of PSY compounds to enter the brain. AUCₗₐₛₜ is the area under the brain concentration-time curve to the last measured brain concentration. This reflects the total drug exposure over time after a dose has been administered. Brain Kₚ (Cₘₐₓ) is the ratio of the maximum observed concentrations of drug in the brain and plasma. Higher Kₚ (Cₘₐₓ) values indicate more delivery of the drug to the brain.

### Example 21: Synthesis of (6-(5-(2-chloro-5-fluorophenyl)-4-methyl-1H-pyrazol-1-yl)-2-azaspiro [3.3]heptan-2-yl) (2-fluoro-5-hydroxyphenyl) methanone [Compound 519], and (6-(3-(2-chloro-5-fluorophenyl)-4-methyl-1H-pyrazol-1-yl)-2-azaspiro[3.3]heptan-2-yl) (2-fluoro-5-hydroxyphenyl) methanone [Compound 520]

### Synthetic Scheme:

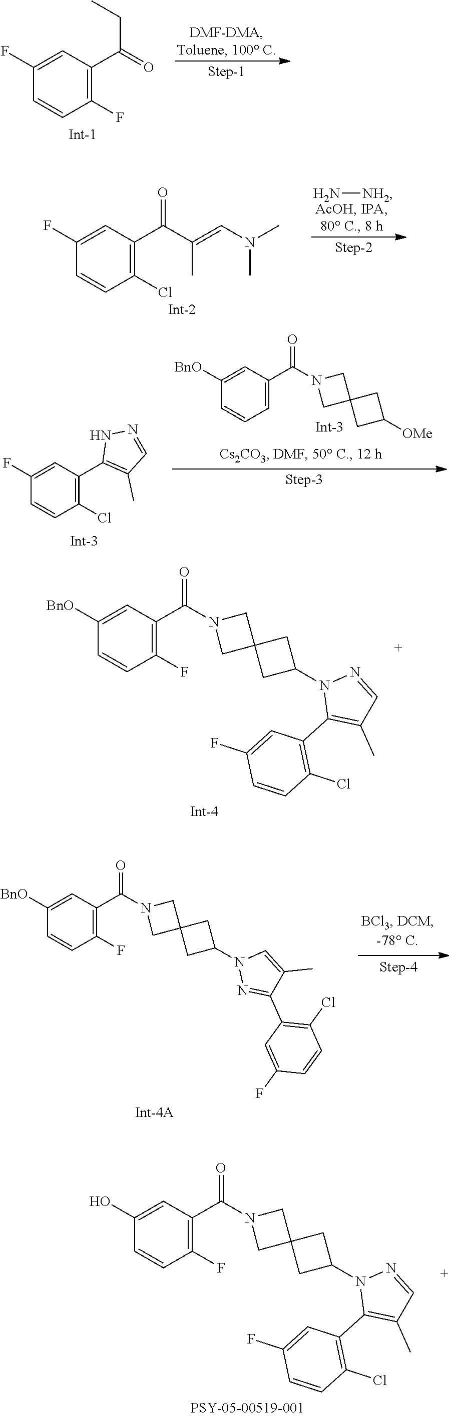

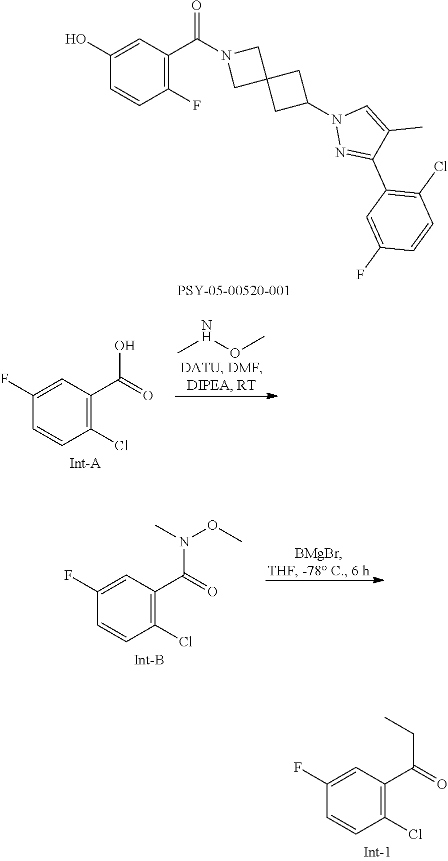

### Step-A: 2-chloro-5-fluoro-N-methoxy-N-methylbenzamide (Int-B)

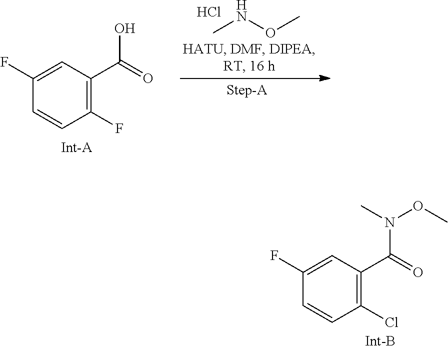

To a stirred solution of 2-chloro-5-fluorobenzoic acid (10.0 g, 57.27 mmol, 1.0 eq.), N,N-di-isopropyl ethylamine (29.4 mL, 171.82 mmol, 3.0 eq.), N,O-dimethyl hydroxylamine hydrochloride (6.142 g, 63.0 mmol, 1.1 eq.) in N,N-Dimethylformamide (50 mL) was added HATU (32.66 g, 85.91 mmol, 1.5 eq.) at 0° C. Stir the reaction at room temperature for 16 h. After completion of reaction as monitored by TLC, the reaction mixture was poured in ice cold water (100 mL) and extracted with ethyl acetate (50 mL*3). The organic layer was dried over Sodium sulfate and concentrated under reduce pressure to get residue. The residue was purified by combiflash using 25% Ethyl acetate in n-hexane as eluent to get 2-chloro-5-fluoro-N-methoxy-N-methylbenzamide (Int-B) (10.9 g, 87.3%).

### Step-B: 1-(2-chloro-5-fluorophenyl) propan-1-one (Int-1)

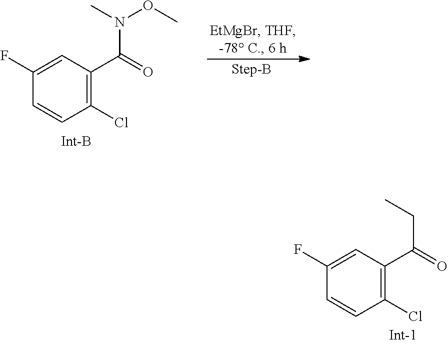

To a stirred solution of 2-chloro-5-fluoro-N-methoxy-N-methylbenzamide (10.9 g, 50.25 mmol, 1.0 eq.) in tetrahydrofuran (50 mL), was slowly added Ethyl magnesium bromide 3M solⁿ in Tetrahydrofuran (25.13 mL, 75.38 mmol, 1.5 eq.) at −78° C. Stir the reaction at room temperature for 6 h. After completion of reaction as monitored by TLC, the reaction mixture was poured in ice cold water (100 mL) and extracted with ethyl acetate (50 mL*3). The organic layer was dried over Sodium sulfate and concentrated under reduce pressure to get residue. The residue was purified by combiflash using 20-25% Ethyl acetate in n-hexane as eluent to get 1-(2-chloro-5-fluorophenyl) propan-1-one (Int-1) (5.28 g, 56.49%).

### Step-1: (E)-1-(2-chloro-5-fluorophenyl)-3-(dimethylamino)-2-methylprop-2-en-1-one (Int-2)

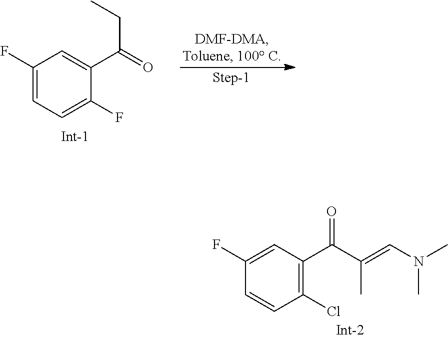 [SMILES: CCC(=O)c1cc(F)ccc1F ; C/C(=C\N(C)C)C(=O)c1cc(F)ccc1Cl]

To a stirred solution of 1-(2-chloro-5-fluorophenyl) propan-1-one (5.28 g, 28.29 mmol, 1.0 eq.) in Toluene (15 mL) was added N,N-Dimethylformamide dimethyl acetal (23.60 g, 198.06 mmol, 7.0 eq.) the reaction mass was heated at 100° C. for 16 h. After completion of reaction as monitored by TLC, the reaction mixture was poured in ice cold water (100 mL) and extracted with ethyl acetate (50 mL*3). The organic layer was washed with brine (100 mL), dried over Sodium sulfate and concentrated under reduce pressure to get residue. The residue was purified by combiflash using 25-30% ethyl acetate in hexane as eluent to get (E)-1-(2-chloro-5-fluorophenyl)-3-(dimethylamino)-2-methylprop-2-en-1-one (Int-2) (5.65 g, 82.62%).

### Step-2:5-(2-chloro-5-fluorophenyl)-4-methyl-1H-pyrazole (Int-3)

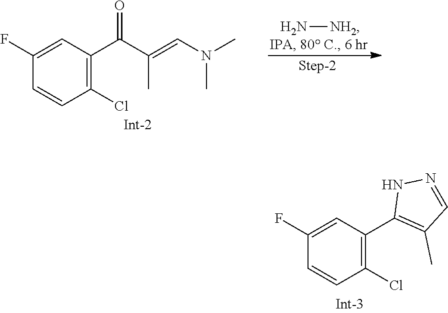

To a stirred solution of (E)-1-(2-chloro-5-fluorophenyl)-3-(dimethylamino)-2-methylprop-2-en-1-one (5.65 g, 23.44 mmol, 1.0 eq.) in Isopropyl alcohol (30 mL) was added hydrazine hydrate (2.0 g, 35.16 mmol, 1.5 eq.) the reaction mass was heated at 80° C. for 16 h. After completion of reaction as monitored by TLC, the reaction mixture was concentrated to get residue. The residue was purified by combiflash using 15-20% ethyl acetate in hexane as eluent to get 5-(2-chloro-S-fluorophenyl)-4-methyl-1H-pyrazole (Int-3) (4.3 g, 87.33%).

### Step-3: (5-(benzyloxy)-2-fluorophenyl) (6-(5-(2-chloro-5-fluorophenyl)-4-methyl-1H-pyrazol-1-yl)-2-azaspiro [3.3] heptan-2-yl) methanone (Int-4) and (5-(benzyloxy)-2-fluorophenyl) (6-(3-(2-chloro-5-fluorophenyl)-4-methyl-1H-pyrazol-1-yl)-2-azaspiro [3.3] heptan-2-yl) methanone (Int-4A)

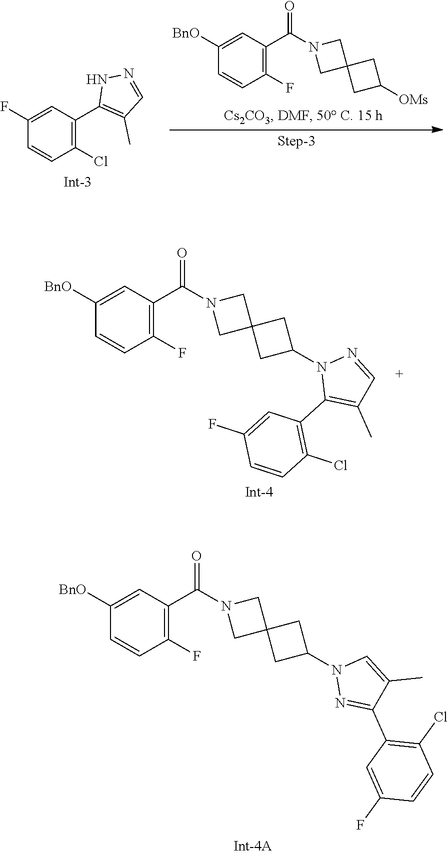 [SMILES: Cc1cn[nH]c1-c1cc(F)ccc1Cl ; CS(=O)(=O)OC1CC2(C1)CN(C(=O)c1cc(OCc3ccccc3)ccc1F)C2 ; Cc1cnn(C2CC3(C2)CN(C(=O)c2cc(OCc4ccccc4)ccc2F)C3)c1-c1cc(F)ccc1Cl ; Cc1cn(C2CC3(C2)CN(C(=O)c2cc(OCc4ccccc4)ccc2F)C3)nc1-c1cc(F)ccc1Cl]

To a well stirred reaction mixture of 5-(2-chloro-5-fluorophenyl)-4-methyl-1H-pyrazole (0.2 g, 0.094 mmol, 1.0 eq.), Cesium carbonate (0.46 g, 1.42 mmol, 1.5 eq.) in N,N-Dimethylformamide (5 mL) was added 2-(5-(benzyloxy)-2-fluorobenzoyl)-2-azaspiro[3.3]heptan-6-yl methane sulfonate (0.43 g, 1.04 mmol, 1.3 eq.). The reaction was heated at 50° C. for 12 h. After completion of reaction as monitored by TLC, the reaction mixture was poured in water (30 mL) and extracted with ethyl acetate (50 mL). The organic layer was washed with brine (30 mL), dried over Sodium sulfate and concentrated under reduce pressure to get residue. The residue was purified by combiflash using 35-40% ethyl acetate in n-hexane as eluent to get mixture of two regio isomers (5-(benzyloxy)-2-fluorophenyl) (6-(5-(2-chloro-5-fluorophenyl)-4-methyl-1H-pyrazol-1-yl)-2-azaspiro[3.3]heptan-2-yl) methanone (Int-4) and (5-(benzyloxy)-2-fluorophenyl) (6-(3-(2-chloro-5-fluorophenyl)-4-methyl-1H-pyrazol-1-yl)-2-azaspiro[3.3]heptan-2-yl) methanone (Int-4A) (0.9 g, 97.63%).

### Step-4: (6-(5-(2-chloro-5-fluorophenyl)-4-methyl-1H-pyrazol-1-yl)-2-azaspiro [3.3] heptan-2-yl) (2-fluoro-5-hydroxyphenyl) methanone (PSY-05-00519-001) and (6-(3-(2-chloro-5-fluorophenyl)-4-methyl-1H-pyrazol-1-yl)-2-azaspiro [3.3]heptan-2-yl) (2-fluoro-5-hydroxyphenyl) methanone (PSY-05-00520-001)

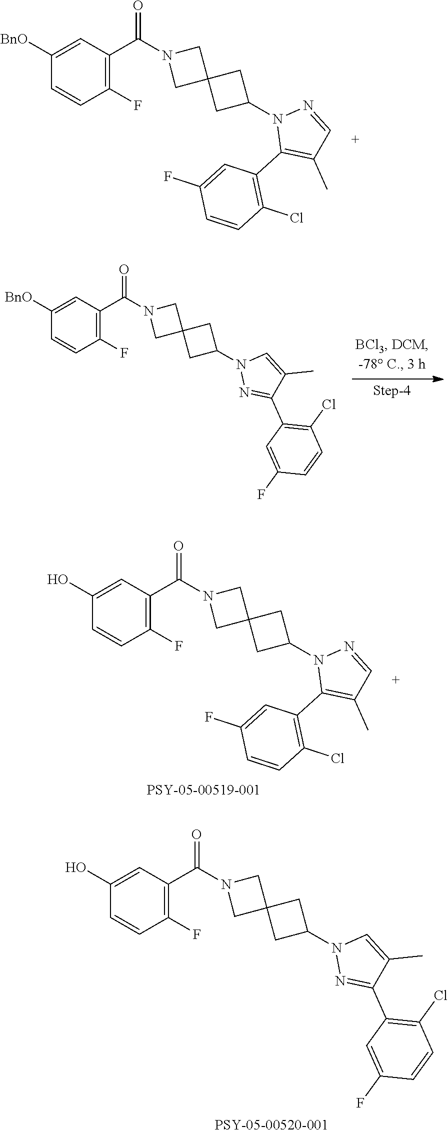 [SMILES: Cc1cnn(C2CC3(C2)CN(C(=O)c2cc(OCc4ccccc4)ccc2F)C3)c1-c1cc(F)ccc1Cl ; Cc1cn(C2CC3(C2)CN(C(=O)c2cc(OCc4ccccc4)ccc2F)C3)nc1-c1cc(F)ccc1Cl ; Cc1cnn(C2CC3(C2)CN(C(=O)c2cc(O)ccc2F)C3)c1-c1cc(F)ccc1Cl ; Cc1cn(C2CC3(C2)CN(C(=O)c2cc(O)ccc2F)C3)nc1-c1cc(F)ccc1Cl]

To a stirred solution of (5-(benzyloxy)-2-fluorophenyl) (6-(5-(2-chloro-5-fluorophenyl)-4-methyl-1H-pyrazol-1-yl)-2-azaspiro[3.3]heptan-2-yl) methanone and (5-(benzyloxy)-2-fluorophenyl) (6-(3-(2-chloro-5-fluorophenyl)-4-methyl-1H-pyrazol-1-yl)-2-azaspiro[3.3]heptan-2-yl) methanone (0.5 g, 0.93 mmol, 1.0 eq.) in dichloromethane (5 mL) was cooled to −78° C., was added BCl₃ 1.0 M Solⁿ in dichloromethane (2.8 mL, 2.80 mmol, 3.0 eq.), then the resulting reaction mixture was stirred at −78° C. for next 2-3 h. The progress of the reaction was monitored by TLC. After completion of reaction, the reaction mixture was quench with Triethylamine (2 mL) and concentrated under vacuum to get crude. The crude was purified by prep-HPLC to get two fractions.

Fraction-1: (6-(5-(2-chloro-5-fluorophenyl)-4-methyl-1H-pyrazol-1-yl)-2-azaspiro [3.3]heptan-2-yl) (2-fluoro-5-hydroxyphenyl) methanone (PSY-05-00519-001) (0.062 g, 15.01%). LCMS: m/z 444.40 [M+1]⁺. NMR: ¹H NMR (400 MHZ, DMSO-d6) δ 9.70 (s, 1H), 7.68 (d, J=5.0 Hz, 1H), 7.58 (dt, J=8.8, 5.6 Hz, 2H), 7.35-7.17 (m, 1H), 7.08 (q, J=9.1 Hz, 1H), 6.85-6.71 (m, 2H), 4.32 (dq, J=24.0, 7.9 Hz, 1H), 4.03 (d, J=10.7 Hz, 4H), 2.68 (q, J=15.2, 13.3 Hz, 4H), 1.81 (d, J=2.6 Hz, 3H).

Fraction-2: (6-(3-(2-chloro-5-fluorophenyl)-4-methyl-1H-pyrazol-1-yl)-2-azaspiro [3.3]heptan-2-yl) (2-fluoro-5-hydroxyphenyl) methanone (PSY-05-00520-001) (0.019 g, 4.76%). LCMS: m/z 444.40 [M+1]⁺. NMR: ¹H NMR (400 MHZ, DMSO-d6) δ 9.80 (s, 1H), 7.68 (d, J=5.0 Hz, 1H), 7.58 (dt, J=8.8, 5.6 Hz, 1H), 7.35-7.17 (m, 2H), 7.08 (q, J=9.1 Hz, 1H), 6.87-6.74 (m, 2H), 4.72 (dq, J=24.0, 7.9 Hz, 1H), 4.13 (d, J=10.7 Hz, 2H), 4.02 (d, J=9.4 Hz, 2H), 2.68 (q, J=15.2, 13.3 Hz, 4H), 1.91 (d, J=2.6 Hz, 3H).

TABLE 3
Compounds, including the value of MAGL inhibition potency measured with a method described in Table C of Example 18.

| | | | MAGL |
|---|---|---|---|
| | | | Potency |
| | | | (Detection |
| Compound | Structure (IUPAC name) | LCMS/NMR | Method) |
| 74 | 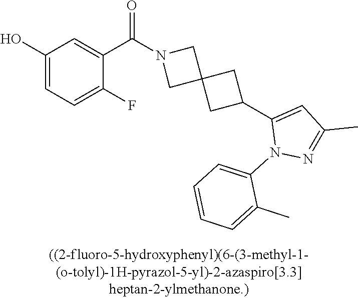 | LCMS: 251.3 m/z [M + H]+; 1H NMR (400 MHz, DMSO- d6) δ: 7.50 (m, 4H), 5.81 (s, 1H), 2.29 (s, 3H), 2.13 (s, 3H). | A (1) A (2) A (3) B (4) A (5) |
| 120 | 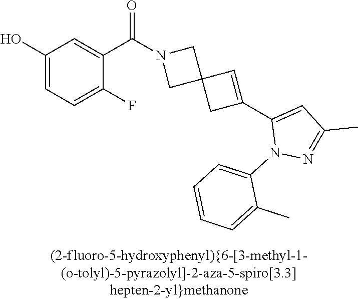 | LCMS: 473.7 m/z [M + H]+; ¹H NMR (400 MHz, Methanol-d₄) δ 7.51- 7.33 (m, 3H), 7.28 (d, J = 7.9 Hz, 1H), 7.00 (t, J = 9.2 Hz, 1H), 6.92-6.78 (m, 2H), 6.35 (s, 1H), 5.39 (s, 1H), 4.29- 4.09 (m, 4H), 2.78 (d, J = 5.6 Hz, 2H), 2.30 (s, 3H), 2.00 (s, 3H). | C (2) |
| 125 | 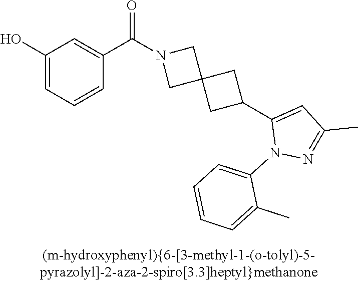 [SMILES: Cc1cc(C2CC3(C2)CN(C(=O)c2cccc(O)c2)C3)n(-c2ccccc2C)n1] | LCMS: 388.4 m/z [M + H]⁺; ¹H NMR (400 MHz, DMSO- d₆) δ 9.63 (d, J = 13.1 Hz, 1H), 7.44-7.26 (m, 3H), 7.17 (dd, J = 19.2, 8.3 Hz, 2H), 6.96 (d, J = 7.6 Hz, 2H), 6.86 (s, 1H), 6.14 (d, J = 12.2 Hz, 1H), 4.19 (d, J = 25.2 Hz, 2H), 3.94 (d, J = 23.0 Hz, 2H), 3.03- 2.94 (m, 1H), 2.33 (s, 3H), 2.27-2.12 (m, 5H), 1.93 (s, 3H) | B (1,2) |
| 126 | 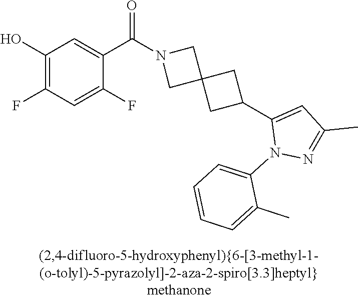 [SMILES: Cc1cc(C2CC3(C2)CN(C(=O)c2cc(O)c(F)cc2F)C3)n(-c2ccccc2C)n1] | LCMS: 424.4 m/z [M + H]⁺, ¹H NMR (400 MHz, Methanol-d₄) δ 7.34 (dt, J =25.4, 8.9 Hz, 3H), 7.18 (t, J = 6.3 Hz, 1H), 7.07-6.91 (m, 2H), 6.18 (d, J = 18.4 Hz, 1H), 4.05 (dd, J = 22.8, 7.5 Hz, 4H), 3.13-3.02 (m, 1H), 2.36 (td, J = 16.6, 14.9, 9.8 Hz, 4H), 2.26 (d, J = 8.4 Hz, 3H), 1.98 (d, J = 2.3 Hz, 3H). | A (1, 2, 3) |
| 127 | 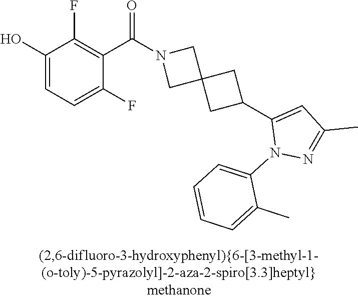 [SMILES: Cc1cc(C2CC3(C2)CN(C(=O)c2c(F)ccc(O)c2F)C3)n(-c2ccccc2C)n1] | LCMS: 424.7 m/z [M + H]⁺; ¹H NMR (400 MHz, Methanol-d₄) δ 7.50- 7.25 (m, 3H), 7.18 (dd, J = 7.6, 5.5 Hz, 1H), 6.91-6.81 (m, 1H), 6.18 (d, J = 21.0 Hz, 1H), 4.10 (d, J = 22.2 Hz, 2H), 3.96 (d, J = 23.3 Hz, 2H), 3.15-3.02 (m, 1H), 2.49-2.29 (m, 4H), 2.26 (d, J = 10.5 Hz, 3H), 1.98 (d, J = 2.8 Hz, 3H). | E (2) |
| 128 | 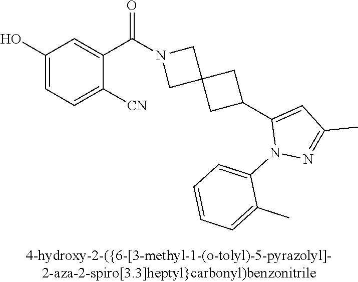 [SMILES: Cc1cc(C2CC3(C2)CN(C(=O)c2cc(O)ccc2C#N)C3)n(-c2ccccc2C)n1] | LCMS: 413.4 m/z [M + H]⁺; ¹H NMR (400 MHz, DMSO- d₆) δ 7.68 (t, J = 7.8 Hz, 1H), 7.43-7.26 (m, 3H), 7.15 (dd, J = 7.8, 3.9 Hz, 1H), 6.98-6.85 (m, 2H), 6.13 (d, J = 19.6 Hz, 1H), 3.99 (dd, J = 24.0, 13.9 Hz, 4H), 2.99 (dt, J = 22.2, 8.4 Hz, 1H), 2.39- 2.28 (m, 2H), 2.23 (dd, J = 12.7, 9.0 Hz, 2H), 2.17 (d, J = 8.5 Hz, 3H), 1.92 (s, 3H). | A (1, 2) |
| 140 | 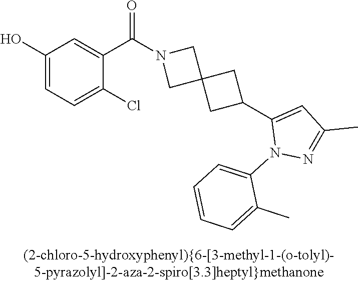 [SMILES: Cc1cc(C2CC3(C2)CN(C(=O)c2cc(O)ccc2Cl)C3)n(-c2ccccc2C)n1] | LCMS: m/z 422.6 [M + 1]⁺; NMR: ¹H NMR (400 MHz, DMSO-d₆) δ 9.95 (s, 1H), 7.40-7.11 (m, 4H), 7.15 (t, J = 7.7 Hz, 1H), 6.80 (t, 1H), 6.67 (d, J = 2.8 Hz, 1H), 6.16-6.10 (s, 1H), 3.97-3.58 (m, 4H), 3.00-2.93 (m, 1H), 2.33-2.15 (m, 7H), 1.91 (s, 3H). | B (2) |
| 141 | 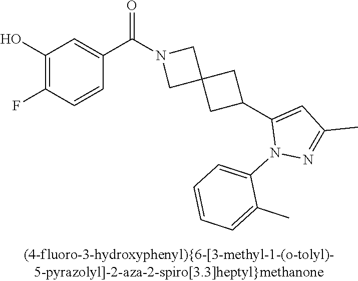 [SMILES: Cc1cc(C2CC3(C2)CN(C(=O)c2ccc(F)c(O)c2)C3)n(-c2ccccc2C)n1] | LCMS: 406.3 m/z [M + H]+; ¹H NMR (400 MHz, DMSO- d₆) δ 10.16 (s, 1H), 7.38 (s, 2H), 7.30 (d, J = 5.9 Hz, 1H), 7.18 (dd, J = 13.5, 8.4 Hz, 3H), 7.02 (s, 1H), 6.15 (d, J = 10.7 Hz, 1H), 4.25 (s, 1H), 4.19 (s, 1H), 3.97 (s, 1H), 3.92 (s, 1H), 3.04-2.94 (m, 1H), 2.23 (d, J = 9.5 Hz, 2H), 2.19 (s, 5H), 1.93 (s, 3H). | B (1) C (2) |
| 185 | 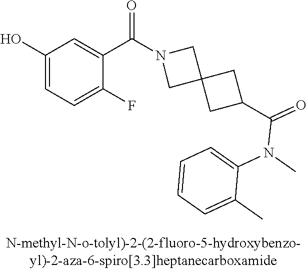 [SMILES: Cc1ccccc1N(C)C(=O)C1CC2(C1)CN(C(=O)c1cc(O)ccc1F)C2] | LCMS: m/z 383.35 [M + 1]⁺ NMR: ¹H NMR (400 MHz, DMSO-d₆) δ 9.62 (d, J = 13.5 Hz, 1H), 7.40-7.23 (m, 3H), 7.16 (s, 1H), 7.17- 6.98 (m, 2H), 6.85- 6.81 (m, 1H), 6.78- 6.69 (m, 1H), 3.97- 3.80 (m, 4H), 3.05 (d, J = 6.1 Hz, 3H), 2.62 (td, J = 15.6, 7.7 Hz, 1H), 2.39- 2.23 (m, 2H), 2.22- 2.05 (m, 3H), 2.00- | B (2, 3) |
| | | 1.90 (m, 2H). | |
| 365 | 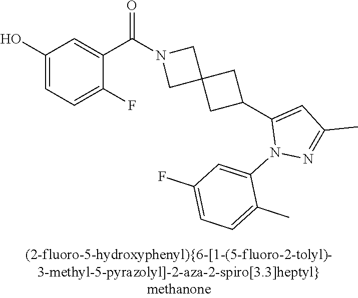 [SMILES: Cc1cc(C2CC3(C2)CN(C(=O)c2cc(O)ccc2F)C3)n(-c2cc(F)ccc2C)n1] | LCMS: 424.1 m/z [M + H]⁺ ¹H NMR (400 MHz, DMSO- d₆) δ 9.64 (s, 1H), 7.66 (s, 1H), 7.20 (s, 2H), 7.06 (s, 1H) 6.83 (t, 1H), 6.75 (s, 1H), 6.28 (s, 1H), 3.99 (d, 2H), 3.82 (d, J = 11.5 Hz, 2H), 3.11 (m, 1H), 2.45 (d, 2H), 2.35 (d, 3H), 2.19 (s, 2H), 1.87 (s, 3H). | A (14) |
| 366 | 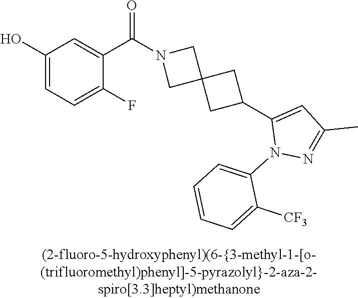 [SMILES: Cc1cc(C2CC3(C2)CN(C(=O)c2cc(O)ccc2F)C3)n(-c2ccccc2C(F)(F)F)n1] | LCMS: 460.0 [M + H]⁺, NMR: ¹H NMR (400 MHz, DMSO- d₆) δ 9.63 (s, 1H), 7.80 (s, 1H), 7.77- 7.67 (m, 3H), 7.08 (t, 1H), 7.82 (d, 1H), 6.76 (s, 1H), 6.28 (d, J = 15.4 Hz, 1H), 3.98 (d, 2H), 3.87 (d, J = 9.0 Hz, 2H), 2.96 (m, J = 8.7 Hz, 1H), 2.25 (d, J = 15.6 Hz, 6H) | A (14) |
| 367 | 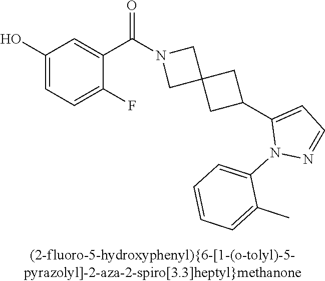 | m/z 422.6 [M + 1]⁺; ¹H NMR (400 MHz, DMSO-d₆) δ 9.95 (s, 1H), 7.43-7.1 (m, 4H), 7.15 (t, J = 7.7 Hz, 1H), 6.80 (t, 1H), 6.67 (d, J = 2.8 Hz, 1H), 6.16-6.10 (s, 1H), 3.98 (s, 1H), 3.92 (s, 1H), 3.82 (s, 1H), 3.76 (s, 1H), 2.99 (dt, J = 20.8, 8.4 Hz, 1H), 2.32-2.19 (m, 2H), 2.17 (d, J = 10.7 Hz, 2H), 2.09 (s, 3H), 1.93 (d. J = 2.2 Hz, 3H). | B (14) |
| 370 | 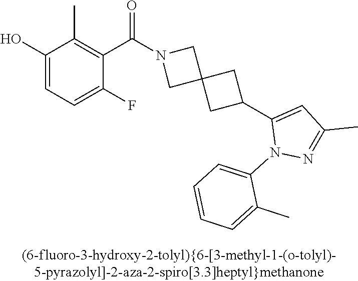 [SMILES: Cc1cc(C2CC3(C2)CN(C(=O)c2c(F)ccc(O)c2C)C3)n(-c2ccccc2C)n1] | LCMS: 420.6 m/z [M + H]⁺ NMR: ¹H NMR (400 MHz, DMSO-d₆) δ 9.54 (d, J = 10.5 Hz, 1H), 7.44-7.35 (m, 2H), 7.37-7.27 (m, 1H), 7.16 (t, J = 8.1 Hz, 1H), 6.84-6.71 (ddd, J = 36.2, 15.5, 8.7 Hz, 2H), 6.12 (d, 1H), 4.01 (s, 2H), 3.95 (s, 2H), 3.05- 2.94 (m, 1H), 2.37 (d, J = 12.5 Hz, 4H), 2.33-2.14 (s, 3H), 2.02-1.91 (s, 6H). | D (14) |
| 371 | 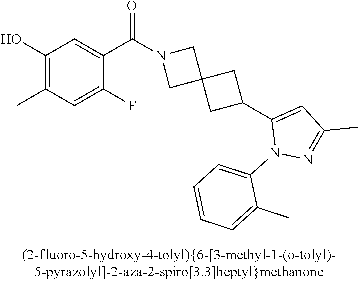 [SMILES: Cc1cc(C2CC3(C2)CN(C(=O)c2cc(O)c(C)cc2F)C3)n(-c2ccccc2C)n1] | LCMS: 420.23 m/z [M + H]⁺¹ ¹H NMR (400 MHz, DMSO- d₆) δ 9.56 (d, J = 9.0 Hz, 1H), 7.39 (d, J = 7.9 Hz, 2H), 7.31 (d, J = 7.9 Hz, 1H), 7.16 (s, J = 7.9 Hz, 1H), 6.99 (t, J = 10.2 Hz, 1H), 6.78 (d, 1H), 6.15 (d, J = 19.5 Hz, 1H), 3.97 (s, 2H), 3.92 (s, 2H), 3.04- 2.95 (m, 1H), 2.35 (s, 2H), 2.26-2.15 (m, 4H), 2.13 (d, J = 4.9 Hz, 3H), 2.20 (d, | B (14) |
| | | 3H), 1.94 (s, 3H). | |
| 373 | 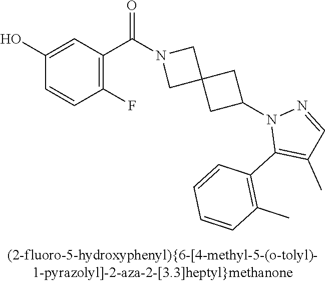 [SMILES: Cc1ccccc1-c1c(C)cnn1C1CC2(C1)CN(C(=O)c1cc(O)ccc1F)C2] | LCMS: m/z 406.2 [M + 1]⁺, NMR: ¹H NMR (400 MHz, DMSO-d₆) δ 9.71 (s, 1H), 7.67 (s, 1H), 7.22-7.37 (m, 4H), 7.10 (m, 1H), 6.90- 6.75 (m, 2H), 4.71 (m, 1H), 4.14 (d, J = 5.8 Hz, 2H), 4.04 (m, 2H), 2.64 (m, 4H), 2.20 (m, 3H), 1.9 (s, 3H). | C (14) |
| 376 | 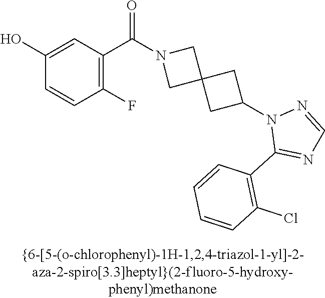 [SMILES: O=C(c1cc(O)ccc1F)N1CC2(CC(n3ncnc3-c3ccccc3Cl)C2)C1] | LCMS: m/z 413.27 [M + 1]⁺. ¹H NMR (400 MHz, DMSO- d₆) δ 9.76 (s, 1H), 8.16-8.14 (d, J = 10.0 Hz, 1H), 7.80- 7.70 (m, 2H), 7.52 (m, 1H), 7.08-7.02 (m, 1H), 6.93 (m, 1H), 6.84(s, 1H), 4.58-4.51 (m, 1H), 4.09 (s, 4H), 2.71 (m, 4H). | B (14) |
| 377 | 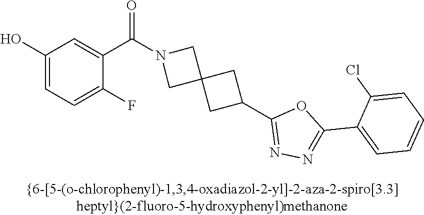 [SMILES: O=C(c1cc(O)ccc1F)N1CC2(CC(c3nnc(-c4ccccc4Cl)o3)C2)C1] | LCMS: 414.3 m/z [M + H]⁺¹ NMR; ¹H NMR (400 MHz, DMSO-d₆) δ 9.69 (s, 1H), 7.98-7.93 (m, 1H), 7.62 (m, 3H), 7.02 (td, J = 9.2, 2.9 Hz, 1H), 6.92-6.79 (m, 2H), 4.31 (d, J = 5.9 Hz, 2H), 4.07 (d, J = 11.5 Hz, 2H), 3.97 (m, J = 10.4 Hz, 1H), 3.41-3.71 | D (14) |
| | | (q, J = 6.6 Hz, 4H). | |
| 378 | 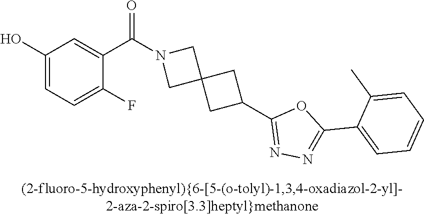 [SMILES: Cc1ccccc1-c1nnc(C2CC3(C2)CN(C(=O)c2cc(O)ccc2F)C3)o1] | LCMS: 394.91 m/z [M + H]⁺¹ NMR: ¹H NMR (400 MHz, DMSO-d₆) δ 9.69 (s, 1H), 7.85 (dd, J = 18.0, 6.8 Hz, 1H), 7.52-7.39 (m, 3H), 7.10 (d, 1H) 6.88 (s, 1H), 6.81 (s, 1H), 4.15 (d, J = 5.1 Hz, 2H), 4.08 (s, 2H), 3.76 (m, 1H) 2.74 (d, J = 9.1 Hz, 1H), 2.62 | D (14) |
| | | (d, J = 10.9 Hz, 3H), | |
| | | 2.44 (s, 3H). | |
| 380 | 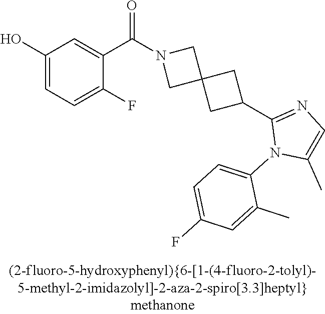 [SMILES: Cc1cc(F)ccc1-n1c(C)cnc1C1CC2(C1)CN(C(=O)c1cc(O)ccc1F)C2] | LCMS: m/z 424.31 [M + 1]⁺ ¹H NMR (400 MHz, DMSO- d₆) δ 9.72 (s, 1H), 7.36-7.29 (m, 3H), 7.07 (q, J = 10.2, 9.8 Hz, 1H), 6.85 (d, J = 6.1 Hz, 1H), 6.79- 6.69 (m, 2H), 4.00- 3.90 (m, 4H), 2.91- 2.85 (m, 1H), 2.38 (d, J = 7.8 Hz, 2H), 2.34-2.24 (m, 2H), 1.89-1.80 (m, 6H). | E (14) |
| 381 | 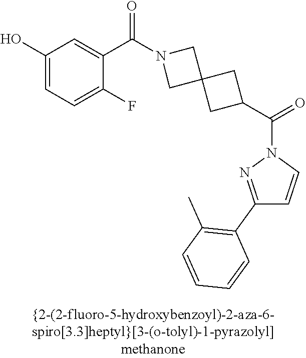 [SMILES: Cc1ccccc1-c1ccn(C(=O)C2CC3(C2)CN(C(=O)c2cc(O)ccc2F)C3)n1] | LCMS: m/z 419.89 [M + 1]+ 1H NMR (400 MHz, DMSO- d6) δ 9.67 (s, 1H), 8.47 (dd, J = 10.0, 2.8 Hz, 1H), 7.64 (d, J = 7.3 Hz, 1H), 7.33 (dd, J = 11.7, 6.1 Hz, 3H), 7.04-7.01 (m, 1H), 7.01-6.94 (m, 1H), 6.90-6.77 (m, 2H), 4.14 (d, J = 6.2 Hz, 3H), 3.99 (d, J = 8.0 Hz, 2H), 2.64- 2.2.54 (m, 7H). | C (14) |
| 401 | 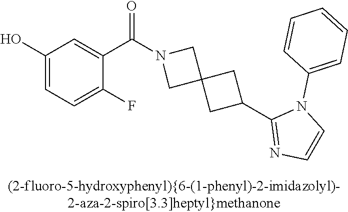 [SMILES: O=C(c1cc(O)ccc1F)N1CC2(CC(c3nccn3-c3ccccc3)C2)C1] | LCMS: m/z 419.89 [M + 1]⁺ ¹H NMR (400 MHz, DMSO- d₆) δ 9.62 (s, 1H), 7.55-7.44 (m, 3H), 7.33 (s, 2H), 7.27 (s, 1H), 7.09-7.03 (m, 1H), 6.96 (d. J = 10.4 Hz, 1H), 6.84 (s, 1H), 6.76 (s, 1H), 4.02 (s, 2H), 3.95 (s, 2H), 2.45 (s, 4H). | D (14) |
| 414 | 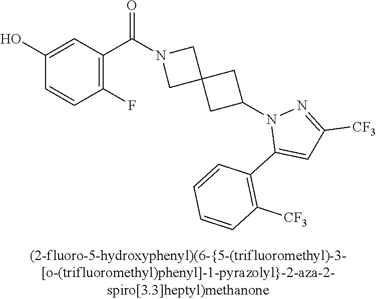 [SMILES: O=C(c1cc(O)ccc1F)N1CC2(CC(n3nc(C(F)(F)F)cc3-c3ccccc3C(F)(F)F)C2)C1] | LCMS: m/z 514.20 [M+1]⁺. NMR: ¹NMR (400 MH_{Z}, DMSO-d₆) δ 9.67 (d, 1H), 7.95 (t, 1H), 7.85-7.77 (m, 3H), 7.06 (s, 2H), 6.84 (s, 1H), 6.75 (s, 1H), 4.92 (m, 1H), 4.22 (m, 2H), 4.03 (s, 2H), 2.68 (s, 4H). | A (14) |
| 473 | 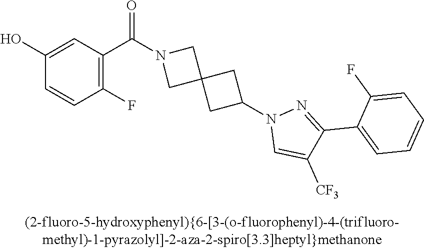 [SMILES: O=C(c1cc(O)ccc1F)N1CC2(CC(n3cc(C(F)(F)F)c(-c4ccccc4F)n3)C2)C1] | LCMS: m/z 464.01 [M + H]⁺, ¹H NMR (400 MHz, DMSO- d₆) δ 9.68 (s, 1H), 8.12 (d, J = 9.7 Hz, 1H), 7.63-7.43 (dq, J = 19.3, 9.9, 7.4 Hz, 4H), 7.08 (q, J = 9.5 Hz, 1H), 6.85 (s, 1H), 6.77 (s, 1H), 4.50 (dt, J = 14.3, 7.5 Hz, 1H), 4.03 (d, J = 9.0 Hz, 4H), 2.79-2.63 (m, 4H). | A (12) |
| 476 | 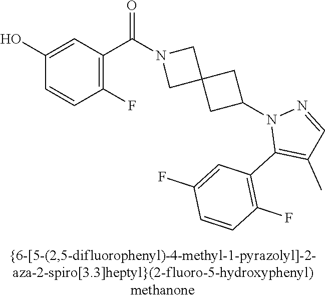 [SMILES: Cc1cnn(C2CC3(C2)CN(C(=O)c2cc(O)ccc2F)C3)c1-c1cc(F)ccc1F] | LCMS: m/z 428.3 [M + 1]⁺. ¹H NMR (400 MHz, DMSO- d₆) δ 9.69 (s, 1H), 7.72 (d. J = 5.9 Hz, 1H), 7.39-7.26 (m, 3H), 7.10 (q, J = 9.1 Hz, 1H), 6.82-6.87 (q, J = 9.1 Hz, 1H), 6.74-6.79 (q, J = 9.1 Hz, 1H), 4.76 (dt, J = 23.3, 7.9 Hz, 1H), 4.15 (d. J = 11.2 Hz, 2H), 4.05 (d, J = 8.7 Hz, 2H), 2.76-2.60 (m, 4H), 1.99 (s, 3H). | B (13) |
| 519 | 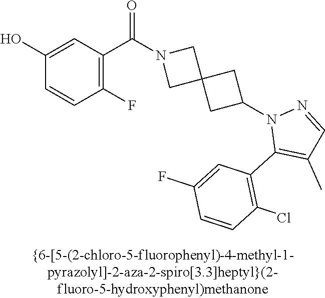 [SMILES: Cc1cnn(C2CC3(C2)CN(C(=O)c2cc(O)ccc2F)C3)c1-c1cc(F)ccc1Cl] | LCMS: m/z 444.40 [M + 1]⁺. NMR: ¹H NMR (400 MHz, DMSO-d₆) δ 9.80 (s, 1H), 7.68 (d, J = 5.0 Hz, 1H), 7.58 (dt, J = 8.8, 5.6 Hz, 1H), 7.35-7.17 (m, 2H), 7.08 (q, J = 9.1 Hz, 1H), 6.87-6.74 (m, 2H), 4.72 (dq, J = 24.0, 7.9 Hz, 1H), 4.13 (d, J = 10.7 Hz, 2H), 4.02 (d, J = 9.4 Hz, 2H), 2.68 (q, J = 15.2, 13.3 Hz, 4H), 1.91 (d, J = 2.6 Hz, 3H). | B (12) |
| 520 | 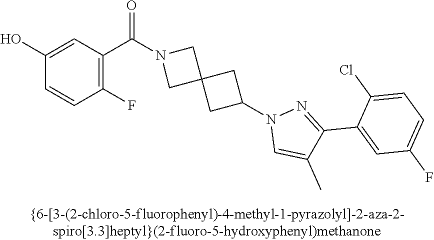 [SMILES: Cc1cn(C2CC3(C2)CN(C(=O)c2cc(O)ccc2F)C3)nc1-c1cc(F)ccc1Cl] | LCMS: m/z 444.40 [M + 1]⁺. NMR: ¹H NMR (400 MHz, DMSO-d₆) δ 9.70 (s, 1H), 7.68 (d, J = 5.0 Hz, 1H), 7.58 (dt, J = 8.8, 5.6 Hz, 2H), 7.35-7.17 (m, 1H), 7.08 (q, J = 9.1 Hz, 1H), 6.85-6.71 (m, 2H), 4.32 (dq, J = 24.0, 7.9 Hz, 1H), 4.03 (d, J = 10.7 Hz, 4H), 2.68 (q, J = 15.2, 13.3 Hz, 4H), | A (12) |
| | | 1.81 (d, J = 2.6 Hz, | |
| | | 3H). | |

TABLE 4
Compounds, including the value of MAGL inhibition potency measured with a method described in
Table C of Example 18.

| | | | MAGL |
|---|---|---|---|
| | | | Potency |
| | Structure | | (Detection |
| Compound | (IUPAC name) | LCMS/NMR | Method) |
| 142 | 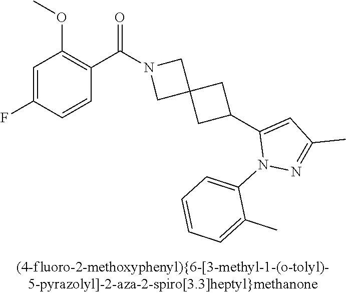 [SMILES: COc1cc(F)ccc1C(=O)N1CC2(CC(c3cc(C)nn3-c3ccccc3C)C2)C1] | LCMS: 420.41 m/z [M + H]+; 1H NMR (400 MHz, DMSO- d6) δ 7.44-7.25 (m, 4H), 7.16 (t, J = 6.5 Hz, 1H), 6.98 (s, 1H), 6.85-6.74 (m, 1H), 6.17 (s, 1H), 3.95 (s, 1H), 3.89 (s, 1H), 3.86-3.75 (m, 5H), 2.99 (dt, J = 25.9, 8.5 Hz, 1H), 2.26-2.43 (m, 3H), 2.26-2.14 (m, 4H), 1.94 (d, J = 2.2 Hz, 3H). | D (2) |
| 143 | 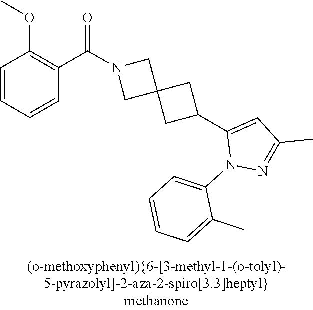 [SMILES: COc1ccccc1C(=O)N1CC2(CC(c3cc(C)nn3-c3ccccc3C)C2)C1] | LCMS: 401.8 m/z [M]+. 1H NMR (400 MHz, Methanol-d4) δ 7.50-7.17 (m, 5H), 7.17-6.96 (m, 3H), 6.23-6.17 (s, 1H), 4.11 (s, 1H), 4.05 (s, 1H), 3.97 (s, 1H), 3.93-3.84 (m, 4H), 3.08 (dq, J = 29.0, 8.5 Hz, 1H), 2.48- 2.35 (m, 3H), 2.32 (dd, J = 27.0, 12.1 Hz, 4H), 2.01 (d, J = 3.1 Hz, 3H). | E (2) |
| 145 | 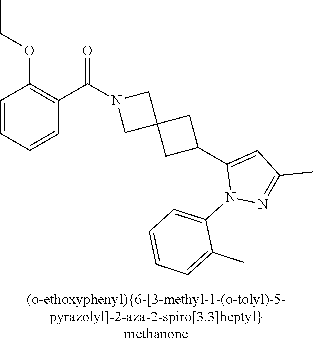 [SMILES: CCOc1ccccc1C(=O)N1CC2(CC(c3cc(C)nn3-c3ccccc3C)C2)C1] | LCMS: 416.74 m/z [M + H]+; 1H NMR (400 MHz, DMSO- d6) δ 7.45-7.24 (m, 3H), 7.20 (d, J = 7.5 Hz, 1H), 7.14 (q, J = 8.1, 7.2 Hz, 1H), 7.08-6.89 (m, 2H), 6.17-6.07 (s, 1H), 4.06 (dq, J = 9.9, 6.9 Hz, 2H), 3.94 (s, 1H), 3.88 (s, 1H), 3.83 (s, 1H), 3.77 (s, 1H), 2.98 (dq, J = 25.0, 8.4 Hz, 1H), 2.39-2.14 (m, 7H), 1.93 (d, J = 1.9 Hz, 3H), 1.30 (dt, J = 9.8, 6.9 Hz, 3H). | E (2) |
| 177 | 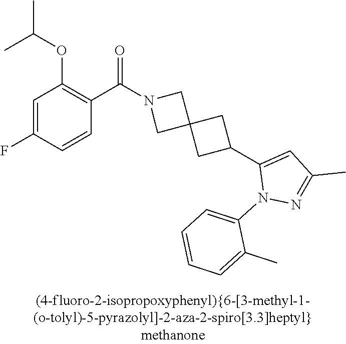 [SMILES: Cc1cc(C2CC3(C2)CN(C(=O)c2ccc(F)cc2OC(C)C)C3)n(-c2ccccc2C)n1] | LCMS: 448.5 m/z [M + H]⁺. ¹H NMR (400 MHz, DMSO- d₆) δ 7.38 (t, J = 8.3 Hz, 2H), 7.33-7.22 (m, 2H), 7.15 (dd, J = 7.9, 4.2 Hz, 1H), 6.97 (dd, J = 11.6, 8.5 Hz, 1H), 6.75 (q, J = 7.6 Hz, 1H), 6.13 (d, J = 17.6 Hz, 1H). 4.70-4.60 (m, 1H), 3.93 (s, 1H), 3.85 (d, J = 15.1 Hz, 2H), 3.78 (s, 1H), 3.00 (dt, J = 18.8, 8.5 Hz, 1H), 2.34-2.16 (m, 4H), 2.16 (s, 3H), 1.93 (s, 3H), 1.24 (dd, J = 11.5, 6.1 Hz, | B (2) |
| | | 6H). | |
| 178 | 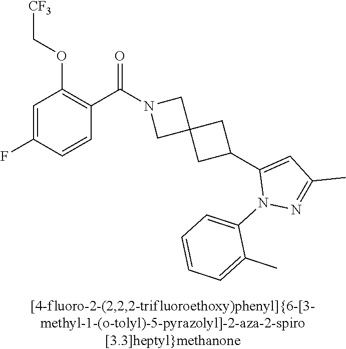 [SMILES: Cc1cc(C2CC3(C2)CN(C(=O)c2ccc(F)cc2OCC(F)(F)F)C3)n(-c2ccccc2C)n1] | LCMS: 488.5 m/z [M+] 1H NMR (400 MHz, DMSO-d6) δ 7.46-7.36 (m, 2H), 7.40-7.25 (m, 2H), 7.16 (s, 1H), 7.21- 7.10 (m, 1H), 6.98- 6.87 (m, 1H), 6.17 (s, 1H), 4.93-4.79 (m, 2H), 3.95 (s, 1H), 3.86 (d, J = 19.9 Hz, 2H), 3.78 (s, 1H), 3.03-2.92 (m, 1H), 2.33 (t, J = 10.4 Hz, 2H), 2.21 (dd, J = 23.9, 9.2 Hz, 5H), 1.93 (d, J = 2.3 Hz, 3H). | A (2, 3, 5) |

TABLE 5
Compounds

| Comparator | Structure | Potency |
|---|---|---|
| 123 | 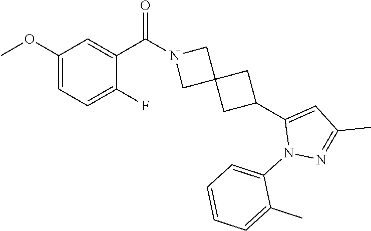 | >10,000 nM (2) |
| 144 | 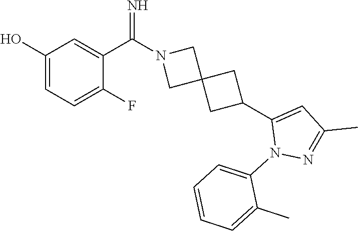 [SMILES: Cc1cc(C2CC3(C2)CN(C(=N)c2cc(O)ccc2F)C3)n(-c2ccccc2C)n1] | >10,000 nM (2) |
| 149 | 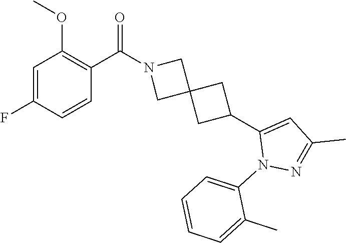 [SMILES: COc1cc(F)ccc1C(=O)N1CC2(CC(c3cc(C)nn3-c3ccccc3C)C2)C1] | >10,000 nM (2) |
| 150 | 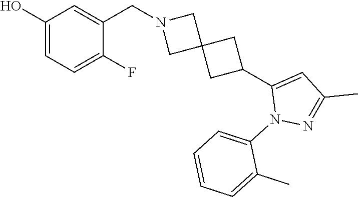 [SMILES: Cc1cc(C2CC3(C2)CN(Cc2cc(O)ccc2F)C3)n(-c2ccccc2C)n1] | >10,000 nM |
| 152, 153 | 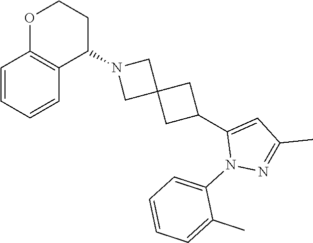 | >10,000 nM (2) |
| | 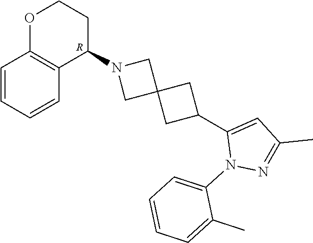 | |
| 154, 155 | 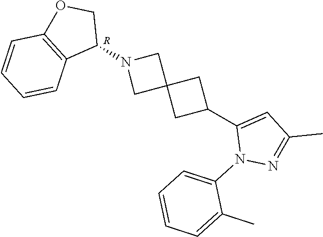 | >10,000 nM (2) |
| | 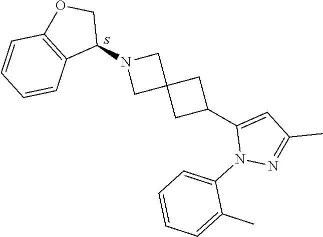 | |
| 179 | 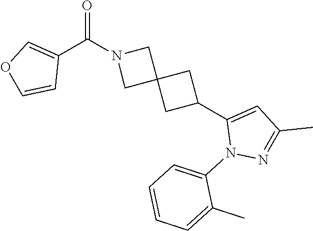 [SMILES: Cc1cc(C2CC3(C2)CN(C(=O)c2ccoc2)C3)n(-c2ccccc2C)n1] | >10,000 nM (2) |
| 181 | 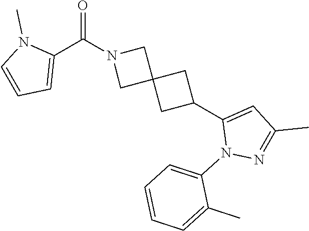 [SMILES: Cc1cc(C2CC3(C2)CN(C(=O)c2cccn2C)C3)n(-c2ccccc2C)n1] | >10,000 nM (2) |
| 182 | 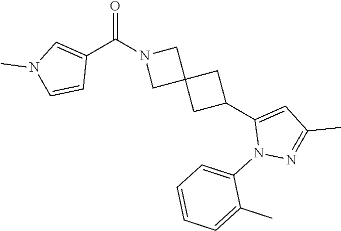 | >10,000 nM (2) |
| 183 | 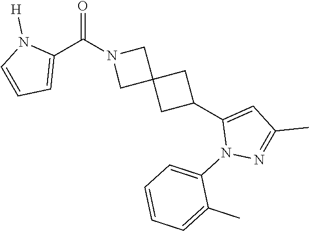 [SMILES: Cc1cc(C2CC3(C2)CN(C(=O)c2ccc[nH]2)C3)n(-c2ccccc2C)n1] | >10,000 nM (2) |
| 184 | 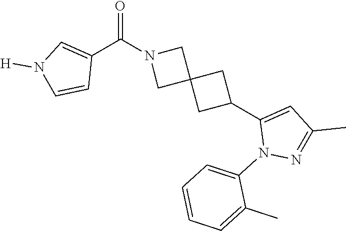 [SMILES: Cc1cc(C2CC3(C2)CN(C(=O)c2cc[nH]c2)C3)n(-c2ccccc2C)n1] | >10,000 nM (2) |
| 188 | 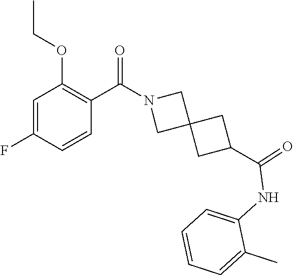 [SMILES: CCOc1cc(F)ccc1C(=O)N1CC2(CC(C(=O)Nc3ccccc3C)C2)C1] | >10,000 nM (2) |
| 198 | 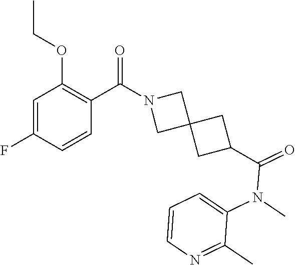 [SMILES: CCOc1cc(F)ccc1C(=O)N1CC2(CC(C(=O)N(C)c3cccnc3C)C2)C1] | >10,000 nM (4) |
| 368 | 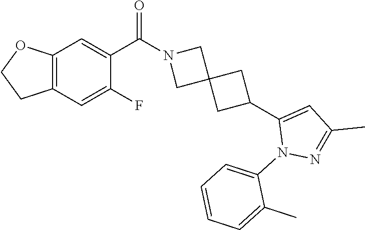 [SMILES: Cc1cc(C2CC3(C2)CN(C(=O)c2cc4c(cc2F)CCO4)C3)n(-c2ccccc2C)n1] | >10,000 nM (14) |
| 384 | 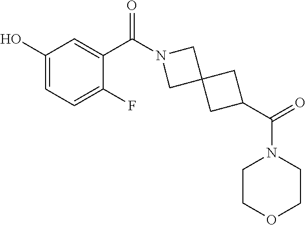 [SMILES: O=C(c1cc(O)ccc1F)N1CC2(CC(C(=O)N3CCOCC3)C2)C1] | >10,000 nM (14) |
| 388 | 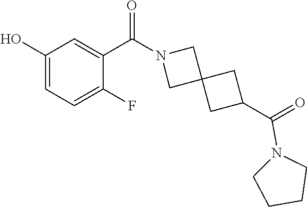 [SMILES: O=C(c1cc(O)ccc1F)N1CC2(CC(C(=O)N3CCCC3)C2)C1] | >10,000 nM (14) |
| 391 | 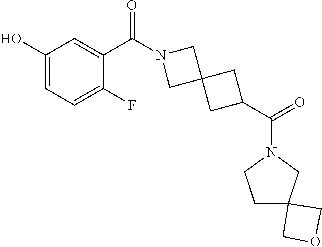 [SMILES: O=C(c1cc(O)ccc1F)N1CC2(CC(C(=O)N3CCC4(COC4)C3)C2)C1] | >10,000 nM (14) |
| 395 | 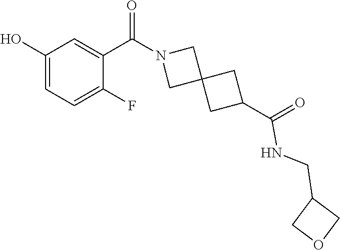 [SMILES: O=C(NCC1COC1)C1CC2(C1)CN(C(=O)c1cc(O)ccc1F)C2] | >10,000 nM (14) |
| 400 | 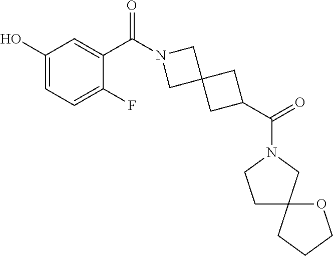 [SMILES: O=C(c1cc(O)ccc1F)N1CC2(CC(C(=O)N3CCC4(CCCO4)C3)C2)C1] | >10,000 nM (14) |
| 402 | 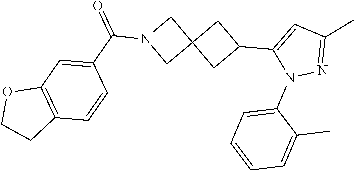 [SMILES: Cc1cc(C2CC3(C2)CN(C(=O)c2ccc4c(c2)OCC4)C3)n(-c2ccccc2C)n1] | >10,000 nM (14) |
| 403 | 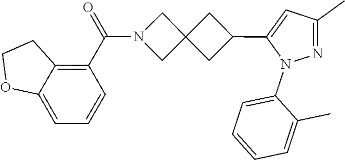 [SMILES: Cc1cc(C2CC3(C2)CN(C(=O)c2cccc4c2CCO4)C3)n(-c2ccccc2C)n1] | >10,000 nM (14) |
| 404 | 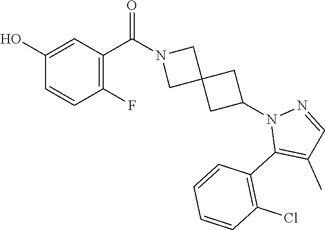 [SMILES: Cc1cnn(C2CC3(C2)CN(C(=O)c2cc(O)ccc2F)C3)c1-c1ccccc1Cl] | >10,000 nM (6) |
| 405 | 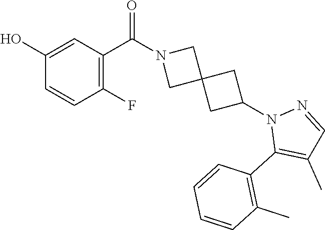 [SMILES: Cc1ccccc1-c1c(C)cnn1C1CC2(C1)CN(C(=O)c1cc(O)ccc1F)C2] | >10,000 nM (6) |
| 406 | 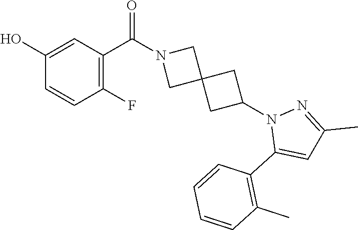 [SMILES: Cc1cc(-c2ccccc2C)n(C2CC3(C2)CN(C(=O)c2cc(O)ccc2F)C3)n1] | >10,000 nM (6) |
| 416 | 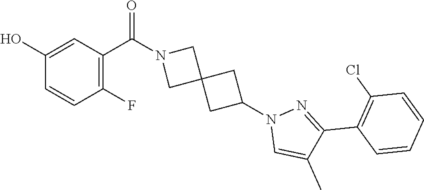 [SMILES: Cc1cn(C2CC3(C2)CN(C(=O)c2cc(O)ccc2F)C3)nc1-c1ccccc1Cl] | >10,000 nM (6) |
| 424 | 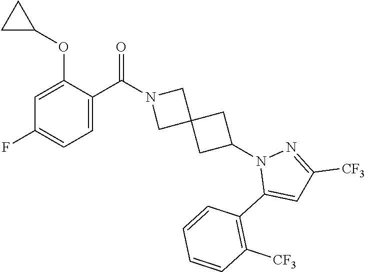 [SMILES: O=C(c1ccc(F)cc1OC1CC1)N1CC2(CC(n3nc(C(F)(F)F)cc3-c3ccccc3C(F)(F)F)C2)C1] | >10,000 nM (6) |
| 427 | 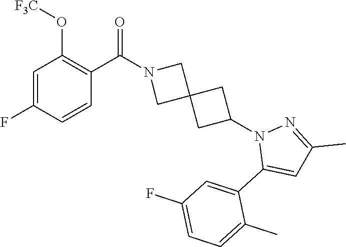 [SMILES: Cc1cc(-c2cc(F)ccc2C)n(C2CC3(C2)CN(C(=O)c2ccc(F)cc2OC(F)(F)F)C3)n1] | >10,000 nM (11) |
| 441 | 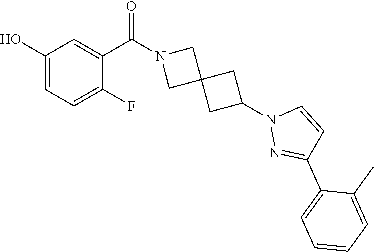 [SMILES: Cc1ccccc1-c1ccn(C2CC3(C2)CN(C(=O)c2cc(O)ccc2F)C3)n1] | >10,000 nM (6) |
| 442 | 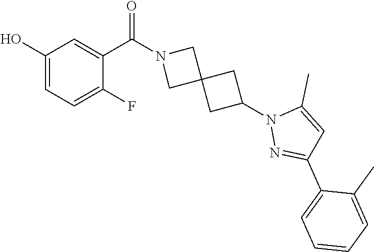 [SMILES: Cc1ccccc1-c1cc(C)n(C2CC3(C2)CN(C(=O)c2cc(O)ccc2F)C3)n1] | >10,000 nM (6) |
| 445 | 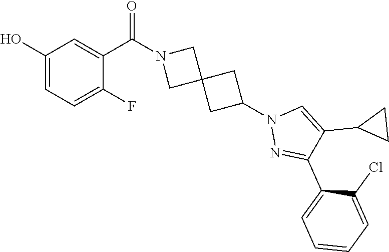 | >10,000 nM (6) |
| 494 | 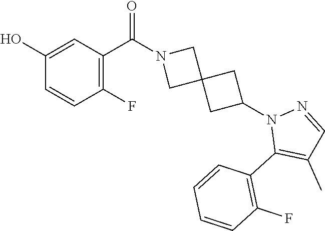 [SMILES: Cc1cnn(C2CC3(C2)CN(C(=O)c2cc(O)ccc2F)C3)c1-c1ccccc1F] | >10,000 nM (12) |
| 495 | 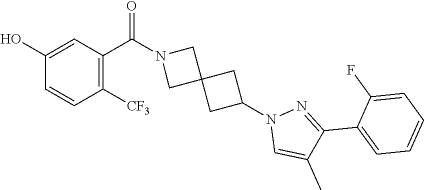 [SMILES: Cc1cn(C2CC3(C2)CN(C(=O)c2cc(O)ccc2C(F)(F)F)C3)nc1-c1ccccc1F] | >10,000 nM (12) |
| 521 | 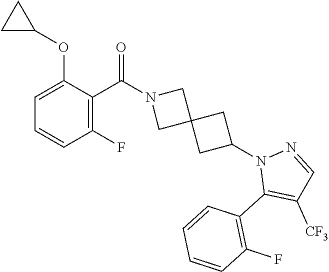 [SMILES: O=C(c1c(F)cccc1OC1CC1)N1CC2(CC(n3ncc(C(F)(F)F)c3-c3ccccc3F)C2)C1] | >10,000 nM (12) |

1.-35. (canceled)

36. A method of treating an anxiety disorder in a subject in need thereof, the method comprising administering to the subject a therapeutically effective amount of a compound selected from the group consisting of:
a. (2,4-difluoro-5-hydroxyphenyl) {6-[3-methyl-1-(o-tolyl)-5-pyrazolyl]-2-aza-2-spiro[3.3]heptyl}methanone (Compound 126), or a pharmaceutically acceptable salt thereof;
b. 4-hydroxy-2-({6-[3-methyl-1-(o-tolyl)-5-pyrazolyl]-2-aza-2-spiro[3.3]heptyl}carbonyl)benzonitrile (Compound 128), or a pharmaceutically acceptable salt thereof;
c. [4-fluoro-2-(2,2,2-trifluoroethoxy)phenyl]{6-[3-methyl-1-(o-tolyl)-5-pyrazolyl]-2-aza-2-spiro[3.3]heptyl}methanone (Compound 178), or a pharmaceutically acceptable salt thereof;
d. (2-fluoro-5-hydroxyphenyl) {6-[1-(5-fluoro-2-tolyl)-3-methyl-5-pyrazolyl]-2-aza-2-spiro[3.3]heptyl}methanone (Compound 365), or a pharmaceutically acceptable salt thereof;
e. (2-fluoro-5-hydroxyphenyl) (6-{3-methyl-1-[o-(trifluoromethyl)phenyl]-5-pyrazolyl}-2-aza-2-spiro[3.3]heptyl) methanone (Compound 366), or a pharmaceutically acceptable salt thereof; and
f. (2-ethoxy-4-fluorophenyl) (6-(3-methyl-1-(o-tolyl)-1H-pyrazol-5-yl)-2-azaspiro[3.3]heptan-2-yl) methanone, or a pharmaceutically acceptable salt thereof.

37. The method of claim 36, wherein the anxiety disorder is selected from the group consisting of: Generalized Anxiety Disorder (GAD) or post-traumatic stress disorder (PTSD).

38.-48. (canceled)

49. The method of claim 37, wherein the anxiety disorder is Generalized Anxiety Disorder (GAD).

50.-51. (canceled)

52. The method of claim 37, wherein the anxiety disorder is post-traumatic stress disorder (PTSD).

53.-54. (canceled)

55. A method of treating Generalized Anxiety Disorder (GAD) or post-traumatic stress disorder (PTSD), the method comprising administering to a subject in need thereof a therapeutically effective amount of a reversible monoacylglycerol lipase (MAGL) inhibitor.

56. The method of claim 49, wherein the compound is (2,4-difluoro-5-hydroxyphenyl) {6-[3-methyl-1-(o-tolyl)-5-pyrazolyl]-2-aza-2-spiro[3.3]heptyl}methanone (Compound 126), or a pharmaceutically acceptable salt thereof.

57. The method of claim 49, wherein the compound is 4-hydroxy-2-({6-[3-methyl-1-(o-tolyl)-5-pyrazolyl]-2-aza-2-spiro[3.3]heptyl}carbonyl)benzonitrile (Compound 128), or a pharmaceutically acceptable salt thereof.

58. The method of claim 49, wherein the compound is [4-fluoro-2-(2,2,2-trifluoroethoxy)phenyl]{6-[3-methyl-1-(o-tolyl)-5-pyrazolyl]-2-aza-2-spiro[3.3]heptyl}methanone (Compound 178), or a pharmaceutically acceptable salt thereof.

59. The method of claim 49, wherein the compound is (2-fluoro-5-hydroxyphenyl) {6-[1-(5-fluoro-2-tolyl)-3-methyl-5-pyrazolyl]-2-aza-2-spiro[3.3]heptyl}methanone (Compound 365), or a pharmaceutically acceptable salt thereof.

60. The method of claim 49, wherein the compound is (2-fluoro-5-hydroxyphenyl) (6-{3-methyl-1-[o-(trifluoromethyl)phenyl]-5-pyrazolyl}-2-aza-2-spiro[3.3]heptyl) methanone (Compound 366), or a pharmaceutically acceptable salt thereof.

61. The method of claim 49, wherein the compound is (2-ethoxy-4-fluorophenyl) (6-(3-methyl-1-(o-tolyl)-1H-pyrazol-5-yl)-2-azaspiro[3.3]heptan-2-yl) methanone, or a pharmaceutically acceptable salt thereof.

62. The method of claim 52, wherein the compound is (2,4-difluoro-5-hydroxyphenyl) {6-[3-methyl-1-(o-tolyl)-5-pyrazolyl]-2-aza-2-spiro[3.3]heptyl}methanone (Compound 126), or a pharmaceutically acceptable salt thereof.

63. The method of claim 52, wherein the compound is 4-hydroxy-2-({6-[3-methyl-1-(o-tolyl)-5-pyrazolyl]-2-aza-2-spiro[3.3]heptyl}carbonyl)benzonitrile (Compound 128), or a pharmaceutically acceptable salt thereof.

64. The method of claim 52, wherein the compound is [4-fluoro-2-(2,2,2-trifluoroethoxy)phenyl]{6-[3-methyl-1-(o-tolyl)-5-pyrazolyl]-2-aza-2-spiro[3.3]heptyl}methanone (Compound 178), or a pharmaceutically acceptable salt thereof.

65. The method of claim 52, wherein the compound is (2-fluoro-5-hydroxyphenyl) {6-[1-(5-fluoro-2-tolyl)-3-methyl-5-pyrazolyl]-2-aza-2-spiro[3.3]heptyl}methanone (Compound 365), or a pharmaceutically acceptable salt thereof.

66. The method of claim 52, wherein the compound is (2-fluoro-5-hydroxyphenyl) (6-{3-methyl-1-[o-(trifluoromethyl)phenyl]-5-pyrazolyl}-2-aza-2-spiro[3.3]heptyl) methanone (Compound 366), or a pharmaceutically acceptable salt thereof.

67. The method of claim 52, wherein the compound is (2-ethoxy-4-fluorophenyl) (6-(3-methyl-1-(o-tolyl)-1H-pyrazol-5-yl)-2-azaspiro[3.3]heptan-2-yl) methanone, or a pharmaceutically acceptable salt thereof.
